SEQUENCE LISTING (77 sequences)

SEQ ID NO: 1 - protein, 793 aa, unidentified
MDGVFAVHNVDNLTDHEPIVLQLSLKTDVLQNSEIVHVPRVSWRKASHDQVRNYKNVLSSNLKSIYLPVKTLLCNDIHCSNPIHFEDINRYSISIHDACISAARDTIPVTCRRGSRGRIPGWTERVKPFRERSLFWHNIWVDCGRPRDGHVAAAMRRTRAAYHLQIKLVRKNEDAVVREKVADSVIHDRGRNFWSEIKKIRGTKTRPPGTVDGLTDTPSIAALFADKYRDLYTSVPYDKDDMQRIIDVVDSKLSCTNFSGDCTFYATEVREAIGHMKRGKNDGSNELLSDHLINANDDLIIHIACLFSTFSVHGAVPANFHSSVILPIPKSRNASAASSNNYRGIALSSLLGKILDYIILERYRDTLSSCEYQFGFKRSSSTNLCTMVLKVTLNYYHDNSTNVFCCFLDATKAFDRVNYCKLFRLLLTRDLPPHIIRLLINIYVNNNVCVSWCNVKSDTFRALNGVKQGAVLSPILYCVYVDNLLQLLEKAGVGCHIGFQFLGALAYADDLVLIAPTASAMRTMLSVCDNFARDFDVIFNASKSKWLAVIPSNRSFMSSMINDCLFEVGGNPIDFVDKFVHLGHIISSNLTDTADVESRRSSFIAEVNNMSCFFSKLDVFTRFSLFRSYCTSYYGSELWKLNCPSIDSFYTSWRRALRTVWRLPFRAHSFLLPLISNVLPFRDEICRRSINFMRSCLFHNSYLVRFISYNSIMFGTKSSFLRHNAYYCASRLGCGLDSIIYGNVCVLAEVFSNEMFADAGMLLELLLLRDGVLHCGLNIDEISTLIIHLCTND

SEQ ID NO: 2 - protein, 795 aa, unidentified
MILSIDNTSDHDPIILRLSLDIKYVSVCNRTSSSRVSWVKASDRDIRNYQYNLASNLQHVTIPSVALLCKDVNCSNFAHRCQLSRYLTDISDACLAAGEASIPHTCSRHSGKRIPGWSEKVEPLRQRSLFWHSMWVECGRPRSGVVADCMRRARASYHYAIRAVRRNEQNIIRERVADALLRDPSRDFWTEVKKIRNSKSGRAVIVDGCSDAPSISQLFASKYRHLYTSVPYQHNDLQSIVSDVESRISFDGDCFIGSQEVMAALSKLKLHKNDGDLGLASDYFINSDPALSVHIALLFTGIVIHGFVPSNLLSSTIVPIPKKSNVNATDSDNYRGIALSSVLGKIFDNVILVKYSDKLSTCNLQFGFKRNSSTHMCTMVLKETISYYVNNNSSVFCTFLDASKAFDRVHYCKLFRLLLGRGLPVCILRVLIQLYVGHSIRVTWAGLVSSCFTALNGVKQGGVLSPVLFCIYVDELLIRLAESGVGCYIGFSFAGALAYADDIVLIAPTPSAMRKLLAICDTYATEYNILFNAQKSNFIAFVPSSRHFLHKAMTNCVFRIGGVQIEHVFSYTHLGHIITSRLDDADDILHRRSSYIGQVNNVVCYFDSLSWTVKLGLHKSYCSSIFGCELWALDSVRDIEKFCVAWRKGLRRVLSLPRAAHSHLLPLLSNSLPVYDEICKRSAKFIVSCQCSDNILVRAVVNYAIAARSKSVLGRNVMLLCRRFHLSFDDFVSGRLLLSGDIFVSHYLNSLSEAQLQSVCFALELLCLREQSFKLNNNMRLNADEISDYLAAVLC

SEQ ID NO: 3 - protein, 1034 aa, unidentified
MLLFIRSVMENSNSVLDNLQLTICSYNCRGFNGFKKDYISGLLESAQIDVLLLQEHWLSDAQLNALNNVGANYLNFGVSGFDTSAVLGGRPYGGCAVLWRSDLLLQVQPLVVSSRRLSAVIFSTDNWSLILINVYMPYEGDEIKTDEFIDLLSIIEDLVLSNSASHVIVGGDFNVEFNRNRMHTALLNSFCDNTGLSPVIQHSSCNIDYTYNFNMSRFNILDHFLLSGTLFDVCVTSAYVLHDIDNTSHHDPIILRLSLDIKYVSVCNRTSSSRVSWVKASDRDIRNYQYNLASNLQHVTIPSVALLCKDVNCSNFAHRCQLSRYLTDISDACLAAGEASIPHTCSRHSGKRIPGWSEKVEPLRQRSLFWHSMWVECGRPRSGVVADCMRRARASYHYAIRAVRRNEQNIIRERVADALLRDPSRDFWTEVKKIRNSKSGRAVIVDGCSDAPSISQLFASKYRHLYTSVPYQHNDLQSIVSDVESRISFDGDCFIGSQEVMAALSKLKLHKNDGDLGLASDYFINSDPALSVHIALLFTGIVIHGFVPSNLLSSTIVPIPKKSNVNATDSDNYRGIALSSVLGKIFDNVILVKYSDKLSTCNLQFGFKRNSSTHMCTMVLKETISYYVNNNSSVFCTFLDASKAFDRVHYCKLFRLLLGRGLPVCILRVLIQLYVGHSIRVTWAGLVSSCFTALNGVKQGGVLSPVLFCIYVDELLIRLAESGVGCYIGFSFAGALAYADDIVLIAPTPSAMRKLLAICDTYATEYNILFNAQKSNFIAFVPSSRHFLHKAMTNCVFRIGGVQIEHVFSYTHLGHIITSRLDDADDILHRRSSYIGQVNNVVCYFDSLSWTVKLGLHKSYCSSIFGCELWALDSVRDIEKFCVAWRKGLRRVLSLPRAAHSHLLPLLSNSLPVYDEICKRSAKFIVSCQCSDNILVRAVVNYAIAARSKSVLGRNVMLLCRRFHLSFDDFVSGRLLLSGDIFVSHYLNSLSEAQLQSVCFALELLCLREQSFKLNNNMRLNADEISDYLAAVLC

SEQ ID NO: 4 - protein, 976 aa, unidentified
MVQNTELADCINITLQTNDSRFIVTSYNMHGFNQGLEGTKEMINKLYPDVIALQEHWLTPTNLDRLGEVSNDYFFIGSSSMNDVVTAGPLFGRPFGGTAILINNKLANATVNVACNDKFTAVLISECLVISAYMPCAGTCDRMSLYSAIISEIQALIEFYPQYKLILCADLNVELDAPSSVADLVNNFICRNNLRRTELANPTASRFTYMHESLQAASYIDYIISSDALDSIAFNVLDLDINLSDHLPIMCVFMCKMSACYSDPSQSVKSKSHVDDVSYFRWDHAALHLYYEQTRVMMEPILDKLNICVNSVCDEDAFIDHSKLEDIYSSVVAALTTSANLCIPKIKHNFFKFWWNQELNELKAAAVTSARAWQQVGKPKHGSVYHKYQKDKLLYKKCIRENQKQESVSFTNELHDALLRKTGKDFWKTWNAKVGVKRKCIEQVDGIVDGATIACNFAKHFEKICQPATATFNTTMKAKFEEMRSMYYTPFCDKTVEFDVQLIGKIISNMSCGKAAGLDELSAEHLKHCHPIVIIILCKLFNLFVHVGYLPLSFGTSYTVPIPKQNGRSHALTVNDFRGISISPVISKIFEHAIFARFGDYFSTSDHQFGFKESLSCSHAIYCVRNVIDHYVKRGSTVNICTVDISKAFDTVNHFVLFIKLMERKLPVQLLDLFVLWFSMSETCVRWGAHDSYFFKLKAGVRQGGVLSPYFFAVSVDDVVDKIAACNAGCYINNLCSAIFLYADDIILLSPSVCGLQRLLGICEAAITELDMKINASKTVCTRVGPRFDSACVGPNLTSGGQLNWVKTCRYLGVHFAAGRSFNCSFEEAKKKFYFSFNSVYSKLGRFASEEVILNLLNVKCVSAMLYATEACPVLSRHKHSLDFVITRVFMKILRTGSPQVVAECQKYFGFLPVSYRIDIRSARFLERFSSSVNSFCIAFGDRARKQLSDIVCKYNISDGLSSGVLRTVINRIYFQ

SEQ ID NO: 5 - protein, 970 aa, unidentified
MTITNDAAQLVVISYNLHGLNQGLPGIREFMTELKPDVIMVQEHWLTSDNLNKLSDISDEYFVIGSSAMDARVSAGPLFGRPFGGTAVFIKNKYINVTVNLVTSERYVVIQLCDWLLINVYLPCIGTSNRILLYSDTLCELQSIIRAHPECNCLVGGDFNTDLNDTRSHTANTVVNGFIAGCNLHRCDCLFPTRLKHTYVNDSMNCYSTIDYMLSSNPEKIVAFNVLDIDLNLSDHLPIISICVFDCNCKMNNPKPTSTENVTHFRWDHAPLQDYYEHTRLGLQPILADLDELIGNKLSYSNVDFLCQVDCIYNRVVSVLQQCSHAYVPKHKKNFYKFWWNQELDELKDKAIASCKMWKDAGKPRHGAIHAKYRQDKLLYKKRIRQERVQETSSFTNELHDALLNKSGRDFWKKWNSKFENKSNKILQVGGTSDTATILNNFAKHFEQVCVPFNATRNEELKSRYKEMRLNYSESSVINDTTVFDVQLVENLLTNMKNGKAGGLDELTSEHLKFCHPVVVIILCKLFNLFVINGHIPDSFGVSYTVPIPKSDGRSRSMTVDDFRGISISPVISKLFELCVLDRYSDYLQTSDHQFGFKKQLGCRHAIFSVRSVIEQYISNGSTVNLCALDLSKAFDRMNQYALFIKLMERRFPVKILTILEQWFSIAETCVRWGSEFSYFFSLLAGVRQGGVLSPVLFAIFIDGIVNRVNATNVGCYNSTVCVSIFLYADDILLLSPTVTGLQTLLTVCENELCELDMRLNVNKSVCMRFGARFKAHCANLVSVQGGALQWASSCRYLGVYFVSGRVFRCCFHSAKCNFFRAFNSVYSKIGCCASEEVILSLLKSKCLPYILYGVEACPVLQRDKHSFDFTLTRTLMKLFMTSSPVIVNECQTQFNLLPLRYQIDIRTVKFLNQYIISRNSICMLYKSHAQSVLDTIFGVYGNNVSSLHDLHNIINEHFYDFNPSTLGYN

SEQ ID NO: 6 - protein, 1020 aa, unidentified
MYNLRITSYNCRSLSALKRDFVKTLLVSCDILFLQEHWLSDDQLTLLGGLDEGFTFTGVSGFGNKEILSGRPYGGCAILWRSALKFSVDFLSVNSRRVSAIKLSNDCCNLLLINVYMPFEDSDCNVNEFFDVLFDIEYLLVNNVDCHYLIGGDFNVDLSRNTVHTALLRSFSDNNGLLYASDFDGANFDFTYQFNMSRFSLIDNFMLSSFLFHNMLQRADVVHDVNNLSDHEPVTIELCMSVTHVDIQRSTVPPLQKVSWTTAAESHVIDYREELVDRLKTVVMPIDSLTCCDRRCSVAKHRSDIAEYANNIADVCIKAGLTTIPLMKPGHRATPGFSEHVKPARDKSMFWHQLWLECGRPRTGHVADCMRRTRAAYHYALRSVKRRRDEITQERFATALLHNNSRNFWSEVKKIRANRMTYNGVVDGHSDATDIVRIFGERYRDLYTSVPYDECNMHEINDTIDEHISDDDDAAFVVSLRDVSDAIAHIKHYKNDVDNMLTSDHFINAPSVLHVHISILFSAMLMHGCVPNLLMYSSIRPIPKGHNLSTCDSNNYRGIAISSIFNKIFDNVVLIKYRHLLSTCDLQFGFKKKHSTQMCTMVLKETLSYYLSNRSNVFCTFLDATKAFDRINYCKLFNLLLCRSLPYCIIRVLLSLYTNNYVYVSWVGSNSSSFRACNGVKQGGVLSPVLFCLYMDGLLNKLSHAGVGCYMGEMFVGALAYADDIVLISPTPSAMNNMLSICDEFSIEYNVLFNPLKSKCMYFYPKSRSTLLLHRYNVCDLQFSINGKAIEFVDSYKHLGHVICSDMTDDIDISEKRGVFIGQANNIICYFAKLSSAIKYRLFTSYCTSFFGCELWRISNDSLDSMCTAWRRAIRRIWSLPYTAHGRFLPVLCNSYNIFDQFCVRILNFIRRCLSDQSSLLVRSIATQALMFHHARSPLGYNFIYCARRYCFSLTDFVNYNNFVSNVEKLNCTQNDDDTIANCRLLRELIDLRDGVLHLSDDVFLTNSELSFMINCVSTL

SEQ ID NO: 7 - protein, 1020 aa, unidentified
MYNLRITSYNCRSLSALKRDFVKTLLVSCDILFLQEHWLSDDQLTLLGGLDEGFTFTGVSGFGNKEILSGRPYGGCAILWRSALKFSVDFLSVNSRRVSAIKLSNDCCNLLLINVYMPFEDSDCNVNEFFDVLFDIEYLLVNNVDCHYLIGGDFNVDLSRNTVHTALLRSFSDNNGLLYASDFDGANFDFTYQFNMSRFSLIDNFMLSSFLFHNMLQRADVVHDVNNLSDHEPVTIELCMSVTHVDIQRSTVPPLQKVSWTTAAESHVIDYREELVDRLKTVVMPIDSLTCCDRRCSVARHRSDIAEYANNIADVCIKAGLTTIPLMKPGHRATPGFSEHVKPARDKSMFWHQLWLECGRPRTGHVADCMRRTRAAYHYALRSVKRRRDEITQERFATALLHNNSRNFWSEVKKIRANRMTYNGVVDGHSDATDIVRIFGERYRDLYTSVPYDECNMHEINDTIDEHISDDDDAAFVVSLRDVSDAIAHIKHYKNDVDNMLTSDHFINAPSVLHVHISILFSAMLMHGCVPNLLMYSSIRPIPKGHNLSTCDSNNYRGIAISSIFNKIFDNVVLIKYRHLLSTCDLQFGFKKKHSTQMCTMVLKETLSYYLSNRSNVFCTFLDATKAFDRINYCKLFNLLLCRSLPYCIIRVLLSLYTNNYVYVSWVGSNSSSFRACNGVKQGGVLSPVLFCLYMDGLLNKLSHAGVGCYMGEMFVGALAYADDIVLISPTPSAMNNMLSICDEFSIEYNVLFNPLKSKCMYFYPKSRSTLLLHRYNVCDLQFSINGKAIEFVDSYKHLGHVICSDMTDDIDISEKRGVFIGQANNIICYFAKLSSAIKYRLFTSYCTSFFGCELWRISNDSLDSMCTAWRRAIRRIWSLPYTAHGRFLPVLCNSYNIFDQFCVRILNFIRRCLSDQSSLLVRSIATQALMFHHARSPLGYNFIYCARRYCFSLTDFVNYNNFVSNVEKLNCTQNDDDTIANCRLLRELIDLRDGVLHLSDDVFLTNSELSFMINCVSTL

SEQ ID NO: 8 - protein, 977 aa, unidentified
MVQNTELADCINITLQTNDSRFIVTSYNMHGFNQGLEGTKEMINKLYPDVIALQEHWLTPTNLDRLGEVSNDYFFIGSSSMNDVVTAGPLFGRPFGGTAILINNKLANATVNVACNDKFTAVLISECLVISAYMPCAGTCDRMSLYSAIISEIQALIEFYPQYKLILCADLNVELDAPSSVADLVNNFICRNNLRRTELANPTASRFTYMHESLQAASYIDYIISSDALDSIAFNVLDLDINLSDHLPIMCVFMCKMSACYSDPSQSVKSKSHVDDVSYFRWDRAALHLYYEQTRVMMEPILDKLNICVNSVCDEDAFIDHSKLEDIYSSVVAALTTSANLCIPKIKHNFFKFWWNQELNELKAAAVTSARAWQQVGKPKHGSVYHKYQKDKLLYKKCIRENQKQESVSFTNELHDALLRKTGKDFWKTWNAKVGVKRKCIEQVDGIVDGATIACNFAKHFEKICQPATATFNTTMKAKFEEMRSMYYTPFCDKTVEFDVQLIGKIISNMSCGKAAGLDELSAEHLKHCHPIVIIILCKLFNLFVHVGYLPLSFGTSYTVPIPKQNGRSHALTVNDFRGISISPVISKIFEHAIFARFGDYFSTSDHQFGFKESLSCSHAIYCVRNVIDHYVKRGSTVNICTVDISKAFDTVNHFVLFIKLMERKLPVQLLDLFVLWFSMSETCVRWGAHDSYFFKLKAGVRQGGVLSPYFFAVSVDDVVDKIAACNAGCYINNLCSAIFLYADDIILLSPSVCGLQRLLGICEAAITELDMKINASKTVCTRVGPRFDSACVGPNLTSGGQLNWVKTCRYLGVHFAAGRSFNCSFEEAKKKFYFSFNSVYSKLGRFASEEVILNLLNVKCVSAMLYATEACPVLSRHKHSLDFVITRVFMKILRTGSPQVVAECQKYFGFLPVSYRIDIRSARFLERFSSSVNSFCIAFGDRARKQLSDILCKYNIPDGLSSGVLRTIINRIFFNS

SEQ ID NO: 9 - protein, 969 aa, unidentified
MTITNDAAQLVVISYNLHGLNQGLPGIREFMTELKPDVIMVQEHWLTSDNLNKLSDISDEYFVIGSSAMDARVSAGPLFGRPFGGTAVFIKNKYINVTVNLVTSERYVVIQLCDWLLINVYLPCIGTSNRILLYSDTLCELQSIIRAHPECNCLVGGDFNTDLNDTRSHTANTVVNGFIAGCNLHRCDCLFPTRLKHTYVNDSMNCYSTIDYMLSSNPEKIVAFNVLDIDLNLSDHLPIITVCVFDSNCKLNNPKPTSTEDVTHFRWDHAPLQDYYEHTRLGLQPILADLDELIGNKLYSNVDFLCQVDCIYNRVVSVLQQCSHAYVPKHKKNFYKFWWNQELDELKDKAIASCKMWKDAGKPRHGAIHAKYRQDKLLYKKRIRQERVQETSSFTNELHDALLNKSGRDFWKKWNSKFENKSNKILQVGGTSDTATILNNFAKHFEQVCVPFNATRNEELKSRYKEMRLNYSESSVINDTTVFDVQLVENLLTNMKNGKAGGLDELTSEHLKFCHPVVVIILCKLFNLFVINGHIPDSFGVSYTVPIPKSDGRSRSMTVDDFRGISISPVISKLFELCVLDRYSDYLQTSDHQFGFKKQLGCRHAIFSVRSVIEQYISNGSTVNLCALDLSKAFDRMNQYALFIKLMERRFPVKILTILEQWFSIAETCVRWGSEFSYFFSLLAGVRQGGVLSPVLFAIFIDGIVNRVNATNVGCYNSTVCVSIFLYADDILLLSPTVTGLQTLLTVCENELCELDMRLNVNKSVCMRFGARFKAHCANLVSVQGGALQWASSCRYLGVYFVSGRVFRCCFHSAKCNFFRAFNSVYSKIGCCASEEVILSLLKSKCLPYILYGVEACPVLQRDKHSFDFTLTRTLMKLFMTSSPVIVNECQTQFNLLPLRYQIDIRTVKFLNQYIISRNSICMLYKSHAQSVLDTIFGVYGNNVSSLHDLHNIINEHFYDFNPSTLGYN

SEQ ID NO: 10 - protein, 1025 aa, unidentified
MVILLLMASYTNAILSIASFNCRGFNQIKKQYICNLLGKCNFLFLQEHWLSERQLGILGNIQSGVLYSGISGFDSSEVLAGRPFGGCAILWHSDVLINVTSVETNSNRICAVLVTTESWKLILINVYLPHEGDDIKSDEFVHCLYLVEDIIGKHSDCHVVVGGDFNVDFNRNWNHTKILNSFCDSNELLSACRSNSNIDYTYNFSMERFSILDHFLLSSTLHNTCIEKISVMHDVDNISDHDPIFLKLKLDVKYIGFSSRIFAPRTSWVKATENDLNRFRHTLSDNLKCITTPTSLLLCHDMKCTDACHHNAIAEYATAISEACLLAAGTCIPHTSNRCTDRRVPGWTERVEPLREKSIFWHKLWMECGRPRDGHVADCMRRTRAGYHYAIRQVRKDEELIVKQRIAEALARDPSRDFWTEIKRIRGNKAGLSRIVDGCIDEASISKLFADKYKCLYASVPYDTVEMQNIQDIVDSQLAASDFFDDSYIINYQDVSDAIFKLNAHKNDGNLGISTDHFLHGGSDLHMHIAFLLTSIVVHGSVPSEFVSSTVIPIPKKPNVNATQSDNYRGIALSSCFCKILDNIILAKNADRLSTSDLQFGFKRKSSTHMCTMVLKETLSYYVSNNSCTFCTFLDATKAFDRVNYCKLFHLLIKRGLPASVIRILIVMYTGHCVRVAWAGLASSFFSAVNGVKQGGVLSPILFCIYMDDLLVKLHKSGVGCYIGTAFVGALAYADDIVLLAPSPSAMRKLLSVCEFYALDFDISFNAGKSNFILVMPNSNQRLRTQMNNCTFSIGGAPVVKVNSYLHLGHIINNQLNDDDDVMYRRNCFIGQANNVLCYFNKLDMCVRIKLFKNFCSSMYGCELWSTDNVEVFCVAWRKALRRVLNLPYDTHSYLLPLLTDTLPVFEEICRRSAKFILKCFNSSSTLVKYVTRHAIDIARYNSNVGKNALFCCNYFKWQLYDFVNGSVSLNRNSFLTFCLNRLSGCEVDNAGSLYEALLVREGNLFVESFTRDDIELIIDAMSRC

SEQ ID NO: 11 - protein, 831 aa, unidentified
MANCNGTIISYNMHGFNQGSELLKSYCANSSVDFLLIQEHWLSPDALHKIDDIAQDYFCFSVSSMTAVLESGPLRGRPFGGLSILVKNMHRQFCSVVALNERYIAVQYNDILIIDVYFPCVSSLNHKDETIDLLVQLDTLVNQSVVKDIIIGGDFNCNLEIESWSSKVICDFMEDNSLSSCNKLAVNVIGAEYTYSNEVLGHYSYLDYFVVSNSLTSSVMSLEICSDDFNLSDHSPVCIEVCNIISDCETTVLCSNKGGKSTKIPDTCYQNRWDHADIVAYYELTRLGLQPILAYLNDCSNSFPPQSAIDYKRYMRACIDLAYNDAVSVLVDAAKNTVPRFKANALKHWWDQELSELKEKAFASHKLWIEAGKPRNGCIFDIRKADKYKYKCLIKRKQLEVRDSITNDLHDALLLKDADHFWKVWKSKFPAKRPNKYALIEGNSDPNIISNTFCNYFSEICTAHPSVNASSNNIFLNRFDGYIGDLNQSTKNIISIDLIEDFILSMKRGKAAGLDSLTIEHIQFAHPAIICILKLLFNYMLEFGIVPEGFRNGLVIPLPKEDSIKKNVKLENFRCITISPVISKIFEHCLMRLFAKYLNSDDAQLGFKKKCGCSHAIYCVKQVVDYYVRGGSTVNVCTLDISKAFDKVNLFVLLCKLMDRNIPNYVINVLYDWFSNNYITVKWLNILSSRCPVNSGVRQGGVLSPVLFAIYVDDILVKLRKSRLGCTIQGLSVNAYMYADDLIILSGSVTDLQKLITLSIEELKCIHLSINPKKCFCMRVGKRFKVNCNNVVVDNYSIQWSSEIRYLGVYLTAGHVLKFNLDYGKKSSIAA

SEQ ID NO: 12 - protein, 926 aa, unidentified
MHGFNQGFTYVNEICNDQAYDIIFIQEHWLYPSTLHKILEISDNYIGFGTSAMQSELEARFLRGRPYGGTAILINKKFKTYCIDSCVFKRVVSVLLGDFLFINVYLPCNDGSAANVNTLGEILANISNIIDAHDVNCIIFGGDLNTDITKVSLHSNMILNFMKDYDLTVCMKHIFGTCDVQFVDTFVCENLNASSCIDFLCMSNSLSDNVSRYKVIDAYNNHSDHLPVSIDVCLPVKCVLYACMNTVCDDVQVHSEHASKCTRSKAKDSRLRWDLGNIQMYYDLTCAHLYPIYECITQVESNMYMYDEQDRSSRIDELYNKLVSTLHNCTKPAIPLVKANTLKHWWTTELNQLKSKSLTSHNIWLDAGKPLRGEIYQSKRHDKLSYKLAIKTAKKEADAAVSDALHTNLISKSSVNFWKTWKSKVCNKVKTCVSVEGCNTDEQAAAKFSDYFSRATSPNSQEYSSAKQNEFNRKLLQYRKVEYANDITAELVALAVAKMQDGKAAGFDNIYVEHIRHSHPLIFSLLAKLFNLMLRAGIVPTLFGNGVVIPIPKENTHKKTHPVDNFRGITLSPILSKVFEHCLLQLMSKYLATSENQFGFKSNTGCTQAVYAVRKVTEYYVANESTMNLCFLDISKGFDKVNNYELMMKLMKRRTPSYFIQILNHWFSISQCTVRWNGVLSAMYRLEAGVRQGGVLSPVLFSVYVDDMLCKLKSMGCSYKALHLSAFMYADDLVLLSPSVSAMQTMLNVCNCELKLLDLKLNVKKSKAVRIGKRFKNNTVNLNIEGQAICWSSEAKYLGVVIVSAPRFKCSFDAAKAKFYRSANSILAKVGHKQNVTVTLHLIATIALPTLTYCIEALHLNKSEVSSLSHPWQSCLFKLFRTFDAEIIKNCCELLNYKTISELYNERVAKFARNLKICNNHVLPLL

SEQ ID NO: 13 - protein, 995 aa, unidentified
MHGFNQGHSVISDFCNPKNVNSMDIICIQEHWLSSTNLSKLANFSNSYFMYGISAMENAEKAGLIKGRPYGGVCILLRKELCKCVKFSKCLERLVCLVIDGYIFINCYFPSIRSDDDLNTVYLLFSEIDDILRDFPGNRVILGGDFNVNIVNKAKYADIFIKKLQNLNLMFCNDIYNSAINFTYCHESLQNYSYIDYFAVSNFMIKDIIDFDILDLGNNISDHNPIVIKVKCQLSDVNVSSKASNYKEFFRWDHANISNYCNLTRTYLQSILEYVNDVSDYLEMHACCYSCSDILKAGKERAYADLNLSASYQTEPCKSCSGNYDIARRCIDSSYNKIVNVLNFSAEKTVPVRKANFYKFWWDDELSQLKYNSIQAHETWVKLNKPKQGIAFDNKVKAKTEYKLAIRQKQSLERDGITNDLHESLLAKSTNVFWKSWNSKFGQKSKVPLVIESSQDNQVISDKFAGYFASINSSNFPSNKNRHKDVFLCKFKDYKGDNWSYRDKINTFMLDYCIKESKLGKSPGADGITIEHLLYSHPMLICLLSKLFNLMLRFSYVPDKFAECIVIPVPKLQNVSKLMKCEDFRPISLSPIISKIFEKCILKLVEEYFVTSDRQFGFKKRLGCTHAISAVKSTVDFFTENGSTVNMCALDLSKAFDKINHFILFNKLMKSNMPLRILLLIICWYSKLVNVVRWNGSLSNVFKTANGIRQGGILSPFLFNIYVDDILFSLKRSKLGCYVGNMCCNSFMYADDLLLLAISVTQLKQMIIMCTNLFEEIDMRLNITKTNCIRVGNRHLSTVIPMVIGDIVVNWKQEMRYLGVCFVSAKKLKLNIIFAKQKFFKSLNSIFSKVGSNASPEVILSLFNSFCVPVLLYGLGPFNLSLNLKTRNSLIKAYDSVFYKTFKTFDNEVTRYCQLMFRVMPLTFKLDLLTINFMLSSQKCDNEIIKFFSHRSFKINMDKLLYKYNIVIDDMYKAKEALFDNFKVKVESNIALGLV

SEQ ID NO: 14 - protein, 1438 aa, unidentified
MTETSHAPVLDQTCTRYQRHNVHEQAMQFVPPNRVVFSFFGPCDIVCHRGAFLSVTSYCKTLSLLQRAHFTRHRNKATGQPFLLLSVYKQQDLQTLTQQGLVPFTQTCNEALSLRGCRVAPSGRVALPYMMYPQGDHRNQPVHNQETHTPLTPGLDANPFAALSVEQQTCGASTSRTSLIVGSMNVNGLFTQTMSSKHQDVCAFVSSHTVDICCLQETWCDMSETEYSSMLASCGYTSFGVSGYSTRTRTGGGTGILVATSISHLAHMLHWESQKHTQTTWIKLNSTQYKARKTAVGSVYLRPVSHTRPGDAERYVTELIALRDDITYLSEHNFDVVLCGDFNARIGNSQVSPHVPQHNEQTRNTQGDRLVTLLEQCNMFVMHNHTSFSPTCMHAGGSSVVDYCITNSSCRARCSDAAINFAGPCVSDHALLTMSLSHATRTRHRKTRRAHAKLWNRRAMHDEQTIAQFHATLETPLTQLSDFIDASTLSPQEATDTFTSKLTAALRDAGATCFGTHQVRPHRAQWWNKDYANLRHECWNLYETHRRSGSEADRAAYEEKRAAKNKMKRNLKRAYVKAQAGHVSKLCTPGLVSKTSWGAAKRLLKLVTRGSSATEHTLPTVIHDGAPTDDLEAVMQVFSSHYHTEMNPVPSATFCEATDTLVKDKLHAWRQAEPEHINNMDDEFTITELDRALGSMHNWKAADHDGMIIELLRAGGPALQLVVLKILNFCWTHETIPTNWKLGTIISLYKAGKKENPSNYRGITLLSVVRKLFCTLLRSRLQDNISLHESQAAFRANRGCMDHVHTLARIVRAANRKDIPVYAFFLDIRKAYDTVWRDGLIYKLLQKGVTGRLGRVISQVLTDTQSRVRFQHRESAYFPLTLGVGQGDPLSTILFDVFIDDLLEELHARPVHHCIPVDSPHLDRIADLTYADDVNALSLTPEGLQGHIDTIDTWLFRWRSQPNVSKSKTMVFNPPQGVPPTVFTMRGSNLDTVQCFKYLGVFFQSNGSWTDHTQHVRTQMNKAVGMWRPVLRCHYLPVAARLRIIYAFVYAPALYGAEVWIAPKSELEKLDTICKSAIRTVFGLHQFDCREEILFADTGLLPVSSLINAAKLCWFTKLLNMPESRFPTAVEAVTLPGDTQRGRVAGGDFGTRIADITTEIRRYAHMKLVMQDPLEHAPHRRRRPIRVSKRLSHTKPDAHLYETITYSSLTRERVKALLWKVYMEKCFERRGRKDGVTGEWMRSVIKHELGSLAPFLHSVECGLVRVLMSARSRLSPVLCRPPQDKHREYSQAAGAFTTYISRAPIGAVLPSPPVAHCVRHVSAVAELVRELLAQPGPYGCGSSVSWSDIAVVLVVGIDGVGGAPGVLGVVARPSPLSELARVVAFCGASPRLRHHVAWMSRSVPVAGVVNVPVDMAMLQRTKAGRQVAPQGKHGAHSS

SEQ ID NO: 15 - protein, 972 aa, unidentified
MAISYTSNNSVRICSFNMHGFNNGVSMTKILCNSFDVILLQEHWLLPSNLSKLGDISHDFTHHSVSAMTTKISEGILYGRPFGGVSILYRNSLTKNISIIDADKEEGRYVTIKLKCVNNEFITVTNVYFPTICAYSDYIVNTSSIMAYLDNLFANEVSCHHVVAGDFNFEYRNDNIGFDLFRSLAVDNNLICCDDLHLNQNIKFTYKHETLPQQSWLDHFFVSAGLTSSIVYCDTIDNGSNLSDHLPICCTINVSLSDSNCTPKLSKVYRDRWDKADLIKYYYQSGIHLQSLTAPPHVTQCSTHCQKAQHLQDINAYYERIIQALKASAIGCVPRMPVNCLKHWWNDDLTRLKNLSIDMHNLWRQVGSPRNGIINEARLKAKLDYKQAIRQAMLDCENKDADIINNKFNQKDSRNFWKCWGAKYRKKVNNTACIDGCTDNSTIANKFKAYFQNTYVDSTCDVNACMEFDKLMSDHSHLQHNDIVEEIRIEDIEKCIELLKPLKSAGHDDVAPEHLIHSHPSLCMHLKLLFSMMLNHNYVPDSFGIGIIIPVVKDKRGNLNSVENYRPITLSPIISKVFESFVLNRFAKFMTCDPLQFGFQRSVGCNNALFAIRQVIQYFNDRDSNVMVASLDACKAFDRVNHFKLFSTLLQRKLPLHIIKVLINWYCKLMVQVRWNNSLSDLFHVKSGVRQGGILSPALFNVYIDCVINKLRASQLGCHIGSLYIAVVLFADDILLLSSSFMELQRMIDLCVESGDEIGLKFNAAKSHCMIIGPHKVLVKPDMMMGNSPTAWSETIKYLGVYIQSDKKFTVDLSLVRRKFFASVNCILRNAAFTSDIVKLELVEKQCFPILLYGLQSFDLKSSVIANVNAWVNSVYRKIFGYHKWESVKECIYLLGRLDVFHTIKLRRINFLKNIQKCNNEVARSLFNYVLHTREFQSCCTLPNNSVINISKSCDNVRKAVFNSFQSKVVGH

SEQ ID NO: 16 - protein, 957 aa, unidentified
MASQTLDTQTANNVCNSVSVMSYNMHGFNQGYSYLNDICMKQTYQVILIQEHWLYPATLHKLANISEHYSFYGASAMQTALDAGFIRGRPYGGTGILLHKDIAKYCVESCAFERVVGVLLGDFLFVNAYFPCWDGSVINLDAANELLANLSNVLDSYQAKHVVFGGDFNANLTKNSVYSNMILEFMSEHQLDICRKVLFGSSNVIFSDTYVNEALNASSCIDFICISSGLSNNVAQYDVHDVFNNHADHLPVSLKLCLPVSSILYNCIASGSSACMFNEAKSRDTQSCDSKGNKLRWDKGNTQLYSDLTYAQLFPIYETLHSIDVESSAYTAQEHRSLINETYTQVVSALHYAANVSIPAMASHTLKHWWCSDLSELKKKSMLSHNEWINAGKPHAGRIHTAKQQSKLQYKRAIKHAKASAENSVSDELHRNLCSRNNVKFWKTWKNKIKRPGTEKLYVEGCNTDAKAAELLADYFNKATSPNSNEYNNSKRVEFEKSFASYAIQANDIDISAGLVESAALKMTAGKAAGIDNISIEHVHYCHIVIYSLLAKLFNLMLCFSCVPDAFGYGVTTPIPKEESHKKIHPVENFRGITLSPVLSKLFEHCLLSVFSDFLQTSDNQFGFKRSTGCTHAIYALRKVTEFFICNESTVNMCFLDISKGFDKVNNFELLLKLMKRKAPSCFIKLLHDWFSISYGSVKWNTSRSDWYKIGAGVRQGGVLSPILFAVYVDAMLEKVKTMGCQYKSLCTGAFMYADDLVLLSPSIYELQHMIALCRNELHSLDLKLNVKKSKALRIGKRYKCKALPLMIDGQAVHWSNEARYLGIVIRSACKFKCNFDPAKVKFYKAANTILAKLGNKCNVTVTLYLVAAVALPPLIYGIEALTLNSSELNSLNHPWNNCLGKMFNTFDKELIKNCCDILGYESCQNVYVKKVEKFLRNMKFIDNAILSALNTEQVNV

SEQ ID NO: 17 - protein, 7 aa, synthetic construct
PKKKRKV

SEQ ID NO: 18 - protein, 16 aa, synthetic construct
KRPAATKKAGQAKKKK

SEQ ID NO: 19 - protein, 9 aa, synthetic construct
PAAKRVKLD

SEQ ID NO: 20 - protein, 11 aa, synthetic construct
RQRRNELKRSP

SEQ ID NO: 21 - protein, 38 aa, synthetic construct
NQSSNFGPMKGGNFGGRSSGPYGGGGQYFAKPRNQGGY

SEQ ID NO: 22 - protein, 42 aa, synthetic construct
RMRIZFKNKGKDTAELRRRRVEVSVELRKAKKDEQILKRRNV

SEQ ID NO: 23 - protein, 8 aa, synthetic construct
VSRKRPRP

SEQ ID NO: 24 - protein, 8 aa, synthetic construct
PPKKARED

SEQ ID NO: 25 - protein, 8 aa, synthetic construct
PQPKKKPL

SEQ ID NO: 26 - protein, 12 aa, synthetic construct
SALIKKKKKMAP

SEQ ID NO: 27 - protein, 5 aa, synthetic construct
DRLRR

SEQ ID NO: 28 - protein, 7 aa, synthetic construct
PKQKKRK

SEQ ID NO: 29 - protein, 10 aa, synthetic construct
RKLKKKIKKL

SEQ ID NO: 30 - protein, 10 aa, synthetic construct
REKKKFLKRR

SEQ ID NO: 31 - protein, 20 aa, synthetic construct
KRKGDEVDGVDEVAKKKSKK

SEQ ID NO: 32 - protein, 17 aa, synthetic construct
RKCLQAGMNLEARKTKK

SEQ ID NO: 33 - protein, 950 aa, unidentified
MRGFFQGLVVVNDLISGYQSPDVILLQEHWLTPANMNLFDEKITTHFVVGKSAMSDRVSAGPLVGRPYGGAAILIRNELRADTECVFCSDRFAVVRICNLLIMSVYLPCAATAARMFIVEDLLQEIWSLRLKYSECSVVIGGDFNADLNKQNDVSNFINSFLTTCLLIRCDSKFLSRQQSTYVNESLGQSSTIDFFVCDVVDDIIDYCVIDPDVNFSDHLPVAVRCKWSRTDNLKRSKSSNSSKVKHLRWDHGDLLSYYSSTMSRLYPLYEYLIRIEGWLVEPSNAVQRETVIRLVDHVYDQLVVALRESANSYIPKHAKKFFKFWWNQELDALKHNAIASSSVWKNAGKPRGGALYLQYKRDKLLYKKRLREEQKAEAACYSNDLHDALLRKSGQDFWKCWNSKFERRSNKVVQVDGITDSAVIADKFAEYFESVCRPFNADRNNLIKSKYNELRSTYTGTPIVEEQWFNVELLSKLVDSMSKGKAAGLDELSSEHLKYSHPVVVCILSKLFNLFVYYSHIPASFGRSFTVPIPKHDGRTHALHVDDFRGISISPVISKLFEMAILDRFSVFFSTSDHQFGFKKNLSCRHAIYCVRSVIDNFVLHGSTVNVCALDLSKAFDRMNHYALFIKLMERSFPCELLAILETWFYISVSCVKWNDNLSSFFVLTAGVRQGGVLSPYLFAIFIDDLVYKVKSLNVGCYISLTCAAIFMYADDILLLSPTVDGLQQLLHVCENELEQLDMKLNVNKSVCIRVGPRFNADCAELRSRNGAVLKWTDSCRYLGVYFVRGRTLKCSFSNAKSRFFRAFNAVFGKVGRAASEETVIELIRAKCIPILLYATEACPFFSRDKQSLEFTVTRLFMKIFQTGSPAVVRDCQRSFNFLPIEMQVIIRTSRFLQAFVATQNSLCSLFQRSASCQLNDIFSKYDRVQTASQLANRIHHTFAMCDSV

SEQ ID NO: 34 - protein, 640 aa, unidentified
SYHYAIRAVRRNEQNIIRERVADALLRDPSRDFWTEVKKIRNSKSGRAVIVDGCSDAPSISQLFASKYRHLYTSVPYQHNDLQSIVSDVESRISFDGDCFIGSQEVMAALSKLKLHKNDGDLGLASDYFINSDPALSVHIALLFTGIVIHGFVPSNLLSSTIVPIPKKSNVNATDSDNYRGIALSSVLGKIFDNVILVKYSDKLSTCNLQFGFKRNSSTHMCTMVLKETISYYVNNNSSVFCTFLDASKAFDRVHYCKLFRLLLGRGLPVCILRVLIQLYVGHSIRVTWAGLVSSCFTALNGVKQGGVLSPVLFCIYVDELLIRLAESGVGCYIGFSFAGALAYADDIVLIAPTPSAMRKLLAICDTYATEYNILFNAQKSNFIAFVPSSRHSLHKAMTNCVFRIGGVQIEHVFSYTHLGHIITSRLDDADDILHRRSSYIGQVNNVVCYFDSLSWTVKLGLHKSYCSSIFGCELWALDSVRDIEKFCVAWRKGLRRVLSLPRAAHSHLLPLLSNSLPVYDEICKRSAKFIVSCQCSDNILVRAVVNYAIAARSKSVLGRNVMLLCRRFHLSFDDFVSGRLLLSGDIFVSHYLNSLSEAQLQSVCFALELLCLREQSFKLNNNMRLNADEISDYLAAVLC

SEQ ID NO: 35 - protein, 486 aa, unidentified
MHGFTPDCMLLSTVVPIQKGKNVNVTDSANYRGISLSSIFAKLFDLLILQRYSDCLCLSDQQFGFRAKRSTDMCSMVLKESISYYVNNGSSVYCTFIDATKAFDRVEYCKLFRQLLSRGLPPVIIRIMLNLYVGHVTRVEWNGIRSRNFSVFNGVKQGGIVSPILFCIYLDGLLQSLATSGVGCYIGTIFVGAMAYADDLVLLAPSANAMRLMLRNCDAFANEYNIRFNANKSKCLFCSARRHSRCNIGAQPVFYIGGNPIEIVDHWPHLGHIISSHLDDEQDIIQRRNAMAGQINNVLNYFVGLDCFVKQKLLTTYCYSLYGSVLWDLRHLCIDSVCTTWRRGLRRVWGLPHNTHSNLLPLLSCSLPVYDELCKRFVLFAQKCLMSDSSLVSSVATYAFIYGRSDSVFGRNVSKCCLRFNVANDDFLSLNKKFMFNNYCSNLEQDTMSTVNLLLELICVRDGLFIMPMFADAAELIEIIRLLCTV

SEQ ID NO: 36 - protein, 802 aa, unidentified
MTITNDAAQLVVISYNLHGLNQGLPGIREFMTELKPDVIMVQEHWLTSDNLNKLSDISDEYFVIGSSAMDARVSAGPLFGRPFGGTAVFIKNKYINVTVNLVTSERYVVIQLCDWLLINVYLPCIGTSNRILLYSDTLCELQSIIRAHPECNCLVGGDFNTDLNDTRSHTANTVVNGFIAGCNLNLSDHLPIITVCVFDSNCKLNNPKPTSTEDVTHFRWDHAPLQDYYEHTRLGLQPILADLDELIGNKLSYSNVDFLCQVDCIYNRVVSVLQQCSHAYVPKHKKNFYKFWWNQELDELKDKAIASCKMWKDAGKPRHGAIHAKYRQDKLLYKKRIRQERVQETSSFTNELHDALLNKSGRDFWKKWNSKFENKSNKILQVGGTSDTATILNNFAKHFEQVCVPFNATRNEELKSRYKEMRLNYSESSVINDTTVFDVQLVENLLTNMKNGKAGGLDELTSEHLKFCHPVVVIILCKLFNLFVINGHIPDSFGVSYTVPIPKSDGRSRSMTVDDFRGISISPVISKLFELCVLDRYSDYLQTSDHQFGFKKQLGCRHAIFSVRSVIEQYISNGSTVNLCALDLSKAFDRMNQYALFIKLMERRFPVKILTILEQWFSIAETCVRWGSEFSYFFSLLAGVRQGGVLSPVLFAIFIDGIVNRVNATNVGCYNSTVCVSIFLYADDILLLSPTVTGLQTLLTVCENELCELDMRLNVNKSVCMRFGARFKSHCANLVSVQGGALQWASSCRYLGVYFVSGRVFRCCFHSAKCNFFRAFNSVYSKIGCCASEEVILSLLKSKCLP

SEQ ID NO: 37 - protein, 950 aa, unidentified
MRGFFQGLVVVNDLISGYQSPDVILLQEHWLTPANMNLFDEKITTHFVVGKSAMSDRVSAGPLVGRPYGGAAILIRNELRADTECVFCSDRFAVVRICNLLIMSVYLPCAGTADRMFIVEDLLQEIWSLRLKYSECSVVIGGDFNADLNKQNDVSNFINSFLTTCLLIRCDSKFLSRQQSTYVNESLGQSSTIDFFVCDVVDDIIDYCVIDPDVNFSDHLPVAVRCKWSRTDNLKRSKSSNSSKVKHLRWDHGDLLSYYSSTMSRLYPLYEYLIKFEGFLVEPTNAVQRETVIRLVDHVYDQLVVALRESANSYIPKHAKKFFKFWWNQELDALKHNAIASSSVWKNAGKPRGGALYLQYKRDKLLYKKRLREEQKAEAACYSNDLHDALLRKSGQDFWKCWNSKFERRSNKVVQVDGITDSAVIADKFAEYFESVCRPFNADRNNLIKSKYNELRSTYTGTPIVEEQWFNVELLSKLVDSMSKGKAAGLDELSSEHLKYSHPVVVCILSKLFNLFVYYSHIPASFGRSFTVPIPKHDGRTHALHVDDFRGISISPVISKLFEMAILDRFSVFFSTSDHQFGFKKNLSCRHAIYCVRSVIDNFVLHGSTVNVCALDLSKAFDRMNHYALFIKLMERSFPCELLAILETWFYISVSCVKWNDNLSSFFVLTAGVRQGGVLSPYLFAIFIDDLVYKVKSLNVGCYISLTCAAIFMYADDILLLSPTVDGLQQLLHVCENELEQLDMKLNVNKSVCIRVGPRFNADCAELRSRNGAVLKWTDSCRYLGVYFVRGRTLKCSFSNAKSRFFRAFNAVFGKVGRAASEETVIELIRAKCIPILLYATEACPFFSRDKQSLEFTVTRLFMKIFQTGSPAVVRDCQRSFNFLPIEMQVIIRTSRFLQAFVATQNSLCSLFQRSASCQLNDIFSKYDRVQTASQLANRIHHTFAMCDSV

SEQ ID NO: 38 - protein, 807 aa, unidentified
MESAQIDVLLLQEHWLSDAQLNALNNVGANYLNFGVSGFDTSAVLGGRPYGGCAVLWRSDLLLQVQPLVVSSRRLSAVIFSTDNWSLILINVYMPYEGDEIKTDEFIDLLSIIEDLVLSNSASHVIVGGDFNVEFNRNRMHTALLNSFCDNTGLSPVIQHSSCNIDYTYNFNMSRFNILDHFLLSGTLFDVCVTSAYVVHDIDNTSDHDPIILRLSLDIKYVSVCNRTSSSRVSWVKASDRDIRNYQYNLASNLQHVTIPSVALLCKDVNCSNFAHRCQLSRYLTDISDACLAAGEASIPHTCSRHSGKRIPGWSEKVEPLRQRSLFWHSMWVECGRPRSGVVADCMRRARASYHYAIRAVRRNEQNIIRERVADALLRDPSRDFWTEVKKIRNSKSGRAVIVDGCSDAPSISQLFASKYRHLYTSVPYQHNDLQSIVSDVESRISFDGDCFIGSQEVMAALSKLKLHKNDGDLGLASDYFINSDPALSVHIALLFTGIVIHGFVPSNLLSSTIVPIPKKSNVNATDSDNYRGIALSSVLGKIFDNVILVKYSDKLSTCNLQFGFKRNSSTHMCTMVLKETISYYVNNNSSVFCTFLDASKAFDRVHYCKLFRLLLGRGLPVCILRVLIQLYVGHSIRVTWAGLVSSCFTALNGVKQGGVLSPVLFCIYVDELLIRLAESGVGCYIGFSFAGALAYADDIVLIAPTPSAMRKLLAICDTYATEYNILFNAQKSNFIAFVPSSRHFLHKAMTNCVFRIGGVQIEHVFSYTHLGHIITSRLDDADDILHRRSSYIGQVNNVVCYFDSLS

SEQ ID NO: 39 - protein, 357 aa, unidentified
GSARCPSIGQPTSRVEPWRLTREGNPTASNVVDGCTTPDSISQLFAAKYKELYTSVPYSADDMSSLNSDINALMSSGFCAGCFVNANEVFAAVKELKLHKNDGNTGLTSDHVKCALPDLSVHIALLLSGVLSHGSIPKDLLLSTVIPIPKNKNGNLADSSNYRGIALSSVFCKIFDKILLARYNDKFVTSELQFGFKAGRSTHMCSMLLKESMLYYKNNNSLVYCAFLDATKAFDRVNYCKLFRLLIKRGLPPPILRALLNFYVGHTVRVSWFGSVSSYFLALNGMKQGGVISPLLFCVYIDDLLCALEKSGVGCYIGLHFTGALAYADDIVLLAPSPTALRRLLSICDVYAAEYCI

SEQ ID NO: 40 - protein, 969 aa, unidentified
MGFQAQTAGGMNNTAIMTSDQNCTSLSALTFNMHGFNQGSVLLKEVCEKQIYDLIFIQEHWLYPSNISKLLEISPHYIGHGISAMESAVDAGFIKGRPYGGCAILVNNKYKNMHADVCTFERVVAILLGDVLLINVYCPCENGSTEGFDALNEILANIANILDTYKAQYVVIGGDFNTDLSKDTTHSVLIKEFLDEYGIKYCKSEMFGSVNIQFERTFACETTGAGSCIDFFCVSDNICNNVMRYKVLDMYSNHSDHRPILCQFCVPVQSSLHQYVSSGCVNFVNERANANVHASKQSAESEQALRWDLGNVQLYNDLTYQYLYPIYDRLMNYDNMHSVNETNINEGKEAACVFIEQSYNDLTNALHHCAANSIPRVQRNAIKHWWNSELTELKKCSITSHNNWLESGKPRSGAVFALKQQDKLRYKQAIKKVKNEADAAISDELHAKLCAKKTTSFWKTWKNKVSNKAKKQISVEGCNTDEMAVCSLAEHFRKATVPNCEIFNNEKKTEFVHKLSEYSVNSSPTAVTAEIVYISLSKVHKGKAPGIDNITAEHIGNSHVVIYSLLAKLFNMMLEYGHVPVSFTNGIIVPIPKDENNKNKYPVDNFRGITLSPVLSKIFEHCLLNTFGDYLLTCDNQFGFKTKVGCSHAVYAVRKITDYFVSGESTVNLCFLDISKGFDKVNKYELLLKLMNRRIPVCFIELLNYWLSMSISTVKWNGFFSQPYSLEAGVRQGGVLSPLLFAVYVNDMLCKLRKMGCSYKWLHFGAIMYADDLVLLAPSVYELQCMIRVCQEQLKLLDLRLNFKKSKALRIGKRYKHKAAQLQLNGEKIPWGTEAKYLGMVIQSAARFKCSFDQAKVKFYRGANAILSKLSNKSNVTLTLYLVSTIALPTLTYGIECLSLNKTEIKSLDYPWYKCFCRVFQTFDAGIIQLCQAILGYKSIKELYENRCQMFLEKIKIYGNFMLREASEL

SEQ ID NO: 41 - protein, 955 aa, unidentified
MNSYSSTTLSAAESSCTTLSITTFNMHGFNQGSTLLIDVCYKETYDVIFIQEHWLYPHNIRKLLDISSKYTGYGISAMQSVVDSGFVKGRPFGGCAILIHNKYINLHVDCSVFERVVAVNVGDFLLTNVYFPCENGSAEGFDTLNEILANVSNILDTYKAPYAIIGGDFNTNLTKRSIHANTVNDFLNAHNMSYCKAKVFGSVEISFEDTFACDKTGASSCIDFVCVSNCLMHNVSKYEVLDVYNNHSDHKPLFCQLCVPVSSTLHQFVASGCRKLMNGNAGDSKCSNDSNCALRWDLGNVQLYTTLTSERLYPIYEKLLNYRYVVDEHNPHESKQLASQRIDKLYNELILALHECAATCIPQVKRNAIKHWWNSELNELKTCSIKSHKSWLEAHKPKSGALYLAKQQAKLRYKQAIKRTRLAADASISDALHVNLTTSSTEKFWKTWKTKVCKKNKQNMLLEGCSTDDIAVCKLADFFSKTTAPNSDEYNVKKKSEFLEQITDYSGQTNYDTINAELVYNSMSKLKKGKSPGIDNITVEHICNSHVVIFALLAKLFNLMLEVRYVPAIFGNGIIVPIPKEDTGKKVHPVDNFRGITLSPVLSKLFEHCLKCIFAEFMSTCDNQFAFKNNMGCSHAIYSVRKVTEYFVMGDSTVNLCFLDISKGFDKVNKYELLLKLIKRNVPVCFIELMHHWFDISKSTVKWNGHFSQSYELKAGVRQGGVLSPLLFAVYVDDMLRKISKMGCTYNWLHLGAIMYADDLVLIASSVYELQCMLRVCQAELNLIDLKLNVKKSKAIRIGKRSKLKAASLKLSEGHIAWSSEAKYLGIVVQSAARFKCNFDHVKIKFYRGANAILSKLSSKANVTVTLHLISSIALPSLTYGLECLALNKTELKSLHYPWHKCLCRIFQTFDEQIINLCQAILGYKSVKDLYEIRACMFLSRVKKCDNFLLRALAA

SEQ ID NO: 42 - protein, 454 aa, unidentified
MRQKSEQLELALDNRGEAPTSRRSGEAPTTAHEAERSGGGHRLMEQVVARANALAALRRVKQNRGSPGVDGMTVGELPQYLAKHWETVREQLLAGSYQPEPVRRQAIPKPGGGTRVLGIPTVLDRFIQQCLLQVLQPRFDPSFSDHSYGFRPGRNAHDAVCAAQQYIQAGRRWVADLDLEKFFDRVNHDVLMERLARRIADRRVLRLIRRYLVAGILHGGVVVERREGTPQGGPLSPLLASVLLDEVDRELERRGLAFARYADDLNVYCGSRRAAHDAMATLKRLFAALRLKVNESKSTVARVWERKFLGYSFWVAPGRVIRPRIAPAALAVMKERVRRITRRTGGRSLEAVAQELREYLTGWKAYFRLAGKPRVFRDLDEWTRHRLRAVQLKQWKRGRTVCRELLARGVPEREARAAAAHARRWWAMAAHSALQTALPNSHFDQLGVPRLAGR

SEQ ID NO: 43 - protein, 462 aa, unidentified
MRADEAEAHASAASTGKGGRNLPGTAAGAEVRAAAGGRTKPEALRLMEAAVERSNMLGAYERVVKNQGAPGVDGLTVTEFKPWLQAHWPKIRQVLLAGEYMPAAVRKVDIPKPQGGVRTLGIPTVLDRLIQQALHQVLQPLFEPEFSESSYGFRPGRNAHQAVEAARSYVAEGKRWVVDLEKFFDRVNHDVLMARVARKVKDERVLKLIRRYLEAGLMEGGMTSARTEGTPQGGPLSPLLSNTLLTDLDRELEKRGHRFCRYADDCNVYVGSRRSGQRVMAKITAFLEQRLKLQVNADKSAVARPWQRKFLGYSVTWHRNPKLKIAPSSRQRLAEKIRQTLRGARGQSLRQVIAQLNPILRGWVAYFRLTEEKGVLEELDGWVRRKLRALLWRHWKRGYPRAQNLMRAGLRPERAWQSATNGHGPWWNGGSSHMNAACPKSWFDHMQLVSLLATQRRFSLVS

SEQ ID NO: 44 - protein, 466 aa, unidentified
MKGGKQKISQDTCLQESRAEPEGYAGGQTFIWMTENNLTNANKPEYGLLEQILSPTNLNRAYKGVRSNRGSGGIDKMEVESLKDYLVDNKETLIQSILDGKYRPNPVRRVEISKEKGTRKLGIPTVVDRVIQQAIAQVLSPTYERQFSENSYGFRPGRNAHQALNRCRDYITDGYIYAVDMDLEKFFDTVNQSKLIEVLSRTVKDGRVVSLIHKYLDAGVVIRNKFEETEMGVPQGSPLSPVLSNIMLNELDKELEKRGHPFVRYADDLIIFCKSKRSADRTLANTVPYIENKLFLKVNREKTTTAYVSGIKFLGYSFYVYKGEGRLRVHPKSIAKMKERIRKLTSRSNGWGYARRKEALRQYITGWVNYFKLADMKKLLVSVDEWYRRRLRLVIWKQWKRVRTRGRYLMKLGIVKHQAWEFANTRKGYWHTAKSPILNRSVTSNRLRQAGYVFFVDYYRVVNGIN

SEQ ID NO: 45 - protein, 271 aa, unidentified
MHNFKSRFELSKGKWVYIQIEDLANHAKDHIRQIRDLWTPPEYFFHLQKGGHIAALRLHTPNEWYGKVDLSKFFNNITRHRVTRSLKRIGYSFRDAEEFAVASTVLVNHATRRYVLPYGFVQSPLLASISLDKSDLGNCLRRLHEETVSVSVYVDDIVVSADSERDVAEALSNIYLAAINSRFPINEEKSRGPSSTLNAFNIELDMHELEITAKRYEKMCGEVLINGTGQVSDAILNYVQTVNPAQAEQMLRDFPGVFGTLSSSASRYDAS

SEQ ID NO: 46 - DNA, 1362 bp, synthetic construct
  source 1..1362: mol_type = other DNA
atgcgccagaaatctgaacagctcgaattggctctggataatcggggcgaagctccgacttcccgtcggtcaggtgaggccccgacaactgcacacgaagcagaacgctctggtggtggtcatcgtttaatggaacaggtcgttgcgcgtgctaatgccctggcagcactgagacgcgtcaagcaaaatcgcggatcacctggagtagatggtatgaccgttggtgagctgccacagtacctggcgaaacattgggaaacggtgcgcgaacagctcctcgcaggttcctatcaaccggagcccgtgagacgccaagcgattccgaaacctggaggcggtacacgagttttgggcattccgacggtccttgatagatttatacaacagtgcctgctgcaggtacttcagccacggtttgatccttccttttccgaccattcatatgggtttcgtccggggcgcaatgcgcatgatgcggtgtgtgcagcacagcaatatattcaggcggggcgtcgttgggtagccgatctggacttggagaaatttttcgatcgggttaaccacgatgtgctcatggaacgtttggcacgccgaattgccgatcgccgtgtgttacgactgattcgccgttacctcgtcgcaggaattctgcacgggggcgtcgtcgtggagcgacgtgaagggacgcctcaaggcggcccactttcgcctttgctggcatcagtcttactggatgaggtggatcgcgagctcgagcgtcgcggactggcgtttgcacgatatgcggatgacctcaatgtctactgcggttcccgtcgcgccgcgcacgatgcaatggcaacccttaaacgcctgtttgctgcactgagattgaaagtgaatgagtcaaagtcaactgtagcccgcgtatgggagcgcaaatttttaggctactcattctgggtggcgccggggcgcgtcatacggccacgaattgcacctgctgcgttggcagtcatgaaagaacgagtgcgtcggataacacgccgtaccggagggcgttcactggaggcagtggcacaagaactgcgagaatacctcacaggctggaaagcgtattttcgattggcgggcaagccccgagtcttccgcgacttggatgaatggactcgtcatcgcctgagagctgtgcagctgaaacagtggaaacggggccgtactgtttgcagagaattattggcacggggggtgcctgaaagagaagcacgtgctgcagctgctcatgcaagacggtggtgggctatggcggctcactctgcactgcagacagctcttccaaactcccactttgatcaattaggtgttccacgcttggcgggtcgt

SEQ ID NO: 47 - DNA, 1386 bp, synthetic construct
  source 1..1386: mol_type = other DNA
atgcgtgctgatgaagccgaagctcatgcatctgcggctagtaccggtaaaggtggccggaatttgcccggtactgccgctggcgcggaggtgcgtgctgcggcaggaggtcggacgaaacccgaagcacttcggctgatggaagcggctgtggaacgttccaatatgctgggcgcgtatgaacgggtagtcaagaatcaaggggctcctggagtcgatggtctgacggttaccgaattcaaaccgtggctgcaagcccattggccgaagatcagacaggttcttctcgccggagaatatatgcctgcggcggtccggaaggtcgatatacccaaaccgcaaggtggtgtacgcacgttgggcattcctactgtcctggacagattgattcagcaagccttgcaccaggttctgcaacctctgtttgagcccgagttttcagaatcatcctatgggttccggcctggtcgcaacgctcaccaggccgtggaagcagcgcgtagttatgttgcggaaggaaaacgatgggtagttgacttagaaaaattcttcgatcgtgttaaccacgatgtactgatggcccgtgttgcccgtaaagtgaaagacgaacgcgtcctgaagctgatacgaagatatttagaggctggcctgatggaaggaggtatgaccagcgctcgaactgaagggactccacagggtggcccgctttcaccgcttttgtcaaatacccttttgaccgacttagatagagagctggaaaaacgaggacatcggttttgtcgttacgcagatgattgcaatgtgtatgtggggagtcgtcgttctggccagagagtaatggcgaaaataaccgcgtttttggagcagcgcctgaaattgcaggtaaatgctgataaatcagcggttgctcgtccatggcagcgtaaatttttggggtattccgttacctggcatcgtaatcctaaactgaaaatcgccccgagttcacgtcaacgccttgctgaaaagattcgtcaaaccttgcgcggtgcgcggggccaatcgttacgccaggttatcgcacagttgaatcccatactccgcggttgggttgcttattttcgccttactgaggagaaaggggtacttgaagagttagatggctgggtacgtcgaaaacttcgggcccttttgtggcgccattggaaacgcggttaccctcgtgcacagaacctcatgcgcgcgggtcttcgtccggaacgggcgtggcagtccgcgactaacggtcatggtccttggtggaacggtggaagttcacatatgaatgcggcgtgcccgaaatcctggttcgaccacatgcagctcgtgtcgctgcttgcaacgcagcggagattttcgttagtgtcc

SEQ ID NO: 48 - DNA, 1398 bp, synthetic construct
  source 1..1398: mol_type = other DNA
atgaagggcggcaagcaaaagatttctcaagacacctgtcttcaagagtcacgcgcggagccggaaggctatgccggtggacagacgttcatctggatgaccgagaacaacctcactaatgcgaataagccggaatatggattactggaacagatccttagcccgactaatttaaaccgggcatataagggagtccgttctaatcgcggttcaggtggtatcgacaagatggaagtggagagcctgaaagattacctggtcgacaacaaagaaacacttatccagtcgatccttgacggtaagtatcgcccaaatcctgtacggcgtgtagagatttccaaggagaaggggacaagaaagttagggatcccgactgtggttgaccgcgtcattcaacaagccatagcgcaagtactgagtccaacctacgagcgccagttcagtgagaactcctatggcttccgtcctggacgcaatgcgcaccaagcattaaaccgttgccgcgattatattacggacgggtatatttatgcagttgatatggatctggaaaagttcttcgacacagtaaatcagtcaaagttaatagaggttctgtctcgtaccgtgaaggatggtagagtagtttccctgatccataagtacctcgatgctggtgttgtgattagaaataagttcgaggaaactgagatgggagttccgcaaggtagcccactctccccggttttaagcaacatcatgctcaacgaattagacaaggagttggaaaagcgcggtcatccattcgttcgatatgctgatgacttaattatattctgcaagtcgaagcgaagtgctgaccgcactttggcaaacaccgtcccgtacatagaaaataaactgttccttaaggtgaatagagaaaagactacgaccgcgtacgtgtcaggtatcaaattcttaggctacagtttctacgtttacaagggagaaggacgccttcgagtccaccccaagtccatagccaagatgaaggaacggataagaaagttgactagtagatccaacgggtggggatacgccagacgtaaggaagcgctgagacaatatattactggttgggtgaattactttaagcttgcggacatgaagaagctgctggttagcgttgatgaatggtatcggcggcgtctgcgtttagtaatttggaagcagtggaagagagttcgaacgcgtggccgttaccttatgaagttaggcattgtgaaacatcaggcatgggagttcgccaacactcggaagggttactggcatacagcaaagagcccaatcctcaaccgcagtgtgacgtcaaaccggctgagacaggcggggtacgtgttctttgtagactattatcgcgttgtgaatgggattaac

SEQ ID NO: 49 - DNA, 1407 bp, synthetic construct
  source 1..1407: mol_type = other DNA
atggctagcgcatggagtcatcctcaattcgaaaaatccggaatgcgccagaaatctgaacagctcgaattggctctggataatcggggcgaagctccgacttcccgtcggtcaggtgaggccccgacaactgcacacgaagcagaacgctctggtggtggtcatcgtttaatggaacaggtcgttgcgcgtgctaatgccctggcagcactgagacgcgtcaagcaaaatcgcggatcacctggagtagatggtatgaccgttggtgagctgccacagtacctggcgaaacattgggaaacggtgcgcgaacagctcctcgcaggttcctatcaaccggagcccgtgagacgccaagcgattccgaaacctggaggcggtacacgagttttgggcattccgacggtccttgatagatttatacaacagtgcctgctgcaggtacttcagccacggtttgatccttccttttccgaccattcatatgggtttcgtccggggcgcaatgcgcatgatgcggtgtgtgcagcacagcaatatattcaggcggggcgtcgttgggtagccgatctggacttggagaaatttttcgatcgggttaaccacgatgtgctcatggaacgtttggcacgccgaattgccgatcgccgtgtgttacgactgattcgccgttacctcgtcgcaggaattctgcacgggggcgtcgtcgtggagcgacgtgaagggacgcctcaaggcggcccactttcgcctttgctggcatcagtcttactggatgaggtggatcgcgagctcgagcgtcgcggactggcgtttgcacgatatgcggatgacctcaatgtctactgcggttcccgtcgcgccgcgcacgatgcaatggcaacccttaaacgcctgtttgctgcactgagattgaaagtgaatgagtcaaagtcaactgtagcccgcgtatgggagcgcaaatttttaggctactcattctgggtggcgccggggcgcgtcatacggccacgaattgcacctgctgcgttggcagtcatgaaagaacgagtgcgtcggataacacgccgtaccggagggcgttcactggaggcagtggcacaagaactgcgagaatacctcacaggctggaaagcgtattttcgattggcgggcaagccccgagtcttccgcgacttggatgaatggactcgtcatcgcctgagagctgtgcagctgaaacagtggaaacggggccgtactgtttgcagagaattattggcacggggggtgcctgaaagagaagcacgtgctgcagctgctcatgcaagacggtggtgggctatggcggctcactctgcactgcagacagctcttccaaactcccactttgatcaattaggtgttccacgcttggcgggtcgttga

SEQ ID NO: 50 - DNA, 1431 bp, synthetic construct
  source 1..1431: mol_type = other DNA
atggctagcgcatggagtcatcctcaattcgaaaaatccggaatgcgtgctgatgaagccgaagctcatgcatctgcggctagtaccggtaaaggtggccggaatttgcccggtactgccgctggcgcggaggtgcgtgctgcggcaggaggtcggacgaaacccgaagcacttcggctgatggaagcggctgtggaacgttccaatatgctgggcgcgtatgaacgggtagtcaagaatcaaggggctcctggagtcgatggtctgacggttaccgaattcaaaccgtggctgcaagcccattggccgaagatcagacaggttcttctcgccggagaatatatgcctgcggcggtccggaaggtcgatatacccaaaccgcaaggtggtgtacgcacgttgggcattcctactgtcctggacagattgattcagcaagccttgcaccaggttctgcaacctctgtttgagcccgagttttcagaatcatcctatgggttccggcctggtcgcaacgctcaccaggccgtggaagcagcgcgtagttatgttgcggaaggaaaacgatgggtagttgacttagaaaaattcttcgatcgtgttaaccacgatgtactgatggcccgtgttgcccgtaaagtgaaagacgaacgcgtcctgaagctgatacgaagatatttagaggctggcctgatggaaggaggtatgaccagcgctcgaactgaagggactccacagggtggcccgctttcaccgcttttgtcaaatacccttttgaccgacttagatagagagctggaaaaacgaggacatcggttttgtcgttacgcagatgattgcaatgtgtatgtggggagtcgtcgttctggccagagagtaatggcgaaaataaccgcgtttttggagcagcgcctgaaattgcaggtaaatgctgataaatcagcggttgctcgtccatggcagcgtaaatttttggggtattccgttacctggcatcgtaatcctaaactgaaaatcgccccgagttcacgtcaacgccttgctgaaaagattcgtcaaaccttgcgcggtgcgcggggccaatcgttacgccaggttatcgcacagttgaatcccatactccgcggttgggttgcttattttcgccttactgaggagaaaggggtacttgaagagttagatggctgggtacgtcgaaaacttcgggcccttttgtggcgccattggaaacgcggttaccctcgtgcacagaacctcatgcgcgcgggtcttcgtccggaacgggcgtggcagtccgcgactaacggtcatggtccttggtggaacggtggaagttcacatatgaatgcggcgtgcccgaaatcctggttcgaccacatgcagctcgtgtcgctgcttgcaacgcagcggagattttcgttagtgtcctga

SEQ ID NO: 51 - DNA, 1443 bp, synthetic construct
  source 1..1443: mol_type = other DNA
atggctagcgcatggagtcatcctcaattcgaaaaatccggaatgaagggcggcaagcaaaagatttctcaagacacctgtcttcaagagtcacgcgcggagccggaaggctatgccggtggacagacgttcatctggatgaccgagaacaacctcactaatgcgaataagccggaatatggattactggaacagatccttagcccgactaatttaaaccgggcatataagggagtccgttctaatcgcggttcaggtggtatcgacaagatggaagtggagagcctgaaagattacctggtcgacaacaaagaaacacttatccagtcgatccttgacggtaagtatcgcccaaatcctgtacggcgtgtagagatttccaaggagaaggggacaagaaagttagggatcccgactgtggttgaccgcgtcattcaacaagccatagcgcaagtactgagtccaacctacgagcgccagttcagtgagaactcctatggcttccgtcctggacgcaatgcgcaccaagcattaaaccgttgccgcgattatattacggacgggtatatttatgcagttgatatggatctggaaaagttcttcgacacagtaaatcagtcaaagttaatagaggttctgtctcgtaccgtgaaggatggtagagtagtttccctgatccataagtacctcgatgctggtgttgtgattagaaataagttcgaggaaactgagatgggagttccgcaaggtagcccactctccccggttttaagcaacatcatgctcaacgaattagacaaggagttggaaaagcgcggtcatccattcgttcgatatgctgatgacttaattatattctgcaagtcgaagcgaagtgctgaccgcactttggcaaacaccgtcccgtacatagaaaataaactgttccttaaggtgaatagagaaaagactacgaccgcgtacgtgtcaggtatcaaattcttaggctacagtttctacgtttacaagggagaaggacgccttcgagtccaccccaagtccatagccaagatgaaggaacggataagaaagttgactagtagatccaacgggtggggatacgccagacgtaaggaagcgctgagacaatatattactggttgggtgaattactttaagcttgcggacatgaagaagctgctggttagcgttgatgaatggtatcggcggcgtctgcgtttagtaatttggaagcagtggaagagagttcgaacgcgtggccgttaccttatgaagttaggcattgtgaaacatcaggcatgggagttcgccaacactcggaagggttactggcatacagcaaagagcccaatcctcaaccgcagtgtgacgtcaaaccggctgagacaggcggggtacgtgttctttgtagactattatcgcgttgtgaatgggattaactga

SEQ ID NO: 52 - DNA, 858 bp, synthetic construct
  source 1..858: mol_type = other DNA
atggctagcgcatggagtcatcctcaattcgaaaaatccggaatgcataatttcaagagtcgtttcgaattaagcaaggggaaatgggtctacattcaaatcgaggatttggcaaatcatgcaaaggaccacatccgacaaatccgagacttgtggaccccgccagagtacttcttccatctgcaaaagggcgggcatatcgcagcactgcggttacatacgcctaatgagtggtatggtaaagttgacttgtcaaaattcttcaataatattacacgtcaccgcgtcacccgcagtttgaagcgcatcggctactcatttcgcgacgccgaggagttcgcagttgcttcaactgtgttagttaatcatgctacacgccggtacgtcttgccgtacgggttcgtgcaatcccctctgcttgcatccatctctttagacaagtcggacttaggcaactgtttgcggcgactgcacgaagagacggtatctgtaagcgtgtacgtggatgacatcgttgtcagtgcagactctgagcgtgacgtggcggaagctttaagtaatatctatttggccgcgatcaactcacggttccctattaacgaggaaaagtcaagagggccgagctcaactttgaacgcctttaatatcgagttggacatgcacgaattagagatcaccgctaaacggtacgagaagatgtgcggggaagttctgattaacgggacaggccaggtttcagacgctattttgaattacgttcagaccgttaatccggctcaagccgaacagatgcttcgtgacttccctggcgtatttggaaccctcagcagtagtgcaagccgctacgatgcttcttga

SEQ ID NO: 53 - DNA, 813 bp, synthetic construct
  source 1..813: mol_type = other DNA
atgcataatttcaagagtcgtttcgaattaagcaaggggaaatgggtctacattcaaatcgaggatttggcaaatcatgcaaaggaccacatccgacaaatccgagacttgtggaccccgccagagtacttcttccatctgcaaaagggcgggcatatcgcagcactgcggttacatacgcctaatgagtggtatggtaaagttgacttgtcaaaattcttcaataatattacacgtcaccgcgtcacccgcagtttgaagcgcatcggctactcatttcgcgacgccgaggagttcgcagttgcttcaactgtgttagttaatcatgctacacgccggtacgtcttgccgtacgggttcgtgcaatcccctctgcttgcatccatctctttagacaagtcggacttaggcaactgtttgcggcgactgcacgaagagacggtatctgtaagcgtgtacgtggatgacatcgttgtcagtgcagactctgagcgtgacgtggcggaagctttaagtaatatctatttggccgcgatcaactcacggttccctattaacgaggaaaagtcaagagggccgagctcaactttgaacgcctttaatatcgagttggacatgcacgaattagagatcaccgctaaacggtacgagaagatgtgcggggaagttctgattaacgggacaggccaggtttcagacgctattttgaattacgttcagaccgttaatccggctcaagccgaacagatgcttcgtgacttccctggcgtatttggaaccctcagcagtagtgcaagccgctacgatgcttct

SEQ ID NO: 54 - DNA, 2982 bp, synthetic construct
  source 1..2982: mol_type = other DNA
atggttcagaacacggagttagccgactgtattaacattacccttcaaaccaacgatagccgattcatcgttacttcctataacatgcatggattcaaccaagggctcgagggaacaaaggagatgatcaataagctctaccccgacgtaattgctttacaagagcactggcttactccgactaacttggatagactcggagaggtgtcaaatgattacttcttcattggttcctcttcgatgaacgacgtagtgacagcaggaccgttattcggccgccccttcggcgggaccgcaatactgattaataataaactcgctaacgcaactgtgaatgtggcttgcaatgataaatttacagcggtattgatctcagagtgtttagtaattagcgcctacatgccgtgcgcaggcacatgcgatcggatgtccttatattcagctataatcagcgagatacaggcattaatcgaattttaccctcaatacaaacttatactgtgtgcagacctcaatgttgagctggatgccccgtcgtcagttgccgacttagtaaataatttcatttgtcgaaataacttacgccggacggagcttgcgaaccccactgcatcacggtttacgtatatgcacgagagccttcaagcggcgtcctacatagactacatcatttcgtccgacgcattagactctatcgcgttcaatgttctcgatttagatattaacctttcggaccacttgccaattatgtgcgtgtttatgtgtaaaatgtcagcatgttattcagacccgtcacaatcggtcaagagcaagagccatgttgatgatgtcagctattttcgttgggatcatgccgcgcttcatctttactacgagcaaacgagagtgatgatggaaccgattctggacaagctgaacatttgtgtgaactctgtatgcgacgaggatgcttttattgaccactctaagctggaagacatatactcatctgtggtcgccgcactgacgaccagtgctaacttgtgtatacctaaaatcaagcataatttcttcaaattctggtggaaccaagagcttaatgaattgaaggcagcggctgttacatctgcacgtgcctggcaacaggtgggtaagccgaagcatgggagcgtatatcacaagtatcaaaaggacaagctcctgtacaagaagtgcattcgtgagaaccaaaagcaggaaagcgttagttttactaacgagttacacgacgcacttctcagaaagacaggtaaggatttctggaagacgtggaacgccaaggtgggcgtgaagcggaagtgtattgaacaagttgatggaatcgtggacggggcgaccattgcgtgcaacttcgctaagcattttgagaagatctgtcagcctgcgactgctacattcaacactaccatgaaggcaaagtttgaggaaatgcgaagtatgtattacacgccattctgcgacaagacggtagaattcgatgttcaattaatcggaaagatcatctctaacatgagctgcgggaaggctgccggactggatgagctttcagctgagcatttgaaacattgccacccgatcgtgatcatcatcttatgcaagcttttcaatctgtttgtacacgtggggtatctccctctgtctttcggtactagttacacagttccaatccctaagcaaaatgggcggtcacatgctctgacggttaacgactttcgcggcattagcattagtcctgtcatatctaagattttcgagcatgctattttcgcgcggttcggagattattttagtacgagcgaccatcaattcggatttaaagagagtctttcctgtagccatgctatttactgcgtgcggaacgtgatcgaccactacgttaagcgcggttctacagtgaacatctgcacggttgatataagcaaagcgttcgacacggttaaccactttgttcttttcataaagttaatggaaagaaagttgcccgttcagctgttagacctgttcgtgctttggttcagtatgagcgaaacctgtgtgcgatggggtgctcatgacagttacttctttaaactcaaggcgggagtacgtcaaggtggtgtcttaagtccgtacttctttgcagtgagcgtcgacgatgtcgtcgataaaatcgctgcgtgcaatgcaggctgttacattaataacttatgctctgctatattcttatacgcagacgacatcatactcctgtctcccagcgtctgtggtttacagagattgctgggcatatgtgaggccgcaataacagaactcgacatgaagattaatgcttccaagacggtctgcacgcgggtcgggccgcggttcgactcggcatgtgtaggtccgaacctgacttccggcggtcaacttaattgggtcaagacgtgccgatatttaggtgtccactttgcagcaggtcgctcatttaattgttcatttgaggaagcgaagaagaagttttacttcagcttcaacagcgtgtacagtaaattaggtcgcttcgcgagcgaggaagtaatcttgaacctgcttaacgtgaaatgcgtttcagcaatgttatacgccacagaggcctgtccagtactgagccgccataagcacagtttagacttcgttattacgagagtcttcatgaagattttgcgaacaggaagcccgcaagtcgtagcggagtgccagaagtatttcggatttctgccagttagctatcgtattgacatccgtagcgcgcgattcctggaacgtttctcctcatcagtaaacagtttctgtatagcttttggtgatagagcccgaaaacaactgtctgacatcgtatgtaaatacaatataagtgacggattatcatctggggtgcttagaaccgtaattaatcggatatacttccaatacccatacgatgttccagattacgctctcgagcaccaccaccaccaccactga

SEQ ID NO: 55 - DNA, 2964 bp, synthetic construct
  source 1..2964: mol_type = other DNA
atgaccattacaaacgacgcggcacaacttgtagttatctcttataatttacacggtttgaatcaaggtctgccggggatacgggagtttatgaccgagctgaagccggacgttatcatggttcaagaacattggttgacgtctgacaacttaaacaagttaagcgatattagtgacgagtattttgtgataggatcctcggctatggacgcgcgagttagcgcgggaccgttatttgggcggccttttggcggtactgccgttttcatcaagaacaagtacattaacgtgacagtaaatttagtaacttccgaacgttacgtcgtgatacagctttgtgactggctgttgataaatgtttatttgccttgcattggtacttcaaaccgcatcttattatactcagacacgctgtgtgaactccaaagtataattcgagcacacccagagtgcaactgtcttgtcggtggcgactttaacaccgatcttaacgacactcgaagtcatactgcgaacacagtcgtcaacgggttcatcgctggttgtaacctgcatagatgtgactgtttatttccaacccgacttaagcacacatacgtgaatgacagtatgaattgttactcgacgattgactatatgcttagcagcaacccagaaaagatcgttgcgttcaacgtattggacatcgacctgaacctgagtgaccacttgcctattatctcaatatgcgtattcgactgtaattgcaagatgaataatccaaagcctacctcgacagaaaatgttacacactttcggtgggaccacgcccctttacaagactactacgagcacacgcgtttgggccttcaacctattctcgcagacttggacgaattaattggaaacaagttgtcgtatagcaatgttgactttctgtgtcaagtggattgcatctacaaccgtgttgtgtctgtgttgcaacagtgctcacacgcctacgtaccgaagcataagaagaacttttacaaattttggtggaatcaagaattggacgaattaaaggataaagctatagctagttgcaaaatgtggaaggacgcggggaagccacgacacggggcaatacatgcgaaataccgccaagacaagctgctctataagaagcggatccggcaagagcgcgttcaagagacgagcagctttaccaacgagctgcacgatgcgctgctgaataaaagtggaagagacttttggaagaaatggaacagcaagttcgaaaataagtcgaataagatcttgcaggtgggtggaacaagtgacacagccactattcttaataattttgctaagcatttcgagcaagtgtgcgtgccctttaatgcgacccggaacgaagagctcaagagccgctacaaggaaatgagactcaactattcagagtctagcgtcataaatgataccaccgtattcgatgttcagttagttgagaacttgcttaccaatatgaagaacggcaaggctggtggtttggacgaattaacgagcgaacatcttaaattttgccatcccgtagtagtcatcatcctttgcaaattgttcaatttgtttgtgataaacgggcacatcccagactccttcggagtgagttacacagtccccataccgaagtctgatggccggtcacggagcatgaccgtggacgactttcgtggaataagcattagcccagttatctccaaactgttcgagctgtgcgtattggatagatattccgactatctccaaacgagcgaccaccaattcggctttaagaagcagctgggttgccggcacgcgatattttcagttcgtagcgtaatcgagcaatacatatcaaacggttctaccgtcaacttgtgtgctctcgacctctctaaagcgttcgaccgaatgaatcaatacgcgctgtttattaagcttatggaaagacgtttcccagtcaaaatattaaccatcttagagcaatggttttccatcgccgagacatgcgtgcgatggggctctgagttctcatacttcttcagccttcttgcgggagtccgacagggtggcgtcctttctccggttttattcgccatcttcattgacggtattgttaaccgtgtaaacgccacgaatgtgggatgctacaattcaacggtgtgtgtttctattttcctctacgcggatgatattttgttactgagccccaccgtgacggggcttcaaacgttgttgacggtctgcgagaatgaactgtgcgagttggacatgcgtcttaacgtaaacaagagcgtttgtatgagatttggcgcgcgttttaaagctcactgcgcaaacctcgttagcgtacagggtggcgcgttacagtgggctagcagttgccgatacctgggagtttacttcgtatccggccgagtcttccgatgctgtttccactccgccaagtgcaatttcttccgtgccttcaacagcgtctactcgaagattggctgctgcgcatctgaagaggtcatcctttctttgctgaaatcaaagtgcctgccgtacatattatacggtgttgaggcttgcccggtgctgcaacgggacaagcactcatttgattttacactcacgcgaacgctcatgaagcttttcatgactagctctcctgtaattgttaacgaatgtcaaacccaattcaaccttctcccattacgttatcagatcgatatccgcacggtgaagttcttaaaccaatatatcatttcccgtaacagtatctgtatgttatataagtcacatgcccaatcggtactggacacaatatttggggtctacggaaataacgtcagctcacttcacgaccttcataatattataaacgagcatttctacgacttcaatccaagcacacttggctataactacccatacgatgttccagattacgctctcgagcaccaccaccaccaccactga

SEQ ID NO: 56 - DNA, 3114 bp, synthetic construct
  source 1..3114: mol_type = other DNA
atgtacaaccttcgcatcactagctacaattgtcggagtttgagcgctcttaaacgtgactttgtgaagacattactcgtctcatgtgacattcttttccttcaagagcactggttatcagatgatcagttaactctccttggcggcttagatgagggctttacctttacgggagtaagtggcttcggaaataaggaaatcctgagcggcagaccatatggtggctgcgcaatactctggcgttccgctctcaaattcagcgtggacttcctgtcagtcaactctcgccgcgtttctgccataaagctttctaatgattgctgtaacttgctcctgattaacgtgtatatgcctttcgaggactctgattgtaacgtgaacgaattcttcgatgtgctctttgacatagaatacttgttggtaaataacgtagattgccattacctgatcggcggcgacttcaacgtagatttgtctagaaatactgtccacaccgcattattgcgttcgttcagcgataacaacggcctgttatacgcttccgatttcgatggtgcgaacttcgacttcacatatcaatttaatatgtcgcggttcagcctgatcgacaatttcatgctgtcttcgttcttgttccataacatgcttcaacgtgcagatgtggtccacgacgtgaacaatttaagtgaccacgagccagtaaccatagagttatgtatgtctgtgactcacgttgatatccaacggtctacggtgccgcctttacagaaagtctcttggaccactgccgccgagagtcatgtaatagactaccgcgaggaactggtagaccgcctcaaaaccgttgtaatgcccatcgattcattaacgtgctgtgaccgtcgctgttcggtcgcgaagcaccgttccgatattgccgagtacgctaacaatattgcggacgtttgtatcaaggcgggacttacgacgatacctttgatgaagccggggcaccgtgcaacgcctggtttctccgaacacgtaaagcctgcgcgtgacaaatcaatgttctggcatcaattgtggttggagtgtgggcgtccgcggacaggacacgtagcagactgcatgcgccggacccgcgccgcctatcactatgcactccgtagcgtgaagagaagacgtgacgagataactcaagagagattcgcgacggcgttactccataacaactcgcggaatttctggtccgaagttaagaaaatcagagcaaatcggatgacatacaacggcgttgttgatgggcacagcgacgcaactgacatcgtccggatcttcggagaacgttaccgcgacctgtacacttccgtaccatacgatgagtgtaatatgcacgagatcaatgacactatagacgagcacataagtgacgatgacgacgcagcctttgtcgtgagtcttcgggatgtttcggacgcgattgcgcatattaagcattataagaacgacgtcgacaatatgctgacctcagatcacttcattaacgcaccatctgtattgcacgtacatatttcgattctgttcagtgcgatgttaatgcacggttgtgtacccaacctgttaatgtatagctctatacgtcctatcccgaagggacacaacctgtcgacatgtgacagcaataattatcggggcatagcaatcagttcgatcttcaacaagattttcgataacgtggtgctcataaagtaccgccatctgctgtcaacttgtgacttacaattcggatttaagaagaaacatagtacgcagatgtgtaccatggtgcttaaggaaacgctttcttactatctctcgaaccgtagtaacgtgttctgcacattcctcgatgcgacaaaagccttcgaccgtataaactactgcaaattattcaatttgttactttgtcgctctctcccctattgcatcatccgcgttttactttccctttatacaaacaattacgtgtacgtgagctgggttggttcaaacagttcatccttcagagcctgcaacggggtcaagcaaggtggggtactgtcaccagttttattttgtttatacatggacgggcttttgaataagctctcacatgctggggtcggctgttatatgggtgagatgtttgttggcgcccttgcatacgcagacgatatcgtgctgattagccccacgccttcagctatgaacaatatgctttccatatgtgacgagttctccatcgagtataatgtcctgtttaacccattaaagagtaagtgtatgtatttctaccctaagagccgctctacgctcttgctgcatcgttacaacgtttgtgacctgcaattctcgataaacggcaaggccattgagttcgttgactcttataagcacttggggcatgttatttgttccgacatgactgacgacatcgacatcagcgaaaagcggggcgtatttattggccaagctaacaacatcatttgctactttgctaagttgtcatctgccataaagtatcgactgttcacgtcgtactgtaccagcttctttggctgtgagctttggagaatatctaacgattcgttggatagcatgtgcacggcgtggcgccgagccattcggagaatttggagtttgccctataccgctcacggacgcttcttgcccgttttatgcaactcttacaacattttcgaccaattctgtgtacggattcttaacttcattagaagatgcctctctgatcaaagtagcttgctggtgcgaagcatcgccacccaggctttgatgttccatcacgcgcgatctcctttaggatataacttcatatattgtgcgcgtcgttattgtttctcattgactgactttgtgaactacaacaacttcgtgtcaaacgtcgagaagctcaattgcactcaaaacgacgacgacacgatagcaaattgtcgcctgctgcgagaattgattgacttacgcgatggtgtgttgcaccttagtgacgacgtctttcttacaaacagcgagctctcattcatgatcaactgcgttagtacactgtacccatacgatgttccagattacgctctcgagcaccaccaccaccaccactga

SEQ ID NO: 57 - DNA, 4368 bp, synthetic construct
  source 1..4368: mol_type = other DNA
atgacagaaactagccatgcacctgtcttggaccaaacatgcacccgttaccagagacacaacgtccacgagcaggctatgcaatttgtgccaccaaaccgtgttgtattcagcttctttggcccttgtgacatcgtatgtcaccgaggcgcttttctttcagtcacctcatattgcaaaacactctcactcttgcagcgggcacacttcactagacaccggaataaggctaccggccaaccgttcctgctgttgtccgtttataagcaacaagacttgcaaacactgacccaacaggggcttgttccgtttacacagacatgcaatgaggcgctgtctcttcggggttgtcgcgtggcgccatctggtcgggtcgctttgccgtatatgatgtacccacagggtgatcaccgtaaccagccagtacacaatcaagagacacacactccccttactccaggccttgatgcgaacccgttcgcggccttatcggtggagcaacaaacgtgtggagctagtacgtctcgcacctcgttaatagttgggtctatgaacgtaaacggactgtttacccagactatgtcctctaagcatcaagatgtttgcgcatttgtttctagccacacagtcgacatatgttgcctgcaggagacatggtgcgacatgagcgagactgagtactcttctatgttagccagttgtggttatacgtcattcggtgtctctggatattctacccgtacacggacaggcggcggtaccggcatcctggtggcaacttctatctctcatctggcccacatgctccactgggagtcacagaagcacacccagaccacctggatcaaactgaacagcactcagtacaaagcgagaaagaccgctgtaggcagcgtttatctccggccggtcagccacacccgaccaggcgacgctgagcggtacgtgaccgaactcattgccctgcgcgatgacattacatatcttagcgagcacaacttcgacgtcgtcctgtgcggcgactttaatgccagaataggtaactctcaggtcagcccgcacgttccgcaacacaatgagcaaacgcgcaatacgcagggtgatcgcttggttactcttctggagcagtgcaacatgttcgttatgcataaccacaccagtttcagcccaacctgcatgcatgcaggcggctcatctgtggtagactactgtatcacgaactcttcatgcagagcacgttgttcagacgctgcaattaacttcgcagggccatgtgtgagtgatcacgctttattgactatgagcttatctcacgcaactagaacgcgtcatcgcaagacgcgccgtgcccacgcaaagttatggaatcgcagagctatgcatgatgagcaaacaatcgcccaatttcacgctacactggaaaccccgcttacacaactttccgacttcatagatgcaagcaccctgagtccccaagaagcaacggataccttcactagtaagttaactgccgctctgcgtgacgctggcgccacttgttttggaactcaccaagttcggccacatagagctcaatggtggaataaagactatgcgaaccttagacatgagtgctggaacctgtacgagacacatcgacgtagtggatccgaggcagaccgtgctgcgtatgaggagaagcgcgcggcaaagaacaaaatgaaacgcaatctgaagcgcgcgtacgttaaggcccaagccgggcacgtgtcaaagttatgtacaccggggcttgtcagcaagactagttggggcgctgccaaaagactcttaaagttagttacacgtggatcatcagccaccgagcatactctccccacggtcattcacgacggtgctcccactgacgatctcgaggctgttatgcaagtcttctcctctcactaccatacggaaatgaaccctgtccccagtgcgacattttgtgaggctacagacactcttgttaaggacaagctgcatgcgtggcgccaggcggagcctgagcatattaataatatggacgatgaattcaccataaccgagttagatcgtgcgcttggttccatgcataactggaaggccgctgatcacgacggaatgataatcgaactgttgcgcgcaggcggacctgcattacaactcgtggtacttaaaatcttgaatttctgttggacgcacgagacgattccaacaaactggaaattaggcaccatcatcagcctgtacaaagccggtaagaaagaaaacccctctaactaccgtgggattacgctcttaagcgtagttcggaagttgttttgtacgttattacggagccgtttacaggataacatctctctccacgagtctcaagccgcgttccgtgcaaaccgtgggtgtatggaccacgtgcacacgcttgcgcgtatcgttcgcgccgcgaatcgtaaggacattccggtgtacgctttcttccttgatatacgcaaggcgtacgacacggtttggcgcgacgggctcatttataagcttctgcaaaagggagtgactggtcggttagggagagtcatttctcaggtacttaccgatacgcaatcccgtgtgcgctttcaacaccgtgaaagcgcttatttcccgcttacattaggtgtaggccaaggggatcctctttctacaattctctttgacgtgtttatagatgacttattagaggagttacatgcacgcccggtgcaccactgtatccctgtagacagcccacatctggaccgcatcgccgacctcacttatgcggatgatgtgaatgcgttgagcttgacccccgagggtttgcagggccacatagacacaatcgacacttggttgttccgatggcggagtcaaccaaatgttagtaagtctaagacgatggtcttcaacccgccgcagggagttccgccaacagtatttactatgcgtggttcaaacctcgatactgtccaatgctttaagtacctgggagtattctttcaatcaaatgggtcctggaccgaccacactcagcacgtacgtacacagatgaataaagccgtgggcatgtggcgtcctgttctccggtgccactacctgcccgtagcagcacgcctgcgcattatatacgcgttcgtctacgcgcctgcgctctacggcgctgaggtctggatcgcacctaagtcagaattggagaagttagatactatttgcaagagtgcgatccgtacagtctttggcttgcaccaattcgactgtcgcgaggagatattatttgccgacaccggattgctcccagtctcgagccttatcaacgcagcgaaactgtgttggttcactaagcttcttaacatgcctgagtcccgtttccccacggccgttgaggctgtgacgttacctggagatacacaacggggccgggttgccggtggcgatttcgggacgcgtattgccgacattaccacggaaattcgtcgttatgcgcacatgaaattagtgatgcaagacccattggagcacgccccgcaccgccgccggcgtcccatacgcgtttcaaagcgtctttctcacaccaagccggacgcgcacctctacgaaactattacatactcgtctctgacccgtgagcgcgtaaaggctctcttgtggaaggtgtacatggaaaagtgtttcgaacggcgcggccgcaaggatggcgtaaccggtgagtggatgcgctctgtcattaagcacgagctgggttcattggcgccgtttcttcacagcgttgagtgtgggttagtgcgtgttctgatgagtgctcgaagtcgtttaagtccagttttatgccggccaccgcaggacaaacaccgggagtactcacaagcagccggcgcctttaccacatacatatcgcgtgccccgattggtgctgtgcttccaagtccaccggtagcgcattgtgtacgtcacgtttcggctgtcgccgagctcgtacgagaactgttagcccaacctggtccttacgggtgcggtagttctgtgtcgtggtctgacatcgcagttgtactggtcgtgggaatcgacggcgttggtggcgcgcctggcgttttgggagtcgtggcccgtccgtcccctctttcagaacttgctcgtgtggtcgcattctgtggagcgagtcccagactccgacaccatgttgcttggatgtcacggagcgttcctgtcgcgggtgtcgtaaacgttccagtggacatggctatgttgcagcggacgaaggccggccggcaagtcgctccccaaggcaagcacggagcacactcttcgtacccatacgatgttccagattacgctctcgagcaccaccaccaccaccactga

SEQ ID NO: 58 - DNA, 2970 bp, synthetic construct
  source 1..2970: mol_type = other DNA
atggcgataagctacacatcaaacaattcggttcggatatgctcttttaatatgcatggctttaacaatggggtgagcatgacgaagatcctttgcaacagctttgatgtgatccttttacaagagcactggcttctgccgtctaacctgtctaagctgggcgatatatcacacgactttacacaccattccgttagcgcgatgacaacaaagatttcagagggcattctgtacggccgaccgtttggtggagtatcaatcttgtacagaaactctcttacaaagaatatcagcattatagatgcggacaaggaagaagggcgctacgttactataaagctgaaatgtgtcaataacgagttcattacggttacgaacgtgtactttccaaccatttgtgcatacagtgactacatagtaaataccagctctatcatggcctacttagacaaccttttcgcaaacgaggtatcctgtcaccatgttgtggcgggtgatttcaatttcgaatatcggaacgataatatcggcttcgacttgttccgttctcttgctgtcgacaacaacttgatatgctgtgatgacctgcatcttaatcaaaatataaaatttacgtataagcacgaaaccctgcctcaacaaagctggcttgaccacttcttcgtttccgccgggctcacaagttctattgtatactgtgataccatagataacggtagcaatctttccgaccatcttccgatctgctgtacaatcaacgtctcactgagcgactccaactgcaccccgaagctttctaaggtgtatcgcgaccgctgggacaaggcagatctgattaagtactactatcagagcgggattcatctccaaagtctcacagccccgccacacgtgacgcaatgctccacacattgtcagaaggctcaacatctccaggatattaacgcgtattacgagcgtataattcaagctctcaaagcttcggcaataggctgcgttccccggatgccggtaaactgcttgaagcactggtggaacgacgatctcacgcgacttaagaacttatctatcgatatgcacaacttgtggcgccaggtcggatccccgcgcaatggcattatcaatgaggcccgcctgaaggcgaaattggattataagcaggccatacgccaggcaatgctcgattgcgagaacaaagacgccgatattattaataacaaatttaaccagaaggattcccgtaatttctggaagtgttggggcgcaaaatatcgcaagaaagtgaacaataccgcgtgcatagacggttgtacggacaattccaccattgccaataagtttaaggcgtactttcaaaacacctacgtggacagcacatgtgacgtgaatgcctgtatggagttcgataagctcatgagcgaccactctcacctgcagcacaatgacatagtggaagaaattcgtattgaggatattgagaaatgcatcgagcttctcaaacccctcaagtcagcaggccatgacgacgtcgccccagagcatctgatccatagtcaccctagtctgtgcatgcacttgaaactcctgttttcaatgatgttaaatcacaactatgttccggatagtttcggaattggaatcattatccctgtagtgaaggacaaacgcgggaatctgaacagtgttgagaactatagacccatcacgctgtctccaatcatatctaaggtctttgagagtttcgttctgaaccgcttcgccaagttcatgacttgcgacccacttcagtttggattccagcgctcggttggatgcaataacgccctgttcgcgatccgccaagtaattcaatattttaacgatcgtgattcaaacgttatggtcgcatcgctggatgcctgtaaggctttcgatcgtgtcaatcactttaagctgttctccactctgctgcaaagaaagcttccattacacattataaaggttttgatcaattggtattgcaaattgatggtgcaagtccgatggaacaactcattaagcgatttgttccatgtgaaatcgggtgtacggcagggtggaattttaagcccggccttgttcaatgtgtacattgactgcgtgatcaacaagctccgggctagccaacttggttgccacatcggatccctctatattgctgtggtcctctttgccgatgacattctcctcttgtcatcatctttcatggaacttcaacgtatgatagacctttgcgtggagtcaggtgacgaaataggtttaaagtttaatgcagcgaagtcacactgcatgattatcggtccgcacaaggtcctggttaagcccgatatgatgatggggaactcacctaccgcctggtcggagacaattaagtatttaggggtgtatattcaaagcgataagaagttcactgttgacctgtcgctggttagacggaaattcttcgcaagcgtcaattgtatactgcgtaatgcggccttcacctcagacattgtgaagctggaattggttgagaagcaatgtttccccattttactgtacgggttacaatctttcgacttgaaatcgtcggtaatcgcaaacgttaacgcctgggtgaactccgtatatcggaagattttcggttaccataaatgggagtcagtgaaggaatgcatttacctgttgggacgtttggacgttttccacacaattaagctgcgtcgtattaatttccttaagaacatccaaaagtgtaacaacgaggtggcacgctcattattcaactacgtcctgcacacgcgtgaatttcaatcttgctgcacgttaccgaacaatagcgtcataaatatatccaagtcgtgcgacaatgtacgtaaggcggtctttaactccttccaatcgaaggtggtgggtcactacccatacgatgttccagattacgctctcgagcaccaccaccaccaccactga

SEQ ID NO: 59 - DNA, 2925 bp, synthetic construct
  source 1..2925: mol_type = other DNA
atggcaagtcagactttagatacacaaacggcaaacaatgtgtgtaactctgtgtcggttatgtcttataatatgcacggctttaatcagggttattcatacttgaacgatatctgcatgaagcagacgtaccaagtgatccttattcaagagcactggctgtacccagctacactgcataagttagccaatattagcgagcactactccttctacggagcgagtgctatgcaaaccgccttagatgcaggctttattcgcggacgtccctacggtggaaccggtatactgcttcacaaagatattgctaaatactgcgttgagagttgcgcattcgagcgagtcgttggggtacttttaggagactttcttttcgtcaatgcatatttcccgtgctgggacgggtccgttatcaacttggacgccgcaaatgaactgttggcaaacctcagtaatgtattggatagctaccaagctaaacacgtcgtattcggcggtgactttaacgcaaaccttacgaagaattcagtttactctaacatgattctcgagttcatgtccgagcatcagctggatatatgtcgtaaggtgctgttcggatcttccaatgtcatattctcggacacctacgttaacgaggcacttaacgcctcttcgtgtatagactttatctgtattagcagcggactgtcaaataacgtagctcaatacgatgtacacgacgtcttcaacaaccacgcagaccatctgcctgtcagcctcaagctgtgtttaccagtcagttccatcctgtataactgcatagcaagtggttcttcagcttgcatgttcaatgaggcaaagtctcgggatactcaatcttgcgattctaagggcaataagctgcgctgggacaaaggcaatacgcaattatactccgaccttacctatgctcagttgtttcccatttacgaaaccttacattcaatcgatgtagaaagcagcgcttacaccgctcaggaacaccgtagcctgataaacgaaacctacactcaagttgtgagcgcccttcattatgcggcaaatgtttccattcctgcaatggcgagtcacacattaaaacactggtggtgtagcgacctgagtgaattgaagaagaaatcgatgttatcacacaacgagtggataaacgcgggcaagccacacgcaggccgcattcacaccgccaagcagcaaagcaagttgcagtacaagcgtgcaatcaagcacgcgaaggcaagcgctgaaaatagtgtgagtgacgagctgcaccgtaacttgtgttctcgcaacaacgttaagttttggaagacgtggaagaacaaaattaaacgaccgggtaccgagaaattgtacgtggaaggttgtaatactgacgctaaggccgctgagctgctcgccgactatttcaacaaggctacgagcccgaacagcaacgaatacaacaacagcaagagagttgagttcgagaagagtttcgcctcttacgcgattcaagcgaacgacattgacatctcagccggccttgtagagtcggctgccttgaagatgactgccgggaaggctgctgggattgacaatatctcaattgaacatgtacactactgccacatcgtaatttacagcttattagctaagctgtttaacttaatgctttgtttctcttgcgtgccggatgccttcggttatggggtcacaacaccgatcccaaaagaggaatctcacaagaaaatacaccctgttgagaacttccgtggtattacactgtcccccgtgctttcaaagttgttcgaacactgcttattatctgtgttttcggactttctccaaacgagtgacaaccaatttgggtttaagcggagcacgggatgtacccacgctatatacgctttgcgtaaggtgactgagttctttatatgcaacgagagcaccgtgaacatgtgcttccttgatattagtaagggattcgacaaggttaacaacttcgagcttcttttgaaacttatgaagcgcaaggcaccctcttgttttataaagctcttacacgactggttctccatctcttacggttctgttaaatggaatacatcgcgaagtgactggtataaaataggtgcaggagtacgccaaggcggggttttaagtcctattttattcgccgtatacgtcgatgcgatgctggagaaggtcaagaccatgggatgccaatacaaaagtctgtgcacgggtgctttcatgtacgctgacgatcttgtgttattatctccaagtatttacgagttacaacacatgattgccttatgccgaaacgagttacacagtttagacctgaagcttaatgttaagaagagcaaggcgttgcggatcggcaagcggtacaagtgcaaagctctccctcttatgatagacggtcaagcagttcattggtctaatgaggctcgttacctgggtatagtgattagatctgcatgtaagttcaagtgtaacttcgatccggctaaggtcaagttctataaggcggccaatacaattttggccaagctcggtaataaatgcaatgtcaccgtcacattgtatctggtcgcggctgtcgcccttccgccccttatctatggtatagaggcattgacgctcaactcaagcgagttgaactccctgaaccacccgtggaataactgtctgggcaaaatgttcaacacgtttgacaaggaactgataaagaattgttgcgatatcctgggttacgaatcctgtcagaacgtgtatgttaagaaggtggagaagttcttaagaaacatgaagtttattgataacgccatactctctgcgcttaacacggaacaagtcaacgtctacccatacgatgttccagattacgctctcgagcaccaccaccaccaccactga

SEQ ID NO: 60 - DNA, 2982 bp, synthetic construct
  source 1..2982: mol_type = other DNA
atggcgaactgtaacggtacgatcattagctataatatgcatgggtttaatcaaggcagtgagttattgaagtcttactgtgcgaatagttcagtggatttcctgctcatacaggagcactggctttctcctgacgctttgcataagatcgatgacattgctcaggactacttttgtttctctgtctcgagtatgacggccgttttagagagcggacctttgcggggcagacccttcggcggcctgagtatccttgttaagaacatgcatcggcaattctgcagtgtagttgccctgaacgagagatatattgctgtccagtacaatgatatcttaataattgacgtctacttcccgtgtgtatcaagtctcaaccataaggatgagacaatagatctgttagtacaactggacactttggttaatcaatctgtggttaaggatatcatcataggcggcgacttcaactgtaatctggagatagagagttggtctagcaaggtaatttgtgacttcatggaagataactctctttcatcatgcaacaagctggctgtgaatgtgataggtgctgagtacacgtacagtaacgaagtattgggtcattatagctaccttgactactttgttgtcagcaattcacttacgagctccgtcatgagccttgagatttgttcagacgacttcaacctctccgaccactcaccggtatgcatcgaagtgtgcaatataatcagcgattgtgagacaacggtcttgtgctccaacaagggcggcaagagtacaaagattccagacacttgttaccagaaccgttgggaccatgctgacattgtggcttactatgaactgaccagactcggtttgcaaccgatattagcttaccttaatgattgtagcaacagtttcccacctcagagtgcaatcgattacaagagatacatgcgtgcgtgcatagacttagcttataatgacgcggtgtccgttctggtagacgctgcaaagaacacagttccgcgctttaaggccaacgcattaaagcactggtgggaccaggagctcagtgaattaaaagagaaagcctttgcgtctcataaactctggattgaagcgggaaagcctcgtaacggatgtatatttgacatccggaaggcagacaagtacaaatacaagtgtttaattaaacgtaagcagcttgaagttcgcgacagtattacgaatgatctccatgatgcactcttactgaaggatgctgaccatttctggaaagtgtggaagtcaaagtttcccgcgaagcggccgaataagtacgcgctgattgagggtaacagcgatcccaacattattagtaacacgttctgcaactatttcagtgagatttgtacggcgcacccaagtgttaatgccagttcgaacaatatcttcttgaatagattcgacggatacataggtgacctcaatcaatcaaccaagaacatcatcagcatcgatttgatagaggactttatactttcgatgaagcgtggaaaggccgcaggcctggattcccttacaatagaacacatccaatttgctcacccagcaataatctgtatactgaagctgctgtttaactacatgttagagttcggaattgtgcccgagggcttccggaacggccttgtcattccccttccaaaagaggattccatcaagaagaacgtgaagctggagaacttccgctgtattacaatttctcccgttatctccaagatctttgagcactgtctgatgcgcctgttcgcaaagtatctgaactccgacgatgcgcaattgggtttcaagaagaagtgcgggtgcagtcacgcgatatactgcgtcaagcaagtggtcgattactatgtacgcggtggaagtactgtgaatgtgtgtacacttgacatttcaaaggcgttcgacaaggttaacttattcgtcctgctgtgcaagctcatggatcgtaatattcctaactacgtcataaatgtgctttacgattggttcagcaacaattatatcaccgttaaatggcttaacatcctttccagtcgatgtccagtaaattcgggagttcggcagggtggagtgctgagcccggtcctgttcgctatatacgtagatgacattctcgtaaagctgcgtaagagtcgtctgggttgtacaatccagggcctgtctgttaatgcttacatgtatgcggatgatctgattatcctgagtggttcagttactgatttgcaaaagcttattacgctttcaattgaagagctgaagtgtattcacctgtctataaatcctaagaagtgcttttgcatgcgagtcggtaaacggttcaaggtaaactgtaacaatgtggtggtcgacaattattcaatccaatggtcttctgaaatacgttacctgggagtttacttaacggcgggccacgttctgaagttcaatttagattatgggaagaaaagttccattgcagctttaaattcgattatgagcaaaacgggcaacaaggctatcgacatagttcttagtcttacacaatcatactgtatcccaatcctcttgtacgcagtcgaaagcatgtgcctgaccacaacagagcgccagcgtttagggtcttcatttaataagctgtataacaagttattctctactttcgatacccagactatcgcctactgccaatactacactggctatcttcctcttgactacgtcattgatctgagatgctggaacttcttacaaaagctctctatatctaataatctcgtactgaacaagctcttccgtttgaacggtaacaatactatcgattcgttgtgcttaaagtacaactgcgaattcaagaactatccgtctcttaaggtgagtatgtgggagaagttcaagttcgtttactccctgtgcccatacccatacgatgttccagattacgctctcgagcaccaccaccaccaccactga

SEQ ID NO: 61 - protein, 976 aa, unidentified
MANCNGTIISYNMHGFNQGSELLKSYCANSSVDFLLIQEHWLSPDALHKIDDIAQDYFCFSVSSMTAVLESGPLRGRPFGGLSILVKNMHRQFCSVVALNERYIAVQYNDILIIDVYFPCVSSLNHKDETIDLLVQLDTLVNQSVVKDIIIGGDFNCNLEIESWSSKVICDFMEDNSLSSCNKLAVNVIGAEYTYSNEVLGHYSYLDYFVVSNSLTSSVMSLEICSDDFNLSDHSPVCIEVCNIISDCETTVLCSNKGGKSTKIPDTCYQNRWDHADIVAYYELTRLGLQPILAYLNDCSNSFPPQSAIDYKRYMRACIDLAYNDAVSVLVDAAKNTVPRFKANALKHWWDQELSELKEKAFASHKLWIEAGKPRNGCIFDIRKADKYKYKCLIKRKQLEVRDSITNDLHDALLLKDADHFWKVWKSKFPAKRPNKYALIEGNSDPNIISNTFCNYFSEICTAHPSVNASSNNIFLNRFDGYIGDLNQSTKNIISIDLIEDFILSMKRGKAAGLDSLTIEHIQFAHPAIICILKLLFNYMLEFGIVPEGFRNGLVIPLPKEDSIKKNVKLENFRCITISPVISKIFEHCLMRLFAKYLNSDDAQLGFKKKCGCSHAIYCVKQVVDYYVRGGSTVNVCTLDISKAFDKVNLFVLLCKLMDRNIPNYVINVLYDWFSNNYITVKWLNILSSRCPVNSGVRQGGVLSPVLFAIYVDDILVKLRKSRLGCTIQGLSVNAYMYADDLIILSGSVTDLQKLITLSIEELKCIHLSINPKKCFCMRVGKRFKVNCNNVVVDNYSIQWSSEIRYLGVYLTAGHVLKFNLDYGKKSSIAALNSIMSKTGNKAIDIVLSLTQSYCIPILLYAVESMCLTTTERQRLGSSFNKLYNKLFSTFDTQTIAYCQYYTGYLPLDYVIDLRCWNFLQKLSISNNLVLNKLFRLNGNNTIDSLCLKYNCEFKNYPSLKVSMWEKFKFVYSLCP

SEQ ID NO: 62 - DNA, 27 bp, synthetic construct
  source 1..27: mol_type = other DNA
actctgtgagcggatcttggcttagcc

SEQ ID NO: 63 - protein, 181 aa, synthetic construct
  VARIANT 37: note = Any amino acid
  VARIANT 41: note = Any amino acid
  VARIANT 42: note = Any amino acid
  VARIANT 49: note = Any amino acid
  VARIANT 51: note = Any amino acid
  VARIANT 79: note = Any amino acid
  VARIANT 85: note = Any amino acid
  VARIANT 87: note = Any amino acid
  VARIANT 118: note = Any amino acid
  VARIANT 119: note = Any amino acid
  VARIANT 120: note = Any amino acid
  VARIANT 126: note = Any amino acid
  VARIANT 127: note = Any amino acid
  VARIANT 128: note = Any amino acid
  VARIANT 133: note = Any amino acid
  VARIANT 137: note = Any amino acid
  VARIANT 142: note = Any amino acid
  VARIANT 160: note = Any amino acid
  VARIANT 161: note = Any amino acid
  VARIANT 165: note = Any amino acid
  VARIANT 181: note = Any amino acid
CTFLDATKAFDRVNYCKLFNLLLCRGLPYCIIRVLJXLYTXXYVRVSWXGXNSSSFRACNGVKQGGVLSPVLFCJYMDXLLNKLXHXGVGCYIGEMFVGALAYADDIVLISPTPSAMXXXLSICDXXXIEYNXLFNXLKSKXMYFYPKSRSTLLYNVCBXXFSIXGKAIEFVDSYKHLGHX

SEQ ID NO: 64 - protein, 181 aa, unidentified
CTFLDASKAFDRVHYCKLFRLLLGRGLPVCILRVLIQLYVGHSIRVTWAGLVSSCFTALNGVKQGGVLSPVLFCIYVDELLIRLAESGVGCYIGFSFAGALAYADDIVLIAPTPSAMRKLLAICDTYATEYNILFNAQKSNFIAFVPSSRHFLHKAMTNCVFRIGGVQIEHVFSYTHLGHI

SEQ ID NO: 65 - protein, 184 aa, unidentified
CTFLDATKAFDRINYCKLFNLLLCRSLPYCIIRVLLSLYTNNYVYVSWVGSNSSSFRACNGVKQGGVLSPVLFCLYMDGLLNKLSHAGVGCYMGEMFVGALAYADDIVLISPTPSAMNNMLSICDEFSIEYNVLFNPLKSKCMYFYPKSRSTLLLHRYNVCDLQFSINGKAIEFVDSYKHLGHV

SEQ ID NO: 66 - protein, 181 aa, unidentified
CTFLDATKAFDRVNYCKLFHLLIKRGLPASVIRILIVMYTGHCVRVAWAGLASSFFSAVNGVKQGGVLSPILFCIYMDDLLVKLHKSGVGCYIGTAFVGALAYADDIVLLAPSPSAMRKLLSVCEFYALDFDISFNAGKSNFILVMPNSNQRLRTQMNNCTFSIGGAPVVKVNSYLHLGHI

SEQ ID NO: 67 - protein, 180 aa, unidentified
AFFLDIRKAYDTVWRDGLIYKLLQKGVTGRLGRVISQVLTDTQSRVRFQHRESAYFPLTLGVGQGDPLSTILFDVFIDDLLEELHARPVHHCIPVDSPHLDRIADLTYADDVNALSLTPEGLQGHIDTIDTWLFRWRSQPNVSKSKTMVFNPPQGVPPTVFTMRGSNLDTVQCFKYLGVF

SEQ ID NO: 68 - protein, 66 aa, synthetic construct
  VARIANT 2: note = Any amino acid
  VARIANT 8: note = Any amino acid
  VARIANT 18: note = Any amino acid
  VARIANT 22: note = Any amino acid
  VARIANT 31: note = Any amino acid
  VARIANT 34: note = Any amino acid
  VARIANT 44: note = Any amino acid
  VARIANT 46: note = Any amino acid
  VARIANT 48: note = Any amino acid
  VARIANT 53: note = Any amino acid
  VARIANT 58: note = Any amino acid
  VARIANT 60: note = Any amino acid
  VARIANT 61: note = Any amino acid
  VARIANT 62: note = Any amino acid
QXSLLVRXVATQALMFHXARSXLGYNFIYCXRRXCFSLTDFVNXNXFXSNVEXLNCTXNXXXTIAN

SEQ ID NO: 69 - protein, 66 aa, unidentified
DNILVRAVVNYAIAARSKSVLGRNVMLLCRRFHLSFDDFVSGRLLLSGDIFVSHYLNSLSEAQLQS

SEQ ID NO: 70 - protein, 66 aa, unidentified
QSSLLVRSIATQALMFHHARSPLGYNFIYCARRYCFSLTDFVNYNNFVSNVEKLNCTQNDDDTIAN

SEQ ID NO: 71 - protein, 67 aa, unidentified
SSTLVKYVTRHAIDIARYNSNVGKNALFCCNYFKWQLYDFVNGSVSLNRNSFLTFCLNRLSGCEVDN

SEQ ID NO: 72 - protein, 70 aa, unidentified
PESRFPTAVEAVTLPGDTQRGRVAGGDFGTRIADITTEIRRYAHMKLVMQDPLEHAPHRRRRPIRVSKRL

SEQ ID NO: 73 - protein, 4 aa, unidentified
YADD

SEQ ID NO: 74 - protein, 4 aa, unidentified
FADD

SEQ ID NO: 75 - DNA, 259 bp, unidentified
  source 1..259: mol_type = unassigned DNA
cctcacgacggctgtagactagctattgagcgactatgggccgtctgcgagttgacttgctgttcgcaattgcagtgaccgcaaggcaacgattgcttggttagaattaggcacgcttcggcgtcaagctcgccagggagtgaggagataggctatgaagctgaacccaagcttggttcgcggctatgtcctgctcgccaccgactgctgtcgtttggcgtggtgggtctttatggtcgtgagcctgatctctaactac

SEQ ID NO: 76 - protein, 184 aa, unidentified; identical to SEQ ID NO: 65

SEQ ID NO: 77 - protein, 66 aa, unidentified; identical to SEQ ID NO: 70

(19) United States
(12) Patent Application Publication
(10) Pub. No.: US 2024/0360477 A1
(43) Pub. Date: Oct. 31, 2024

(54) SYSTEMS AND METHODS FOR TRANSPOSING CARGO NUCLEOTIDE SEQUENCES

(71) Applicant: Metagenomi, Inc., Emeryville, CA (US)

(72) Inventors: Brian C. THOMAS, Berkeley, CA (US); Christopher BROWN, Albany, CA (US); Daniela S.A. GOLTSMAN, Oakland, CA (US); Sarah LAPERRIERE, Berkeley, CA (US); Cindy CASTELLE, San Francisco, CA (US); Lisa ALEXANDER, Albany, CA (US); Mary Kaitlyn Chiu, San Francisco, CA (US); Morayma TEMOCHE-DIAZ, Oakland, CA (US); Anu THOMAS, Albany, CA (US)

(21) Appl. No.: 18/598,724

(22) Filed: Mar. 7, 2024

Related U.S. Application Data
(63) Continuation of application No. PCT/US2022/076057, filed on Sep. 7, 2022.
(60) Provisional application No. 63/241,954, filed on Sep. 8, 2021.

(51) Int. Cl. C12N 15/90; C12N 9/12

(52) U.S. Cl. CPC C12N 15/907; C12N 9/1276; C12N 15/90; C12Y 207/07049; C12N 2800/90

(57) ABSTRACT

The present disclosure provides systems and methods for transposing a cargo nucleotide sequence to a target nucleic acid site. These systems and methods may comprise a first double-stranded nucleic acid comprising the cargo nucleotide sequence, wherein the cargo nucleotide sequence is configured to interact with a retrotransposase, and the retrotransposase, wherein said retrotransposase is configured to transpose the cargo nucleotide sequence to the target nucleic acid site.

### CROSS-REFERENCE

This application is a continuation of International Application No. PCT/US2022/076057, entitled “SYSTEMS AND METHODS FOR TRANSPOSING CARGO NUCLEOTIDE SEQUENCES”, filed on Sep. 7, 2022, which claims the benefit of U.S. Provisional Application No. 63/241,954, entitled “SYSTEMS AND METHODS FOR TRNAPOSING CARGO NUCLEOTIDE SEQUENCES”, filed on Sep. 8, 2021, which is incorporated by reference herein in its entirety.

### SEQUENCE LISTING

The instant application contains a Sequence Listing which has been submitted electronically in XML format and is hereby incorporated by reference in its entirety. Said XML copy, created on Feb. 1, 2024, is named 55921-734_302_SL.xml and is 139,299 bytes in size.

### BACKGROUND

Transposable elements are movable DNA sequences which play a crucial role in gene function and evolution. While transposable elements are found in nearly all forms of life, their prevalence varies among organisms, with a large proportion of the eukaryotic genome encoding for transposable elements (at least 45% in humans).

### SUMMARY

While the foundational research on transposable elements was conducted in the 1940s, their potential utility in DNA manipulation and gene editing applications has only been recognized in recent years.

In some aspects, the present disclosure provides for an engineered retrotransposase system, comprising:(a) a double-stranded nucleic acid comprising a cargo nucleotide sequence, wherein said cargo nucleotide sequence is configured to interact with a retrotransposase; and (b) a retrotransposase, wherein: (i) said retrotransposase is configured to transpose said cargo nucleotide sequence to a target nucleic acid locus; and (ii) said retrotransposase is derived from an uncultivated microorganism. In some embodiments, said retrotransposase comprises a sequence having at least 75% sequence identity to any one of SEQ ID NOs: 1-16. In some embodiments, said retrotransposase comprises a reverse transcriptase domain. In some embodiments, said retrotransposase further comprises one or more zinc finger domains. In some embodiments, said retrotransposase further comprises an endonuclease domain. In some embodiments, said retrotransposase has less than 80% sequence identity to a known retrotransposase. In some embodiments, said cargo nucleotide sequence is flanked by a 3′ untranslated region (UTR) and a 5′ untranslated region (UTR). In some embodiments, said retrotransposase is configured to transpose said cargo nucleotide sequence via a ribonucleic acid polynucleotide intermediate. In some embodiments, said retrotransposase comprises one or more nuclear localization sequences (NLSs) proximal to an N- or C-terminus of said retrotransposase. In some embodiments, said NLS comprises a sequence at least 80% identical to a sequence from the group consisting of SEQ ID NO: 17-32. In some embodiments, said sequence identity is determined by a BLASTP, CLUSTALW, MUSCLE, MAFFT, or CLUSTALW with the parameters of the Smith-Waterman homology search algorithm. In some embodiments, said sequence identity is determined by said BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In some aspects, the present disclosure provides for an engineered retrotransposase system, comprising:(a) a double-stranded nucleic acid comprising a cargo nucleotide sequence, wherein said cargo nucleotide sequence is configured to interact with a retrotransposase; and (b) a retrotransposase, wherein: (i) said retrotransposase is configured to transpose said cargo nucleotide sequence to a target nucleic acid locus; and (ii) said retrotransposase comprises a sequence having at least 75% sequence identity to any one of SEQ ID NOs: 1-16 In some embodiments, said retrotransposase is derived from an uncultivated microorganism. In some embodiments, said retrotransposase comprises a reverse transcriptase domain. In some embodiments, said retrotransposase further comprises one or more zinc finger domains. In some embodiments, said retrotransposase further comprises an endonuclease domain. In some embodiments, said retrotransposase has less than 80% sequence identity to a known retrotransposase. In some embodiments, said cargo nucleotide sequence is flanked by a 3′ untranslated region (UTR) and a 5′ untranslated region (UTR). In some embodiments, said retrotransposase is configured to transpose said cargo nucleotide sequence via a ribonucleic acid polynucleotide intermediate. In some embodiments, said sequence identity is determined by a BLASTP, CLUSTALW, MUSCLE, MAFFT, or CLUSTALW with the parameters of the Smith-Waterman homology search algorithm. In some embodiments, said sequence identity is determined by said BLASTP homology search algorithm using parameters of a wordlength (W) of 3, an expectation (E) of 10, and a BLOSUM62 scoring matrix setting gap costs at existence of 11, extension of 1, and using a conditional compositional score matrix adjustment.

In some aspects, the present disclosure provides for a deoxyribonucleic acid polynucleotide encoding said engineered retrotransposase system of any one of the aspects or embodiments described herein.

In some aspects, the present disclosure provides for a nucleic acid comprising an engineered nucleic acid sequence optimized for expression in an organism, wherein said nucleic acid encodes a retrotransposase, and wherein said retrotransposase is derived from an uncultivated microorganism, wherein said organism is not said uncultivated microorganism. In some embodiments, said retrotransposase comprises a variant having at least 75% sequence identity to any one of SEQ ID NOs: 1-16. In some embodiments, said retrotransposase comprises a sequence encoding one or more nuclear localization sequences (NLSs) proximal to an N- or C-terminus of said retrotransposase. In some embodiments, said NLS comprises a sequence selected from SEQ ID NOs: 17-32. In some embodiments, said NLS comprises SEQ ID NO: 18.

In some embodiments, said NLS is proximal to said N-terminus of said retrotransposase. In some embodiments, said NLS comprises SEQ ID NO: 17. In some embodiments, said NLS is proximal to said C-terminus of said retrotransposase. In some embodiments, said organism is prokaryotic, bacterial, eukaryotic, fungal, plant, mammalian, rodent, or human.

In some aspects, the present disclosure provides for a vector comprising said nucleic acid of any one of the aspects or embodiments described herein. In some embodiments, the method further comprises a nucleic acid encoding a cargo nucleotide sequence configured to form a complex with said retrotransposase. In some embodiments, said vector is a plasmid, a minicircle, a CELiD, an adeno-associated virus (AAV) derived virion, or a lentivirus.

In some aspects, the present disclosure provides for a cell comprising said vector of any one of any one of the aspects or embodiments described herein.

In some aspects, the present disclosure provides for a method of manufacturing a retrotransposase, comprising cultivating said cell of any one of the aspects or embodiments described herein.

In some aspects, the present disclosure provides for a method for binding, nicking, cleaving, marking, modifying, or transposing a double-stranded deoxyribonucleic acid polynucleotide, comprising: (a) contacting said double-stranded deoxyribonucleic acid polynucleotide with a retrotransposase configured to transpose said cargo nucleotide sequence to a target nucleic acid locus; and (b) wherein said retrotransposase comprises a sequence having at least 75% sequence identity to any one of SEQ ID NOs: 1-16. In some embodiments, said retrotransposase is derived from an uncultivated microorganism. In some embodiments, said retrotransposase comprises a reverse transcriptase domain. In some embodiments, said retrotransposase further comprises one or more zinc finger domains. In some embodiments, said retrotransposase further comprises an endonuclease domain. In some embodiments, said retrotransposase has less than 80% sequence identity to a known retrotransposase. In some embodiments, said cargo nucleotide sequence is flanked by a 3′ untranslated region (UTR)and a 5′ untranslated region (UTR). In some embodiments, said double-stranded deoxyribonucleic acid polynucleotide is transposed via a ribonucleic acid polynucleotide intermediate. In some embodiments, said double-stranded deoxyribonucleic acid polynucleotide is a eukaryotic, plant, fungal, mammalian, rodent, or human double-stranded deoxyribonucleic acid polynucleotide.

In some aspects, the present disclosure provides for a method of modifying a target nucleic acid locus, said method comprising delivering to said target nucleic acid locus said engineered retrotransposase system of any one of the aspects or embodiments described herein, wherein said retrotransposase is configured to transpose said cargo nucleotide sequence to said target nucleic acid locus, and wherein said complex is configured such that upon binding of said complex to said target nucleic acid locus, said complex modifies said target nucleic acid locus. In some embodiments, said target nucleic acid locus comprises binding, nicking, cleaving, marking, modifying, or transposing said target nucleic acid locus. In some embodiments, said target nucleic acid locus comprises deoxyribonucleic acid (DNA). In some embodiments, said target nucleic acid locus comprises genomic DNA, viral DNA, or bacterial DNA. In some embodiments, said target nucleic acid locus is in vitro. In some embodiments, said target nucleic acid locus is within a cell. In some embodiments, said cell is a prokaryotic cell, a bacterial cell, a eukaryotic cell, a fungal cell, a plant cell, an animal cell, a mammalian cell, a rodent cell, a primate cell, a human cell, or a primary cell. In some embodiments, said cell is a primary cell. In some embodiments, said primary cell is a T cell. In some embodiments, said primary cell is a hematopoietic stem cell (HSC). In some embodiments, delivering said engineered retrotransposase system to said target nucleic acid locus comprises delivering the nucleic acid of any one of the aspects or embodiments described herein or the vector of any one of the aspects or embodiments described herein. In some embodiments, delivering said engineered retrotransposase system to said target nucleic acid locus comprises delivering a nucleic acid comprising an open reading frame encoding said retrotransposase. In some embodiments, said nucleic acid comprises a promoter to which said open reading frame encoding said retrotransposase is operably linked. In some embodiments, delivering said engineered retrotransposase system to said target nucleic acid locus comprises delivering a capped mRNA containing said open reading frame encoding said retrotransposase. In some embodiments, delivering said engineered retrotransposase system to said target nucleic acid locus comprises delivering a translated polypeptide. In some embodiments, said retrotransposase does not induce a break at or proximal to said target nucleic acid locus.

In some aspects, the present disclosure provides for a host cell comprising an open reading frame encoding a heterologous retrotransposase having at least 75% sequence identity to any one of SEQ ID NOs: 1-16 or a variant thereof. In some embodiments, said host cell is an E. coli cell. In some embodiments, said E. coli cell is a λDE3 lysogen or said E. coli cell is a BL21(DE3) strain. In some embodiments, said E. coli cell has an ompT lon genotype. In some embodiments, said open reading frame is operably linked to a T7 promoter sequence, a T7-lac promoter sequence, a lac promoter sequence, a tac promoter sequence, a trc promoter sequence, a ParaBAD promoter sequence, a PrhaBAD promoter sequence, a T5 promoter sequence, a cspA promoter sequence, an araPBAD promoter, a strong leftward promoter from phage lambda (pL promoter), or any combination thereof. In some embodiments, said open reading frame comprises a sequence encoding an affinity tag linked in-frame to a sequence encoding said retrotransposase. In some embodiments, said affinity tag is an immobilized metal affinity chromatography (IMAC) tag. In some embodiments, said IMAC tag is a polyhistidine tag. In some embodiments, said affinity tag is a myc tag, a human influenza hemagglutinin (HA) tag, a maltose binding protein (MBP) tag, a glutathione S-transferase (GST) tag, a streptavidin tag, a FLAG tag, or any combination thereof. In some embodiments, said affinity tag is linked in-frame to said sequence encoding said retrotransposase via a linker sequence encoding a protease cleavage site. In some embodiments, said protease cleavage site is a tobacco etch virus (TEV) protease cleavage site, a PreScission® protease cleavage site, a Thrombin cleavage site, a Factor Xa cleavage site, an enterokinase cleavage site, or any combination thereof. In some embodiments, said open reading frame is codon-optimized for expression in said host cell. In some embodiments, said open reading frame is provided on a vector. In some embodiments, said open reading frame is integrated into a genome of said host cell.

In some aspects, the present disclosure provides for a culture comprising the host cell of any one of the aspects or embodiments described herein in compatible liquid medium.

In some aspects, the present disclosure provides for a method of producing a retrotransposase, comprising cultivating the host cell of any one of the aspects or embodiments described herein in compatible growth medium. In some embodiments, the method further comprising inducing expression of said retrotransposase by addition of an additional chemical agent or an increased amount of a nutrient. In some embodiments, said additional chemical agent or increased amount of a nutrient comprises Isopropyl β-D-1-thiogalactopyranoside (IPTG) or additional amounts of lactose. In some embodiments, the method further comprising isolating said host cell after said cultivation and lysing said host cell to produce a protein extract. In some embodiments, the method further comprises subjecting said protein extract to IMAC, or ion-affinity chromatography. In some embodiments, said open reading frame comprises a sequence encoding an IMAC affinity tag linked in-frame to a sequence encoding said retrotransposase. In some embodiments, said IMAC affinity tag is linked in-frame to said sequence encoding said retrotransposase via a linker sequence encoding protease cleavage site. In some embodiments, said protease cleavage site comprises a tobacco etch virus (TEV) protease cleavage site, a PreScission® protease cleavage site, a Thrombin cleavage site, a Factor Xa cleavage site, an enterokinase cleavage site, or any combination thereof. In some embodiments, the method further comprises cleaving said IMAC affinity tag by contacting a protease corresponding to said protease cleavage site to said retrotransposase. In some embodiments, the method further comprises performing subtractive IMAC affinity chromatography to remove said affinity tag from a composition comprising said retrotransposase.

In some aspects, the present disclosure provides for a method of disrupting a locus in a cell, comprising contacting to said cell a composition comprising: (a) a double-stranded nucleic acid comprising a cargo nucleotide sequence, wherein said cargo nucleotide sequence is configured to interact with a retrotransposase; and(b) a retrotransposase, wherein: (i) said retrotransposase is configured to transpose said cargo nucleotide sequence to a target nucleic acid locus; (ii) said retrotransposase comprises a sequence having at least 75% sequence identity to any one of SEQ ID NOs: 1-16; and (iii) said retrotransposase has at least equivalent transposition activity to a known retrotransposase in a cell. In some embodiments, said transposition activity is measured in vitro by introducing said retrotransposase to cells comprising said target nucleic acid locus and detecting transposition of said target nucleic acid locus in said cells. In some embodiments, said composition comprises 20 pmoles or less of said retrotransposase. In some embodiments, said composition comprises 1 pmol or less of said retrotransposase.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the genomic context of a bacterial retrotransposon. MG140-34 is a predicted retrotransposase (arrow) encoding a reverse transcriptase domain. Regions flanking the retrotransposase display secondary structure that possibly represent binding sites for the retrotransposase (Secondary structure boxes and zoomed images).

FIGS. 2A and 2B depicts multiple sequence alignment (MSA) of MG retrotransposase protein sequences of the family MG140. FIG. 2A depicts MSA of the reverse transcriptase domain. Conserved catalytic residues D, QG, [Y/F]ADD, and LG are highlighted on the consensus sequence. FIG. 2B depicts MSA of a Zn-finger and endonuclease catalytic residue. Zn-finger motifs (CX[2-3]C) and nuclease catalytic residue are highlighted on the consensus sequence. Figure discloses SEQ ID NOS 63-65, 76, 66-70, 77, and 71-72, respectively, in order of appearance.

FIGS. 3A and 3B depicts a phylogenetic gene tree of MG and reference retrotransposase genes. FIG. 3A depicts microbial MG retrotransposases (black branches on clade 4) are more closely related to Eukaryotic than viral retrotransposases (grey branches on clade 6). Clade 1: Telomerase reverse transcriptases; clade 2: Group II intron reverse transcriptases; clade 3: Eukaryotic R1 type retrotransposases; clade 4: microbial and Eukaryotic R2 retrotransposases; clade 5: Eukaryotic retrovirus-related reverse transcriptases; and clade 6: viral reverse transcriptases. FIG. 3B depicts Clades 3 and 4 from the phylogenetic gene tree from (A). Some microbial MG retrotransposases contain multiple Zn-finger motifs (vertical rectangles), the conserved RVT_1 reverse transcriptase domain, and APE/RLE or other endonuclease domains (top and bottom panel). Some microbial MG retrotransposases lack an endonuclease domain (mid-panel). Figure discloses SEQ ID NOS 73, 74, 73, 73, and 74, respectively, in order of appearance.

FIG. 4 depicts a phylogenetic tree inferred from a multiple sequence alignment of the reverse transcriptase domain from diverse enzymes. RT sequences were derived from DNA, as well as RNA assemblies. Reference RTs were included in the tree for classification purposes.

FIG. 5A depicts a phylogenetic tree inferred from a multiple sequence alignment of RT domains identified from novel families of RTs (MG148). FIG. 5B depicts genomic context of MG140-34-R2 RT. Predicted genes not associated with the RT are displayed as white arrows. FIG. 5C depicts nucleotide sequence alignment of four members of the MG148 family indicating conserved regions (boxes underneath the sequence) upstream of the RT (arrow annotated over the consensus sequence).

FIG. 6 depicts screening of in vitro activity of RTns family of enzymes by qPCR (MG148). Activity was detected by qPCR using primers that amplify the full-length cDNA product derived from a primer extension reaction containing the respective RT. Samples are derived from RT reactions containing 100 nM substrate. The negative control is a no-template water in the PURExpress reaction. Positive control: R2Tg (Taeniopygia guttata), a previously described retrotransposon. Active candidates, defined as at least 10-fold signal above the negative control, are marked in dark grey while candidates inactive in these conditions are in light grey.

FIG. 7A depicts a phylogenetic tree inferred from a multiple sequence alignment of full-length Group II intron RTs identified novel sequences of Class C. FIG. 7B depicts a summary table of the MG153 family of Group II introns. AAI: average pairwise amino acid identity of family members to reference Group II intron sequences.

FIGS. 8A and 8B depicts screening of in vitro activity of GII intron Class C candidates MG153-22, MG153-23, and MG153-24 by primer extension assay. FIG. 8A lane numbers correspond to the following: 1-PURExpress no template control, 2-MMLV control RT, 3-TGIRT-III control RT, 4-MarathonRT control RT, 5-7 correspond to novel candidates MG153-22 through 24. Numbering in bold corresponds to gel lanes with active novel candidates. Results are representative of two independent experiments. FIG. 8B depicts detection of full-length cDNA production by qPCR. Dark grey bars correspond to RTs that generate product at least 10-fold above background. Results were determined from two technical replicates.

FIG. 9 depicts screening to assess the ability of indicated control RTs and GII intron Class C candidates to synthesize cDNA in mammalian cells. Detection of 542 bp PCR products by D1000 TapeStation for MG153-23. Lanes not relevant for the described experiment are covered by black boxes.

FIG. 10 depicts genomic context of the MG160-7 retron-like single-domain RT. The region upstream from the RT (dotted box) is conserved across MG160 members and folds into secondary structures (inset) that may be required for activity and function. Figure discloses SEQ ID NO: 75.

FIGS. 11A and 11B depicts screening of in vitro activity of retron-like candidate MG160-7 by primer extension assay. FIG. 11A lane numbers correspond to the following samples: 1-PURExpress no template control, 2-MMLV control RT, 3-TGIRT-III control RT, 4: MG160-7.

FIG. 11B depicts quantification of full-length cDNA production by qPCR. Dark grey bars correspond to RTs that generate product at least 10-fold above background. Results were determined from two technical replicates.

FIG. 12 depicts a screening of the ability of MG153 GII derived RTs to synthesize cDNA in mammalian cells. Detection of 542 bp cDNA synthesis PCR products were assayed by Tagman qPCR. cDNA activity was normalized to the activity TGIRT control where TGIRT represents a value of 1. Y axis is shown in log 10 scale.

FIGS. 13A and 13B depicts protein expression of MG153 GII derived RTs by immunoblots. FIG. 13A: Cells were transfected with plasmids containing the candidate RTs and protein expression was evaluated by immunoblot, detecting the HA peptide fused to the N termini of the RTs. All lanes were normalized to total protein concentration. Lanes not relevant for the described experiment in FIG. 13A are covered by black boxes. FIG. 13B: Table of expected molecular sizes for tested RTs.

FIG. 14 depicts relative activity of MG153-23 GII derived RT normalized to protein expression. cDNA synthesis was detected by Tagman qPCR, protein expression was detected by immunoblots. Activity relative to TGIRT was normalized per total protein concentration. Y axis is shown in a linear scale.

### BRIEF DESCRIPTION OF THE SEQUENCE LISTING

The Sequence Listing filed herewith provides exemplary polynucleotide and polypeptide sequences for use in methods, compositions, and systems according to the disclosure. Below are exemplary descriptions of sequences therein.

### MG140

SEQ ID NOs: 1-16 show the full-length peptide sequences of MG140 transposition proteins.

### MG148

SEQ ID NOs: 32-41 show the full-length peptide sequences of MG148 reverse transcriptase proteins.

SEQ ID NOs: 25-31 show the nucleotide sequences of genes encoding HA-His-tagged MG148 reverse transcriptase proteins.

### MG153

SEQ ID NOs: 42-44 show the full-length peptide sequences of MG153 reverse transcriptase proteins.

SEQ ID NOs: 17-19 show the nucleotide sequences of E. coli codon optimized genes encoding MG153 reverse transcriptase proteins.

SEQ ID NOs: 20-23 show the nucleotide sequences of genes encoding strep-tagged MG153 reverse transcriptase proteins.

### MG160

SEQ ID NO: 45 shows the full-length peptide sequence of an MG160 reverse transcriptase protein.

SEQ ID NO: 24 shows the nucleotide sequence of an E. coli codon optimized gene encoding an MG160 reverse transcriptase protein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The practice of some methods disclosed herein employ, unless otherwise indicated, techniques of immunology, biochemistry, chemistry, molecular biology, microbiology, cell biology, genomics, and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)) (which is entirely incorporated by reference herein).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% f a given value.

As used herein, a “cell” generally refers to a biological cell. A cell may be the basic structural, functional and/or biological unit of a living organism. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, fems, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g., kelp), a fungal cell (e.g.,, a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “nucleotide,” as used herein, generally refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [αS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled, such as using moieties comprising optically detectable moieties (e.g., fluorophores). Labeling may also be carried out with quantum dots. Detectable labels may include, for example, radioactive isotopes, fluorescent labels, chemiluminescent labels, bioluminescent labels, and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Il.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. A polynucleotide may comprise one or more analogs (e.g., altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol-containing nucleotides, biotin-linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

The terms “transfection” or “transfected” generally refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88 (which is entirely incorporated by reference herein).

The terms “peptide,” “polypeptide,” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some embodiments, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

As used herein, the “non-native” can generally refer to a nucleic acid or polypeptide sequence that is not found in a native nucleic acid or protein. Non-native may refer to affinity tags. Non-native may refer to fusions. Non-native may refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that may also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide.

The term “promoter”, as used herein, generally refers to the regulatory DNA region which controls transcription or expression of a gene, and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may generally refer to a promoter that contains all the basic elements to promote transcriptional expression of an operably linked polynucleotide. Eukaryotic basal promoters can contain a TATA-box and/or a CAAT box.

The term “expression”, as used herein, generally refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, “operably linked”, “operable linkage”, “operatively linked”, or grammatical equivalents thereof generally refer to juxtaposition of genetic elements, e.g., a promoter, an enhancer, a polyadenylation sequence, etc., wherein the elements are in a relationship permitting them to operate in the expected manner. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, generally refers to a macromolecule or association of macromolecules that comprises or associates with a polynucleotide and which may be used to mediate delivery of the polynucleotide to a cell. Examples of vectors include plasmids, viral vectors, liposomes, and other gene delivery vehicles. The vector generally comprises genetic elements, e.g., regulatory elements, operatively linked to a gene to facilitate expression of the gene in a target.

As used herein, “an expression cassette” and “a nucleic acid cassette” are used interchangeably generally to refer to a combination of nucleic acid sequences or elements that are expressed together or are operably linked for expression. In some embodiments, an expression cassette refers to the combination of regulatory elements and a gene or genes to which they are operably linked for expression.

A “functional fragment” of a DNA or protein sequence generally refers to a fragment that retains a biological activity (either functional or structural) that is substantially similar to a biological activity of the full-length DNA or protein sequence. A biological activity of a DNA sequence may be its ability to influence expression in a manner known to be attributed to the full-length sequence.

As used herein, an “engineered” object generally indicates that the object has been modified by human intervention. According to non-limiting examples: a nucleic acid may be modified by changing its sequence to a sequence that does not occur in nature; a nucleic acid may be modified by ligating it to a nucleic acid that it does not associate with in nature such that the ligated product possesses a function not present in the original nucleic acid; an engineered nucleic acid may synthesized in vitro with a sequence that does not exist in nature; a protein may be modified by changing its amino acid sequence to a sequence that does not exist in nature; an engineered protein may acquire a new function or property. An “engineered” system comprises at least one engineered component.

As used herein, “synthetic” and “artificial” can generally be used interchangeably to refer to a protein or a domain thereof that has low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains.

As used herein, the term “transposable element” refers to a DNA sequence that can move from one location in the genome to another (i.e., they can be “transposed”). Transposable elements can be generally divided into two classes. Class I transposable elements, or “retrotransposons”, are transposed via transcription and translation of an RNA intermediate which is subsequently reincorporated into its new location into the genome via reverse transcription (a process mediated by a reverse transcriptase). Class II transposable elements, or “DNA transposons”, are transposed via a complex of single- or double-stranded DNA flanked on either side by a transposase. Further features of this family of enzymes can be found, e.g. in Nature Education 2008, 1 (1), 204; and Genome Biology 2018, 19 (199), 1-12; each of which is incorporated herein by reference.

As used herein, the term “retrotransposons” refers to Class I transposable elements that function according to a two-part “copy and paste” mechanism involving an RNA intermediate. “Retrotransposase” refers to an enzyme responsible for transposition of a retrotransposon. In some embodiments, a retrotransposase comprises a reverse transcriptase domain. In some embodiments, a retrotransposase further comprises one or more zinc finger domains. In some embodiments, a retrotransposase further comprises an endonuclease domain.

The term “sequence identity” or “percent identity” in the context of two or more nucleic acids or polypeptide sequences, generally refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov); CLUSTALW with the Smith-Waterman homology search algorithm parameters with a match of 2, a mismatch of −1, and a gap of −1; MUSCLE with default parameters; MAFFT with parameters of a retree of 2 and max iterations of 1000; Novafold with default parameters; HMMER hmmalign with default parameters.

The term “optimally aligned” in the context of two or more nucleic acids or polypeptide sequences, generally refers to two (e.g., in a pairwise alignment) or more (e.g., in a multiple sequence alignment) sequences that have been aligned to maximal correspondence of amino acids residues or nucleotides, for example, as determined by the alignment producing a highest or “optimized” percent identity score.

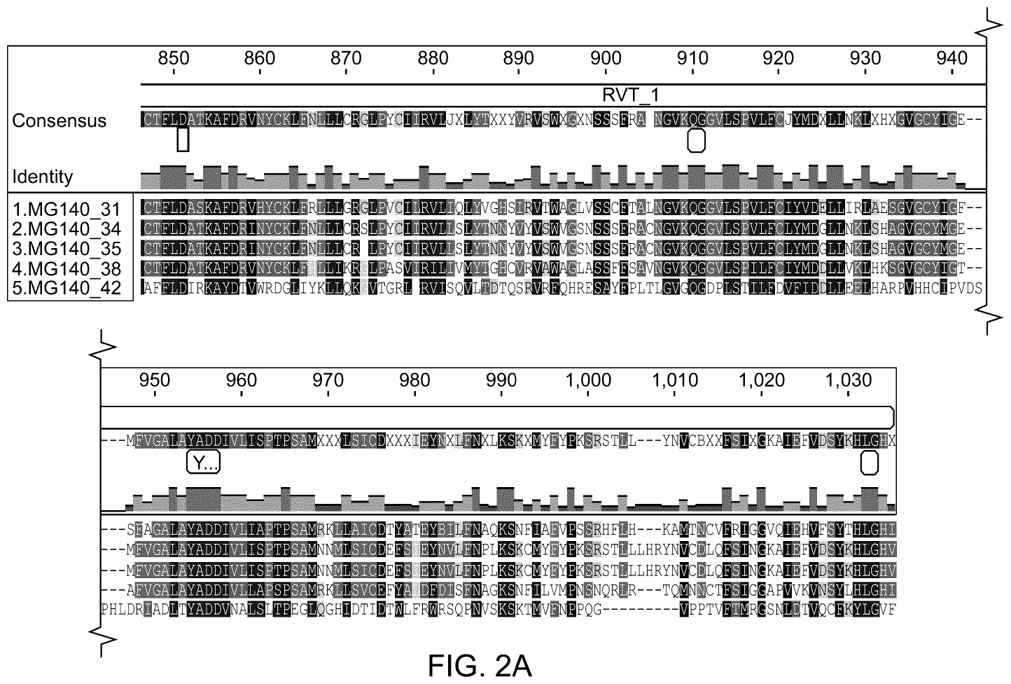

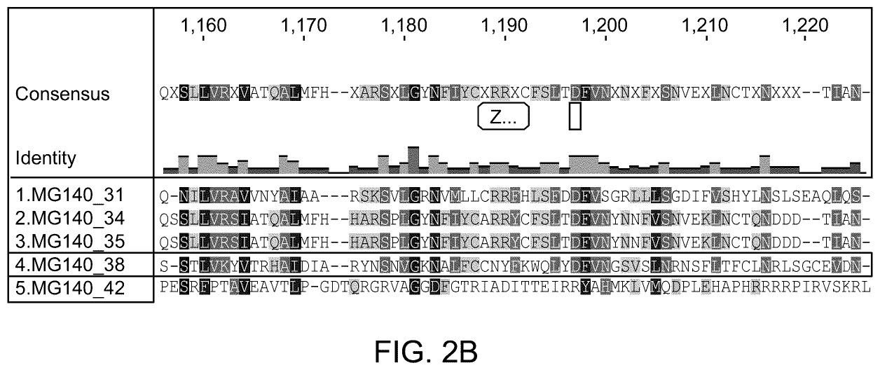

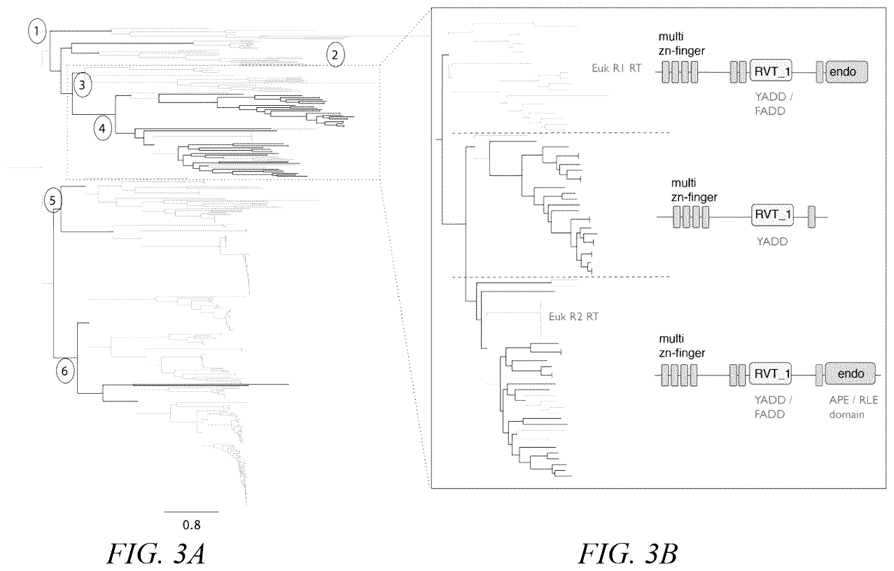

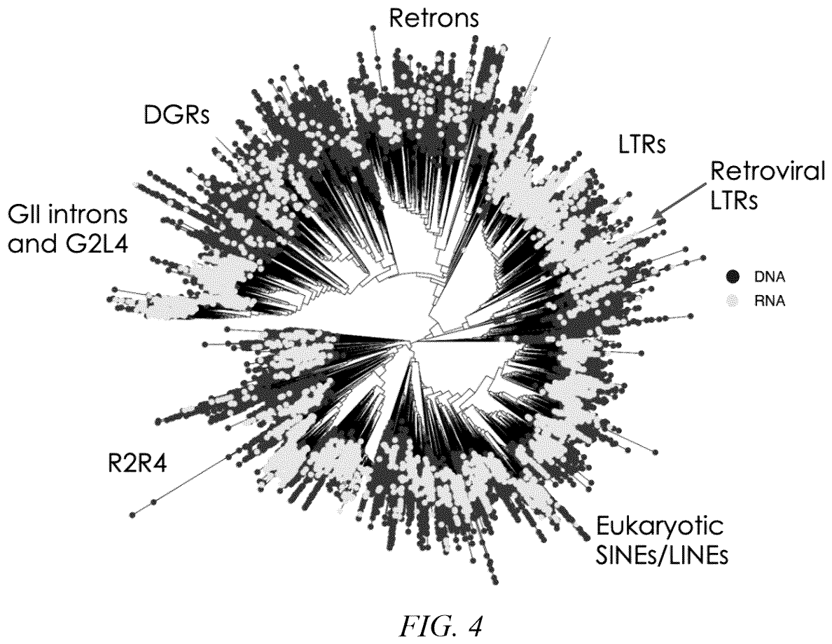

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally, or alternatively, by comparing aligned sequences of homologous proteins from different species, conservative substitutions can be identified by locating amino acid residues that have been mutated between species (e.g., non-conserved residues) without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99% identity to any one of the retrotransposase protein sequences described herein (e.g. MG140 family retrotransposases described herein, or any other family retrotransposase described herein). In some embodiments, such conservatively substituted variants are functional variants. Such functional variants can encompass sequences with substitutions such that the activity of one or more critical active site residues of the retrotransposase are not disrupted. In some embodiments, a functional variant of any of the proteins described herein lacks substitution of at least one of the conserved or functional residues called out in FIG. 2. In some embodiments, a functional variant of any of the proteins described herein lacks substitution of all of the conserved or functional residues called out in FIG. 2.

Also included in the current disclosure are variants of any of the enzymes described herein with substitution of one or more catalytic residues to decrease or eliminate activity of the enzyme (e.g. decreased-activity variants). In some embodiments, a decreased activity variant as a protein described herein comprises a disrupting substitution of at least one, at least two, or all three catalytic residues called out in FIG. 2.

Conservative substitution tables providing functionally similar amino acids are available from a variety of references (see, for e.g., Creighton, Proteins: Structures and Molecular Properties (W H Freeman & Co.; 2nd edition (December 1993)). The following eight groups each contain amino acids that are conservative substitutions for one another: 1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)

### Overview

The discovery of new transposable elements with unique functionality and structure may offer the potential to further disrupt deoxyribonucleic acid (DNA) editing technologies, improving speed, specificity, functionality, and ease of use. Relative to the predicted prevalence of transposable elements in microbes and the sheer diversity of microbial species, relatively few functionally characterized transposable elements exist in the literature. This is partly because a huge number of microbial species may not be readily cultivated in laboratory conditions. Metagenomic sequencing from natural environmental niches containing large numbers of microbial species may offer the potential to drastically increase the number of new transposable elements known and speed the discovery of new oligonucleotide editing functionalities.

Transposable elements are deoxyribonucleic acid sequences that can change position within a genome, often resulting in the generation or amelioration of mutations. In eukaryotes, a great proportion of the genome, and a large share of the mass of cellular DNA, is attributable to transposable elements. Although transposable elements are “selfish genes” which propagate themselves at the expense of other genes, they have been found to serve various important functions and to be crucial to genome evolution. Based on their mechanism, transposable elements are classified as either Class I “retrotransposons” or Class II “DNA transposons”. Class I transposable elements, also referred to as retrotransposons, function according to a two-part “copy and paste” mechanism involving an RNA intermediate. First, the retrotransposon is transcribed. The resulting RNA is subsequently converted back to DNA by reverse transcriptase (generally encoded by the retrotransposon itself), and the reverse transcribed retrotransposon is integrated into its new position in the genome by integrase. Retrotransposons are further classified into three orders. Retrotransposons with long terminal repeats (“LTRs”) encode reverse transcriptase and are flanked by long strands of repeating DNA. Retrotransposons with long interspersed nuclear elements (“LINEs”) encode reverse transcriptase, lack LTRs, and are transcribed by RNA polymerase II. Retrotransposons with short interspersed nuclear elements (“SINEs”) are transcribed by RNA polymerase III but lack reverse transcriptase, instead relying on the reverse transcription machinery of other transposable elements (e.g. LINEs).

Class II transposable elements, also referred to as DNA transposons, function according to mechanisms that do not involve an RNA intermediate. Many DNA transposons display a “cut and paste” mechanism in which transposase binds terminal inverted repeats (“TIRs”) flanking the transposon, cleaves the transposon from the donor region, and inserts it into the target region of the genome. Others, referred to as “helitrons”, display a “rolling circle” mechanism involving a single-stranded DNA intermediate and mediated by an undocumented protein believed to possess HUH endonuclease function and 5′ to 3′ helicase activity. First, a circular strand of DNA is nicked to create two single DNA strands. The protein remains attached to the 5′ phosphate of the nicked strand, leaving the 3′ hydroxyl end of the complementary strand exposed and thus allowing a polymerase to replicate the non-nicked strand. Once replication is complete, the new strand disassociates and is itself replicated along with the original template strand. Still other DNA transposons, “Polintons”, are theorized to undergo a “self-synthesis” mechanism. The transposition is initiated by an integrase's excision of a single-stranded extra-chromosomal Polinton element, which forms a racket-like structure. The Polinton undergoes replication with DNA polymerase B, and the double stranded Polinton is inserted into the genome by the integrase. Additionally, some DNA transposons, such as those in the IS200/IS605 family, proceed via a “peel and paste” mechanism in which TnpA excises a piece of single-stranded DNA (as a circular “transposon joint”) from the lagging strand template of the donor gene and reinserts it into the replication fork of the target gene.

While transposable elements have found some use as biological tools, documented transposable elements do not encompass the full range of possible biodiversity and targetability, and may not represent all possible activities. Here, thousands of genomic fragments were mined from numerous metagenomes for transposable elements. The documented diversity of transposable elements may have been expanded and novel systems may have been developed into highly targetable, compact, and precise gene editing agents.

### MG Enzymes

In some aspects, the present disclosure provides for novel retrotransposases. These candidates may represent one or more novel subtypes and some sub-families may have been identified. These retrotransposases are less than about 1,500 amino acids in length. These retrotransposases may simplify delivery and may extend therapeutic applications.

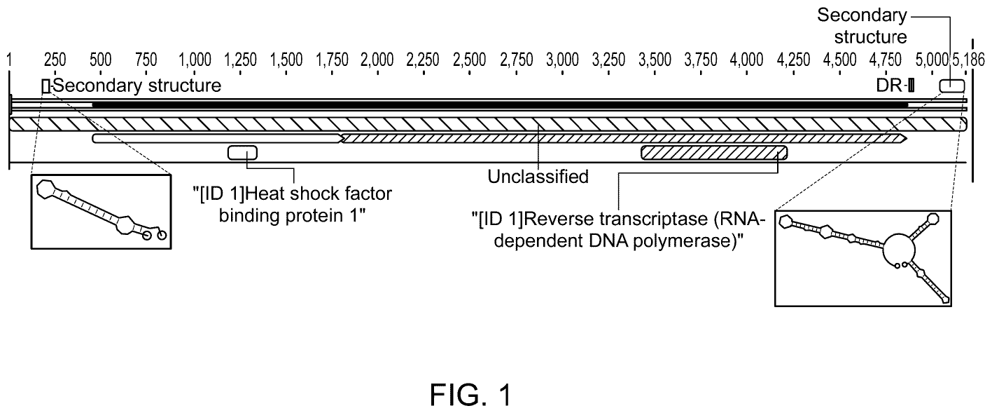

In some aspects, the present disclosure provides for a novel retrotransposase. Such a retrotransposase may be MG140 as described herein (see FIGS. 1 and 2).

In one aspect, the present disclosure provides for an engineered retrotransposase system discovered through metagenomic sequencing. In some embodiments, the metagenomic sequencing is conducted on samples. In some embodiments, the samples may be collected from a variety of environments. Such environments may be a human microbiome, an animal microbiome, environments with high temperatures, environments with low temperatures. Such environments may include sediment.

In one aspect, the present disclosure provides for an engineered retrotransposase system comprising a retrotransposase. In some embodiments, the retrotransposase is derived from an uncultivated microorganism. The retrotransposase may be configured to bind a 3′ untranslated region (UTR). The retrotransposase may bind a 5′ untranslated region (UTR).

In one aspect, the present disclosure provides for an engineered retrotransposase system comprising a retrotransposase. In some embodiments, the retrotransposase has at least about 70% sequence identity to any one of SEQ ID NOs: 1-16. In some embodiments, the retrotransposase has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-16.

In some embodiments, the retrotransposase comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-16. In some embodiments, the retrotransposase may be substantially identical to any one of SEQ ID NOs: 1-16.

In some embodiments, the retrotransposase comprises a reverse transcriptase domain. In some embodiments, the retrotransposase further comprises one or more zinc finger domains. In some embodiments, the retrotransposase further comprises an endonuclease finger domain.

In some embodiments, the retrotransposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a known or documented retrotransposase.

In some embodiments, the cargo nucleotide sequence is flanked by a 3′ untranslated region (UTR) and a 5′ untranslated region (UTR).

In some embodiments, the retrotransposase is configured to transpose the cargo nucleotide sequence as single-stranded deoxyribonucleic acid polynucleotide. In some embodiments, the retrotransposase is configured to transpose the cargo nucleotide sequence as double-stranded deoxyribonucleic acid polynucleotide. In some embodiments, the retrotransposase is configured to transpose said cargo nucleotide sequence via a ribonucleic acid polynucleotide intermediate.

In some embodiments, the retrotransposase comprises a sequence complementary to a eukaryotic, fungal, plant, mammalian, or human genomic polynucleotide sequence. In some embodiments, the retrotransposase comprises a sequence complementary to a eukaryotic genomic polynucleotide sequence. In some embodiments, the retrotransposase comprises a sequence complementary to a fungal genomic polynucleotide sequence. In some embodiments, the retrotransposase comprises a sequence complementary to a plant genomic polynucleotide sequence. In some embodiments, the retrotransposase comprises a sequence complementary to a mammalian genomic polynucleotide sequence. In some embodiments, the retrotransposase comprises a sequence complementary to a human genomic polynucleotide sequence.

In some embodiments, the retrotransposase may comprise a variant having one or more nuclear localization sequences (NLSs). The NLS may be proximal to the N- or C-terminus of the retrotransposase. The NLS may be appended N-terminal or C-terminal to any one ofSEQ ID NOs: 17-32, or to a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 17-32. In some embodiments, the NLS may comprise a sequence substantially identical to any one of SEQ ID NOs: 17-32. In some embodiments, the NLS may comprise a sequence substantially identical to SEQ ID NO: 17. In some embodiments, the NLS may comprise a sequence substantially identical to SEQ ID NO: 18.

TABLE 1
Example NLS Sequences that may be used with retrotransposases according to the
disclosure

| Source | NLS amino acid sequence | SEQ ID NO: |
|---|---|---|
| SV40 | PKKKRKV | 17 |
| nucleoplasmin | KRPAATKKAGQAKKKK | 18 |
| bipartite NLS | | |
| c-myc NLS | PAAKRVKLD | 19 |
| c-myc NLS | RQRRNELKRSP | 20 |
| hRNPA1 M9 NLS | NQSSNFGPMKGGNFGGRSSGPYGGGGQYFAKPRNQGGY | 21 |
| Importin-alpha IBB | RMRIZFKNKGKDTAELRRRRVEVSVELRKAKKDEQILKRRNV | 22 |
| domain | | |
| Myoma T protein | VSRKRPRP | 23 |
| Myoma T protein | PPKKARED | 24 |
| p53 | PQPKKKPL | 25 |
| mouse c-abl IV | SALIKKKKKMAP | 26 |
| influenza virus NS1 | DRLRR | 27 |
| influenza virus NS1 | PKQKKRK | 28 |
| Hepatitis virus delta | RKLKKKIKKL | 29 |
| antigen | | |
| mouse Mx1 protein | REKKKELKRR | 30 |
| human poly(ADP- | KRKGDEVDGVDEVAKKKSKK | 31 |
| ribose) polymerase | | |
| steroid hormone | RKCLQAGMNLEARKTKK | 32 |
| receptors (human) | | |
| glucocorticoid | | |

In some embodiments, sequence may be determined by a BLASTP, CLUSTALW, MUSCLE, or MAFFT algorithm, or a CLUSTALW algorithm with the Smith-Waterman homology search algorithm parameters. The sequence identity may be determined by the BLASTP homology search algorithm using parameters of a wordlength (W) of 3, an expectation (E) of 10, and a BLOSUM62 scoring matrix selling gap costs at existence of 11, extension of 1, and using a conditional compositional score matrix adjustment.

In one aspect, the present disclosure provides a deoxyribonucleic acid polynucleotide encoding the engineered retrotransposase system described herein.

In one aspect, the present disclosure provides a nucleic acid comprising an engineered nucleic acid sequence. In some embodiments, the engineered nucleic acid sequence is optimized for expression in an organism. In some embodiments, the retrotransposase is derived from an uncultivated microorganism. In some embodiments, the organism is not the uncultivated organism.

In some embodiments, the retrotransposase has at least about 70% sequence identity to any one of SEQ ID NOs: 1-16. In some embodiments, the retrotransposase has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-16.

In some embodiments, the retrotransposase comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% sequence identity to any one of SEQ ID NOs: 1-16. In some embodiments, the retrotransposase may be substantially identical to any one of SEQ ID NOs: 1-16.

In some embodiments, the retrotransposase comprises a reverse transcriptase domain. In some embodiments, the retrotransposase further comprises one or more zinc finger domains. In some embodiments, the retrotransposase further comprises an endonuclease finger domain.

In some embodiments, the retrotransposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a known or documented retrotransposase.

In some embodiments, the cargo nucleotide sequence is flanked by a 3′ untranslated region (UTR)and a 5′ untranslated region (UTR).

In some embodiments, the retrotransposase is configured to transpose the cargo nucleotide sequence as single-stranded deoxyribonucleic acid polynucleotide. In some embodiments, the retrotransposase is configured to transpose the cargo nucleotide sequence as double-stranded deoxyribonucleic acid polynucleotide. In some embodiments, the retrotransposase is configured to transpose said cargo nucleotide sequence via a ribonucleic acid polynucleotide intermediate.

In some embodiments, the retrotransposase comprises a sequence complementary to a eukaryotic, fungal, plant, mammalian, or human genomic polynucleotide sequence. In some embodiments, the retrotransposase comprises a sequence complementary to a eukaryotic genomic polynucleotide sequence. In some embodiments, the retrotransposase comprises a sequence complementary to a fungal genomic polynucleotide sequence. In some embodiments, the retrotransposase comprises a sequence complementary to a plant genomic polynucleotide sequence. In some embodiments, the retrotransposase comprises a sequence complementary to a mammalian genomic polynucleotide sequence. In some embodiments, the retrotransposase comprises a sequence complementary to a human genomic polynucleotide sequence.

In some embodiments, the retrotransposase may comprise a variant having one or more nuclear localization sequences (NLSs). The NLS may be proximal to the N- or C-terminus of the retrotransposase. The NLS may be appended N-terminal or C-terminal to any one of SEQ ID NOs: 17-32, or to a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 17-32. In some embodiments, the NLS may comprise a sequence substantially identical to any one of SEQ ID NOs: 17-32. In some embodiments, the NLS may comprise a sequence substantially identical to SEQ ID NO: 17. In some embodiments, the NLS may comprise a sequence substantially identical to SEQ ID NO: 18.

In some embodiments, the organism is prokaryotic. In some embodiments, the organism is bacterial. In some embodiments, the organism is eukaryotic. In some embodiments, the organism is fungal. In some embodiments, the organism is a plant. In some embodiments, the organism is mammalian. In some embodiments, the organism is a rodent. In some embodiments, the organism is human.

In one aspect, the present disclosure provides an engineered vector. In some embodiments, the engineered vector comprises a nucleic acid sequence encoding a retrotransposase. In some embodiments, the retrotransposase is derived from an uncultivated microorganism.

In some embodiments, the engineered vector comprises a nucleic acid described herein. In some embodiments, the nucleic acid described herein is a deoxyribonucleic acid polynucleotide described herein. In some embodiments, the vector is a plasmid, a minicircle, a CELiD, an adeno-associated virus (AAV) derived virion, or a lentivirus.

In one aspect, the present disclosure provides a cell comprising a vector described herein.

In one aspect, the present disclosure provides a method of manufacturing a retrotransposase. In some embodiments, the method comprises cultivating the cell.

In one aspect, the present disclosure provides a method for binding, nicking, cleaving, marking, modifying, or transposing a double-stranded deoxyribonucleic acid polynucleotide. The method may comprise contacting the double-stranded deoxyribonucleic acid polynucleotide with a retrotransposase. In some embodiments, the cargo nucleotide sequence is flanked by a 3′ untranslated region (UTR) and a 5′ untranslated region (UTR).

In some embodiments, the retrotransposase comprises a reverse transcriptase domain. In some embodiments, the retrotransposase further comprises one or more zinc finger domains. In some embodiments, the retrotransposase further comprises an endonuclease finger domain.

In some embodiments, the retrotransposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a known or documented retrotransposase.

In some embodiments, the retrotransposase is configured to transpose the cargo nucleotide sequence as single-stranded deoxyribonucleic acid polynucleotide. In some embodiments, the retrotransposase is configured to transpose the cargo nucleotide sequence as double-stranded deoxyribonucleic acid polynucleotide. In some embodiments, the retrotransposase is configured to transpose said cargo nucleotide sequence via a ribonucleic acid polynucleotide intermediate.

In some embodiments, the retrotransposase is derived from an uncultivated microorganism. In some embodiments, the double-stranded deoxyribonucleic acid polynucleotide is a eukaryotic, plant, fungal, mammalian, rodent, or human double-stranded deoxyribonucleic acid polynucleotide.

In one aspect, the present disclosure provides a method of modifying a target nucleic acid locus. The method may comprise delivering to the target nucleic acid locus the engineered retrotransposase system described herein. In some embodiments, the complex is configured such that upon binding of the complex to the target nucleic acid locus, the complex modifies the target nucleic acid locus.

In some embodiments, modifying the target nucleic acid locus comprises binding, nicking, cleaving, marking, modifying, or transposing the target nucleic acid locus. In some embodiments, the target nucleic acid locus comprises deoxyribonucleic acid (DNA) or ribonucleic acid (RNA). In some embodiments, the target nucleic acid comprises genomic DNA, viral DNA, viral RNA, or bacterial DNA. In some embodiments, the target nucleic acid locus is in vitro. In some embodiments, the target nucleic acid locus is within a cell. In some embodiments, the cell is a prokaryotic cell, a bacterial cell, a eukaryotic cell, a fungal cell, a plant cell, an animal cell, a mammalian cell, a rodent cell, a primate cell, or a human cell. In some embodiments, the cell is a primary cell. In some embodiments, the primary cell is a T cell. In some embodiments, the primary cell is a hematopoietic stem cell (HSC).

In some embodiments, delivery of the engineered retrotransposase system to the target nucleic acid locus comprises delivering the nucleic acid described herein or the vector described herein. In some embodiments, delivery of engineered retrotransposase system to the target nucleic acid locus comprises delivering a nucleic acid comprising an open reading frame encoding the retrotransposase. In some embodiments, the nucleic acid comprises a promoter. In some embodiments, the open reading frame encoding the retrotransposase is operably linked to the promoter.

In some embodiments, delivery of the engineered retrotransposase system to the target nucleic acid locus comprises delivering a capped mRNA containing the open reading frame encoding the retrotransposase. In some embodiments, delivery of the engineered retrotransposase system to the target nucleic acid locus comprises delivering a translated polypeptide. In some embodiments, delivery of the engineered retrotransposase system to the target nucleic acid locus comprises delivering a deoxyribonucleic acid (DNA) encoding the engineered guide RNA operably linked to a ribonucleic acid (RNA) pol III promoter.

In some embodiments, the retrotransposase does not induce a break at or proximal to said target nucleic acid locus.

In one aspect, the present disclosure provides a host cell comprising an open reading frame encoding a heterologous retrotransposase. In some embodiments, the retrotransposase has at least about 70% sequence identity to any one of SEQ ID NOs: 1-16. In some embodiments, the retrotransposase has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-16.

In some embodiments, the retrotransposase comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-16. In some embodiments, the retrotransposase may be substantially identical to any one of SEQ ID NOs: 1-16.

In some embodiments, the retrotransposase comprises a reverse transcriptase domain. In some embodiments, the retrotransposase further comprises one or more zinc finger domains. In some embodiments, the retrotransposase further comprises an endonuclease finger domain.

In some embodiments, the retrotransposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a known or documented retrotransposase.

In some embodiments, the cargo nucleotide sequence is flanked by a 3′ untranslated region (UTR)and a 5′ untranslated region (UTR).

In some embodiments, the retrotransposase is configured to transpose the cargo nucleotide sequence as double-stranded deoxyribonucleic acid polynucleotide. In some embodiments, the retrotransposase is configured to transpose the cargo nucleotide sequence as double-stranded deoxyribonucleic acid polynucleotide. In some embodiments, the retrotransposase is configured to transpose said cargo nucleotide sequence via a ribonucleic acid polynucleotide intermediate.

In some embodiments, the host cell is an E. coli cell. In some embodiments, the E. coli cell is a λDE3 lysogen or the E. coli cell is a BL21(DE3) strain. In some embodiments, the E. coli cell has an ompT lon genotype.

In some embodiments, the open reading frame is operably linked to a T7 promoter sequence, a T7-lac promoter sequence, a lac promoter sequence, a tac promoter sequence, a trc promoter sequence, a ParaBAD promoter sequence, a PrhaBAD promoter sequence, a T5 promoter sequence, a cspA promoter sequence, an araPBAD promoter, a strong leftward promoter from phage lambda (pL promoter), or any combination thereof.

In some embodiments, the open reading frame comprises a sequence encoding an affinity tag linked in-frame to a sequence encoding the retrotransposase. In some embodiments, the affinity tag is an immobilized metal affinity chromatography (IMAC) tag. In some embodiments, the IMAC tag is a polyhistidine tag. In some embodiments, the affinity tag is a myc tag, a human influenza hemagglutinin (HA) tag, a maltose binding protein (MBP) tag, a glutathione S-transferase (GST) tag, a streptavidin tag, a FLAG tag, or any combination thereof.

In some embodiments, the affinity tag is linked in-frame to the sequence encoding the retrotransposase via a linker sequence encoding a protease cleavage site. In some embodiments, the protease cleavage site is a tobacco etch virus (TEV) protease cleavage site, a PreScission® protease cleavage site, a Thrombin cleavage site, a Factor Xa cleavage site, an enterokinase cleavage site, or any combination thereof.

In some embodiments, the open reading frame is codon-optimized for expression in the host cell. In some embodiments, the open reading frame is provided on a vector. In some embodiments, the open reading frame is integrated into a genome of the host cell.

In one aspect, the present disclosure provides a culture comprising a host cell described herein in compatible liquid medium.

In one aspect, the present disclosure provides a method of producing a retrotransposase, comprising cultivating a host cell described herein in compatible growth medium. In some embodiments, the method further comprises inducing expression of the retrotransposase by addition of an additional chemical agent or an increased amount of a nutrient. In some embodiments, the additional chemical agent or increased amount of a nutrient comprises Isopropyl β-D-1-thiogalactopyranoside (IPTG) or additional amounts of lactose. In some embodiments, the method further comprises isolating the host cell after the cultivation and lysing the host cell to produce a protein extract. In some embodiments, the method further comprises subjecting the protein extract to IMAC, or ion-affinity chromatography. In some embodiments, the open reading frame comprises a sequence encoding an IMAC affinity tag linked in-frame to a sequence encoding the retrotransposase. In some embodiments, the IMAC affinity tag is linked in-frame to the sequence encoding the retrotransposase via a linker sequence encoding protease cleavage site. In some embodiments, the protease cleavage site comprises a tobacco etch virus (TEV) protease cleavage site, a PreScission® protease cleavage site, a Thrombin cleavage site, a Factor Xa cleavage site, an enterokinase cleavage site, or any combination thereof. In some embodiments, the method further comprises cleaving the IMAC affinity tag by contacting a protease corresponding to the protease cleavage site to the retrotransposase. In some embodiments, the method further comprises performing subtractive IMAC affinity chromatography to remove the affinity tag from a composition comprising the retrotransposase.

In one aspect, the present disclosure provides a method of disrupting a locus in a cell. In some embodiments, the method comprises contacting to the cell a composition comprising a retrotransposase. In some embodiments, the retrotransposase has at least equivalent transposition activity to a known or documented retrotransposase in a cell. In some embodiments, the retrotransposase has at least about 70% sequence identity to any one of SEQ ID NOs: 1-16. In some embodiments, the retrotransposase has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-16.

In some embodiments, the retrotransposase comprises a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 1-16. In some embodiments, the retrotransposase may be substantially identical to any one of SEQ ID NOs: 1-16.

In some embodiments, the retrotransposase comprises a reverse transcriptase domain. In some embodiments, the retrotransposase further comprises one or more zinc finger domains. In some embodiments, the retrotransposase further comprises an endonuclease finger domain.

In some embodiments, the retrotransposase has less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, or less than about 5% sequence identity to a known or documented retrotransposase.

In some embodiments, the cargo nucleotide sequence is flanked by a 3′ untranslated region (UTR) and a 5′ untranslated region (UTR).

In some embodiments, the retrotransposase is configured to transpose the cargo nucleotide sequence as double-stranded deoxyribonucleic acid polynucleotide. In some embodiments, the retrotransposase is configured to transpose the cargo nucleotide sequence as single-stranded deoxyribonucleic acid polynucleotide. In some embodiments, the retrotransposase is configured to transpose said cargo nucleotide sequence via a ribonucleic acid polynucleotide intermediate.

In some embodiments, the retrotransposase comprises a sequence complementary to a eukaryotic, fungal, plant, mammalian, or human genomic polynucleotide sequence. In some embodiments, the retrotransposase comprises a sequence complementary to a eukaryotic genomic polynucleotide sequence. In some embodiments, the retrotransposase comprises a sequence complementary to a fungal genomic polynucleotide sequence. In some embodiments, the retrotransposase comprises a sequence complementary to a plant genomic polynucleotide sequence. In some embodiments, the retrotransposase comprises a sequence complementary to a mammalian genomic polynucleotide sequence. In some embodiments, the retrotransposase comprises a sequence complementary to a human genomic polynucleotide sequence.

In some embodiments, the retrotransposase may comprise a variant having one or more nuclear localization sequences (NLSs). The NLS may be proximal to the N- or C-terminus of the retrotransposase. The NLS may be appended N-terminal or C-terminal to any one of SEQ ID NOs: 17-32, or to a variant having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 17-32. In some embodiments, the NLS may comprise a sequence substantially identical to any one of SEQ ID NOs: 17-32. In some embodiments, the NLS may comprise a sequence substantially identical to SEQ ID NO: 17. In some embodiments, the NLS may comprise a sequence substantially identical to SEQ ID NO: 18.

In some embodiments, the transposition activity is measured in vitro by introducing the retrotransposase to cells comprising the target nucleic acid locus and detecting transposition of the target nucleic acid locus in the cells. In some embodiments, the composition comprises 20 pmoles or less of the retrotransposase. In some embodiments, the composition comprises 1 pmol or less of the retrotransposase.

Systems of the present disclosure may be used for various applications, such as, for example, nucleic acid editing (e.g., gene editing), binding to a nucleic acid molecule (e.g., sequence-specific binding). Such systems may be used, for example, for addressing (e.g., removing or replacing) a genetically inherited mutation that may cause a disease in a subject, inactivating a gene in order to ascertain its function in a cell, as a diagnostic tool to detect disease-causing genetic elements (e.g. via cleavage of reverse-transcribed viral RNA or an amplified DNA sequence encoding a disease-causing mutation), as deactivated enzymes in combination with a probe to target and detect a specific nucleotide sequence (e.g. sequence encoding antibiotic resistance int bacteria), to render viruses inactive or incapable of infecting host cells by targeting viral genomes, to add genes or amend metabolic pathways to engineer organisms to produce valuable small molecules, macromolecules, or secondary metabolites, to establish a gene drive element for evolutionary selection, to detect cell perturbations by foreign small molecules and nucleotides as a biosensor.

### EXAMPLES

In accordance with IUPAC conventions, the following abbreviations are used throughout the examples:
A=adenine
C=cytosine
G=guanine
T=thymine
R=adenine or guanine
Y=cytosine or thymine
S=guanine or cytosine
W=adenine or thymine
K=guanine or thymine
M=adenine or cytosine
B=C,G, orT
D=A, G, or T
H=A, C,orT
V=A, C, or G

### Example 1—a Method of Metagenomic Analysis for New Proteins

Metagenomic samples were collected from sediment, soil, and animals. Deoxyribonucleic acid (DNA) was extracted with a Zymobiomics DNA mini-prep kit and sequenced on an Illumina HiSeq® 2500. Samples were collected with consent of property owners. Additional raw sequence data from public sources included animal microbiomes, sediment, soil, hot springs, hydrothermal vents, marine, peat bogs, permafrost, and sewage sequences. Metagenomic sequence data was searched using Hidden Markov Models generated based on documented retrotransposase protein sequences to identify new retrotransposases. Novel retrotransposase proteins identified by the search were aligned to documented proteins to identify potential active sites. This metagenomic workflow resulted in the delineation of the MG140 family described herein.

### Example 2—Discovery of MG140 Family of Retrotransposases

Analysis of the data from the metagenomic analysis of Example 1 revealed a new cluster of undescribed putative retrotransposase systems comprising 1 family (MG140). The corresponding protein sequences for these new enzymes and their subdomains are presented as SEQ ID NOs: 1-16.

### Example 3—Integration of Reverse Transcribed DNA In Vitro Activity (Prophetic)

Integrase activity can be interrogated via expression in an E. coli lysate-based expression system (for example, myTXTL, Arbor Biosciences). The required components for in vitro testing are three plasmids: an expression plasmid with the retrotransposon gene(s) under a T7 promoter, a target plasmid, and a donor plasmid which contains the required 5′ and 3′ UTR sequences recognized by the retrotransposase around a selection marker gene (e.g. Tet resistance gene). The lysate-based expression products, target DNA, and donor plasmid are incubated to allow for transposition to occur. Transposition is detected via PCR. In addition, the transposition product will be tagmented with T5 and sequenced via NGS to determine the insertion sites on a population of transposition events. Alternatively, the in vitro transposition products can be transformed into E. coli under antibiotic (e.g. Tet) selection, where growth requires the selection marker to be stably inserted into a plasmid. Either single colonies or a population of E. coli can be sequenced to determine the insertion sites.

Integration efficiency can be measured via ddPCR or qPCR of the experimental output of target DNA with integrated cargo, normalized to the amount of unmodified target DNA also measured via ddPCR.

This assay may also be conducted with purified protein components rather than from lysate-based expression. In this case, the proteins are expressed in E. coli protease-deficient B strain under T7 inducible promoter, the cells are lysed using sonication, and the His-tagged protein of interest is purified using HisTrap FF (GE Lifescience) Ni-NTA affinity chromatography on the AKTA Avant FPLC (GE Lifescience). Purity is determined using densitometry in ImageLab software (Bio-Rad) of the protein bands resolved on SDS-PAGE and InstantBlue Ultrafast (Sigma-Aldrich) coomassie stained acrylamide gels (Bio-Rad). The protein is desalted in storage buffer composed of 50 mM Tris-HCl, 300 mM NaCl, 1 mM TCEP, 5% glycerol; pH 7.5 (or other buffers as determined for maximum stability) and stored at −80° C. After purification the transposon gene(s) are added to the target DNA and donor plasmid as described above in a reaction buffer, for example 26 mM HEPES pH 7.5, 4.2 mM TRIS pH 8, 50 μg/mL BSA, 2 mM ATP, 2.1 mM DTT, 0.05 mM EDTA, 0.2 mM MgCl₂, 30-200 mM NaCl, 21 mM KCl, 1.35% glycerol, (final pH 7.5) supplemented with 15 mM MgOAc2.

### Example 4—Retrotransposon End Verification Via Gel Shift (Prophetic)

The retrotransposon ends are tested for retrotransposase binding via an electrophoretic mobility shift assay (EMSA). In this case, a target DNA fragment (100-500 bp) is end-labeled with FAM via PCR with FAM-labeled primers. The 3′ UTR RNA and 5′ UTR RNA are generated in vitro using T7 RNA polymerase and purified. The retrotransposase proteins are synthesized in an in vitro transcription/translation system (e.g. PURExpress). After synthesis, 1 μL of protein is added to 50 nM of the labeled DNA and 100 ng of the 3′ or 5′ UTR RNA in a 10 piL reaction in binding buffer (e.g. 20 mM HEPES pH 7.5, 2.5 mM Tris pH 7.5, 10 mM NaCl, 0.0625 mM EDTA, 5 mM TCEP, 0.005% BSA, 1 μg/mL poly(dI-dC), and 5% glycerol). The binding is incubated at 300 for 40 minutes, then 2 μL of 6× loading buffer (60 mM KCl, 10 mM Tris pH 7.6, 50% glycerol) is added. The binding reaction is separated on a 5% TBE gel and visualized. Shifts of the 3′ or 5′ UTR in the presence of retrotransposase protein and target DNA can be attributed to successful binding and are indicative of retrotransposase activity. This assay can also be performed with retrotransposase truncations or mutations, as well as using E. coli extract or purified protein.

### Example 5—Cleavage of Target DNA Verification (Prophetic)

To confirm that the retrotransposase is involved in cleavage of target DNA, short (˜140 bp) DNA fragments are labelled at both ends with FAM via PCR with FAM-labeled primers. In vitro transcription/translation retrotransposase products are pre-incubated with 1 μg of RNase A (negative control), or 3′ UTR, 5′ UTR or non-specific RNA fragments (control), followed by incubating with labeled target DNA at 37° C. The DNA is then analyzed on a denaturing gel. Cleavage of one or both strands of DNA can result in labelled fragments of various sizes, which migrate at different rates on the gel.

### Example 6—Integrase Activity in E. coli (Prophetic)

Engineered E. coli strains are transformed with a plasmid expressing the retrotransposon genes and a plasmid containing a temperature-sensitive origin of replication with a selectable marker flanked by 5′ and 3′ UTR of the retrotransposon required for integration. Transformants induced for expression of these genes are then screened for transfer of the marker to a genomic target by selection at restrictive temperature for plasmid replication and the marker integration in the genome is confirmed by PCR.

Integrations are screened using an unbiased approach. In brief, purified gDNA is tagmented with Tn5, and DNA of interest is then PCR amplified using primers specific to the Tn5 tagmentation and the selectable marker. The amplicons are then prepared for NGS sequencing. Analysis of the resulting sequences is trimmed of the transposon sequences and flanking sequences are mapped to the genome to determine insertion position, and insertion rates are determined.

### Example 7—Integration of Reverse Transcribed DNA into Mammalian Genomes (Prophetic)

To show targeting and cleavage activity in mammalian cells, the integrase proteins are purified in E. coli or sf9 cells with 2 NLS peptides either in the N, C or both terminus of the protein sequence. A plasmid containing a selectable neomycin resistance marker (NeoR) or a fluorescent marker flanked by the 5′ and 3′ UTR regions required for transposition and under control of a CMV promoter are synthesized. Cells are be transfected with the plasmid, recovered for 4-6 hours for RNA transcription, and subsequently electroporated with purified integrase proteins. Antibiotic resistance integration into the genome is quantified by G418-resistant colony counts (selection to start 7 days post-transfection), and positive transposition by the fluorescent marker is assayed by fluorescence activated cell cytometry. 7-10 days after the second transfection, genomic DNA is extracted and used for the preparation of an NGS library. Off target frequency is assayed by fragmenting the genome and preparing amplicons of the transposon marker and flanking DNA for NGS library preparation. At least 40 different target sites are chosen for testing each targeting system's activity.

Integration in mammalian cells can also be assessed via RNA delivery. An RNA encoding the retrotransposase with 2 NLS is designed, and cap and polyA tail are added. A second RNA is designed containing a selectable neomycin resistance marker (NeoR) or a fluorescent marker flanked by the 5′ and 3′ UTR regions. The RNA constructs are introduced into mammalian cells via Lipofectamine™ RNAiMAX or TransIT®-mRNA transfection reagent. 10 days post-transfection, genomic DNA is extracted to measure transposition efficiency using ddPCR and NGS.

### Example 8—Bioinformatic Discovery of RTs

An extensive assembly-driven metagenomic database of microbial, viral, and eukaryotic genomes was mined to retrieve predicted proteins with reverse transcriptase function. Over 4.5 million RT proteins were predicted on the basis of having a hit to the PFam domains PF00078 and PF07727, of which 3.4 million had a significant e-value (<1×10⁻⁵). After filtering for complete ORFs with an RT domain coverage of ≥70%, and with predicted catalytic residues ([F/Y]XDD), nearly half a million proteins were retained for further analysis. The RT domains were extracted from this set of proteins, as well as from reference sequences retrieved from public databases. The domain sequences were clustered at 50% identity over 80% coverage with MMseqs2 easy-cluster (Bioinformatics 2016 May 1; 32(9):1323-30), representative sequences (26,824 in total) were aligned with MAFFT with parameters—globalpair—large (Bioinformatics 2016; 32: 3246-3251), and the domain alignment was used to infer a phylogenetic tree with FastTree2 (Plos One 2010; 5: e9490). Phylogenetic analysis of RT domains suggest that many different classes of RTs with high sequence diversity were recovered (FIG. 4).

### Example 9—Non-LTR Retrotransposons (MG148 Family)

### Retrotransposon-Associated RT Bioinformatic Analysis

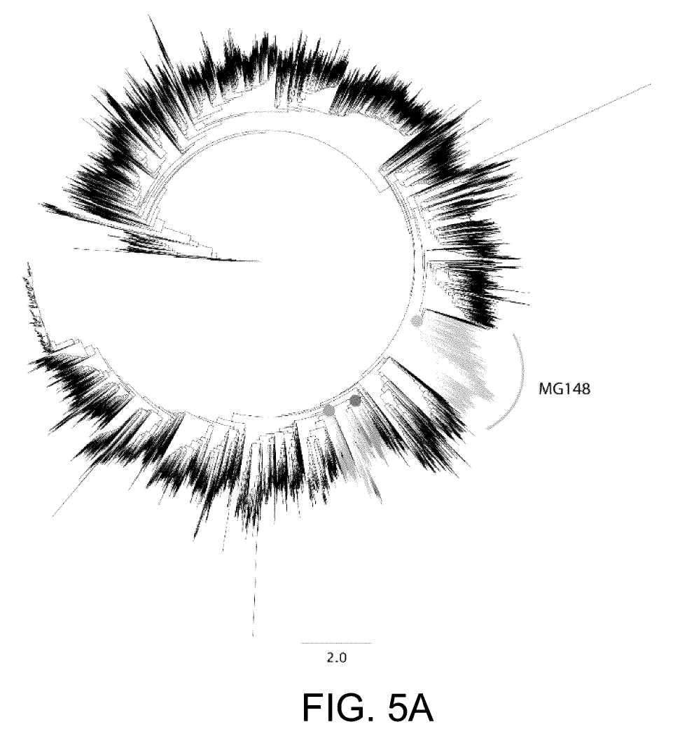

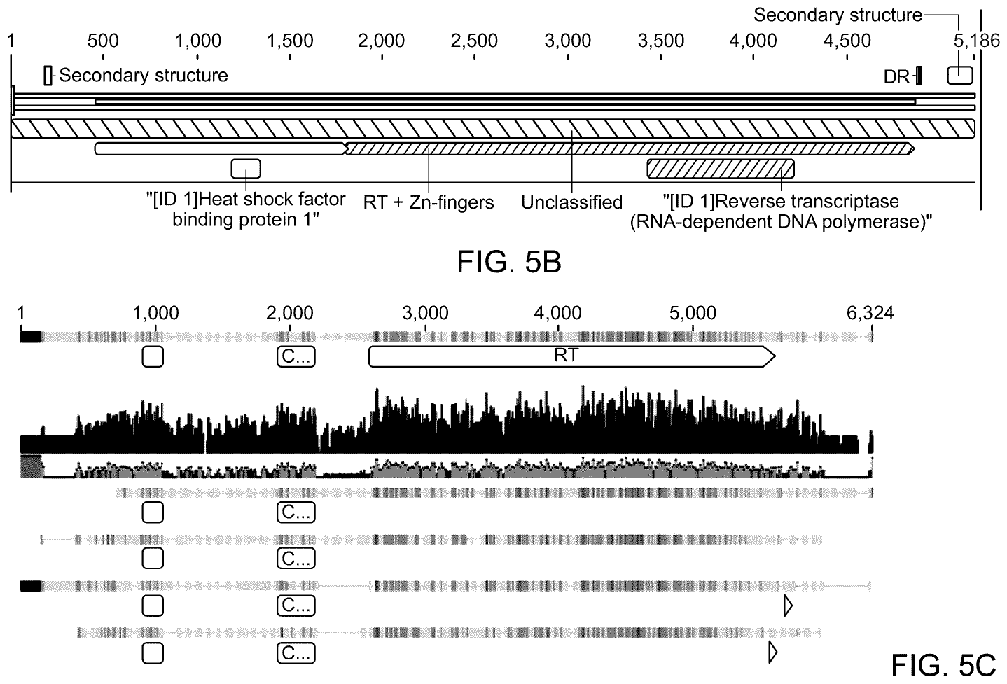

The MG148 family of retrotransposon-associated RTs includes extremely divergent RT homologs, predicted to be active by the presence of all expected catalytic residues and multiple Zn-binding ribbon motifs (FIGS. 5A and 5B). Alignment at the nucleotide level for several family members uncovered conserved regions within the 5′ UTR, which are possibly involved in RT function, activity, or mobilization (FIG. 5C).

Testing the In Vitro Activity of Retrotransposon RTs by qPCR

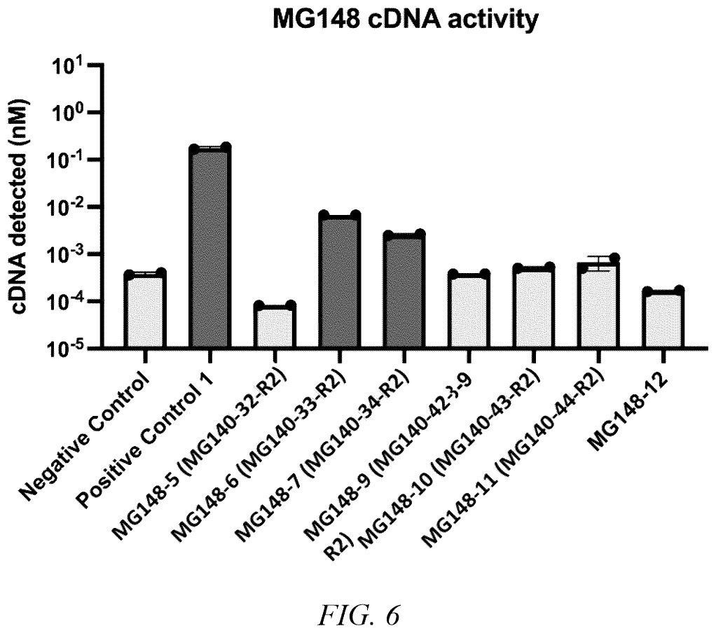

The in vitro activity of retrotransposon RTs was assessed by a primer extension reaction containing RT enzyme derived from a cell-free expression system (PURExpress, NEB) and 100 nM of RNA template (200 nt) annealed to a DNA primer in reaction buffer containing 40 mM Tris-HCl (pH 7.5), 0.2 M NaCl, 10 mM MgCl₂, 1 mM TCEP, and 0.5 mM dNTPs. The resulting full-length cDNA product was quantified by qPCR by extrapolating values from a standard curve generated with the DNA template of known concentrations. MG148 family members MG140-33-R2 through MG140-34-R2 (SEQ ID NOs: 5-6), MG140-42-R2 through MG140-44-R2 (SEQ ID NOs: 14-16), and MG148-12 (SEQ ID NO: 32) are active at cDNA synthesis as determined by primer extension (FIG. 6).

### Example 10—Group II Intron RTs (MG153 Family)

### Group II Intron Bioinformatic Analysis

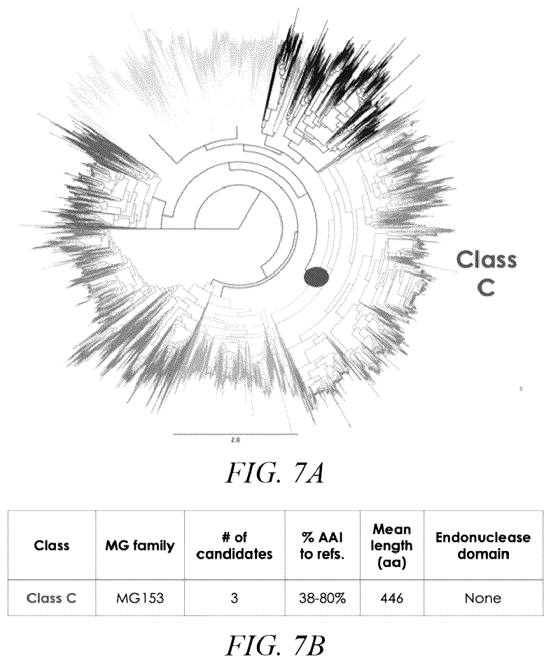

Group II introns are capable of integrating large cargo into a target site via reverse transcription of an RNA template. RT domains from Group II introns were identified and delineated in the phylogenetic tree in FIG. 4. Over 10,000 unique full-length Group II intron proteins containing RT domains from contigs with >2 kb of sequence flanking the RT enzyme were aligned with MAFFT with parameters—globalpair—large. A phylogenetic tree was inferred from this alignment and Group II intron families were further identified (FIG. 7). Group II introns of Class C were identified, and their domain architecture includes an RT domain predicted to be active, as well as a maturase domain involved in intron mobilization. Some Group II intron proteins contain an additional endonuclease domain likely involved in target recognition and cleavage. Many candidates from all families identified were nominated for laboratory characterization.

### Testing the In Vitro Activity of Group II Intron RTs Class C

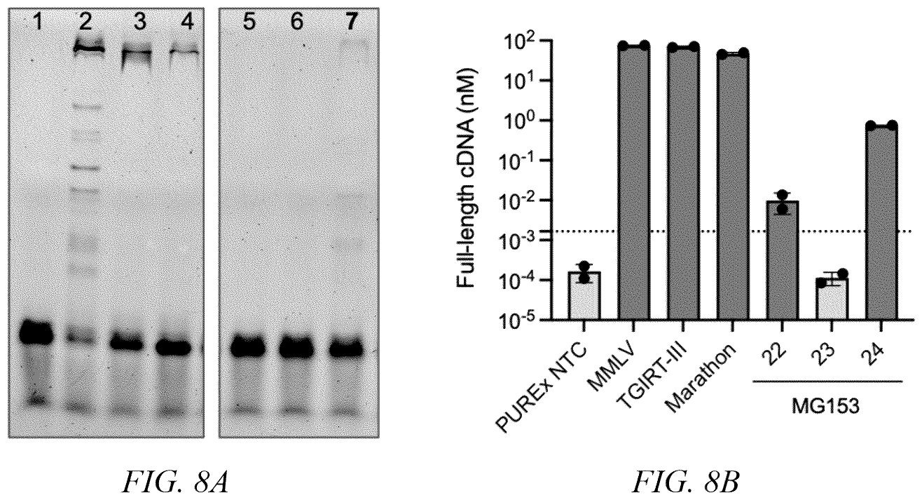

The in vitro activity of GII intron Class C (MG153) RTs was assessed by a primer extension reaction containing RT enzyme derived from a cell-free expression system (PURExpress, NEB). Expression constructs were codon-optimized for E. coli and contained an N-terminal single Strep tag. Expression of the RT was confirmed by SDS-PAGE analysis. The substrate for the reaction was 100 nM of RNA template (200 nt) annealed to a 5′-FAM labeled primer. The reaction buffer contained the following components: 50 mM Tris-HCl (pH 8.0), 75 mM KCl, 3 mM MgCl₂, 10 mM DTT, and 0.5 mM dNTPs. Following incubation at 37° C. for 1 h, the reaction was quenched via incubation with RnaseH (NEB), followed by the addition of 2× RNA loading dye (NEB). The resulting cDNA product(s) were separated on a 10% denaturing polyacrylamide gel and were visualized using a ChemiDoc on the Gel Green setting. RT activity was also assessed by qPCR with primers that amplify the full-length cDNA product. Products from the primer extension assay were diluted to ensure cDNA concentrations were within the linear range of detection. The amount of cDNA was quantified by extrapolating values from a standard curve generated with the DNA template of known concentrations. By detection of cDNA products on a denaturing gel and by qPCR, the following GII intron class C candidates are active under these experimental conditions: MG153-22 through MG153-24 (SEQ ID NOs: 42-44). (FIG. 8).

Human Cells cDNA Synthesis Results

The ability of these enzymes to produce cDNA in a mammalian environment was tested by expressing them in mammalian cells and detecting cDNA synthesis by PCR, followed by agarose electrophoresis and D1000 TapeStation. Reverse transcriptases were cloned in a plasmid for mammalian expression under the CMV promoter as fusion proteins having MS2 coat protein (MCP) at the N terminus, in addition to a flag-HA tag (FH). MCP is a protein derived from the MS2 bacteriophage that recognizes a 20 nucleotide RNA stem loop with high affinity (subnanomolar Kd). By fusing the RTs with MCP and having the MS2 loops in the RNA template, it is ensured that once the RT is translated, it finds the RNA template and starts cDNA synthesis from the DNA primer hybridized to the RNA template.

A plasmid containing MCP fused to the RT candidate under CMV promoter was cloned and isolated for transfection in HEK293T cells. Transfection was performed using lipofectamine 2000. mRNA codifying nanoluciferase was made using mMESSAGE mMACHINE (Thermo Fisher) according to the manufacturer instructions. In order to degrade any DNA template left in the mRNA preparation, the reaction was treated with Turbo DNase (Thermo Fisher) for 1 hour, and the mRNA is cleaned using MEGAclear Transcription Clean-Up kit (Thermo Fisher). The mRNA was hybridized to a complementary DNA primer in 10 mM Tris pH 7.5, 50 mM NaCl at 95° C. for 2 min and cooled to 4° C. at the rate of 0.1° C./s. The mRNA/DNA hybrid was transfected into HEK293T cells using Lipofectamine Messenger Max 6 hours after the plasmid containing the MCP-RT fusion was transfected. 18 hours post mRNA/DNA transfection, cells were lysed using QuickExtra DNA Extraction Solution (Lucigen), 100 μL of quick extract was added per 24 well in a 24 well plate. The nanoluciferase is ˜500 bp long, primers to amplify products of 100 bp and 542 bp from the newly synthesized cDNA were designed. cDNA was amplified using the set of primers mentioned above, and PCR products were detected by agarose gel electrophoresis or DNA Tape Station.

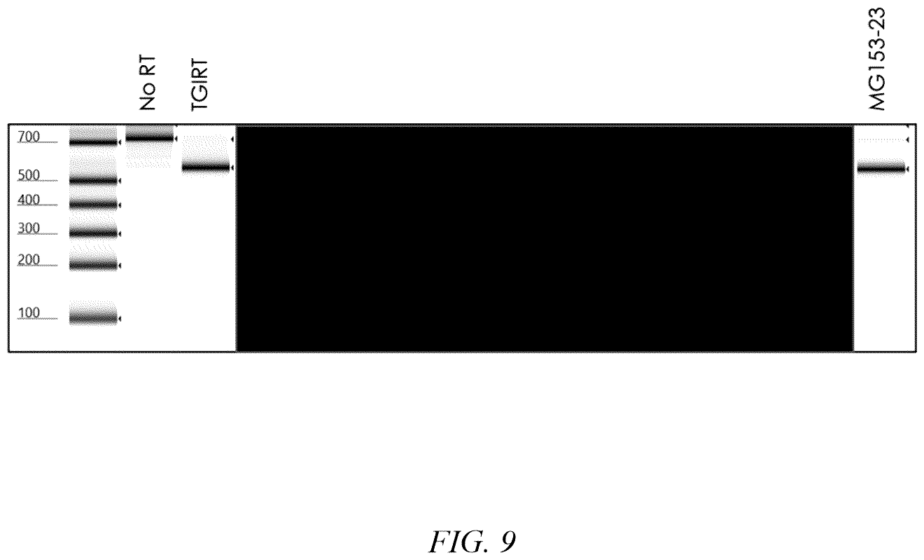

Activity for the control GII intron RTs TGIRT was detected (FIG. 9), as shown by the presence of a 500 bp DNA product. Moreover, cDNA synthesis activity for a novel GII intron derived RT, MG153-23 (SEQ ID NO: 43), was also shown (FIG. 9). Altogether, this shows that these newly discovered RTs are expressed, fold properly, and are active inside living mammalian cells, opening options for their biotechnological applications.

Human Cells RT Expression and cDNA Synthesis Results

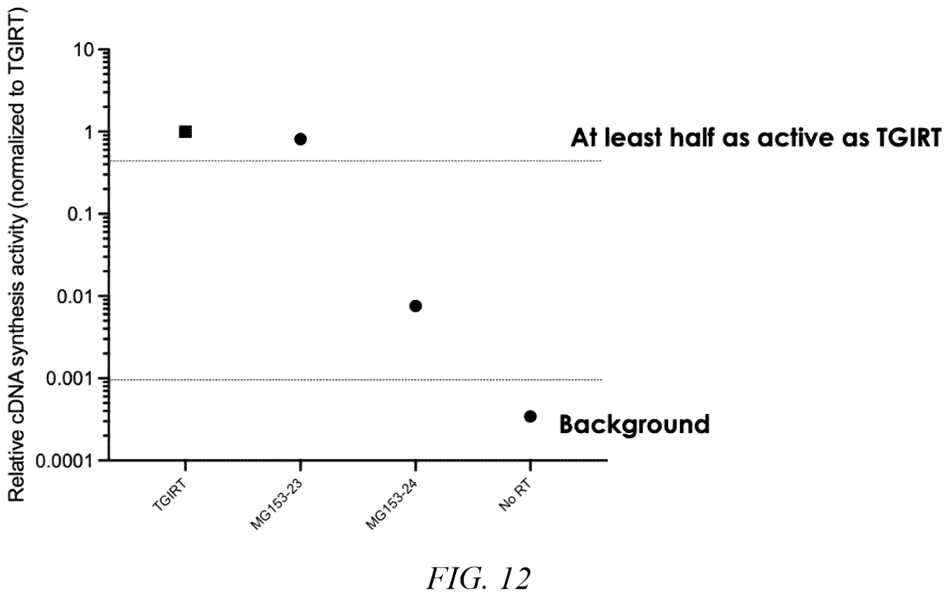

The ability of novel GII RTs to synthesize cDNA in a mammalian cell environment was tested as previously described with a small modification. cDNA synthesis was previously detected using PCR and analyzed by agarose gel electrophoresis and/or TapeStation. In order to have a quantitative readout, a Tagman qPCR assay was developed using Taqman qPCR primers previously described with a Tagman probe “ACTCTGTGAGCGGATCTTGGCTTAGCC” (SEQ ID NO: 62). MG153-23 and MG153-24 RTs were active to various degrees, with MG153-23 nearly as active as the TGIRT control (FIG. 12).

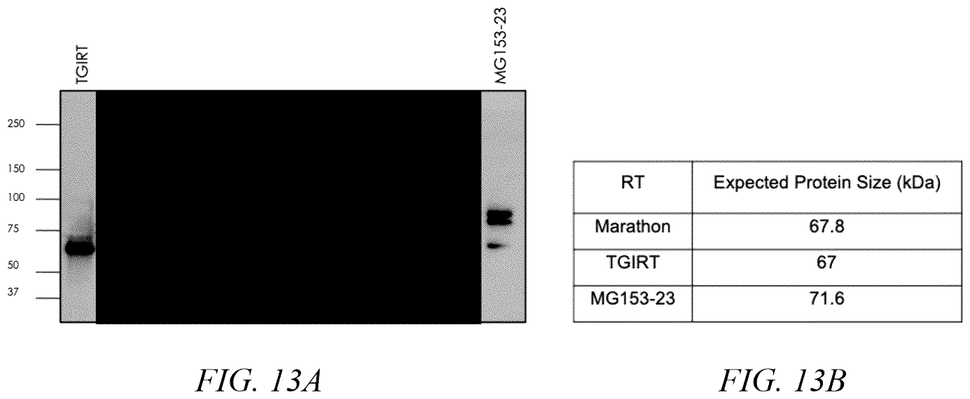

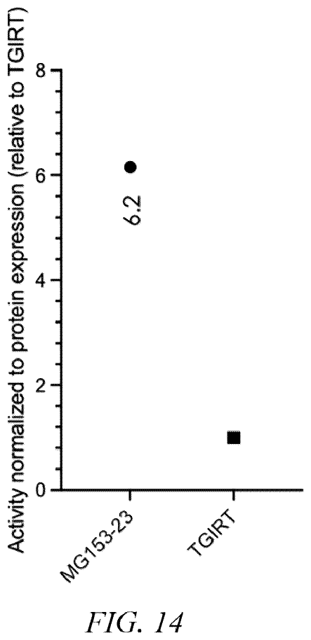

In order to understand protein expression and stability of the GII RTs in mammalian cells, immunoblots were performed. Briefly, transfected cells were lysed with RIPA lysis buffer (Thermo Fisher) supplemented with protease inhibitors (80 μL per well in a 24 well format). The lysate was centrifuged at 14,000 g for 10 min at 4° C. in order to remove insoluble aggregates. Proteins were quantified using BCA. 3 or 10 μg of total protein was loaded per lane in a 4-12% polyacrylamide SDS gel (Thermo Fisher). All lanes were normalized to the same amount of protein. Proteins were transferred to a PVDF membrane using the iBlot gel transfer system (Invitrogen). Proteins were detected by using a rabbit HA antibody (Cell Signaling), using an HRP-based detection method. Results indicate that MG153-23 is expressed in human cells, as given by the intensity of the band (FIG. 13). When normalizing cDNA synthesis by the quantified expression, the MG153-23 RTs outperformed the TGIRT control by over six-fold (FIG. 14).

### Example 11—Retron-Like RTs (MG160 Family)

### Retron Bioinformatic Analysis

Bacterial retrons are DNA elements of approximately 2000 bp in length that encode an RT-coding gene (ret) and a contiguous non-coding RNA containing inverted sequences, the msr and msd. Retrons employ a unique mechanism for RT-DNA synthesis, in which the ncRNA template folds into a conserved secondary structure, insulated between two inverted repeats (a1/a2). The retron RT recognizes the folded ncRNA, and reverse transcription is initiated from a conserved guanosine 2′OH adjacent to the inverted repeats, forming a 2′-5′ linkage between the template RNA and the nascent cDNA strand. In some retrons, this 2′-5′ linkage persists into the mature form of processed RT-DNA, while in others an exonuclease cleaves the DNA product resulting in a free 5′ end. Moreover, the RT only targets the msr-msd derived from the same retron as its RNA template, providing specificity that may avoid off-target reverse transcription.

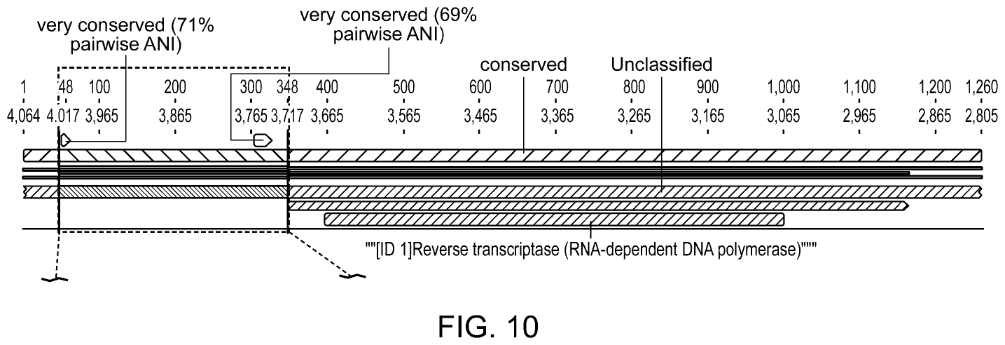

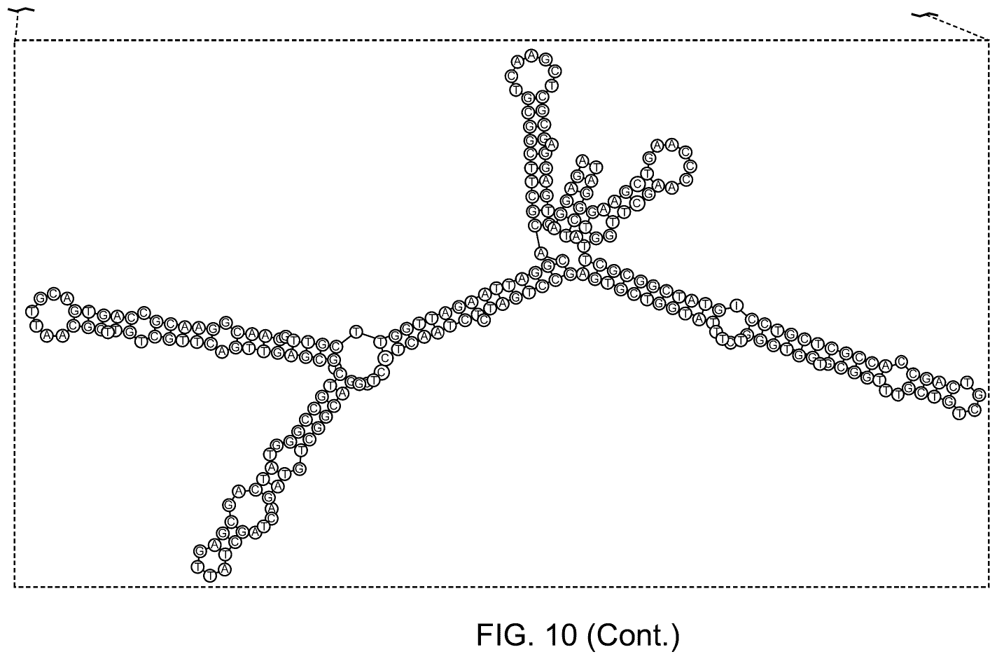

A divergent group of “retron-like” single-domain RT sequences were identified within the retron clade in FIG. 4. The single-domain RTs of the MG160 family range between 250 and 300 aa and are predicted to be active based on the presence of expected RT catalytic residues [F/Y]XDD. The 5′ UTR of the MG160 family are conserved among family members and fold into conserved secondary structures (FIG. 10) that are likely important for element activity or mobilization.

Testing the In Vitro Activity of the MG160 family of Retron-Like RTs

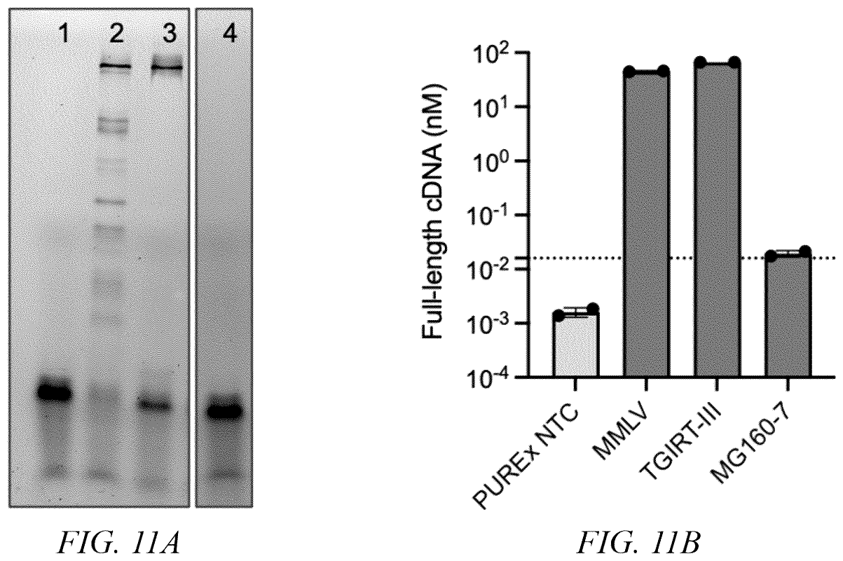

The in vitro activity of retron-like RTs (MG160 family) was assessed by a primer extension reaction containing RT enzyme derived from a cell-free expression system (PURExpress, NEB). Expression constructs were codon-optimized for E. coli and contained an N-terminal single Strep tag. The substrate for the reaction was 100 nM of RNA template (200 nt) annealed to a 5′-FAM labeled primer. The reaction buffer contained the following components: 50 mM Tris-HCl (pH 8.0), 75 mM KCl, 3 mM MgCl₂, 10 mM DTT, and 0.5 mM dNTPs. Following incubation at 37° C. for 1 h, the reaction was quenched via incubation with RnaseH (NEB), followed by the addition of 2× RNA loading dye (NEB). The resulting cDNA product(s) were separated on a 10% denaturing polyacrylamide gel and were visualized using a ChemiDoc on the Gel Green setting. RT activity was also assessed by qPCR with primers that amplify the full-length cDNA product. Products from the primer extension assay were diluted to ensure cDNA concentrations were within the linear range of detection. The amount of cDNA was quantified by extrapolating values from a standard curve generated with the DNA template of known concentrations. By gel analysis and by qPCR, MG160-7 (SEQ ID NO: 45) is active (FIG. 11).

### Example 12—Cell-Free Expression of Retron RTs and In Vitro Transcription of Retron ncRNAs (Prophetic)

Retron RTs are produced in a cell-free expression system (PURExpress) by incubating 10 ng/μL of a DNA template encoding the E. coli-optimized gene with an N-terminal single Strep tag with the PURExpress components for 2 h at 37° C. All tested retron RTs are expressed as indicated by SDS-PAGE analysis.

The retron ncRNAs are generated using the HiScribe T7 in vitro transcription kit (NEB) and a DNA template encoding the respective ncRNA gene following a T7 promoter. The reaction is then incubated with DNase-I to eliminate the DNA template and purified by an RNA cleanup kit (Monarch). Quantity of the ncRNA is determined by nanodrop and the purity assessed by TapeStation RNA analysis.

### Example 13—Testing Retron RT In Vitro Activity (Prophetic)

The retron RT enzyme is produced in a cell-free expression system using a construct containing an E. coli codon-optimized gene with an N-terminal single Strep tag as described above. Expression of the enzyme is confirmed by SDS-PAGE analysis. Retron RT activity on a general template is determined by a primer extension assay as described above, containing a 200 nt RNA annealed to a 5′-FAM labeled DNA primer. The resulting cDNA product(s) are detected on a denaturing polyacrylamide gel or by qPCR with primers specific for the full-length cDNA product.

Retron RT in vitro activity on its own ncRNA is assessed in a reaction containing buffer, dNTPs, the retron RT produced from a cell-free expression system, and the refolded ncRNA. RT activity before and after purification of the RT from the cell-free expression system via the N-terminal single Strep tag is compared. After incubation, half of the reaction is treated with RNase A/T1. Products before and after RNase A/T1 treatment are evaluated on a denaturing polyacrylamide gel and visualized by SYBR gold staining. RNase A/T1 should digest away the RNA template and result in a mass shift towards a smaller product containing only the ssDNA. Since RNase H is expected to improve homogeneity of the 5′ and 3′ ssDNA boundaries, the impact of RNase H on the distribution of products is also evaluated by gel analysis. The covalent linkage between the ncRNA template and ssDNA is confirmed by incubating the RT product with a 5′ to 3′ ssDNA exonuclease (RecJ) before or after treatment with a debranching enzyme (DBR1). RecJ should only be able to degrade the ssDNA after DBR1 has removed the 2′-5′ phosphodiester linkage between the RNA and ssDNA.

### Example 14—Determining Retron Msr-Msd Boundaries by NGS (Prophetic)

The msr-msd boundaries are determined by unbiased ligation of adapter sequences to the 5′ and 3′ end of the msDNA product after removal of the 2′-5′ phosphodiester linkage by DBR1. The resulting ligated product is PCR-amplified, library prepped, and subjected to next generation sequencing. Sequencing reads are aligned to the reference sequence to determine the 5′ and 3′ boundaries of the msd. The impact of the presence of RNase H in the RT reaction on the homogeneity of 5′ and 3′ msd boundaries is also evaluated.

### Example 15—Systemic Evaluation of Insertion Sequences into the Msd on RT Activity (Prophetic)

Sequences of distinct length, predicted secondary structure, and GC-content are inserted into the msd at select insertion sites informed by the msd boundaries determined by NGS and secondary structure predictions of the ncRNA. The impact of these insertion sequences on RT activity are assessed by gel analysis or NGS as described above.

### Example 16—Testing the In Vitro Activity of RTs (Prophetic)

RT activity is assessed using a primer extension assay containing the RT derived from a cell-free expression system and an RNA template annealed to a DNA primer as described above. The resulting cDNA product(s) are detected by a denaturing polyacrylamide gel and qPCR as described above. Detection of cDNA drop-off products on the denaturing gel provides a relative assessment of processivity for novel candidates.

### Example 17—Evaluating the Priming Requirements of RTs (Prophetic)

Primer length preference is determined by testing the RT's activity on an RNA template annealed to 5′-FAM labeled DNA primers of either 6, 8, 10, 13, 16, or 20 nucleotides in length. The RT is derived from a cell-free expression system as described above. After incubating the reaction, the reaction is quenched via the addition of RNase H. The size distribution of cDNA products is analyzed on a denaturing polyacrylamide gel as described above. Optimal primer length is determined as the length that enables the RT to convert the most primer into cDNA product. The experimentally determined optimal primer length is then used in subsequent experiments, such as fidelity and processivity assays, to further characterize the RT in vitro.

### Example 18—Evaluating RT Fidelity (Prophetic)

To account for errors introduced during PCR and sequencing, RT fidelity is assessed by a primer extension assay as described above with the exception that a 14-nt unique molecular identifier (UMI) barcode is included in the primer for the reverse transcription reaction. The resulting full-length cDNA product is PCR-amplified, library-prepped, and subjected to next-generation sequencing. Barcodes with >5 reads are analyzed. After aligning to the reference sequence, mutations, insertions, and deletions are counted only if the error is present in all sequence reads with the same barcode. Errors present in one but not all sequencing reads are considered to be introduced during PCR or sequencing. Further analysis of substitution, insertion, and deletion profile is performed, in addition to identification of mutation hotspots within the RNA template. The fidelity measurements will also be performed with modified bases, e.g. pseudouridine, in the template.

### Example 19—Determining the Processivity Coefficient of RTs (Prophetic)

RT processivity is evaluated using a primer extension assay containing the RT enzyme derived from a cell-free expression system as described above and RNA templates between 1.6 kb-6.6 kb in length annealed to either a 5′-FAM labeled primer (for gel analysis) or an unlabeled primer (for sequencing analysis).

Reverse transcription reactions are performed under single cycle conditions to prevent rebinding of RT enzymes that have dropped off the RNA template during cDNA synthesis. The optimal trap molecule and concentration to achieve single cycle conditions are experimentally determined. The selected condition should provide sufficient inhibition of cDNA synthesis if incubated prior to reaction initiation but otherwise should not impact the velocity of the reaction. Optimal trap molecules to test include unrelated RNA templates and unrelated RNA templates annealed to DNA primers of various lengths.

Once single cycle reaction conditions have been optimized, processivity is evaluated by initiating the reaction with the addition of dNTPs and the selected trap molecule after pre-equilibrating the RT with the RNA template annealed to a DNA primer in the reaction buffer. After incubating the reaction, the reaction is quenched by the addition of RnaseH. The size distribution of cDNA products is analyzed on a denaturing polyacrylamide gel as described above and/or subjected to PCR and library prepped for long-read sequencing. From these experiments, a processivity coefficient is quantified as the template length which yields 50% f the full-length cDNA product. The median length of the cDNA product from the single cycle primer extension reaction is used to estimate the probability that the RT will dissociate on the tested template. From this, the probability that the RT will dissociate at each nucleotide position is calculated, assuming that each dissociation is an independent event and that the probability of dissociation is equal at all nucleotide positions. The processivity coefficient representing the length of template required for 50% f RT dissociated is then determined as 1/(2*Pd), where Pd is the probability of dissociation at each nucleotide.

### Example 20—Systematic Analysis of Challenge Structures on Primer Extension (Prophetic)

To evaluate the impact of challenging templates on RT activity, a primer extension reaction is conducted as stated above, with modifications. The RNA template contains one of the following challenge motifs at fixed distance (100-300 nt) downstream of the primer binding site: homopolymeric stretches, thermodynamically stable GC-rich stem loop, pseudoknot, tRNA, GII intron, and RNA template containing base or backbone modifications (i.e. pseudouridine, phosphothiorate bonds). After quenching the reaction, the size distribution of cDNA products is analyzed by denaturing polyacrylamide gel. An adapter sequence is also unbiasedly ligated to the 3′ ends of the cDNA products using T4 ligase. The ligated product(s) are then PCR-amplified, and library prepped for next generation sequencing to identify both sites of RT misincorporation/insertions/deletions and sites of RT drop-off with single nucleotide resolution. Extent of RT drop-off at a given position is quantified by comparing the number of sequencing reads corresponding to the drop-off product to the number of sequencing reads corresponding to the full-length product.

### Example 21—Evaluating Non-Templated Base Additions (Prophetic)

Non-templated addition of bases to the 5′ end of the cDNA product is evaluated by next generation sequencing. Primer extension reactions containing the RT derived from the cell-free expression system and RNA template are conducted as described above. Systematic analysis of different RNA template lengths and sequence motifs at the 5′ end are tested. An adapter sequence is unbiasedly ligated to the 3′ ends of the resulting cDNA products by T4 ligase, resulting in capture of all cDNA products despite the potential heterogeneous nature of their 3′ ends. The ligated product(s) are then PCR-amplified, and library prepped for next generation sequencing. Comparison of the expected full-length cDNA reference sequence to experimentally produced cDNA sequences that are longer than full-length enable identification of both the type and number of base additions to the 5′-end that were not templated by the RNA.

### Example 22—Determining 5′ and 3′ UTR Requirements for Activity and Processivity for R2-Like Systems (Prophetic)

Proteins of interest are purified via a Twin-strep tag after IPTG-induced overexpression in E. coli. Purified proteins are tested against 1 kb and 4 kb cargos flanked by the 3′ UTRs identified from their native contexts and the 5′ UTRs plus 400 bp past the start codon. The 5′ and 3′ flanking sequences' effect on activity is assayed via qPCR to sections near the end of the template to determine if cargos with these native features are preferred substrates.

### Example 23—Human Cells cDNA Synthesis Results (Prophetic)

The ability of these enzymes to produce cDNA in a mammalian environment is tested by expressing them in mammalian cells and detecting cDNA synthesis by PCR, followed by agarose electrophoresis and D1000 TapeStation. Reverse transcriptases are cloned in a plasmid for mammalian expression under the CMV promoter as fusion proteins having MS2 coat protein (MCP) at the N terminus, in addition to a flag-HA tag (FH). MCP is a protein derived from the MS2 bacteriophage that recognizes a 20 nucleotide RNA stem loop with high affinity (subnanomolar Kd). By fusing the RTs with MCP and having the MS2 loops in the RNA template, it is ensured that once the RT is translated, it finds the RNA template and starts cDNA synthesis from the DNA primer hybridized to the RNA template.

A plasmid containing MCP fused to the RT candidate under CMV promoter is cloned and isolated for transfection in HEK293T cells. Transfection is performed using lipofectamine 2000. mRNA codifying nanoluciferase is made using mMESSAGE mMACHINE (Thermo Fisher) according to the manufacturer instructions. In order to degrade any DNA template left in the mRNA preparation, the reaction is treated with Turbo DNase (Thermo Fisher) for 1 hour and the mRNA is cleaned using MEGAclear Transcription Clean-Up kit (Thermo Fisher). The mRNA is hybridized to a complementary DNA primer in 10 mM Tris pH 7.5, 50 mM NaCl at 95° C. for 2 min and cooled to 4° C. at the rate of 0.1° C./s. The mRNA/DNA hybrid is transfected into HEK293T cells using Lipofectamine Messenger Max 6 hours after the plasmid containing the MCP-RT fusion was transfected. 18 hours post mRNA/DNA transfection, cells are lysed using QuickExtra DNA Extraction Solution (Lucigen), 100 μL of quick extract is added per 24 well in a 24 well plate. The nanoluciferase is ˜500 bp long, primers to amplify products of 100 bp and 542 bp from the newly synthesized cDNA are designed. cDNA is amplified using the set of primers mentioned above and PCR products are detected by agarose gel electrophoresis or DNA Tape Station.

### Example 24—RT cDNA Synthesis Activity can be Harnessed for Multiple Applications (Prophetic)

Processes dependent on RNA important in RNA biology, such as expression, processing, modifications, and half-life, as well as quality control steps in biotechnology, require a crucial step: conversion of RNA to cDNA. Therefore, multiple RTs have been used for the production of cDNA libraries over the years. Commercially available RTs used for these purposes include the MMLV RT, AMV RT, and GsI-IIC RT (TGIRT). The first two represent retroviral RTs, while the latter is a GII intron-derived RT. GII intron-derived RTs, as well as non-LTR derived RTs, show several advantages compared to their retroviral counterparts. For example, they are more processive, reading through structural and modified RNAs. Structural and/or modified RNAs can't be properly reverse transcribed by retroviral RTs, as they create early termination products that can be misinterpreted as RNA fragments. In addition, the ability to template switch of some RTs can be harnessed for early adaptor addition, removing the adaptor ligation step during library preparation. Therefore, highly processive RTs are suitable for the generation of libraries with complex RNA. Further, some highly processive RTs are generally smaller than currently used retroviral RTs, making their production and associated downstream steps easier. Data disclosed herein demonstrates that several novel RTs described herein outperform the commercially available TGIRT enzyme, some with over six-fold its cDNA synthesis activity. As such, many of these novel RTs show great promise for their commercial application for cDNA synthesis kits.

TABLE 2
Protein and nucleic acid sequences referred to herein

| | SEQ | | | | | |
|---|---|---|---|---|---|---|
| | ID | | | | Other | |
| Cat. | NO: | Description | Type | Organism | Information | Sequence |
| MG140 | 1 | MG140-29- | protein | unknown | uncultivated | MDGVFAVHNVDNLTDHEPIVLQ |
| transposition | | R2 | | | organism | LSLKTDVLQNSEIVHVPRVSWR |
| proteins | | transposition | | | | KASHDQVRNYKNVLSSNLKSIY |
| | | protein | | | | LPVKTLLCNDIHCSNPIHFEDI |
| | | | | | | NRYSISIHDACISAARDTIPVT |
| | | | | | | CRRGSRGRIPGWTERVKPFRER |
| | | | | | | SLFWHNIWVDCGRPRDGHVAAA |
| | | | | | | MRRTRAAYHLQIKLVRKNEDAV |
| | | | | | | VREKVADSVIHDRGRNEWSEIK |
| | | | | | | KIRGTKTRPPGTVDGLTDTPSI |
| | | | | | | AALFADKYRDLYTSVPYDKDDM |
| | | | | | | QRIIDVVDSKLSCTNFSGDCTF |
| | | | | | | YATEVREAIGHMKRGKNDGSNE |
| | | | | | | LLSDHLINANDDLIIHIACLES |
| | | | | | | TFSVHGAVPANFHSSVILPIPK |
| | | | | | | SRNASAASSNNYRGIALSSLLG |
| | | | | | | KILDYIILERYRDTLSSCEYQF |
| | | | | | | GFKRSSSTNLCTMVLKVTLNYY |
| | | | | | | HDNSTNVFCCFLDATKAFDRVN |
| | | | | | | YCKLFRLLLTRDLPPHIIRLLI |
| | | | | | | NIYVNNNVCVSWCNVKSDTFRA |
| | | | | | | LNGVKQGAVLSPILYCVYVDNL |
| | | | | | | LQLLEKAGVGCHIGFQFLGALA |
| | | | | | | YADDLVLIAPTASAMRTMLSVC |
| | | | | | | DNFARDEDVIENASKSKWLAVI |
| | | | | | | PSNRSFMSSMINDCLFEVGGNP |
| | | | | | | IDFVDKFVHLGHIISSNLTDTA |
| | | | | | | DVESRRSSFIAEVNNMSCFFSK |
| | | | | | | LDVETRESLERSYCTSYYGSEL |
| | | | | | | WKLNCPSIDSFYTSWRRALRTV |
| | | | | | | WRLPFRAHSFLLPLISNVLPER |
| | | | | | | DEICRRSINFMRSCLFHNSYLV |
| | | | | | | RFISYNSIMFGTKSSFLRHNAY |
| | | | | | | YCASRLGCGLDSIIYGNVCVLA |
| | | | | | | EVFSNEMFADAGMLLELLLLRD |
| | | | | | | GVLHCGLNIDEISTLIIHLCTN |
| | | | | | | D |
| MG140 | 2 | MG140-30- | protein | unknown | uncultivated | MILSIDNTSDHDPIILRLSLDI |
| transposition | | R2 | | | organism | KYVSVCNRTSSSRVSWVKASDR |
| proteins | | transposition | | | | DIRNYQYNLASNLQHVTIPSVA |
| | | protein | | | | LLCKDVNCSNFAHRCQLSRYLT |
| | | | | | | DISDACLAAGEASIPHTCSRHS |
| | | | | | | GKRIPGWSEKVEPLRQRSLFWH |
| | | | | | | SMWVECGRPRSGVVADCMRRAR |
| | | | | | | ASYHYAIRAVRRNEQNIIRERV |
| | | | | | | ADALLRDPSRDFWTEVKKIRNS |
| | | | | | | KSGRAVIVDGCSDAPSISQLFA |
| | | | | | | SKYRHLYTSVPYQHNDLQSIVS |
| | | | | | | DVESRISEDGDCFIGSQEVMAA |
| | | | | | | LSKLKLHKNDGDLGLASDYFIN |
| | | | | | | SDPALSVHIALLFTGIVIHGFV |
| | | | | | | PSNLLSSTIVPIPKKSNVNATD |
| | | | | | | SDNYRGIALSSVLGKIFDNVIL |
| | | | | | | VKYSDKLSTCNLQFGFKRNSST |
| | | | | | | HMCTMVLKETISYYVNNNSSVE |
| | | | | | | CTFLDASKAFDRVHYCKLERLL |
| | | | | | | LGRGLPVCILRVLIQLYVGHSI |
| | | | | | | RVTWAGLVSSCFTALNGVKQGG |
| | | | | | | VLSPVLFCIYVDELLIRLAESG |
| | | | | | | VGCYIGESFAGALAYADDIVLI |
| | | | | | | APTPSAMRKLLAICDTYATEYN |
| | | | | | | ILFNAQKSNFIAFVPSSRHFLH |
| | | | | | | KAMTNCVFRIGGVQIEHVESYT |
| | | | | | | HLGHIITSRLDDADDILHRRSS |
| | | | | | | YIGQVNNVVCYFDSLSWTVKLG |
| | | | | | | LHKSYCSSIFGCELWALDSVRD |
| | | | | | | IEKFCVAWRKGLRRVLSLPRAA |
| | | | | | | HSHLLPLLSNSLPVYDEICKRS |
| | | | | | | AKFIVSCQCSDNILVRAVVNYA |
| | | | | | | IAARSKSVLGRNVMLLCRRFHL |
| | | | | | | SFDDFVSGRLLLSGDIFVSHYL |
| | | | | | | NSLSEAQLQSVCFALELLCLRE |
| | | | | | | QSFKLNNNMRLNADEISDYLAA |
| | | | | | | VLC |
| MG140 | 3 | MG140-31- | protein | unknown | uncultivated | MLLFIRSVMENSNSVLDNLQLT |
| transposition | | R2 | | | organism | ICSYNCRGENGFKKDYISGLLE |
| proteins | | transposition | | | | SAQIDVLLLQEHWLSDAQLNAL |
| | | protein | | | | NNVGANYLNFGVSGEDTSAVLG |
| | | | | | | GRPYGGCAVLWRSDLLLQVQPL |
| | | | | | | VVSSRRLSAVIFSTDNWSLILI |
| | | | | | | NVYMPYEGDEIKTDEFIDLLSI |
| | | | | | | IEDLVLSNSASHVIVGGDENVE |
| | | | | | | ENRNRMHTALLNSFCDNTGLSP |
| | | | | | | VIQHSSCNIDYTYNENMSRENI |
| | | | | | | LDHFLLSGTLFDVCVTSAYVLH |
| | | | | | | DIDNTSHHDPIILRLSLDIKYV |
| | | | | | | SVCNRTSSSRVSWVKASDRDIR |
| | | | | | | NYQYNLASNLQHVTIPSVALLC |
| | | | | | | KDVNCSNFAHRCQLSRYLTDIS |
| | | | | | | DACLAAGEASIPHTCSRHSGKR |
| | | | | | | IPGWSEKVEPLRQRSLFWHSMW |
| | | | | | | VECGRPRSGVVADCMRRARASY |
| | | | | | | HYAIRAVRRNEQNIIRERVADA |
| | | | | | | LLRDPSRDFWTEVKKIRNSKSG |
| | | | | | | RAVIVDGCSDAPSISQLFASKY |
| | | | | | | RHLYTSVPYQHNDLQSIVSDVE |
| | | | | | | SRISFDGDCFIGSQEVMAALSK |
| | | | | | | LKLHKNDGDLGLASDYFINSDP |
| | | | | | | ALSVHIALLFTGIVIHGFVPSN |
| | | | | | | LLSSTIVPIPKKSNVNATDSDN |
| | | | | | | YRGIALSSVLGKIFDNVILVKY |
| | | | | | | SDKLSTCNLQFGEKRNSSTHMC |
| | | | | | | TMVLKETISYYVNNNSSVFCTF |
| | | | | | | LDASKAFDRVHYCKLERLLLGR |
| | | | | | | GLPVCILRVLIQLYVGHSIRVT |
| | | | | | | WAGLVSSCFTALNGVKQGGVLS |
| | | | | | | PVLFCIYVDELLIRLAESGVGC |
| | | | | | | YIGESFAGALAYADDIVLIAPT |
| | | | | | | PSAMRKLLAICDTYATEYNILF |
| | | | | | | NAQKSNFIAFVPSSRHFLHKAM |
| | | | | | | TNCVFRIGGVQIEHVFSYTHLG |
| | | | | | | HIITSRLDDADDILHRRSSYIG |
| | | | | | | QVNNVVCYFDSLSWTVKLGLHK |
| | | | | | | SYCSSIFGCELWALDSVRDIEK |
| | | | | | | FCVAWRKGLRRVLSLPRAAHSH |
| | | | | | | LLPLLSNSLPVYDEICKRSAKF |
| | | | | | | IVSCQCSDNILVRAVVNYAIAA |
| | | | | | | RSKSVLGRNVMLLCRRFHLSED |
| | | | | | | DFVSGRLLLSGDIFVSHYLNSL |
| | | | | | | SEAQLQSVCFALELLCLREQSF |
| | | | | | | KLNNNMRLNADEISDYLAAVLC |
| MG140 | 4 | MG140-32- | protein | unknown | uncultivated | MVQNTELADCINITLQTNDSRF |
| transposition | | R2 | | | organism | IVTSYNMHGENQGLEGTKEMIN |
| proteins | | transposition | | | | KLYPDVIALQEHWLTPTNLDRL |
| | | protein | | | | GEVSNDYFFIGSSSMNDVVTAG |
| | | | | | | PLFGRPFGGTAILINNKLANAT |
| | | | | | | VNVACNDKFTAVLISECLVISA |
| | | | | | | YMPCAGTCDRMSLYSAIISEIQ |
| | | | | | | ALIEFYPQYKLILCADLNVELD |
| | | | | | | APSSVADLVNNFICRNNLRRTE |
| | | | | | | LANPTASRFTYMHESLQAASYI |
| | | | | | | DYIISSDALDSIAFNVLDLDIN |
| | | | | | | LSDHLPIMCVFMCKMSACYSDP |
| | | | | | | SQSVKSKSHVDDVSYFRWDHAA |
| | | | | | | LHLYYEQTRVMMEPILDKLNIC |
| | | | | | | VNSVCDEDAFIDHSKLEDIYSS |
| | | | | | | VVAALTTSANLCIPKIKHNFFK |
| | | | | | | FWWNQELNELKAAAVTSARAWQ |
| | | | | | | QVGKPKHGSVYHKYQKDKLLYK |
| | | | | | | KCIRENQKQESVSFTNELHDAL |
| | | | | | | LRKTGKDFWKTWNAKVGVKRKC |
| | | | | | | IEQVDGIVDGATIACNFAKHFE |
| | | | | | | KICQPATATFNTTMKAKFEEMR |
| | | | | | | SMYYTPFCDKTVEFDVQLIGKI |
| | | | | | | ISNMSCGKAAGLDELSAEHLKH |
| | | | | | | CHPIVIIILCKLENLFVHVGYL |
| | | | | | | PLSFGTSYTVPIPKQNGRSHAL |
| | | | | | | TVNDERGISISPVISKIFEHAI |
| | | | | | | FARFGDYFSTSDHQFGFKESLS |
| | | | | | | CSHAIYCVRNVIDHYVKRGSTV |
| | | | | | | NICTVDISKAFDTVNHFVLFIK |
| | | | | | | LMERKLPVQLLDLFVLWESMSE |
| | | | | | | TCVRWGAHDSYFFKLKAGVRQG |
| | | | | | | GVLSPYFFAVSVDDVVDKIAAC |
| | | | | | | NAGCYINNLCSAIFLYADDIIL |
| | | | | | | LSPSVCGLQRLLGICEAAITEL |
| | | | | | | DMKINASKTVCTRVGPREDSAC |
| | | | | | | VGPNLTSGGQLNWVKTCRYLGV |
| | | | | | | HFAAGRSENCSFEEAKKKFYES |
| | | | | | | FNSVYSKLGRFASEEVILNLLN |
| | | | | | | VKCVSAMLYATEACPVLSRHKH |
| | | | | | | SLDFVITRVEMKILRTGSPQVV |
| | | | | | | AECQKYFGFLPVSYRIDIRSAR |
| | | | | | | FLERFSSSVNSFCIAFGDRARK |
| | | | | | | QLSDIVCKYNISDGLSSGVLRT |
| | | | | | | VINRIYFQ |
| MG140 | 5 | MG140-33- | protein | unknown | uncultivated | MTITNDAAQLVVISYNLHGLNQ |
| transposition | | R2 | | | organism | GLPGIREFMTELKPDVIMVQEH |
| proteins | | transposition | | | | WLTSDNLNKLSDISDEYFVIGS |
| | | protein | | | | SAMDARVSAGPLFGRPFGGTAV |
| | | | | | | FIKNKYINVTVNLVTSERYVVI |
| | | | | | | QLCDWLLINVYLPCIGTSNRIL |
| | | | | | | LYSDTLCELQSIIRAHPECNCL |
| | | | | | | VGGDENTDLNDTRSHTANTVVN |
| | | | | | | GFIAGCNLHRCDCLFPTRLKHT |
| | | | | | | YVNDSMNCYSTIDYMLSSNPEK |
| | | | | | | IVAFNVLDIDLNLSDHLPIISI |
| | | | | | | CVFDCNCKMNNPKPTSTENVTH |
| | | | | | | FRWDHAPLQDYYEHTRLGLQPI |
| | | | | | | LADLDELIGNKLSYSNVDFLCQ |
| | | | | | | VDCIYNRVVSVLQQCSHAYVPK |
| | | | | | | HKKNFYKFWWNQELDELKDKAI |
| | | | | | | ASCKMWKDAGKPRHGAIHAKYR |
| | | | | | | QDKLLYKKRIRQERVQETSSFT |
| | | | | | | NELHDALLNKSGRDFWKKWNSK |
| | | | | | | FENKSNKILQVGGTSDTATILN |
| | | | | | | NFAKHFEQVCVPFNATRNEELK |
| | | | | | | SRYKEMRLNYSESSVINDTTVF |
| | | | | | | DVQLVENLLTNMKNGKAGGLDE |
| | | | | | | LTSEHLKFCHPVVVIILCKLFN |
| | | | | | | LFVINGHIPDSFGVSYTVPIPK |
| | | | | | | SDGRSRSMTVDDERGISISPVI |
| | | | | | | SKLFELCVLDRYSDYLQTSDHQ |
| | | | | | | FGFKKQLGCRHAIFSVRSVIEQ |
| | | | | | | YISNGSTVNLCALDLSKAFDRM |
| | | | | | | NQYALFIKLMERRFPVKILTIL |
| | | | | | | EQWFSIAETCVRWGSEFSYFFS |
| | | | | | | LLAGVRQGGVLSPVLFAIFIDG |
| | | | | | | IVNRVNATNVGCYNSTVCVSIF |
| | | | | | | LYADDILLLSPTVTGLQTLLTV |
| | | | | | | CENELCELDMRLNVNKSVCMRF |
| | | | | | | GARFKAHCANLVSVQGGALQWA |
| | | | | | | SSCRYLGVYFVSGRVFRCCFHS |
| | | | | | | AKCNFFRAFNSVYSKIGCCASE |
| | | | | | | EVILSLLKSKCLPYILYGVEAC |
| | | | | | | PVLQRDKHSFDFTLTRTLMKLF |
| | | | | | | MTSSPVIVNECQTQFNLLPLRY |
| | | | | | | QIDIRTVKFLNQYIISRNSICM |
| | | | | | | LYKSHAQSVLDTIFGVYGNNVS |
| | | | | | | SLHDLHNIINEHFYDENPSTLG |
| | | | | | | YN |
| MG140 | 6 | MG140-34- | protein | unknown | uncultivated | MYNLRITSYNCRSLSALKRDFV |
| transposition | | R2 | | | organism | KTLLVSCDILFLQEHWLSDDQL |
| proteins | | transposition | | | | TLLGGLDEGFTFTGVSGEGNKE |
| | | protein | | | | ILSGRPYGGCAILWRSALKFSV |
| | | | | | | DFLSVNSRRVSAIKLSNDCCNL |
| | | | | | | LLINVYMPFEDSDCNVNEFFDV |
| | | | | | | LFDIEYLLVNNVDCHYLIGGDF |
| | | | | | | NVDLSRNTVHTALLRSFSDNNG |
| | | | | | | LLYASDEDGANFDFTYQFNMSR |
| | | | | | | FSLIDNEMLSSFLFHNMLQRAD |
| | | | | | | VVHDVNNLSDHEPVTIELCMSV |
| | | | | | | THVDIQRSTVPPLQKVSWTTAA |
| | | | | | | ESHVIDYREELVDRLKTVVMPI |
| | | | | | | DSLTCCDRRCSVAKHRSDIAEY |
| | | | | | | ANNIADVCIKAGLTTIPLMKPG |
| | | | | | | HRATPGFSEHVKPARDKSMFWH |
| | | | | | | QLWLECGRPRTGHVADCMRRTR |
| | | | | | | AAYHYALRSVKRRRDEITQERE |
| | | | | | | ATALLHNNSRNEWSEVKKIRAN |
| | | | | | | RMTYNGVVDGHSDATDIVRIFG |
| | | | | | | ERYRDLYTSVPYDECNMHEIND |
| | | | | | | TIDEHISDDDDAAFVVSLRDVS |
| | | | | | | DAIAHIKHYKNDVDNMLTSDHF |
| | | | | | | INAPSVLHVHISILFSAMLMHG |
| | | | | | | CVPNLLMYSSIRPIPKGHNLST |
| | | | | | | CDSNNYRGIAISSIENKIEDNV |
| | | | | | | VLIKYRHLLSTCDLQFGEKKKH |
| | | | | | | STQMCTMVLKETLSYYLSNRSN |
| | | | | | | VFCTELDATKAFDRINYCKLFN |
| | | | | | | LLLCRSLPYCIIRVLLSLYTNN |
| | | | | | | YVYVSWVGSNSSSFRACNGVKQ |
| | | | | | | GGVLSPVLFCLYMDGLLNKLSH |
| | | | | | | AGVGCYMGEMFVGALAYADDIV |
| | | | | | | LISPTPSAMNNMLSICDEFSIE |
| | | | | | | YNVLFNPLKSKCMYFYPKSRST |
| | | | | | | LLLHRYNVCDLQFSINGKAIEF |
| | | | | | | VDSYKHLGHVICSDMTDDIDIS |
| | | | | | | EKRGVFIGQANNIICYFAKLSS |
| | | | | | | AIKYRLFTSYCTSFFGCELWRI |
| | | | | | | SNDSLDSMCTAWRRAIRRIWSL |
| | | | | | | PYTAHGRFLPVLCNSYNIFDQF |
| | | | | | | CVRILNFIRRCLSDQSSLLVRS |
| | | | | | | IATQALMFHHARSPLGYNFIYC |
| | | | | | | ARRYCFSLTDFVNYNNFVSNVE |
| | | | | | | KLNCTQNDDDTIANCRLLRELI |
| | | | | | | DLRDGVLHLSDDVELTNSELSF |
| | | | | | | MINCVSTL |
| MG140 | 7 | MG140-35- | protein | unknown | uncultivated | MYNLRITSYNCRSLSALKRDFV |
| transposition | | R2 | | | organism | KTLLVSCDILFLQEHWLSDDQL |
| proteins | | transposition | | | | TLLGGLDEGFTFTGVSGFGNKE |
| | | protein | | | | ILSGRPYGGCAILWRSALKFSV |
| | | | | | | DFLSVNSRRVSAIKLSNDCCNL |
| | | | | | | LLINVYMPFEDSDCNVNEFFDV |
| | | | | | | LFDIEYLLVNNVDCHYLIGGDE |
| | | | | | | NVDLSRNTVHTALLRSFSDNNG |
| | | | | | | LLYASDFDGANFDFTYQFNMSR |
| | | | | | | FSLIDNEMLSSFLFHNMLQRAD |
| | | | | | | VVHDVNNLSDHEPVTIELCMSV |
| | | | | | | THVDIQRSTVPPLQKVSWTTAA |
| | | | | | | ESHVIDYREELVDRLKTVVMPI |
| | | | | | | DSLTCCDRRCSVARHRSDIAEY |
| | | | | | | ANNIADVCIKAGLTTIPLMKPG |
| | | | | | | HRATPGFSEHVKPARDKSMFWH |
| | | | | | | QLWLECGRPRTGHVADCMRRTR |
| | | | | | | AAYHYALRSVKRRRDEITQERE |
| | | | | | | ATALLHNNSRNFWSEVKKIRAN |
| | | | | | | RMTYNGVVDGHSDATDIVRIFG |
| | | | | | | ERYRDLYTSVPYDECNMHEIND |
| | | | | | | TIDEHISDDDDAAFVVSLRDVS |
| | | | | | | DAIAHIKHYKNDVDNMLTSDHF |
| | | | | | | INAPSVLHVHISILFSAMLMHG |
| | | | | | | CVPNLLMYSSIRPIPKGHNLST |
| | | | | | | CDSNNYRGIAISSIENKIEDNV |
| | | | | | | VLIKYRHLLSTCDLQFGFKKKH |
| | | | | | | STQMCTMVLKETLSYYLSNRSN |
| | | | | | | VFCTFLDATKAFDRINYCKLEN |
| | | | | | | LLLCRSLPYCIIRVLLSLYTNN |
| | | | | | | YVYVSWVGSNSSSFRACNGVKQ |
| | | | | | | GGVLSPVLFCLYMDGLLNKLSH |
| | | | | | | AGVGCYMGEMFVGALAYADDIV |
| | | | | | | LISPTPSAMNNMLSICDEFSIE |
| | | | | | | YNVLENPLKSKCMYFYPKSRST |
| | | | | | | LLLHRYNVCDLQESINGKAIEF |
| | | | | | | VDSYKHLGHVICSDMTDDIDIS |
| | | | | | | EKRGVFIGQANNIICYFAKLSS |
| | | | | | | AIKYRLFTSYCTSFFGCELWRI |
| | | | | | | SNDSLDSMCTAWRRAIRRIWSL |
| | | | | | | PYTAHGRFLPVLCNSYNIFDQF |
| | | | | | | CVRILNFIRRCLSDQSSLLVRS |
| | | | | | | IATQALMFHHARSPLGYNFIYC |
| | | | | | | ARRYCFSLTDFVNYNNFVSNVE |
| | | | | | | KLNCTQNDDDTIANCRLLRELI |
| | | | | | | DLRDGVLHLSDDVELTNSELSF |
| | | | | | | MINCVSTL |
| MG140 | 8 | MG140-36- | protein | unknown | uncultivated | MVQNTELADCINITLQTNDSRE |
| transposition | | R2 | | | organism | IVTSYNMHGENQGLEGTKEMIN |
| proteins | | transposition | | | | KLYPDVIALQEHWLTPTNLDRL |
| | | protein | | | | GEVSNDYFFIGSSSMNDVVTAG |
| | | | | | | PLFGRPEGGTAILINNKLANAT |
| | | | | | | VNVACNDKFTAVLISECLVISA |
| | | | | | | YMPCAGTCDRMSLYSAIISEIQ |
| | | | | | | ALIEFYPQYKLILCADLNVELD |
| | | | | | | APSSVADLVNNFICRNNLRRTE |
| | | | | | | LANPTASRFTYMHESLQAASYI |
| | | | | | | DYIISSDALDSIAFNVLDLDIN |
| | | | | | | LSDHLPIMCVFMCKMSACYSDP |
| | | | | | | SQSVKSKSHVDDVSYFRWDRAA |
| | | | | | | LHLYYEQTRVMMEPILDKLNIC |
| | | | | | | VNSVCDEDAFIDHSKLEDIYSS |
| | | | | | | VVAALTTSANLCIPKIKHNFFK |
| | | | | | | FWWNQELNELKAAAVTSARAWQ |
| | | | | | | QVGKPKHGSVYHKYQKDKLLYK |
| | | | | | | KCIRENQKQESVSFTNELHDAL |
| | | | | | | LRKTGKDFWKTWNAKVGVKRKC |
| | | | | | | IEQVDGIVDGATIACNFAKHFE |
| | | | | | | KICQPATATENTTMKAKFEEMR |
| | | | | | | SMYYTPFCDKTVEFDVQLIGKI |
| | | | | | | ISNMSCGKAAGLDELSAEHLKH |
| | | | | | | CHPIVIIILCKLENLFVHVGYL |
| | | | | | | PLSFGTSYTVPIPKQNGRSHAL |
| | | | | | | TVNDERGISISPVISKIFEHAI |
| | | | | | | FARFGDYFSTSDHQFGFKESLS |
| | | | | | | CSHAIYCVRNVIDHYVKRGSTV |
| | | | | | | NICTVDISKAFDTVNHFVLFIK |
| | | | | | | LMERKLPVQLLDLFVLWFSMSE |
| | | | | | | TCVRWGAHDSYFFKLKAGVRQG |
| | | | | | | GVLSPYFFAVSVDDVVDKIAAC |
| | | | | | | NAGCYINNLCSAIFLYADDIIL |
| | | | | | | LSPSVCGLQRLLGICEAAITEL |
| | | | | | | DMKINASKTVCTRVGPREDSAC |
| | | | | | | VGPNLTSGGQLNWVKTCRYLGV |
| | | | | | | HFAAGRSFNCSFEEAKKKFYFS |
| | | | | | | FNSVYSKLGRFASEEVILNLLN |
| | | | | | | VKCVSAMLYATEACPVLSRHKH |
| | | | | | | SLDFVITRVEMKILRTGSPQVV |
| | | | | | | AECQKYFGFLPVSYRIDIRSAR |
| | | | | | | FLERFSSSVNSFCIAFGDRARK |
| | | | | | | QLSDILCKYNIPDGLSSGVLRT |
| | | | | | | IINRIFFNS |
| MG140 | 9 | MG140-37- | protein | unknown | uncultivated | MTITNDAAQLVVISYNLHGLNQ |
| transposition | | R2 | | | organism | GLPGIREFMTELKPDVIMVQEH |
| proteins | | transposition | | | | WLTSDNLNKLSDISDEYFVIGS |
| | | protein | | | | SAMDARVSAGPLFGRPFGGTAV |
| | | | | | | FIKNKYINVTVNLVTSERYVVI |
| | | | | | | QLCDWLLINVYLPCIGTSNRIL |
| | | | | | | LYSDTLCELQSIIRAHPECNCL |
| | | | | | | VGGDENTDLNDTRSHTANTVVN |
| | | | | | | GFIAGCNLHRCDCLFPTRLKHT |
| | | | | | | YVNDSMNCYSTIDYMLSSNPEK |
| | | | | | | IVAFNVLDIDLNLSDHLPIITV |
| | | | | | | CVFDSNCKLNNPKPTSTEDVTH |
| | | | | | | FRWDHAPLQDYYEHTRLGLQPI |
| | | | | | | LADLDELIGNKLYSNVDFLCQV |
| | | | | | | DCIYNRVVSVLQQCSHAYVPKH |
| | | | | | | KKNFYKFWWNQELDELKDKAIA |
| | | | | | | SCKMWKDAGKPRHGAIHAKYRQ |
| | | | | | | DKLLYKKRIRQERVQETSSFTN |
| | | | | | | ELHDALLNKSGRDFWKKWNSKE |
| | | | | | | ENKSNKILQVGGTSDTATILNN |
| | | | | | | FAKHFEQVCVPFNATRNEELKS |
| | | | | | | RYKEMRLNYSESSVINDTTVED |
| | | | | | | VQLVENLLTNMKNGKAGGLDEL |
| | | | | | | TSEHLKFCHPVVVIILCKLENL |
| | | | | | | FVINGHIPDSFGVSYTVPIPKS |
| | | | | | | DGRSRSMTVDDERGISISPVIS |
| | | | | | | KLFELCVLDRYSDYLQTSDHQF |
| | | | | | | GFKKQLGCRHAIFSVRSVIEQY |
| | | | | | | ISNGSTVNLCALDLSKAFDRMN |
| | | | | | | QYALFIKLMERRFPVKILTILE |
| | | | | | | QWFSIAETCVRWGSEFSYFFSL |
| | | | | | | LAGVRQGGVLSPVLFAIFIDGI |
| | | | | | | VNRVNATNVGCYNSTVCVSIFL |
| | | | | | | YADDILLLSPTVTGLQTLLTVC |
| | | | | | | ENELCELDMRLNVNKSVCMRFG |
| | | | | | | ARFKAHCANLVSVQGGALQWAS |
| | | | | | | SCRYLGVYFVSGRVFRCCFHSA |
| | | | | | | KCNFFRAFNSVYSKIGCCASEE |
| | | | | | | VILSLLKSKCLPYILYGVEACP |
| | | | | | | VLQRDKHSFDFTLTRTLMKLEM |
| | | | | | | TSSPVIVNECQTQFNLLPLRYQ |
| | | | | | | IDIRTVKELNQYIISRNSICML |
| | | | | | | YKSHAQSVLDTIFGVYGNNVSS |
| | | | | | | LHDLHNIINEHFYDENPSTLGY |
| | | | | | | N |
| MG140 | 10 | MG140-38- | protein | unknown | uncultivated | MVILLLMASYTNAILSIASENC |
| transposition | | R2 | | | organism | RGFNQIKKQYICNLLGKCNFLF |
| proteins | | transposition | | | | LQEHWLSERQLGILGNIQSGVL |
| | | protein | | | | YSGISGFDSSEVLAGRPFGGCA |
| | | | | | | ILWHSDVLINVTSVETNSNRIC |
| | | | | | | AVLVTTESWKLILINVYLPHEG |
| | | | | | | DDIKSDEFVHCLYLVEDIIGKH |
| | | | | | | SDCHVVVGGDFNVDENRNWNHT |
| | | | | | | KILNSFCDSNELLSACRSNSNI |
| | | | | | | DYTYNFSMERESILDHFLLSST |
| | | | | | | LHNTCIEKISVMHDVDNISDHD |
| | | | | | | PIFLKLKLDVKYIGFSSRIFAP |
| | | | | | | RTSWVKATENDLNRFRHTLSDN |
| | | | | | | LKCITTPTSLLLCHDMKCTDAC |
| | | | | | | HHNAIAEYATAISEACLLAAGT |
| | | | | | | CIPHTSNRCTDRRVPGWTERVE |
| | | | | | | PLREKSIFWHKLWMECGRPRDG |
| | | | | | | HVADCMRRTRAGYHYAIRQVRK |
| | | | | | | DEELIVKQRIAEALARDPSRDE |
| | | | | | | WTEIKRIRGNKAGLSRIVDGCI |
| | | | | | | DEASISKLFADKYKCLYASVPY |
| | | | | | | DTVEMQNIQDIVDSQLAASDEF |
| | | | | | | DDSYIINYQDVSDAIFKLNAHK |
| | | | | | | NDGNLGISTDHFLHGGSDLHMH |
| | | | | | | IAFLLTSIVVHGSVPSEFVSST |
| | | | | | | VIPIPKKPNVNATQSDNYRGIA |
| | | | | | | LSSCFCKILDNIILAKNADRLS |
| | | | | | | TSDLQFGFKRKSSTHMCTMVLK |
| | | | | | | ETLSYYVSNNSCTFCTFLDATK |
| | | | | | | AFDRVNYCKLFHLLIKRGLPAS |
| | | | | | | VIRILIVMYTGHCVRVAWAGLA |
| | | | | | | SSFFSAVNGVKQGGVLSPILFC |
| | | | | | | IYMDDLLVKLHKSGVGCYIGTA |
| | | | | | | FVGALAYADDIVLLAPSPSAMR |
| | | | | | | KLLSVCEFYALDEDISENAGKS |
| | | | | | | NFILVMPNSNQRLRTQMNNCTF |
| | | | | | | SIGGAPVVKVNSYLHLGHIINN |
| | | | | | | QLNDDDDVMYRRNCFIGQANNV |
| | | | | | | LCYFNKLDMCVRIKLFKNFCSS |
| | | | | | | MYGCELWSTDNVEVFCVAWRKA |
| | | | | | | LRRVLNLPYDTHSYLLPLLTDT |
| | | | | | | LPVFEEICRRSAKFILKCENSS |
| | | | | | | STLVKYVTRHAIDIARYNSNVG |
| | | | | | | KNALFCCNYFKWQLYDFVNGSV |
| | | | | | | SLNRNSFLTFCLNRLSGCEVDN |
| | | | | | | AGSLYEALLVREGNLEVESFTR |
| | | | | | | DDIELIIDAMSRC |
| MG140 | 11 | MG140-39- | protein | unknown | uncultivated | MANCNGTIISYNMHGENQGSEL |
| transposition | | R2 | | | organism | LKSYCANSSVDFLLIQEHWLSP |
| proteins | | transposition | | | | DALHKIDDIAQDYFCFSVSSMT |
| | | protein | | | | AVLESGPLRGRPFGGLSILVKN |
| | | | | | | MHRQFCSVVALNERYIAVQYND |
| | | | | | | ILIIDVYFPCVSSLNHKDETID |
| | | | | | | LLVQLDTLVNQSVVKDIIIGGD |
| | | | | | | FNCNLEIESWSSKVICDEMEDN |
| | | | | | | SLSSCNKLAVNVIGAEYTYSNE |
| | | | | | | VLGHYSYLDYFVVSNSLTSSVM |
| | | | | | | SLEICSDDENLSDHSPVCIEVC |
| | | | | | | NIISDCETTVLCSNKGGKSTKI |
| | | | | | | PDTCYQNRWDHADIVAYYELTR |
| | | | | | | LGLQPILAYLNDCSNSFPPQSA |
| | | | | | | IDYKRYMRACIDLAYNDAVSVL |
| | | | | | | VDAAKNTVPREKANALKHWWDQ |
| | | | | | | ELSELKEKAFASHKLWIEAGKP |
| | | | | | | RNGCIFDIRKADKYKYKCLIKR |
| | | | | | | KQLEVRDSITNDLHDALLLKDA |
| | | | | | | DHFWKVWKSKFPAKRPNKYALI |
| | | | | | | EGNSDPNIISNTFCNYFSEICT |
| | | | | | | AHPSVNASSNNIFLNREDGYIG |
| | | | | | | DLNQSTKNIISIDLIEDFILSM |
| | | | | | | KRGKAAGLDSLTIEHIQFAHPA |
| | | | | | | IICILKLLFNYMLEFGIVPEGE |
| | | | | | | RNGLVIPLPKEDSIKKNVKLEN |
| | | | | | | FRCITISPVISKIFEHCLMRLF |
| | | | | | | AKYLNSDDAQLGFKKKCGCSHA |
| | | | | | | IYCVKQVVDYYVRGGSTVNVCT |
| | | | | | | LDISKAFDKVNLFVLLCKLMDR |
| | | | | | | NIPNYVINVLYDWESNNYITVK |
| | | | | | | WLNILSSRCPVNSGVRQGGVLS |
| | | | | | | PVLFAIYVDDILVKLRKSRLGC |
| | | | | | | TIQGLSVNAYMYADDLIILSGS |
| | | | | | | VTDLQKLITLSIEELKCIHLSI |
| | | | | | | NPKKCFCMRVGKRFKVNCNNVV |
| | | | | | | VDNYSIQWSSEIRYLGVYLTAG |
| | | | | | | HVLKENLDYGKKSSIAA |
| MG140 | 12 | MG140-40- | protein | unknown | uncultivated | MHGENQGFTYVNEICNDQAYDI |
| transposition | | R2 | | | organism | IFIQEHWLYPSTLHKILEISDN |
| proteins | | transposition | | | | YIGFGTSAMQSELEARFLRGRP |
| | | protein | | | | YGGTAILINKKFKTYCIDSCVF |
| | | | | | | KRVVSVLLGDELFINVYLPCND |
| | | | | | | GSAANVNTLGEILANISNIIDA |
| | | | | | | HDVNCIIFGGDLNTDITKVSLH |
| | | | | | | SNMILNEMKDYDLTVCMKHIFG |
| | | | | | | TCDVQFVDTFVCENLNASSCID |
| | | | | | | FLCMSNSLSDNVSRYKVIDAYN |
| | | | | | | NHSDHLPVSIDVCLPVKCVLYA |
| | | | | | | CMNTVCDDVQVHSEHASKCTRS |
| | | | | | | KAKDSRLRWDLGNIQMYYDLTC |
| | | | | | | AHLYPIYECITQVESNMYMYDE |
| | | | | | | QDRSSRIDELYNKLVSTLHNCT |
| | | | | | | KPAIPLVKANTLKHWWTTELNQ |
| | | | | | | LKSKSLTSHNIWLDAGKPLRGE |
| | | | | | | IYQSKRHDKLSYKLAIKTAKKE |
| | | | | | | ADAAVSDALHTNLISKSSVNFW |
| | | | | | | KTWKSKVCNKVKTCVSVEGCNT |
| | | | | | | DEQAAAKFSDYFSRATSPNSQE |
| | | | | | | YSSAKQNEFNRKLLQYRKVEYA |
| | | | | | | NDITAELVALAVAKMQDGKAAG |
| | | | | | | FDNIYVEHIRHSHPLIFSLLAK |
| | | | | | | LFNLMLRAGIVPTLFGNGVVIP |
| | | | | | | IPKENTHKKTHPVDNERGITLS |
| | | | | | | PILSKVFEHCLLQLMSKYLATS |
| | | | | | | ENQFGFKSNTGCTQAVYAVRKV |
| | | | | | | TEYYVANESTMNLCFLDISKGF |
| | | | | | | DKVNNYELMMKLMKRRTPSYFI |
| | | | | | | QILNHWFSISQCTVRWNGVLSA |
| | | | | | | MYRLEAGVRQGGVLSPVLESVY |
| | | | | | | VDDMLCKLKSMGCSYKALHLSA |
| | | | | | | FMYADDLVLLSPSVSAMQTMLN |
| | | | | | | VCNCELKLLDLKLNVKKSKAVR |
| | | | | | | IGKRFKNNTVNLNIEGQAICWS |
| | | | | | | SEAKYLGVVIVSAPRFKCSEDA |
| | | | | | | AKAKFYRSANSILAKVGHKQNV |
| | | | | | | TVTLHLIATIALPTLTYCIEAL |
| | | | | | | HLNKSEVSSLSHPWQSCLFKLF |
| | | | | | | RTFDAEIIKNCCELLNYKTISE |
| | | | | | | LYNERVAKFARNLKICNNHVLP |
| | | | | | | LL |
| MG140 | 13 | MG140-41- | protein | unknown | uncultivated | MHGFNQGHSVISDFCNPKNVNS |
| transposition | | R2 | | | organism | MDIICIQEHWLSSTNLSKLANF |
| proteins | | transposition | | | | SNSYFMYGISAMENAEKAGLIK |
| | | protein | | | | GRPYGGVCILLRKELCKCVKFS |
| | | | | | | KCLERLVCLVIDGYIFINCYFP |
| | | | | | | SIRSDDDLNTVYLLESEIDDIL |
| | | | | | | RDFPGNRVILGGDENVNIVNKA |
| | | | | | | KYADIFIKKLQNLNLMFCNDIY |
| | | | | | | NSAINFTYCHESLQNYSYIDYF |
| | | | | | | AVSNFMIKDIIDFDILDLGNNI |
| | | | | | | SDHNPIVIKVKCQLSDVNVSSK |
| | | | | | | ASNYKEFFRWDHANISNYCNLT |
| | | | | | | RTYLQSILEYVNDVSDYLEMHA |
| | | | | | | CCYSCSDILKAGKERAYADLNL |
| | | | | | | SASYQTEPCKSCSGNYDIARRC |
| | | | | | | IDSSYNKIVNVLNFSAEKTVPV |
| | | | | | | RKANFYKFWWDDELSQLKYNSI |
| | | | | | | QAHETWVKLNKPKQGIAFDNKV |
| | | | | | | KAKTEYKLAIRQKQSLERDGIT |
| | | | | | | NDLHESLLAKSTNVFWKSWNSK |
| | | | | | | FGQKSKVPLVIESSQDNQVISD |
| | | | | | | KFAGYFASINSSNFPSNKNRHK |
| | | | | | | DVFLCKFKDYKGDNWSYRDKIN |
| | | | | | | TFMLDYCIKESKLGKSPGADGI |
| | | | | | | TIEHLLYSHPMLICLLSKLENL |
| | | | | | | MLRFSYVPDKFAECIVIPVPKL |
| | | | | | | QNVSKLMKCEDFRPISLSPIIS |
| | | | | | | KIFEKCILKLVEEYFVTSDRQF |
| | | | | | | GFKKRLGCTHAISAVKSTVDFF |
| | | | | | | TENGSTVNMCALDLSKAFDKIN |
| | | | | | | HFILFNKLMKSNMPLRILLLII |
| | | | | | | CWYSKLVNVVRWNGSLSNVEKT |
| | | | | | | ANGIRQGGILSPFLENIYVDDI |
| | | | | | | LFSLKRSKLGCYVGNMCCNSFM |
| | | | | | | YADDLLLLAISVTQLKQMIIMC |
| | | | | | | TNLFEEIDMRLNITKTNCIRVG |
| | | | | | | NRHLSTVIPMVIGDIVVNWKQE |
| | | | | | | MRYLGVCFVSAKKLKLNIIFAK |
| | | | | | | QKFFKSLNSIFSKVGSNASPEV |
| | | | | | | ILSLENSFCVPVLLYGLGPENL |
| | | | | | | SLNLKTRNSLIKAYDSVFYKTF |
| | | | | | | KTFDNEVTRYCQLMFRVMPLTF |
| | | | | | | KLDLLTINEMLSSQKCDNEIIK |
| | | | | | | FFSHRSFKINMDKLLYKYNIVI |
| | | | | | | DDMYKAKEALEDNFKVKVESNI |
| | | | | | | ALGLV |
| MG140 | 14 | MG140-42- | protein | unknown | uncultivated | MTETSHAPVLDQTCTRYQRHNV |
| transposition | | R2 | | | organism | HEQAMQFVPPNRVVFSFFGPCD |
| proteins | | transposition | | | | IVCHRGAFLSVTSYCKTLSLLQ |
| | | protein | | | | RAHFTRHRNKATGQPFLLLSVY |
| | | | | | | KQQDLQTLTQQGLVPFTQTCNE |
| | | | | | | ALSLRGCRVAPSGRVALPYMMY |
| | | | | | | PQGDHRNQPVHNQETHTPLTPG |
| | | | | | | LDANPFAALSVEQQTCGASTSR |
| | | | | | | TSLIVGSMNVNGLFTQTMSSKH |
| | | | | | | QDVCAFVSSHTVDICCLQETWC |
| | | | | | | DMSETEYSSMLASCGYTSFGVS |
| | | | | | | GYSTRTRTGGGTGILVATSISH |
| | | | | | | LAHMLHWESQKHTQTTWIKLNS |
| | | | | | | TQYKARKTAVGSVYLRPVSHTR |
| | | | | | | PGDAERYVTELIALRDDITYLS |
| | | | | | | EHNEDVVLCGDENARIGNSQVS |
| | | | | | | PHVPQHNEQTRNTQGDRLVTLL |
| | | | | | | EQCNMFVMHNHTSFSPTCMHAG |
| | | | | | | GSSVVDYCITNSSCRARCSDAA |
| | | | | | | INFAGPCVSDHALLTMSLSHAT |
| | | | | | | RTRHRKTRRAHAKLWNRRAMHD |
| | | | | | | EQTIAQFHATLETPLTQLSDFI |
| | | | | | | DASTLSPQEATDTFTSKLTAAL |
| | | | | | | RDAGATCFGTHQVRPHRAQWWN |
| | | | | | | KDYANLRHECWNLYETHRRSGS |
| | | | | | | EADRAAYEEKRAAKNKMKRNLK |
| | | | | | | RAYVKAQAGHVSKLCTPGLVSK |
| | | | | | | TSWGAAKRLLKLVTRGSSATEH |
| | | | | | | TLPTVIHDGAPTDDLEAVMQVE |
| | | | | | | SSHYHTEMNPVPSATFCEATDT |
| | | | | | | LVKDKLHAWRQAEPEHINNMDD |
| | | | | | | EFTITELDRALGSMHNWKAADH |
| | | | | | | DGMIIELLRAGGPALQLVVLKI |
| | | | | | | LNFCWTHETIPTNWKLGTIISL |
| | | | | | | YKAGKKENPSNYRGITLLSVVR |
| | | | | | | KLFCTLLRSRLQDNISLHESQA |
| | | | | | | AFRANRGCMDHVHTLARIVRAA |
| | | | | | | NRKDIPVYAFFLDIRKAYDTVW |
| | | | | | | RDGLIYKLLQKGVTGRLGRVIS |
| | | | | | | QVLTDTQSRVRFQHRESAYFPL |
| | | | | | | TLGVGQGDPLSTILFDVFIDDL |
| | | | | | | LEELHARPVHHCIPVDSPHLDR |
| | | | | | | IADLTYADDVNALSLTPEGLQG |
| | | | | | | HIDTIDTWLFRWRSQPNVSKSK |
| | | | | | | TMVFNPPQGVPPTVFTMRGSNL |
| | | | | | | DTVQCFKYLGVFFQSNGSWTDH |
| | | | | | | TQHVRTQMNKAVGMWRPVLRCH |
| | | | | | | YLPVAARLRIIYAFVYAPALYG |
| | | | | | | AEVWIAPKSELEKLDTICKSAI |
| | | | | | | RTVFGLHQFDCREEILFADTGL |
| | | | | | | LPVSSLINAAKLCWFTKLLNMP |
| | | | | | | ESRFPTAVEAVTLPGDTQRGRV |
| | | | | | | AGGDFGTRIADITTEIRRYAHM |
| | | | | | | KLVMQDPLEHAPHRRRRPIRVS |
| | | | | | | KRLSHTKPDAHLYETITYSSLT |
| | | | | | | RERVKALLWKVYMEKCFERRGR |
| | | | | | | KDGVTGEWMRSVIKHELGSLAP |
| | | | | | | FLHSVECGLVRVLMSARSRLSP |
| | | | | | | VLCRPPQDKHREYSQAAGAFTT |
| | | | | | | YISRAPIGAVLPSPPVAHCVRH |
| | | | | | | VSAVAELVRELLAQPGPYGCGS |
| | | | | | | SVSWSDIAVVLVVGIDGVGGAP |
| | | | | | | GVLGVVARPSPLSELARVVAFC |
| | | | | | | GASPRLRHHVAWMSRSVPVAGV |
| | | | | | | VNVPVDMAMLQRTKAGRQVAPQ |
| | | | | | | GKHGAHSS |
| MG140 | 15 | MG140-43- | protein | unknown | uncultivated | MAISYTSNNSVRICSENMHGEN |
| transposition | | R2 | | | organism | NGVSMTKILCNSFDVILLQEHW |
| proteins | | transposition | | | | LLPSNLSKLGDISHDFTHHSVS |
| | | protein | | | | AMTTKISEGILYGRPFGGVSIL |
| | | | | | | YRNSLTKNISIIDADKEEGRYV |
| | | | | | | TIKLKCVNNEFITVTNVYFPTI |
| | | | | | | CAYSDYIVNTSSIMAYLDNLFA |
| | | | | | | NEVSCHHVVAGDENFEYRNDNI |
| | | | | | | GFDLFRSLAVDNNLICCDDLHL |
| | | | | | | NQNIKFTYKHETLPQQSWLDHF |
| | | | | | | FVSAGLTSSIVYCDTIDNGSNL |
| | | | | | | SDHLPICCTINVSLSDSNCTPK |
| | | | | | | LSKVYRDRWDKADLIKYYYQSG |
| | | | | | | IHLQSLTAPPHVTQCSTHCQKA |
| | | | | | | QHLQDINAYYERIIQALKASAI |
| | | | | | | GCVPRMPVNCLKHWWNDDLTRL |
| | | | | | | KNLSIDMHNLWRQVGSPRNGII |
| | | | | | | NEARLKAKLDYKQAIRQAMLDC |
| | | | | | | ENKDADIINNKFNQKDSRNFWK |
| | | | | | | CWGAKYRKKVNNTACIDGCTDN |
| | | | | | | STIANKFKAYFQNTYVDSTCDV |
| | | | | | | NACMEFDKLMSDHSHLQHNDIV |
| | | | | | | EEIRIEDIEKCIELLKPLKSAG |
| | | | | | | HDDVAPEHLIHSHPSLCMHLKL |
| | | | | | | LFSMMLNHNYVPDSFGIGIIIP |
| | | | | | | VVKDKRGNLNSVENYRPITLSP |
| | | | | | | IISKVFESFVLNRFAKEMTCDP |
| | | | | | | LQFGFQRSVGCNNALFAIRQVI |
| | | | | | | QYENDRDSNVMVASLDACKAFD |
| | | | | | | RVNHFKLFSTLLQRKLPLHIIK |
| | | | | | | VLINWYCKLMVQVRWNNSLSDL |
| | | | | | | FHVKSGVRQGGILSPALENVYI |
| | | | | | | DCVINKLRASQLGCHIGSLYIA |
| | | | | | | VVLFADDILLLSSSEMELQRMI |
| | | | | | | DLCVESGDEIGLKENAAKSHCM |
| | | | | | | IIGPHKVLVKPDMMMGNSPTAW |
| | | | | | | SETIKYLGVYIQSDKKFTVDLS |
| | | | | | | LVRRKFFASVNCILRNAAFTSD |
| | | | | | | IVKLELVEKQCFPILLYGLQSE |
| | | | | | | DLKSSVIANVNAWVNSVYRKIF |
| | | | | | | GYHKWESVKECIYLLGRLDVFH |
| | | | | | | TIKLRRINFLKNIQKCNNEVAR |
| | | | | | | SLFNYVLHTREFQSCCTLPNNS |
| | | | | | | VINISKSCDNVRKAVENSFQSK |
| | | | | | | VVGH |
| MG140 | 16 | MG140-44- | protein | unknown | uncultivated | MASQTLDTQTANNVCNSVSVMS |
| transposition | | R2 | | | organism | YNMHGFNQGYSYLNDICMKQTY |
| proteins | | transposition | | | | QVILIQEHWLYPATLHKLANIS |
| | | protein | | | | EHYSFYGASAMQTALDAGFIRG |
| | | | | | | RPYGGTGILLHKDIAKYCVESC |
| | | | | | | AFERVVGVLLGDFLFVNAYFPC |
| | | | | | | WDGSVINLDAANELLANLSNVL |
| | | | | | | DSYQAKHVVEGGDENANLTKNS |
| | | | | | | VYSNMILEFMSEHQLDICRKVL |
| | | | | | | FGSSNVIFSDTYVNEALNASSC |
| | | | | | | IDFICISSGLSNNVAQYDVHDV |
| | | | | | | FNNHADHLPVSLKLCLPVSSIL |
| | | | | | | YNCIASGSSACMENEAKSRDTQ |
| | | | | | | SCDSKGNKLRWDKGNTQLYSDL |
| | | | | | | TYAQLFPIYETLHSIDVESSAY |
| | | | | | | TAQEHRSLINETYTQVVSALHY |
| | | | | | | AANVSIPAMASHTLKHWWCSDL |
| | | | | | | SELKKKSMLSHNEWINAGKPHA |
| | | | | | | GRIHTAKQQSKLQYKRAIKHAK |
| | | | | | | ASAENSVSDELHRNLCSRNNVK |
| | | | | | | FWKTWKNKIKRPGTEKLYVEGC |
| | | | | | | NTDAKAAELLADYENKATSPNS |
| | | | | | | NEYNNSKRVEFEKSFASYAIQA |
| | | | | | | NDIDISAGLVESAALKMTAGKA |
| | | | | | | AGIDNISIEHVHYCHIVIYSLL |
| | | | | | | AKLFNLMLCFSCVPDAFGYGVT |
| | | | | | | TPIPKEESHKKIHPVENERGIT |
| | | | | | | LSPVLSKLFEHCLLSVESDELQ |
| | | | | | | TSDNQFGFKRSTGCTHAIYALR |
| | | | | | | KVTEFFICNESTVNMCELDISK |
| | | | | | | GFDKVNNFELLLKLMKRKAPSC |
| | | | | | | FIKLLHDWESISYGSVKWNTSR |
| | | | | | | SDWYKIGAGVRQGGVLSPILFA |
| | | | | | | VYVDAMLEKVKTMGCQYKSLCT |
| | | | | | | GAFMYADDLVLLSPSIYELQHM |
| | | | | | | IALCRNELHSLDLKLNVKKSKA |
| | | | | | | LRIGKRYKCKALPLMIDGQAVH |
| | | | | | | WSNEARYLGIVIRSACKFKCNE |
| | | | | | | DPAKVKFYKAANTILAKLGNKC |
| | | | | | | NVTVTLYLVAAVALPPLIYGIE |
| | | | | | | ALTLNSSELNSLNHPWNNCLGK |
| | | | | | | MENTEDKELIKNCCDILGYESC |
| | | | | | | QNVYVKKVEKFLRNMKFIDNAI |
| | | | | | | LSALNTEQVNV |
| MG153 E. | 46 | MG153-22 | nucleo- | artificial | | ATGCGCCAGAAATCTGAACAGC |
| coli codon- | | | tide | sequence | | TCGAATTGGCTCTGGATAATCG |
| optimized | | | | | | GGGCGAAGCTCCGACTTCCCGT |
| genes | | | | | | CGGTCAGGTGAGGCCCCGACAA |
| | | | | | | CTGCACACGAAGCAGAACGCTC |
| | | | | | | TGGTGGTGGTCATCGTTTAATG |
| | | | | | | GAACAGGTCGTTGCGCGTGCTA |
| | | | | | | ATGCCCTGGCAGCACTGAGACG |
| | | | | | | CGTCAAGCAAAATCGCGGATCA |
| | | | | | | CCTGGAGTAGATGGTATGACCG |
| | | | | | | TTGGTGAGCTGCCACAGTACCT |
| | | | | | | GGCGAAACATTGGGAAACGGTG |
| | | | | | | CGCGAACAGCTCCTCGCAGGTT |
| | | | | | | CCTATCAACCGGAGCCCGTGAG |
| | | | | | | ACGCCAAGCGATTCCGAAACCT |
| | | | | | | GGAGGCGGTACACGAGTTTTGG |
| | | | | | | GCATTCCGACGGTCCTTGATAG |
| | | | | | | ATTTATACAACAGTGCCTGCTG |
| | | | | | | CAGGTACTTCAGCCACGGTTTG |
| | | | | | | ATCCTTCCTTTTCCGACCATTC |
| | | | | | | ATATGGGTTTCGTCCGGGGCGC |
| | | | | | | AATGCGCATGATGCGGTGTGTG |
| | | | | | | CAGCACAGCAATATATTCAGGC |
| | | | | | | GGGGCGTCGTTGGGTAGCCGAT |
| | | | | | | CTGGACTTGGAGAAATTTTTCG |
| | | | | | | ATCGGGTTAACCACGATGTGCT |
| | | | | | | CATGGAACGTTTGGCACGCCGA |
| | | | | | | ATTGCCGATCGCCGTGTGTTAC |
| | | | | | | GACTGATTCGCCGTTACCTCGT |
| | | | | | | CGCAGGAATTCTGCACGGGGGC |
| | | | | | | GTCGTCGTGGAGCGACGTGAAG |
| | | | | | | GGACGCCTCAAGGCGGCCCACT |
| | | | | | | TTCGCCTTTGCTGGCATCAGTC |
| | | | | | | TTACTGGATGAGGTGGATCGCG |
| | | | | | | AGCTCGAGCGTCGCGGACTGGC |
| | | | | | | GTTTGCACGATATGCGGATGAC |
| | | | | | | CTCAATGTCTACTGCGGTTCCC |
| | | | | | | GTCGCGCCGCGCACGATGCAAT |
| | | | | | | GGCAACCCTTAAACGCCTGTTT |
| | | | | | | GCTGCACTGAGATTGAAAGTGA |
| | | | | | | ATGAGTCAAAGTCAACTGTAGC |
| | | | | | | CCGCGTATGGGAGCGCAAATTT |
| | | | | | | TTAGGCTACTCATTCTGGGTGG |
| | | | | | | CGCCGGGGCGCGTCATACGGCC |
| | | | | | | ACGAATTGCACCTGCTGCGTTG |
| | | | | | | GCAGTCATGAAAGAACGAGTGC |
| | | | | | | GTCGGATAACACGCCGTACCGG |
| | | | | | | AGGGCGTTCACTGGAGGCAGTG |
| | | | | | | GCACAAGAACTGCGAGAATACC |
| | | | | | | TCACAGGCTGGAAAGCGTATTT |
| | | | | | | TCGATTGGCGGGCAAGCCCCGA |
| | | | | | | GTCTTCCGCGACTTGGATGAAT |
| | | | | | | GGACTCGTCATCGCCTGAGAGC |
| | | | | | | TGTGCAGCTGAAACAGTGGAAA |
| | | | | | | CGGGGCCGTACTGTTTGCAGAG |
| | | | | | | AATTATTGGCACGGGGGGTGCC |
| | | | | | | TGAAAGAGAAGCACGTGCTGCA |
| | | | | | | GCTGCTCATGCAAGACGGTGGT |
| | | | | | | GGGCTATGGCGGCTCACTCTGC |
| | | | | | | ACTGCAGACAGCTCTTCCAAAC |
| | | | | | | TCCCACTTTGATCAATTAGGTG |
| | | | | | | TTCCACGCTTGGCGGGTCGT |
| MG153 E. | 47 | MG153-23 | nucleo- | artificial | | ATGCGTGCTGATGAAGCCGAAG |
| coli codon- | | | tide | sequence | | CTCATGCATCTGCGGCTAGTAC |
| optimized | | | | | | CGGTAAAGGTGGCCGGAATTTG |
| genes | | | | | | CCCGGTACTGCCGCTGGCGCGG |
| | | | | | | AGGTGCGTGCTGCGGCAGGAGG |
| | | | | | | TCGGACGAAACCCGAAGCACTT |
| | | | | | | CGGCTGATGGAAGCGGCTGTGG |
| | | | | | | AACGTTCCAATATGCTGGGCGC |
| | | | | | | GTATGAACGGGTAGTCAAGAAT |
| | | | | | | CAAGGGGCTCCTGGAGTCGATG |
| | | | | | | GTCTGACGGTTACCGAATTCAA |
| | | | | | | ACCGTGGCTGCAAGCCCATTGG |
| | | | | | | CCGAAGATCAGACAGGTTCTTC |
| | | | | | | TCGCCGGAGAATATATGCCTGC |
| | | | | | | GGCGGTCCGGAAGGTCGATATA |
| | | | | | | CCCAAACCGCAAGGTGGTGTAC |
| | | | | | | GCACGTTGGGCATTCCTACTGT |
| | | | | | | CCTGGACAGATTGATTCAGCAA |
| | | | | | | GCCTTGCACCAGGTTCTGCAAC |
| | | | | | | CTCTGTTTGAGCCCGAGTTTTC |
| | | | | | | AGAATCATCCTATGGGTTCCGG |
| | | | | | | CCTGGTCGCAACGCTCACCAGG |
| | | | | | | CCGTGGAAGCAGCGCGTAGTTA |
| | | | | | | TGTTGCGGAAGGAAAACGATGG |
| | | | | | | GTAGTTGACTTAGAAAAATTCT |
| | | | | | | TCGATCGTGTTAACCACGATGT |
| | | | | | | ACTGATGGCCCGTGTTGCCCGT |
| | | | | | | AAAGTGAAAGACGAACGCGTCC |
| | | | | | | TGAAGCTGATACGAAGATATTT |
| | | | | | | AGAGGCTGGCCTGATGGAAGGA |
| | | | | | | GGTATGACCAGCGCTCGAACTG |
| | | | | | | AAGGGACTCCACAGGGTGGCCC |
| | | | | | | GCTTTCACCGCTTTTGTCAAAT |
| | | | | | | ACCCTTTTGACCGACTTAGATA |
| | | | | | | GAGAGCTGGAAAAACGAGGACA |
| | | | | | | TCGGTTTTGTCGTTACGCAGAT |
| | | | | | | GATTGCAATGTGTATGTGGGGA |
| | | | | | | GTCGTCGTTCTGGCCAGAGAGT |
| | | | | | | AATGGCGAAAATAACCGCGTTT |
| | | | | | | TTGGAGCAGCGCCTGAAATTGC |
| | | | | | | AGGTAAATGCTGATAAATCAGC |
| | | | | | | GGTTGCTCGTCCATGGCAGCGT |
| | | | | | | AAATTTTTGGGGTATTCCGTTA |
| | | | | | | CCTGGCATCGTAATCCTAAACT |
| | | | | | | GAAAATCGCCCCGAGTTCACGT |
| | | | | | | CAACGCCTTGCTGAAAAGATTC |
| | | | | | | GTCAAACCTTGCGCGGTGCGCG |
| | | | | | | GGGCCAATCGTTACGCCAGGTT |
| | | | | | | ATCGCACAGTTGAATCCCATAC |
| | | | | | | TCCGCGGTTGGGTTGCTTATTT |
| | | | | | | TCGCCTTACTGAGGAGAAAGGG |
| | | | | | | GTACTTGAAGAGTTAGATGGCT |
| | | | | | | GGGTACGTCGAAAACTTCGGGC |
| | | | | | | CCTTTTGTGGCGCCATTGGAAA |
| | | | | | | CGCGGTTACCCTCGTGCACAGA |
| | | | | | | ACCTCATGCGCGCGGGTCTTCG |
| | | | | | | TCCGGAACGGGCGTGGCAGTCC |
| | | | | | | GCGACTAACGGTCATGGTCCTT |
| | | | | | | GGTGGAACGGTGGAAGTTCACA |
| | | | | | | TATGAATGCGGCGTGCCCGAAA |
| | | | | | | TCCTGGTTCGACCACATGCAGC |
| | | | | | | TCGTGTCGCTGCTTGCAACGCA |
| | | | | | | GCGGAGATTTTCGTTAGTGTCC |
| MG153 E. | 48 | MG153-24 | nucleo- | artificial | | ATGAAGGGCGGCAAGCAAAAGA |
| coli codon- | | | tide | sequence | | TTTCTCAAGACACCTGTCTTCA |
| optimized | | | | | | AGAGTCACGCGCGGAGCCGGAA |
| genes | | | | | | GGCTATGCCGGTGGACAGACGT |
| | | | | | | TCATCTGGATGACCGAGAACAA |
| | | | | | | CCTCACTAATGCGAATAAGCCG |
| | | | | | | GAATATGGATTACTGGAACAGA |
| | | | | | | TCCTTAGCCCGACTAATTTAAA |
| | | | | | | CCGGGCATATAAGGGAGTCCGT |
| | | | | | | TCTAATCGCGGTTCAGGTGGTA |
| | | | | | | TCGACAAGATGGAAGTGGAGAG |
| | | | | | | CCTGAAAGATTACCTGGTCGAC |
| | | | | | | AACAAAGAAACACTTATCCAGT |
| | | | | | | CGATCCTTGACGGTAAGTATCG |
| | | | | | | CCCAAATCCTGTACGGCGTGTA |
| | | | | | | GAGATTTCCAAGGAGAAGGGGA |
| | | | | | | CAAGAAAGTTAGGGATCCCGAC |
| | | | | | | TGTGGTTGACCGCGTCATTCAA |
| | | | | | | CAAGCCATAGCGCAAGTACTGA |
| | | | | | | GTCCAACCTACGAGCGCCAGTT |
| | | | | | | CAGTGAGAACTCCTATGGCTTC |
| | | | | | | CGTCCTGGACGCAATGCGCACC |
| | | | | | | AAGCATTAAACCGTTGCCGCGA |
| | | | | | | TTATATTACGGACGGGTATATT |
| | | | | | | TATGCAGTTGATATGGATCTGG |
| | | | | | | AAAAGTTCTTCGACACAGTAAA |
| | | | | | | TCAGTCAAAGTTAATAGAGGTT |
| | | | | | | CTGTCTCGTACCGTGAAGGATG |
| | | | | | | GTAGAGTAGTTTCCCTGATCCA |
| | | | | | | TAAGTACCTCGATGCTGGTGTT |
| | | | | | | GTGATTAGAAATAAGTTCGAGG |
| | | | | | | AAACTGAGATGGGAGTTCCGCA |
| | | | | | | AGGTAGCCCACTCTCCCCGGTT |
| | | | | | | TTAAGCAACATCATGCTCAACG |
| | | | | | | AATTAGACAAGGAGTTGGAAAA |
| | | | | | | GCGCGGTCATCCATTCGTTCGA |
| | | | | | | TATGCTGATGACTTAATTATAT |
| | | | | | | TCTGCAAGTCGAAGCGAAGTGC |
| | | | | | | TGACCGCACTTTGGCAAACACC |
| | | | | | | GTCCCGTACATAGAAAATAAAC |
| | | | | | | TGTTCCTTAAGGTGAATAGAGA |
| | | | | | | AAAGACTACGACCGCGTACGTG |
| | | | | | | TCAGGTATCAAATTCTTAGGCT |
| | | | | | | ACAGTTTCTACGTTTACAAGGG |
| | | | | | | AGAAGGACGCCTTCGAGTCCAC |
| | | | | | | CCCAAGTCCATAGCCAAGATGA |
| | | | | | | AGGAACGGATAAGAAAGTTGAC |
| | | | | | | TAGTAGATCCAACGGGTGGGGA |
| | | | | | | TACGCCAGACGTAAGGAAGCGC |
| | | | | | | TGAGACAATATATTACTGGTTG |
| | | | | | | GGTGAATTACTTTAAGCTTGCG |
| | | | | | | GACATGAAGAAGCTGCTGGTTA |
| | | | | | | GCGTTGATGAATGGTATCGGCG |
| | | | | | | GCGTCTGCGTTTAGTAATTTGG |
| | | | | | | AAGCAGTGGAAGAGAGTTCGAA |
| | | | | | | CGCGTGGCCGTTACCTTATGAA |
| | | | | | | GTTAGGCATTGTGAAACATCAG |
| | | | | | | GCATGGGAGTTCGCCAACACTC |
| | | | | | | GGAAGGGTTACTGGCATACAGC |
| | | | | | | AAAGAGCCCAATCCTCAACCGC |
| | | | | | | AGTGTGACGTCAAACCGGCTGA |
| | | | | | | GACAGGCGGGGTACGTGTTCTT |
| | | | | | | TGTAGACTATTATCGCGTTGTG |
| | | | | | | AATGGGATTAAC |
| MG153 | 49 | NStrep- | nucleot | artificial | | ATGGCTAGCGCATGGAGTCATC |
| Strep tagged | | MG153-22 | ide | sequence | | CTCAATTCGAAAAATCCGGAAT |
| genes | | | | | | GCGCCAGAAATCTGAACAGCTC |
| | | | | | | GAATTGGCTCTGGATAATCGGG |
| | | | | | | GCGAAGCTCCGACTTCCCGTCG |
| | | | | | | GTCAGGTGAGGCCCCGACAACT |
| | | | | | | GCACACGAAGCAGAACGCTCTG |
| | | | | | | GTGGTGGTCATCGTTTAATGGA |
| | | | | | | ACAGGTCGTTGCGCGTGCTAAT |
| | | | | | | GCCCTGGCAGCACTGAGACGCG |
| | | | | | | TCAAGCAAAATCGCGGATCACC |
| | | | | | | TGGAGTAGATGGTATGACCGTT |
| | | | | | | GGTGAGCTGCCACAGTACCTGG |
| | | | | | | CGAAACATTGGGAAACGGTGCG |
| | | | | | | CGAACAGCTCCTCGCAGGTTCC |
| | | | | | | TATCAACCGGAGCCCGTGAGAC |
| | | | | | | GCCAAGCGATTCCGAAACCTGG |
| | | | | | | AGGCGGTACACGAGTTTTGGGC |
| | | | | | | ATTCCGACGGTCCTTGATAGAT |
| | | | | | | TTATACAACAGTGCCTGCTGCA |
| | | | | | | GGTACTTCAGCCACGGTTTGAT |
| | | | | | | CCTTCCTTTTCCGACCATTCAT |
| | | | | | | ATGGGTTTCGTCCGGGGCGCAA |
| | | | | | | TGCGCATGATGCGGTGTGTGCA |
| | | | | | | GCACAGCAATATATTCAGGCGG |
| | | | | | | GGCGTCGTTGGGTAGCCGATCT |
| | | | | | | GGACTTGGAGAAATTTTTCGAT |
| | | | | | | CGGGTTAACCACGATGTGCTCA |
| | | | | | | TGGAACGTTTGGCACGCCGAAT |
| | | | | | | TGCCGATCGCCGTGTGTTACGA |
| | | | | | | CTGATTCGCCGTTACCTCGTCG |
| | | | | | | CAGGAATTCTGCACGGGGGCGT |
| | | | | | | CGTCGTGGAGCGACGTGAAGGG |
| | | | | | | ACGCCTCAAGGCGGCCCACTTT |
| | | | | | | CGCCTTTGCTGGCATCAGTCTT |
| | | | | | | ACTGGATGAGGTGGATCGCGAG |
| | | | | | | CTCGAGCGTCGCGGACTGGCGT |
| | | | | | | TTGCACGATATGCGGATGACCT |
| | | | | | | CAATGTCTACTGCGGTTCCCGT |
| | | | | | | CGCGCCGCGCACGATGCAATGG |
| | | | | | | CAACCCTTAAACGCCTGTTTGC |
| | | | | | | TGCACTGAGATTGAAAGTGAAT |
| | | | | | | GAGTCAAAGTCAACTGTAGCCC |
| | | | | | | GCGTATGGGAGCGCAAATTTTT |
| | | | | | | AGGCTACTCATTCTGGGTGGCG |
| | | | | | | CCGGGGCGCGTCATACGGCCAC |
| | | | | | | GAATTGCACCTGCTGCGTTGGC |
| | | | | | | AGTCATGAAAGAACGAGTGCGT |
| | | | | | | CGGATAACACGCCGTACCGGAG |
| | | | | | | GGCGTTCACTGGAGGCAGTGGC |
| | | | | | | ACAAGAACTGCGAGAATACCTC |
| | | | | | | ACAGGCTGGAAAGCGTATTTTC |
| | | | | | | GATTGGCGGGCAAGCCCCGAGT |
| | | | | | | CTTCCGCGACTTGGATGAATGG |
| | | | | | | ACTCGTCATCGCCTGAGAGCTG |
| | | | | | | TGCAGCTGAAACAGTGGAAACG |
| | | | | | | GGGCCGTACTGTTTGCAGAGAA |
| | | | | | | TTATTGGCACGGGGGGTGCCTG |
| | | | | | | AAAGAGAAGCACGTGCTGCAGC |
| | | | | | | TGCTCATGCAAGACGGTGGTGG |
| | | | | | | GCTATGGCGGCTCACTCTGCAC |
| | | | | | | TGCAGACAGCTCTTCCAAACTC |
| | | | | | | CCACTTTGATCAATTAGGTGTT |
| | | | | | | CCACGCTTGGCGGGTCGTTGA |
| MG153 | 50 | NStrep- | nucleo- | artificial | | ATGGCTAGCGCATGGAGTCATC |
| Strep tagged | | MG153-23 | tide | sequence | | CTCAATTCGAAAAATCCGGAAT |
| genes | | | | | | GCGTGCTGATGAAGCCGAAGCT |
| | | | | | | CATGCATCTGCGGCTAGTACCG |
| | | | | | | GTAAAGGTGGCCGGAATTTGCC |
| | | | | | | CGGTACTGCCGCTGGCGCGGAG |
| | | | | | | GTGCGTGCTGCGGCAGGAGGTC |
| | | | | | | GGACGAAACCCGAAGCACTTCG |
| | | | | | | GCTGATGGAAGCGGCTGTGGAA |
| | | | | | | CGTTCCAATATGCTGGGCGCGT |
| | | | | | | ATGAACGGGTAGTCAAGAATCA |
| | | | | | | AGGGGCTCCTGGAGTCGATGGT |
| | | | | | | CTGACGGTTACCGAATTCAAAC |
| | | | | | | CGTGGCTGCAAGCCCATTGGCC |
| | | | | | | GAAGATCAGACAGGTTCTTCTC |
| | | | | | | GCCGGAGAATATATGCCTGCGG |
| | | | | | | CGGTCCGGAAGGTCGATATACC |
| | | | | | | CAAACCGCAAGGTGGTGTACGC |
| | | | | | | ACGTTGGGCATTCCTACTGTCC |
| | | | | | | TGGACAGATTGATTCAGCAAGC |
| | | | | | | CTTGCACCAGGTTCTGCAACCT |
| | | | | | | CTGTTTGAGCCCGAGTTTTCAG |
| | | | | | | AATCATCCTATGGGTTCCGGCC |
| | | | | | | TGGTCGCAACGCTCACCAGGCC |
| | | | | | | GTGGAAGCAGCGCGTAGTTATG |
| | | | | | | TTGCGGAAGGAAAACGATGGGT |
| | | | | | | AGTTGACTTAGAAAAATTCTTC |
| | | | | | | GATCGTGTTAACCACGATGTAC |
| | | | | | | TGATGGCCCGTGTTGCCCGTAA |
| | | | | | | AGTGAAAGACGAACGCGTCCTG |
| | | | | | | AAGCTGATACGAAGATATTTAG |
| | | | | | | AGGCTGGCCTGATGGAAGGAGG |
| | | | | | | TATGACCAGCGCTCGAACTGAA |
| | | | | | | GGGACTCCACAGGGTGGCCCGC |
| | | | | | | TTTCACCGCTTTTGTCAAATAC |
| | | | | | | CCTTTTGACCGACTTAGATAGA |
| | | | | | | GAGCTGGAAAAACGAGGACATC |
| | | | | | | GGTTTTGTCGTTACGCAGATGA |
| | | | | | | TTGCAATGTGTATGTGGGGAGT |
| | | | | | | CGTCGTTCTGGCCAGAGAGTAA |
| | | | | | | TGGCGAAAATAACCGCGTTTTT |
| | | | | | | GGAGCAGCGCCTGAAATTGCAG |
| | | | | | | GTAAATGCTGATAAATCAGCGG |
| | | | | | | TTGCTCGTCCATGGCAGCGTAA |
| | | | | | | ATTTTTGGGGTATTCCGTTACC |
| | | | | | | TGGCATCGTAATCCTAAACTGA |
| | | | | | | AAATCGCCCCGAGTTCACGTCA |
| | | | | | | ACGCCTTGCTGAAAAGATTCGT |
| | | | | | | CAAACCTTGCGCGGTGCGCGGG |
| | | | | | | GCCAATCGTTACGCCAGGTTAT |
| | | | | | | CGCACAGTTGAATCCCATACTC |
| | | | | | | CGCGGTTGGGTTGCTTATTTTC |
| | | | | | | GCCTTACTGAGGAGAAAGGGGT |
| | | | | | | ACTTGAAGAGTTAGATGGCTGG |
| | | | | | | GTACGTCGAAAACTTCGGGCCC |
| | | | | | | TTTTGTGGCGCCATTGGAAACG |
| | | | | | | CGGTTACCCTCGTGCACAGAAC |
| | | | | | | CTCATGCGCGCGGGTCTTCGTC |
| | | | | | | CGGAACGGGCGTGGCAGTCCGC |
| | | | | | | GACTAACGGTCATGGTCCTTGG |
| | | | | | | TGGAACGGTGGAAGTTCACATA |
| | | | | | | TGAATGCGGCGTGCCCGAAATC |
| | | | | | | CTGGTTCGACCACATGCAGCTC |
| | | | | | | GTGTCGCTGCTTGCAACGCAGC |
| | | | | | | GGAGATTTTCGTTAGTGTCCTG |
| | | | | | | A |
| MG153 | 51 | NStrep- | nucleot | artificial | | ATGGCTAGCGCATGGAGTCATC |
| Strep tagged | | MG153-24 | ide | sequence | | CTCAATTCGAAAAATCCGGAAT |
| genes | | | | | | GAAGGGCGGCAAGCAAAAGATT |
| | | | | | | TCTCAAGACACCTGTCTTCAAG |
| | | | | | | AGTCACGCGCGGAGCCGGAAGG |
| | | | | | | CTATGCCGGTGGACAGACGTTC |
| | | | | | | ATCTGGATGACCGAGAACAACC |
| | | | | | | TCACTAATGCGAATAAGCCGGA |
| | | | | | | ATATGGATTACTGGAACAGATC |
| | | | | | | CTTAGCCCGACTAATTTAAACC |
| | | | | | | GGGCATATAAGGGAGTCCGTTC |
| | | | | | | TAATCGCGGTTCAGGTGGTATC |
| | | | | | | GACAAGATGGAAGTGGAGAGCC |
| | | | | | | TGAAAGATTACCTGGTCGACAA |
| | | | | | | CAAAGAAACACTTATCCAGTCG |
| | | | | | | ATCCTTGACGGTAAGTATCGCC |
| | | | | | | CAAATCCTGTACGGCGTGTAGA |
| | | | | | | GATTTCCAAGGAGAAGGGGACA |
| | | | | | | AGAAAGTTAGGGATCCCGACTG |
| | | | | | | TGGTTGACCGCGTCATTCAACA |
| | | | | | | AGCCATAGCGCAAGTACTGAGT |
| | | | | | | CCAACCTACGAGCGCCAGTTCA |
| | | | | | | GTGAGAACTCCTATGGCTTCCG |
| | | | | | | TCCTGGACGCAATGCGCACCAA |
| | | | | | | GCATTAAACCGTTGCCGCGATT |
| | | | | | | ATATTACGGACGGGTATATTTA |
| | | | | | | TGCAGTTGATATGGATCTGGAA |
| | | | | | | AAGTTCTTCGACACAGTAAATC |
| | | | | | | AGTCAAAGTTAATAGAGGTTCT |
| | | | | | | GTCTCGTACCGTGAAGGATGGT |
| | | | | | | AGAGTAGTTTCCCTGATCCATA |
| | | | | | | AGTACCTCGATGCTGGTGTTGT |
| | | | | | | GATTAGAAATAAGTTCGAGGAA |
| | | | | | | ACTGAGATGGGAGTTCCGCAAG |
| | | | | | | GTAGCCCACTCTCCCCGGTTTT |
| | | | | | | AAGCAACATCATGCTCAACGAA |
| | | | | | | TTAGACAAGGAGTTGGAAAAGC |
| | | | | | | GCGGTCATCCATTCGTTCGATA |
| | | | | | | TGCTGATGACTTAATTATATTC |
| | | | | | | TGCAAGTCGAAGCGAAGTGCTG |
| | | | | | | ACCGCACTTTGGCAAACACCGT |
| | | | | | | CCCGTACATAGAAAATAAACTG |
| | | | | | | TTCCTTAAGGTGAATAGAGAAA |
| | | | | | | AGACTACGACCGCGTACGTGTC |
| | | | | | | AGGTATCAAATTCTTAGGCTAC |
| | | | | | | AGTTTCTACGTTTACAAGGGAG |
| | | | | | | AAGGACGCCTTCGAGTCCACCC |
| | | | | | | CAAGTCCATAGCCAAGATGAAG |
| | | | | | | GAACGGATAAGAAAGTTGACTA |
| | | | | | | GTAGATCCAACGGGTGGGGATA |
| | | | | | | CGCCAGACGTAAGGAAGCGCTG |
| | | | | | | AGACAATATATTACTGGTTGGG |
| | | | | | | TGAATTACTTTAAGCTTGCGGA |
| | | | | | | CATGAAGAAGCTGCTGGTTAGC |
| | | | | | | GTTGATGAATGGTATCGGCGGC |
| | | | | | | GTCTGCGTTTAGTAATTTGGAA |
| | | | | | | GCAGTGGAAGAGAGTTCGAACG |
| | | | | | | CGTGGCCGTTACCTTATGAAGT |
| | | | | | | TAGGCATTGTGAAACATCAGGC |
| | | | | | | ATGGGAGTTCGCCAACACTCGG |
| | | | | | | AAGGGTTACTGGCATACAGCAA |
| | | | | | | AGAGCCCAATCCTCAACCGCAG |
| | | | | | | TGTGACGTCAAACCGGCTGAGA |
| | | | | | | CAGGCGGGGTACGTGTTCTTTG |
| | | | | | | TAGACTATTATCGCGTTGTGAA |
| | | | | | | TGGGATTAACTGA |
| MG153 | 52 | NStrep- | nucleo- | artificial | | ATGGCTAGCGCATGGAGTCATC |
| Strep tagged | | MG160-7 | tide | sequence | | CTCAATTCGAAAAATCCGGAAT |
| genes | | | | | | GCATAATTTCAAGAGTCGTTTC |
| | | | | | | GAATTAAGCAAGGGGAAATGGG |
| | | | | | | TCTACATTCAAATCGAGGATTT |
| | | | | | | GGCAAATCATGCAAAGGACCAC |
| | | | | | | ATCCGACAAATCCGAGACTTGT |
| | | | | | | GGACCCCGCCAGAGTACTTCTT |
| | | | | | | CCATCTGCAAAAGGGCGGGCAT |
| | | | | | | ATCGCAGCACTGCGGTTACATA |
| | | | | | | CGCCTAATGAGTGGTATGGTAA |
| | | | | | | AGTTGACTTGTCAAAATTCTTC |
| | | | | | | AATAATATTACACGTCACCGCG |
| | | | | | | TCACCCGCAGTTTGAAGCGCAT |
| | | | | | | CGGCTACTCATTTCGCGACGCC |
| | | | | | | GAGGAGTTCGCAGTTGCTTCAA |
| | | | | | | CTGTGTTAGTTAATCATGCTAC |
| | | | | | | ACGCCGGTACGTCTTGCCGTAC |
| | | | | | | GGGTTCGTGCAATCCCCTCTGC |
| | | | | | | TTGCATCCATCTCTTTAGACAA |
| | | | | | | GTCGGACTTAGGCAACTGTTTG |
| | | | | | | CGGCGACTGCACGAAGAGACGG |
| | | | | | | TATCTGTAAGCGTGTACGTGGA |
| | | | | | | TGACATCGTTGTCAGTGCAGAC |
| | | | | | | TCTGAGCGTGACGTGGCGGAAG |
| | | | | | | CTTTAAGTAATATCTATTTGGC |
| | | | | | | CGCGATCAACTCACGGTTCCCT |
| | | | | | | ATTAACGAGGAAAAGTCAAGAG |
| | | | | | | GGCCGAGCTCAACTTTGAACGC |
| | | | | | | CTTTAATATCGAGTTGGACATG |
| | | | | | | CACGAATTAGAGATCACCGCTA |
| | | | | | | AACGGTACGAGAAGATGTGCGG |
| | | | | | | GGAAGTTCTGATTAACGGGACA |
| | | | | | | GGCCAGGTTTCAGACGCTATTT |
| | | | | | | TGAATTACGTTCAGACCGTTAA |
| | | | | | | TCCGGCTCAAGCCGAACAGATG |
| | | | | | | CTTCGTGACTTCCCTGGCGTAT |
| | | | | | | TTGGAACCCTCAGCAGTAGTGC |
| | | | | | | AAGCCGCTACGATGCTTCTTGA |
| MG160 E. | 53 | MG160-7 | nucleo- | artificial | | ATGCATAATTTCAAGAGTCGTT |
| coli codon- | | | tide | sequence | | TCGAATTAAGCAAGGGGAAATG |
| optimized | | | | | | GGTCTACATTCAAATCGAGGAT |
| genes | | | | | | TTGGCAAATCATGCAAAGGACC |
| | | | | | | ACATCCGACAAATCCGAGACTT |
| | | | | | | GTGGACCCCGCCAGAGTACTTC |
| | | | | | | TTCCATCTGCAAAAGGGCGGGC |
| | | | | | | ATATCGCAGCACTGCGGTTACA |
| | | | | | | TACGCCTAATGAGTGGTATGGT |
| | | | | | | AAAGTTGACTTGTCAAAATTCT |
| | | | | | | TCAATAATATTACACGTCACCG |
| | | | | | | CGTCACCCGCAGTTTGAAGCGC |
| | | | | | | ATCGGCTACTCATTTCGCGACG |
| | | | | | | CCGAGGAGTTCGCAGTTGCTTC |
| | | | | | | AACTGTGTTAGTTAATCATGCT |
| | | | | | | ACACGCCGGTACGTCTTGCCGT |
| | | | | | | ACGGGTTCGTGCAATCCCCTCT |
| | | | | | | GCTTGCATCCATCTCTTTAGAC |
| | | | | | | AAGTCGGACTTAGGCAACTGTT |
| | | | | | | TGCGGCGACTGCACGAAGAGAC |
| | | | | | | GGTATCTGTAAGCGTGTACGTG |
| | | | | | | GATGACATCGTTGTCAGTGCAG |
| | | | | | | ACTCTGAGCGTGACGTGGCGGA |
| | | | | | | AGCTTTAAGTAATATCTATTTG |
| | | | | | | GCCGCGATCAACTCACGGTTCC |
| | | | | | | CTATTAACGAGGAAAAGTCAAG |
| | | | | | | AGGGCCGAGCTCAACTTTGAAC |
| | | | | | | GCCTTTAATATCGAGTTGGACA |
| | | | | | | TGCACGAATTAGAGATCACCGC |
| | | | | | | TAAACGGTACGAGAAGATGTGC |
| | | | | | | GGGGAAGTTCTGATTAACGGGA |
| | | | | | | CAGGCCAGGTTTCAGACGCTAT |
| | | | | | | TTTGAATTACGTTCAGACCGTT |
| | | | | | | AATCCGGCTCAAGCCGAACAGA |
| | | | | | | TGCTTCGTGACTTCCCTGGCGT |
| | | | | | | ATTTGGAACCCTCAGCAGTAGT |
| | | | | | | GCAAGCCGCTACGATGCTTCT |
| MG148 HA- | 54 | MG148-5- | nucleo- | artificial | | ATGGTTCAGAACACGGAGTTAG |
| His tagged | | HA-His | tide | sequence | | CCGACTGTATTAACATTACCCT |
| genes | | | | | | TCAAACCAACGATAGCCGATTC |
| | | | | | | ATCGTTACTTCCTATAACATGC |
| | | | | | | ATGGATTCAACCAAGGGCTCGA |
| | | | | | | GGGAACAAAGGAGATGATCAAT |
| | | | | | | AAGCTCTACCCCGACGTAATTG |
| | | | | | | CTTTACAAGAGCACTGGCTTAC |
| | | | | | | TCCGACTAACTTGGATAGACTC |
| | | | | | | GGAGAGGTGTCAAATGATTACT |
| | | | | | | TCTTCATTGGTTCCTCTTCGAT |
| | | | | | | GAACGACGTAGTGACAGCAGGA |
| | | | | | | CCGTTATTCGGCCGCCCCTTCG |
| | | | | | | GCGGGACCGCAATACTGATTAA |
| | | | | | | TAATAAACTCGCTAACGCAACT |
| | | | | | | GTGAATGTGGCTTGCAATGATA |
| | | | | | | AATTTACAGCGGTATTGATCTC |
| | | | | | | AGAGTGTTTAGTAATTAGCGCC |
| | | | | | | TACATGCCGTGCGCAGGCACAT |
| | | | | | | GCGATCGGATGTCCTTATATTC |
| | | | | | | AGCTATAATCAGCGAGATACAG |
| | | | | | | GCATTAATCGAATTTTACCCTC |
| | | | | | | AATACAAACTTATACTGTGTGC |
| | | | | | | AGACCTCAATGTTGAGCTGGAT |
| | | | | | | GCCCCGTCGTCAGTTGCCGACT |
| | | | | | | TAGTAAATAATTTCATTTGTCG |
| | | | | | | AAATAACTTACGCCGGACGGAG |
| | | | | | | CTTGCGAACCCCACTGCATCAC |
| | | | | | | GGTTTACGTATATGCACGAGAG |
| | | | | | | CCTTCAAGCGGCGTCCTACATA |
| | | | | | | GACTACATCATTTCGTCCGACG |
| | | | | | | CATTAGACTCTATCGCGTTCAA |
| | | | | | | TGTTCTCGATTTAGATATTAAC |
| | | | | | | CTTTCGGACCACTTGCCAATTA |
| | | | | | | TGTGCGTGTTTATGTGTAAAAT |
| | | | | | | GTCAGCATGTTATTCAGACCCG |
| | | | | | | TCACAATCGGTCAAGAGCAAGA |
| | | | | | | GCCATGTTGATGATGTCAGCTA |
| | | | | | | TTTTCGTTGGGATCATGCCGCG |
| | | | | | | CTTCATCTTTACTACGAGCAAA |
| | | | | | | CGAGAGTGATGATGGAACCGAT |
| | | | | | | TCTGGACAAGCTGAACATTTGT |
| | | | | | | GTGAACTCTGTATGCGACGAGG |
| | | | | | | ATGCTTTTATTGACCACTCTAA |
| | | | | | | GCTGGAAGACATATACTCATCT |
| | | | | | | GTGGTCGCCGCACTGACGACCA |
| | | | | | | GTGCTAACTTGTGTATACCTAA |
| | | | | | | AATCAAGCATAATTTCTTCAAA |
| | | | | | | TTCTGGTGGAACCAAGAGCTTA |
| | | | | | | ATGAATTGAAGGCAGCGGCTGT |
| | | | | | | TACATCTGCACGTGCCTGGCAA |
| | | | | | | CAGGTGGGTAAGCCGAAGCATG |
| | | | | | | GGAGCGTATATCACAAGTATCA |
| | | | | | | AAAGGACAAGCTCCTGTACAAG |
| | | | | | | AAGTGCATTCGTGAGAACCAAA |
| | | | | | | AGCAGGAAAGCGTTAGTTTTAC |
| | | | | | | TAACGAGTTACACGACGCACTT |
| | | | | | | CTCAGAAAGACAGGTAAGGATT |
| | | | | | | TCTGGAAGACGTGGAACGCCAA |
| | | | | | | GGTGGGCGTGAAGCGGAAGTGT |
| | | | | | | ATTGAACAAGTTGATGGAATCG |
| | | | | | | TGGACGGGGCGACCATTGCGTG |
| | | | | | | CAACTTCGCTAAGCATTTTGAG |
| | | | | | | AAGATCTGTCAGCCTGCGACTG |
| | | | | | | CTACATTCAACACTACCATGAA |
| | | | | | | GGCAAAGTTTGAGGAAATGCGA |
| | | | | | | AGTATGTATTACACGCCATTCT |
| | | | | | | GCGACAAGACGGTAGAATTCGA |
| | | | | | | TGTTCAATTAATCGGAAAGATC |
| | | | | | | ATCTCTAACATGAGCTGCGGGA |
| | | | | | | AGGCTGCCGGACTGGATGAGCT |
| | | | | | | TTCAGCTGAGCATTTGAAACAT |
| | | | | | | TGCCACCCGATCGTGATCATCA |
| | | | | | | TCTTATGCAAGCTTTTCAATCT |
| | | | | | | GTTTGTACACGTGGGGTATCTC |
| | | | | | | CCTCTGTCTTTCGGTACTAGTT |
| | | | | | | ACACAGTTCCAATCCCTAAGCA |
| | | | | | | AAATGGGCGGTCACATGCTCTG |
| | | | | | | ACGGTTAACGACTTTCGCGGCA |
| | | | | | | TTAGCATTAGTCCTGTCATATC |
| | | | | | | TAAGATTTTCGAGCATGCTATT |
| | | | | | | TTCGCGCGGTTCGGAGATTATT |
| | | | | | | TTAGTACGAGCGACCATCAATT |
| | | | | | | CGGATTTAAAGAGAGTCTTTCC |
| | | | | | | TGTAGCCATGCTATTTACTGCG |
| | | | | | | TGCGGAACGTGATCGACCACTA |
| | | | | | | CGTTAAGCGCGGTTCTACAGTG |
| | | | | | | AACATCTGCACGGTTGATATAA |
| | | | | | | GCAAAGCGTTCGACACGGTTAA |
| | | | | | | CCACTTTGTTCTTTTCATAAAG |
| | | | | | | TTAATGGAAAGAAAGTTGCCCG |
| | | | | | | TTCAGCTGTTAGACCTGTTCGT |
| | | | | | | GCTTTGGTTCAGTATGAGCGAA |
| | | | | | | ACCTGTGTGCGATGGGGTGCTC |
| | | | | | | ATGACAGTTACTTCTTTAAACT |
| | | | | | | CAAGGCGGGAGTACGTCAAGGT |
| | | | | | | GGTGTCTTAAGTCCGTACTTCT |
| | | | | | | TTGCAGTGAGCGTCGACGATGT |
| | | | | | | CGTCGATAAAATCGCTGCGTGC |
| | | | | | | AATGCAGGCTGTTACATTAATA |
| | | | | | | ACTTATGCTCTGCTATATTCTT |
| | | | | | | ATACGCAGACGACATCATACTC |
| | | | | | | CTGTCTCCCAGCGTCTGTGGTT |
| | | | | | | TACAGAGATTGCTGGGCATATG |
| | | | | | | TGAGGCCGCAATAACAGAACTC |
| | | | | | | GACATGAAGATTAATGCTTCCA |
| | | | | | | AGACGGTCTGCACGCGGGTCGG |
| | | | | | | GCCGCGGTTCGACTCGGCATGT |
| | | | | | | GTAGGTCCGAACCTGACTTCCG |
| | | | | | | GCGGTCAACTTAATTGGGTCAA |
| | | | | | | GACGTGCCGATATTTAGGTGTC |
| | | | | | | CACTTTGCAGCAGGTCGCTCAT |
| | | | | | | TTAATTGTTCATTTGAGGAAGC |
| | | | | | | GAAGAAGAAGTTTTACTTCAGC |
| | | | | | | TTCAACAGCGTGTACAGTAAAT |
| | | | | | | TAGGTCGCTTCGCGAGCGAGGA |
| | | | | | | AGTAATCTTGAACCTGCTTAAC |
| | | | | | | GTGAAATGCGTTTCAGCAATGT |
| | | | | | | TATACGCCACAGAGGCCTGTCC |
| | | | | | | AGTACTGAGCCGCCATAAGCAC |
| | | | | | | AGTTTAGACTTCGTTATTACGA |
| | | | | | | GAGTCTTCATGAAGATTTTGCG |
| | | | | | | AACAGGAAGCCCGCAAGTCGTA |
| | | | | | | GCGGAGTGCCAGAAGTATTTCG |
| | | | | | | GATTTCTGCCAGTTAGCTATCG |
| | | | | | | TATTGACATCCGTAGCGCGCGA |
| | | | | | | TTCCTGGAACGTTTCTCCTCAT |
| | | | | | | CAGTAAACAGTTTCTGTATAGC |
| | | | | | | TTTTGGTGATAGAGCCCGAAAA |
| | | | | | | CAACTGTCTGACATCGTATGTA |
| | | | | | | AATACAATATAAGTGACGGATT |
| | | | | | | ATCATCTGGGGTGCTTAGAACC |
| | | | | | | GTAATTAATCGGATATACTTCC |
| | | | | | | AATACCCATACGATGTTCCAGA |
| | | | | | | TTACGCTCTCGAGCACCACCAC |
| | | | | | | CACCACCACTGA |
| MG148 HA- | 55 | MG148-6- | nucleo- | artificial | | ATGACCATTACAAACGACGCGG |
| His tagged | | HA-His | tide | sequence | | CACAACTTGTAGTTATCTCTTA |
| genes | | | | | | TAATTTACACGGTTTGAATCAA |
| | | | | | | GGTCTGCCGGGGATACGGGAGT |
| | | | | | | TTATGACCGAGCTGAAGCCGGA |
| | | | | | | CGTTATCATGGTTCAAGAACAT |
| | | | | | | TGGTTGACGTCTGACAACTTAA |
| | | | | | | ACAAGTTAAGCGATATTAGTGA |
| | | | | | | CGAGTATTTTGTGATAGGATCC |
| | | | | | | TCGGCTATGGACGCGCGAGTTA |
| | | | | | | GCGCGGGACCGTTATTTGGGCG |
| | | | | | | GCCTTTTGGCGGTACTGCCGTT |
| | | | | | | TTCATCAAGAACAAGTACATTA |
| | | | | | | ACGTGACAGTAAATTTAGTAAC |
| | | | | | | TTCCGAACGTTACGTCGTGATA |
| | | | | | | CAGCTTTGTGACTGGCTGTTGA |
| | | | | | | TAAATGTTTATTTGCCTTGCAT |
| | | | | | | TGGTACTTCAAACCGCATCTTA |
| | | | | | | TTATACTCAGACACGCTGTGTG |
| | | | | | | AACTCCAAAGTATAATTCGAGC |
| | | | | | | ACACCCAGAGTGCAACTGTCTT |
| | | | | | | GTCGGTGGCGACTTTAACACCG |
| | | | | | | ATCTTAACGACACTCGAAGTCA |
| | | | | | | TACTGCGAACACAGTCGTCAAC |
| | | | | | | GGGTTCATCGCTGGTTGTAACC |
| | | | | | | TGCATAGATGTGACTGTTTATT |
| | | | | | | TCCAACCCGACTTAAGCACACA |
| | | | | | | TACGTGAATGACAGTATGAATT |
| | | | | | | GTTACTCGACGATTGACTATAT |
| | | | | | | GCTTAGCAGCAACCCAGAAAAG |
| | | | | | | ATCGTTGCGTTCAACGTATTGG |
| | | | | | | ACATCGACCTGAACCTGAGTGA |
| | | | | | | CCACTTGCCTATTATCTCAATA |
| | | | | | | TGCGTATTCGACTGTAATTGCA |
| | | | | | | AGATGAATAATCCAAAGCCTAC |
| | | | | | | CTCGACAGAAAATGTTACACAC |
| | | | | | | TTTCGGTGGGACCACGCCCCTT |
| | | | | | | TACAAGACTACTACGAGCACAC |
| | | | | | | GCGTTTGGGCCTTCAACCTATT |
| | | | | | | CTCGCAGACTTGGACGAATTAA |
| | | | | | | TTGGAAACAAGTTGTCGTATAG |
| | | | | | | CAATGTTGACTTTCTGTGTCAA |
| | | | | | | GTGGATTGCATCTACAACCGTG |
| | | | | | | TTGTGTCTGTGTTGCAACAGTG |
| | | | | | | CTCACACGCCTACGTACCGAAG |
| | | | | | | CATAAGAAGAACTTTTACAAAT |
| | | | | | | TTTGGTGGAATCAAGAATTGGA |
| | | | | | | CGAATTAAAGGATAAAGCTATA |
| | | | | | | GCTAGTTGCAAAATGTGGAAGG |
| | | | | | | ACGCGGGGAAGCCACGACACGG |
| | | | | | | GGCAATACATGCGAAATACCGC |
| | | | | | | CAAGACAAGCTGCTCTATAAGA |
| | | | | | | AGCGGATCCGGCAAGAGCGCGT |
| | | | | | | TCAAGAGACGAGCAGCTTTACC |
| | | | | | | AACGAGCTGCACGATGCGCTGC |
| | | | | | | TGAATAAAAGTGGAAGAGACTT |
| | | | | | | TTGGAAGAAATGGAACAGCAAG |
| | | | | | | TTCGAAAATAAGTCGAATAAGA |
| | | | | | | TCTTGCAGGTGGGTGGAACAAG |
| | | | | | | TGACACAGCCACTATTCTTAAT |
| | | | | | | AATTTTGCTAAGCATTTCGAGC |
| | | | | | | AAGTGTGCGTGCCCTTTAATGC |
| | | | | | | GACCCGGAACGAAGAGCTCAAG |
| | | | | | | AGCCGCTACAAGGAAATGAGAC |
| | | | | | | TCAACTATTCAGAGTCTAGCGT |
| | | | | | | CATAAATGATACCACCGTATTC |
| | | | | | | GATGTTCAGTTAGTTGAGAACT |
| | | | | | | TGCTTACCAATATGAAGAACGG |
| | | | | | | CAAGGCTGGTGGTTTGGACGAA |
| | | | | | | TTAACGAGCGAACATCTTAAAT |
| | | | | | | TTTGCCATCCCGTAGTAGTCAT |
| | | | | | | CATCCTTTGCAAATTGTTCAAT |
| | | | | | | TTGTTTGTGATAAACGGGCACA |
| | | | | | | TCCCAGACTCCTTCGGAGTGAG |
| | | | | | | TTACACAGTCCCCATACCGAAG |
| | | | | | | TCTGATGGCCGGTCACGGAGCA |
| | | | | | | TGACCGTGGACGACTTTCGTGG |
| | | | | | | AATAAGCATTAGCCCAGTTATC |
| | | | | | | TCCAAACTGTTCGAGCTGTGCG |
| | | | | | | TATTGGATAGATATTCCGACTA |
| | | | | | | TCTCCAAACGAGCGACCACCAA |
| | | | | | | TTCGGCTTTAAGAAGCAGCTGG |
| | | | | | | GTTGCCGGCACGCGATATTTTC |
| | | | | | | AGTTCGTAGCGTAATCGAGCAA |
| | | | | | | TACATATCAAACGGTTCTACCG |
| | | | | | | TCAACTTGTGTGCTCTCGACCT |
| | | | | | | CTCTAAAGCGTTCGACCGAATG |
| | | | | | | AATCAATACGCGCTGTTTATTA |
| | | | | | | AGCTTATGGAAAGACGTTTCCC |
| | | | | | | AGTCAAAATATTAACCATCTTA |
| | | | | | | GAGCAATGGTTTTCCATCGCCG |
| | | | | | | AGACATGCGTGCGATGGGGCTC |
| | | | | | | TGAGTTCTCATACTTCTTCAGC |
| | | | | | | CTTCTTGCGGGAGTCCGACAGG |
| | | | | | | GTGGCGTCCTTTCTCCGGTTTT |
| | | | | | | ATTCGCCATCTTCATTGACGGT |
| | | | | | | ATTGTTAACCGTGTAAACGCCA |
| | | | | | | CGAATGTGGGATGCTACAATTC |
| | | | | | | AACGGTGTGTGTTTCTATTTTC |
| | | | | | | CTCTACGCGGATGATATTTTGT |
| | | | | | | TACTGAGCCCCACCGTGACGGG |
| | | | | | | GCTTCAAACGTTGTTGACGGTC |
| | | | | | | TGCGAGAATGAACTGTGCGAGT |
| | | | | | | TGGACATGCGTCTTAACGTAAA |
| | | | | | | CAAGAGCGTTTGTATGAGATTT |
| | | | | | | GGCGCGCGTTTTAAAGCTCACT |
| | | | | | | GCGCAAACCTCGTTAGCGTACA |
| | | | | | | GGGTGGCGCGTTACAGTGGGCT |
| | | | | | | AGCAGTTGCCGATACCTGGGAG |
| | | | | | | TTTACTTCGTATCCGGCCGAGT |
| | | | | | | CTTCCGATGCTGTTTCCACTCC |
| | | | | | | GCCAAGTGCAATTTCTTCCGTG |
| | | | | | | CCTTCAACAGCGTCTACTCGAA |
| | | | | | | GATTGGCTGCTGCGCATCTGAA |
| | | | | | | GAGGTCATCCTTTCTTTGCTGA |
| | | | | | | AATCAAAGTGCCTGCCGTACAT |
| | | | | | | ATTATACGGTGTTGAGGCTTGC |
| | | | | | | CCGGTGCTGCAACGGGACAAGC |
| | | | | | | ACTCATTTGATTTTACACTCAC |
| | | | | | | GCGAACGCTCATGAAGCTTTTC |
| | | | | | | ATGACTAGCTCTCCTGTAATTG |
| | | | | | | TTAACGAATGTCAAACCCAATT |
| | | | | | | CAACCTTCTCCCATTACGTTAT |
| | | | | | | CAGATCGATATCCGCACGGTGA |
| | | | | | | AGTTCTTAAACCAATATATCAT |
| | | | | | | TTCCCGTAACAGTATCTGTATG |
| | | | | | | TTATATAAGTCACATGCCCAAT |
| | | | | | | CGGTACTGGACACAATATTTGG |
| | | | | | | GGTCTACGGAAATAACGTCAGC |
| | | | | | | TCACTTCACGACCTTCATAATA |
| | | | | | | TTATAAACGAGCATTTCTACGA |
| | | | | | | CTTCAATCCAAGCACACTTGGC |
| | | | | | | TATAACTACCCATACGATGTTC |
| | | | | | | CAGATTACGCTCTCGAGCACCA |
| | | | | | | CCACCACCACCACTGA |
| MG148 HA- | 56 | MG148-7- | nucleo- | artificial | | ATGTACAACCTTCGCATCACTA |
| His tagged | | HA-His | tide | sequence | | GCTACAATTGTCGGAGTTTGAG |
| genes | | | | | | CGCTCTTAAACGTGACTTTGTG |
| | | | | | | AAGACATTACTCGTCTCATGTG |
| | | | | | | ACATTCTTTTCCTTCAAGAGCA |
| | | | | | | CTGGTTATCAGATGATCAGTTA |
| | | | | | | ACTCTCCTTGGCGGCTTAGATG |
| | | | | | | AGGGCTTTACCTTTACGGGAGT |
| | | | | | | AAGTGGCTTCGGAAATAAGGAA |
| | | | | | | ATCCTGAGCGGCAGACCATATG |
| | | | | | | GTGGCTGCGCAATACTCTGGCG |
| | | | | | | TTCCGCTCTCAAATTCAGCGTG |
| | | | | | | GACTTCCTGTCAGTCAACTCTC |
| | | | | | | GCCGCGTTTCTGCCATAAAGCT |
| | | | | | | TTCTAATGATTGCTGTAACTTG |
| | | | | | | CTCCTGATTAACGTGTATATGC |
| | | | | | | CTTTCGAGGACTCTGATTGTAA |
| | | | | | | CGTGAACGAATTCTTCGATGTG |
| | | | | | | CTCTTTGACATAGAATACTTGT |
| | | | | | | TGGTAAATAACGTAGATTGCCA |
| | | | | | | TTACCTGATCGGCGGCGACTTC |
| | | | | | | AACGTAGATTTGTCTAGAAATA |
| | | | | | | CTGTCCACACCGCATTATTGCG |
| | | | | | | TTCGTTCAGCGATAACAACGGC |
| | | | | | | CTGTTATACGCTTCCGATTTCG |
| | | | | | | ATGGTGCGAACTTCGACTTCAC |
| | | | | | | ATATCAATTTAATATGTCGCGG |
| | | | | | | TTCAGCCTGATCGACAATTTCA |
| | | | | | | TGCTGTCTTCGTTCTTGTTCCA |
| | | | | | | TAACATGCTTCAACGTGCAGAT |
| | | | | | | GTGGTCCACGACGTGAACAATT |
| | | | | | | TAAGTGACCACGAGCCAGTAAC |
| | | | | | | CATAGAGTTATGTATGTCTGTG |
| | | | | | | ACTCACGTTGATATCCAACGGT |
| | | | | | | CTACGGTGCCGCCTTTACAGAA |
| | | | | | | AGTCTCTTGGACCACTGCCGCC |
| | | | | | | GAGAGTCATGTAATAGACTACC |
| | | | | | | GCGAGGAACTGGTAGACCGCCT |
| | | | | | | CAAAACCGTTGTAATGCCCATC |
| | | | | | | GATTCATTAACGTGCTGTGACC |
| | | | | | | GTCGCTGTTCGGTCGCGAAGCA |
| | | | | | | CCGTTCCGATATTGCCGAGTAC |
| | | | | | | GCTAACAATATTGCGGACGTTT |
| | | | | | | GTATCAAGGCGGGACTTACGAC |
| | | | | | | GATACCTTTGATGAAGCCGGGG |
| | | | | | | CACCGTGCAACGCCTGGTTTCT |
| | | | | | | CCGAACACGTAAAGCCTGCGCG |
| | | | | | | TGACAAATCAATGTTCTGGCAT |
| | | | | | | CAATTGTGGTTGGAGTGTGGGC |
| | | | | | | GTCCGCGGACAGGACACGTAGC |
| | | | | | | AGACTGCATGCGCCGGACCCGC |
| | | | | | | GCCGCCTATCACTATGCACTCC |
| | | | | | | GTAGCGTGAAGAGAAGACGTGA |
| | | | | | | CGAGATAACTCAAGAGAGATTC |
| | | | | | | GCGACGGCGTTACTCCATAACA |
| | | | | | | ACTCGCGGAATTTCTGGTCCGA |
| | | | | | | AGTTAAGAAAATCAGAGCAAAT |
| | | | | | | CGGATGACATACAACGGCGTTG |
| | | | | | | TTGATGGGCACAGCGACGCAAC |
| | | | | | | TGACATCGTCCGGATCTTCGGA |
| | | | | | | GAACGTTACCGCGACCTGTACA |
| | | | | | | CTTCCGTACCATACGATGAGTG |
| | | | | | | TAATATGCACGAGATCAATGAC |
| | | | | | | ACTATAGACGAGCACATAAGTG |
| | | | | | | ACGATGACGACGCAGCCTTTGT |
| | | | | | | CGTGAGTCTTCGGGATGTTTCG |
| | | | | | | GACGCGATTGCGCATATTAAGC |
| | | | | | | ATTATAAGAACGACGTCGACAA |
| | | | | | | TATGCTGACCTCAGATCACTTC |
| | | | | | | ATTAACGCACCATCTGTATTGC |
| | | | | | | ACGTACATATTTCGATTCTGTT |
| | | | | | | CAGTGCGATGTTAATGCACGGT |
| | | | | | | TGTGTACCCAACCTGTTAATGT |
| | | | | | | ATAGCTCTATACGTCCTATCCC |
| | | | | | | GAAGGGACACAACCTGTCGACA |
| | | | | | | TGTGACAGCAATAATTATCGGG |
| | | | | | | GCATAGCAATCAGTTCGATCTT |
| | | | | | | CAACAAGATTTTCGATAACGTG |
| | | | | | | GTGCTCATAAAGTACCGCCATC |
| | | | | | | TGCTGTCAACTTGTGACTTACA |
| | | | | | | ATTCGGATTTAAGAAGAAACAT |
| | | | | | | AGTACGCAGATGTGTACCATGG |
| | | | | | | TGCTTAAGGAAACGCTTTCTTA |
| | | | | | | CTATCTCTCGAACCGTAGTAAC |
| | | | | | | GTGTTCTGCACATTCCTCGATG |
| | | | | | | CGACAAAAGCCTTCGACCGTAT |
| | | | | | | AAACTACTGCAAATTATTCAAT |
| | | | | | | TTGTTACTTTGTCGCTCTCTCC |
| | | | | | | CCTATTGCATCATCCGCGTTTT |
| | | | | | | ACTTTCCCTTTATACAAACAAT |
| | | | | | | TACGTGTACGTGAGCTGGGTTG |
| | | | | | | GTTCAAACAGTTCATCCTTCAG |
| | | | | | | AGCCTGCAACGGGGTCAAGCAA |
| | | | | | | GGTGGGGTACTGTCACCAGTTT |
| | | | | | | TATTTTGTTTATACATGGACGG |
| | | | | | | GCTTTTGAATAAGCTCTCACAT |
| | | | | | | GCTGGGGTCGGCTGTTATATGG |
| | | | | | | GTGAGATGTTTGTTGGCGCCCT |
| | | | | | | TGCATACGCAGACGATATCGTG |
| | | | | | | CTGATTAGCCCCACGCCTTCAG |
| | | | | | | CTATGAACAATATGCTTTCCAT |
| | | | | | | ATGTGACGAGTTCTCCATCGAG |
| | | | | | | TATAATGTCCTGTTTAACCCAT |
| | | | | | | TAAAGAGTAAGTGTATGTATTT |
| | | | | | | CTACCCTAAGAGCCGCTCTACG |
| | | | | | | CTCTTGCTGCATCGTTACAACG |
| | | | | | | TTTGTGACCTGCAATTCTCGAT |
| | | | | | | AAACGGCAAGGCCATTGAGTTC |
| | | | | | | GTTGACTCTTATAAGCACTTGG |
| | | | | | | GGCATGTTATTTGTTCCGACAT |
| | | | | | | GACTGACGACATCGACATCAGC |
| | | | | | | GAAAAGCGGGGCGTATTTATTG |
| | | | | | | GCCAAGCTAACAACATCATTTG |
| | | | | | | CTACTTTGCTAAGTTGTCATCT |
| | | | | | | GCCATAAAGTATCGACTGTTCA |
| | | | | | | CGTCGTACTGTACCAGCTTCTT |
| | | | | | | TGGCTGTGAGCTTTGGAGAATA |
| | | | | | | TCTAACGATTCGTTGGATAGCA |
| | | | | | | TGTGCACGGCGTGGCGCCGAGC |
| | | | | | | CATTCGGAGAATTTGGAGTTTG |
| | | | | | | CCCTATACCGCTCACGGACGCT |
| | | | | | | TCTTGCCCGTTTTATGCAACTC |
| | | | | | | TTACAACATTTTCGACCAATTC |
| | | | | | | TGTGTACGGATTCTTAACTTCA |
| | | | | | | TTAGAAGATGCCTCTCTGATCA |
| | | | | | | AAGTAGCTTGCTGGTGCGAAGC |
| | | | | | | ATCGCCACCCAGGCTTTGATGT |
| | | | | | | TCCATCACGCGCGATCTCCTTT |
| | | | | | | AGGATATAACTTCATATATTGT |
| | | | | | | GCGCGTCGTTATTGTTTCTCAT |
| | | | | | | TGACTGACTTTGTGAACTACAA |
| | | | | | | CAACTTCGTGTCAAACGTCGAG |
| | | | | | | AAGCTCAATTGCACTCAAAACG |
| | | | | | | ACGACGACACGATAGCAAATTG |
| | | | | | | TCGCCTGCTGCGAGAATTGATT |
| | | | | | | GACTTACGCGATGGTGTGTTGC |
| | | | | | | ACCTTAGTGACGACGTCTTTCT |
| | | | | | | TACAAACAGCGAGCTCTCATTC |
| | | | | | | ATGATCAACTGCGTTAGTACAC |
| | | | | | | TGTACCCATACGATGTTCCAGA |
| | | | | | | TTACGCTCTCGAGCACCACCAC |
| | | | | | | CACCACCACTGA |
| MG148 HA- | 57 | MG148-9- | nucleo- | artificial | | ATGACAGAAACTAGCCATGCAC |
| His tagged | | HA-His | tide | sequence | | CTGTCTTGGACCAAACATGCAC |
| genes | | | | | | CCGTTACCAGAGACACAACGTC |
| | | | | | | CACGAGCAGGCTATGCAATTTG |
| | | | | | | TGCCACCAAACCGTGTTGTATT |
| | | | | | | CAGCTTCTTTGGCCCTTGTGAC |
| | | | | | | ATCGTATGTCACCGAGGCGCTT |
| | | | | | | TTCTTTCAGTCACCTCATATTG |
| | | | | | | CAAAACACTCTCACTCTTGCAG |
| | | | | | | CGGGCACACTTCACTAGACACC |
| | | | | | | GGAATAAGGCTACCGGCCAACC |
| | | | | | | GTTCCTGCTGTTGTCCGTTTAT |
| | | | | | | AAGCAACAAGACTTGCAAACAC |
| | | | | | | TGACCCAACAGGGGCTTGTTCC |
| | | | | | | GTTTACACAGACATGCAATGAG |
| | | | | | | GCGCTGTCTCTTCGGGGTTGTC |
| | | | | | | GCGTGGCGCCATCTGGTCGGGT |
| | | | | | | CGCTTTGCCGTATATGATGTAC |
| | | | | | | CCACAGGGTGATCACCGTAACC |
| | | | | | | AGCCAGTACACAATCAAGAGAC |
| | | | | | | ACACACTCCCCTTACTCCAGGC |
| | | | | | | CTTGATGCGAACCCGTTCGCGG |
| | | | | | | CCTTATCGGTGGAGCAACAAAC |
| | | | | | | GTGTGGAGCTAGTACGTCTCGC |
| | | | | | | ACCTCGTTAATAGTTGGGTCTA |
| | | | | | | TGAACGTAAACGGACTGTTTAC |
| | | | | | | CCAGACTATGTCCTCTAAGCAT |
| | | | | | | CAAGATGTTTGCGCATTTGTTT |
| | | | | | | CTAGCCACACAGTCGACATATG |
| | | | | | | TTGCCTGCAGGAGACATGGTGC |
| | | | | | | GACATGAGCGAGACTGAGTACT |
| | | | | | | CTTCTATGTTAGCCAGTTGTGG |
| | | | | | | TTATACGTCATTCGGTGTCTCT |
| | | | | | | GGATATTCTACCCGTACACGGA |
| | | | | | | CAGGCGGCGGTACCGGCATCCT |
| | | | | | | GGTGGCAACTTCTATCTCTCAT |
| | | | | | | CTGGCCCACATGCTCCACTGGG |
| | | | | | | AGTCACAGAAGCACACCCAGAC |
| | | | | | | CACCTGGATCAAACTGAACAGC |
| | | | | | | ACTCAGTACAAAGCGAGAAAGA |
| | | | | | | CCGCTGTAGGCAGCGTTTATCT |
| | | | | | | CCGGCCGGTCAGCCACACCCGA |
| | | | | | | CCAGGCGACGCTGAGCGGTACG |
| | | | | | | TGACCGAACTCATTGCCCTGCG |
| | | | | | | CGATGACATTACATATCTTAGC |
| | | | | | | GAGCACAACTTCGACGTCGTCC |
| | | | | | | TGTGCGGCGACTTTAATGCCAG |
| | | | | | | AATAGGTAACTCTCAGGTCAGC |
| | | | | | | CCGCACGTTCCGCAACACAATG |
| | | | | | | AGCAAACGCGCAATACGCAGGG |
| | | | | | | TGATCGCTTGGTTACTCTTCTG |
| | | | | | | GAGCAGTGCAACATGTTCGTTA |
| | | | | | | TGCATAACCACACCAGTTTCAG |
| | | | | | | CCCAACCTGCATGCATGCAGGC |
| | | | | | | GGCTCATCTGTGGTAGACTACT |
| | | | | | | GTATCACGAACTCTTCATGCAG |
| | | | | | | AGCACGTTGTTCAGACGCTGCA |
| | | | | | | ATTAACTTCGCAGGGCCATGTG |
| | | | | | | TGAGTGATCACGCTTTATTGAC |
| | | | | | | TATGAGCTTATCTCACGCAACT |
| | | | | | | AGAACGCGTCATCGCAAGACGC |
| | | | | | | GCCGTGCCCACGCAAAGTTATG |
| | | | | | | GAATCGCAGAGCTATGCATGAT |
| | | | | | | GAGCAAACAATCGCCCAATTTC |
| | | | | | | ACGCTACACTGGAAACCCCGCT |
| | | | | | | TACACAACTTTCCGACTTCATA |
| | | | | | | GATGCAAGCACCCTGAGTCCCC |
| | | | | | | AAGAAGCAACGGATACCTTCAC |
| | | | | | | TAGTAAGTTAACTGCCGCTCTG |
| | | | | | | CGTGACGCTGGCGCCACTTGTT |
| | | | | | | TTGGAACTCACCAAGTTCGGCC |
| | | | | | | ACATAGAGCTCAATGGTGGAAT |
| | | | | | | AAAGACTATGCGAACCTTAGAC |
| | | | | | | ATGAGTGCTGGAACCTGTACGA |
| | | | | | | GACACATCGACGTAGTGGATCC |
| | | | | | | GAGGCAGACCGTGCTGCGTATG |
| | | | | | | AGGAGAAGCGCGCGGCAAAGAA |
| | | | | | | CAAAATGAAACGCAATCTGAAG |
| | | | | | | CGCGCGTACGTTAAGGCCCAAG |
| | | | | | | CCGGGCACGTGTCAAAGTTATG |
| | | | | | | TACACCGGGGCTTGTCAGCAAG |
| | | | | | | ACTAGTTGGGGCGCTGCCAAAA |
| | | | | | | GACTCTTAAAGTTAGTTACACG |
| | | | | | | TGGATCATCAGCCACCGAGCAT |
| | | | | | | ACTCTCCCCACGGTCATTCACG |
| | | | | | | ACGGTGCTCCCACTGACGATCT |
| | | | | | | CGAGGCTGTTATGCAAGTCTTC |
| | | | | | | TCCTCTCACTACCATACGGAAA |
| | | | | | | TGAACCCTGTCCCCAGTGCGAC |
| | | | | | | ATTTTGTGAGGCTACAGACACT |
| | | | | | | CTTGTTAAGGACAAGCTGCATG |
| | | | | | | CGTGGCGCCAGGCGGAGCCTGA |
| | | | | | | GCATATTAATAATATGGACGAT |
| | | | | | | GAATTCACCATAACCGAGTTAG |
| | | | | | | ATCGTGCGCTTGGTTCCATGCA |
| | | | | | | TAACTGGAAGGCCGCTGATCAC |
| | | | | | | GACGGAATGATAATCGAACTGT |
| | | | | | | TGCGCGCAGGCGGACCTGCATT |
| | | | | | | ACAACTCGTGGTACTTAAAATC |
| | | | | | | TTGAATTTCTGTTGGACGCACG |
| | | | | | | AGACGATTCCAACAAACTGGAA |
| | | | | | | ATTAGGCACCATCATCAGCCTG |
| | | | | | | TACAAAGCCGGTAAGAAAGAAA |
| | | | | | | ACCCCTCTAACTACCGTGGGAT |
| | | | | | | TACGCTCTTAAGCGTAGTTCGG |
| | | | | | | AAGTTGTTTTGTACGTTATTAC |
| | | | | | | GGAGCCGTTTACAGGATAACAT |
| | | | | | | CTCTCTCCACGAGTCTCAAGCC |
| | | | | | | GCGTTCCGTGCAAACCGTGGGT |
| | | | | | | GTATGGACCACGTGCACACGCT |
| | | | | | | TGCGCGTATCGTTCGCGCCGCG |
| | | | | | | AATCGTAAGGACATTCCGGTGT |
| | | | | | | ACGCTTTCTTCCTTGATATACG |
| | | | | | | CAAGGCGTACGACACGGTTTGG |
| | | | | | | CGCGACGGGCTCATTTATAAGC |
| | | | | | | TTCTGCAAAAGGGAGTGACTGG |
| | | | | | | TCGGTTAGGGAGAGTCATTTCT |
| | | | | | | CAGGTACTTACCGATACGCAAT |
| | | | | | | CCCGTGTGCGCTTTCAACACCG |
| | | | | | | TGAAAGCGCTTATTTCCCGCTT |
| | | | | | | ACATTAGGTGTAGGCCAAGGGG |
| | | | | | | ATCCTCTTTCTACAATTCTCTT |
| | | | | | | TGACGTGTTTATAGATGACTTA |
| | | | | | | TTAGAGGAGTTACATGCACGCC |
| | | | | | | CGGTGCACCACTGTATCCCTGT |
| | | | | | | AGACAGCCCACATCTGGACCGC |
| | | | | | | ATCGCCGACCTCACTTATGCGG |
| | | | | | | ATGATGTGAATGCGTTGAGCTT |
| | | | | | | GACCCCCGAGGGTTTGCAGGGC |
| | | | | | | CACATAGACACAATCGACACTT |
| | | | | | | GGTTGTTCCGATGGCGGAGTCA |
| | | | | | | ACCAAATGTTAGTAAGTCTAAG |
| | | | | | | ACGATGGTCTTCAACCCGCCGC |
| | | | | | | AGGGAGTTCCGCCAACAGTATT |
| | | | | | | TACTATGCGTGGTTCAAACCTC |
| | | | | | | GATACTGTCCAATGCTTTAAGT |
| | | | | | | ACCTGGGAGTATTCTTTCAATC |
| | | | | | | AAATGGGTCCTGGACCGACCAC |
| | | | | | | ACTCAGCACGTACGTACACAGA |
| | | | | | | TGAATAAAGCCGTGGGCATGTG |
| | | | | | | GCGTCCTGTTCTCCGGTGCCAC |
| | | | | | | TACCTGCCCGTAGCAGCACGCC |
| | | | | | | TGCGCATTATATACGCGTTCGT |
| | | | | | | CTACGCGCCTGCGCTCTACGGC |
| | | | | | | GCTGAGGTCTGGATCGCACCTA |
| | | | | | | AGTCAGAATTGGAGAAGTTAGA |
| | | | | | | TACTATTTGCAAGAGTGCGATC |
| | | | | | | CGTACAGTCTTTGGCTTGCACC |
| | | | | | | AATTCGACTGTCGCGAGGAGAT |
| | | | | | | ATTATTTGCCGACACCGGATTG |
| | | | | | | CTCCCAGTCTCGAGCCTTATCA |
| | | | | | | ACGCAGCGAAACTGTGTTGGTT |
| | | | | | | CACTAAGCTTCTTAACATGCCT |
| | | | | | | GAGTCCCGTTTCCCCACGGCCG |
| | | | | | | TTGAGGCTGTGACGTTACCTGG |
| | | | | | | AGATACACAACGGGGCCGGGTT |
| | | | | | | GCCGGTGGCGATTTCGGGACGC |
| | | | | | | GTATTGCCGACATTACCACGGA |
| | | | | | | AATTCGTCGTTATGCGCACATG |
| | | | | | | AAATTAGTGATGCAAGACCCAT |
| | | | | | | TGGAGCACGCCCCGCACCGCCG |
| | | | | | | CCGGCGTCCCATACGCGTTTCA |
| | | | | | | AAGCGTCTTTCTCACACCAAGC |
| | | | | | | CGGACGCGCACCTCTACGAAAC |
| | | | | | | TATTACATACTCGTCTCTGACC |
| | | | | | | CGTGAGCGCGTAAAGGCTCTCT |
| | | | | | | TGTGGAAGGTGTACATGGAAAA |
| | | | | | | GTGTTTCGAACGGCGCGGCCGC |
| | | | | | | AAGGATGGCGTAACCGGTGAGT |
| | | | | | | GGATGCGCTCTGTCATTAAGCA |
| | | | | | | CGAGCTGGGTTCATTGGCGCCG |
| | | | | | | TTTCTTCACAGCGTTGAGTGTG |
| | | | | | | GGTTAGTGCGTGTTCTGATGAG |
| | | | | | | TGCTCGAAGTCGTTTAAGTCCA |
| | | | | | | GTTTTATGCCGGCCACCGCAGG |
| | | | | | | ACAAACACCGGGAGTACTCACA |
| | | | | | | AGCAGCCGGCGCCTTTACCACA |
| | | | | | | TACATATCGCGTGCCCCGATTG |
| | | | | | | GTGCTGTGCTTCCAAGTCCACC |
| | | | | | | GGTAGCGCATTGTGTACGTCAC |
| | | | | | | GTTTCGGCTGTCGCCGAGCTCG |
| | | | | | | TACGAGAACTGTTAGCCCAACC |
| | | | | | | TGGTCCTTACGGGTGCGGTAGT |
| | | | | | | TCTGTGTCGTGGTCTGACATCG |
| | | | | | | CAGTTGTACTGGTCGTGGGAAT |
| | | | | | | CGACGGCGTTGGTGGCGCGCCT |
| | | | | | | GGCGTTTTGGGAGTCGTGGCCC |
| | | | | | | GTCCGTCCCCTCTTTCAGAACT |
| | | | | | | TGCTCGTGTGGTCGCATTCTGT |
| | | | | | | GGAGCGAGTCCCAGACTCCGAC |
| | | | | | | ACCATGTTGCTTGGATGTCACG |
| | | | | | | GAGCGTTCCTGTCGCGGGTGTC |
| | | | | | | GTAAACGTTCCAGTGGACATGG |
| | | | | | | CTATGTTGCAGCGGACGAAGGC |
| | | | | | | CGGCCGGCAAGTCGCTCCCCAA |
| | | | | | | GGCAAGCACGGAGCACACTCTT |
| | | | | | | CGTACCCATACGATGTTCCAGA |
| | | | | | | TTACGCTCTCGAGCACCACCAC |
| | | | | | | CACCACCACTGA |
| MG148 HA- | 58 | MG148-10- | nucleo- | artificial | | ATGGCGATAAGCTACACATCAA |
| His tagged | | HA-His | tide | sequence | | ACAATTCGGTTCGGATATGCTC |
| genes | | | | | | TTTTAATATGCATGGCTTTAAC |
| | | | | | | AATGGGGTGAGCATGACGAAGA |
| | | | | | | TCCTTTGCAACAGCTTTGATGT |
| | | | | | | GATCCTTTTACAAGAGCACTGG |
| | | | | | | CTTCTGCCGTCTAACCTGTCTA |
| | | | | | | AGCTGGGCGATATATCACACGA |
| | | | | | | CTTTACACACCATTCCGTTAGC |
| | | | | | | GCGATGACAACAAAGATTTCAG |
| | | | | | | AGGGCATTCTGTACGGCCGACC |
| | | | | | | GTTTGGTGGAGTATCAATCTTG |
| | | | | | | TACAGAAACTCTCTTACAAAGA |
| | | | | | | ATATCAGCATTATAGATGCGGA |
| | | | | | | CAAGGAAGAAGGGCGCTACGTT |
| | | | | | | ACTATAAAGCTGAAATGTGTCA |
| | | | | | | ATAACGAGTTCATTACGGTTAC |
| | | | | | | GAACGTGTACTTTCCAACCATT |
| | | | | | | TGTGCATACAGTGACTACATAG |
| | | | | | | TAAATACCAGCTCTATCATGGC |
| | | | | | | CTACTTAGACAACCTTTTCGCA |
| | | | | | | AACGAGGTATCCTGTCACCATG |
| | | | | | | TTGTGGCGGGTGATTTCAATTT |
| | | | | | | CGAATATCGGAACGATAATATC |
| | | | | | | GGCTTCGACTTGTTCCGTTCTC |
| | | | | | | TTGCTGTCGACAACAACTTGAT |
| | | | | | | ATGCTGTGATGACCTGCATCTT |
| | | | | | | AATCAAAATATAAAATTTACGT |
| | | | | | | ATAAGCACGAAACCCTGCCTCA |
| | | | | | | ACAAAGCTGGCTTGACCACTTC |
| | | | | | | TTCGTTTCCGCCGGGCTCACAA |
| | | | | | | GTTCTATTGTATACTGTGATAC |
| | | | | | | CATAGATAACGGTAGCAATCTT |
| | | | | | | TCCGACCATCTTCCGATCTGCT |
| | | | | | | GTACAATCAACGTCTCACTGAG |
| | | | | | | CGACTCCAACTGCACCCCGAAG |
| | | | | | | CTTTCTAAGGTGTATCGCGACC |
| | | | | | | GCTGGGACAAGGCAGATCTGAT |
| | | | | | | TAAGTACTACTATCAGAGCGGG |
| | | | | | | ATTCATCTCCAAAGTCTCACAG |
| | | | | | | CCCCGCCACACGTGACGCAATG |
| | | | | | | CTCCACACATTGTCAGAAGGCT |
| | | | | | | CAACATCTCCAGGATATTAACG |
| | | | | | | CGTATTACGAGCGTATAATTCA |
| | | | | | | AGCTCTCAAAGCTTCGGCAATA |
| | | | | | | GGCTGCGTTCCCCGGATGCCGG |
| | | | | | | TAAACTGCTTGAAGCACTGGTG |
| | | | | | | GAACGACGATCTCACGCGACTT |
| | | | | | | AAGAACTTATCTATCGATATGC |
| | | | | | | ACAACTTGTGGCGCCAGGTCGG |
| | | | | | | ATCCCCGCGCAATGGCATTATC |
| | | | | | | AATGAGGCCCGCCTGAAGGCGA |
| | | | | | | AATTGGATTATAAGCAGGCCAT |
| | | | | | | ACGCCAGGCAATGCTCGATTGC |
| | | | | | | GAGAACAAAGACGCCGATATTA |
| | | | | | | TTAATAACAAATTTAACCAGAA |
| | | | | | | GGATTCCCGTAATTTCTGGAAG |
| | | | | | | TGTTGGGGCGCAAAATATCGCA |
| | | | | | | AGAAAGTGAACAATACCGCGTG |
| | | | | | | CATAGACGGTTGTACGGACAAT |
| | | | | | | TCCACCATTGCCAATAAGTTTA |
| | | | | | | AGGCGTACTTTCAAAACACCTA |
| | | | | | | CGTGGACAGCACATGTGACGTG |
| | | | | | | AATGCCTGTATGGAGTTCGATA |
| | | | | | | AGCTCATGAGCGACCACTCTCA |
| | | | | | | CCTGCAGCACAATGACATAGTG |
| | | | | | | GAAGAAATTCGTATTGAGGATA |
| | | | | | | TTGAGAAATGCATCGAGCTTCT |
| | | | | | | CAAACCCCTCAAGTCAGCAGGC |
| | | | | | | CATGACGACGTCGCCCCAGAGC |
| | | | | | | ATCTGATCCATAGTCACCCTAG |
| | | | | | | TCTGTGCATGCACTTGAAACTC |
| | | | | | | CTGTTTTCAATGATGTTAAATC |
| | | | | | | ACAACTATGTTCCGGATAGTTT |
| | | | | | | CGGAATTGGAATCATTATCCCT |
| | | | | | | GTAGTGAAGGACAAACGCGGGA |
| | | | | | | ATCTGAACAGTGTTGAGAACTA |
| | | | | | | TAGACCCATCACGCTGTCTCCA |
| | | | | | | ATCATATCTAAGGTCTTTGAGA |
| | | | | | | GTTTCGTTCTGAACCGCTTCGC |
| | | | | | | CAAGTTCATGACTTGCGACCCA |
| | | | | | | CTTCAGTTTGGATTCCAGCGCT |
| | | | | | | CGGTTGGATGCAATAACGCCCT |
| | | | | | | GTTCGCGATCCGCCAAGTAATT |
| | | | | | | CAATATTTTAACGATCGTGATT |
| | | | | | | CAAACGTTATGGTCGCATCGCT |
| | | | | | | GGATGCCTGTAAGGCTTTCGAT |
| | | | | | | CGTGTCAATCACTTTAAGCTGT |
| | | | | | | TCTCCACTCTGCTGCAAAGAAA |
| | | | | | | GCTTCCATTACACATTATAAAG |
| | | | | | | GTTTTGATCAATTGGTATTGCA |
| | | | | | | AATTGATGGTGCAAGTCCGATG |
| | | | | | | GAACAACTCATTAAGCGATTTG |
| | | | | | | TTCCATGTGAAATCGGGTGTAC |
| | | | | | | GGCAGGGTGGAATTTTAAGCCC |
| | | | | | | GGCCTTGTTCAATGTGTACATT |
| | | | | | | GACTGCGTGATCAACAAGCTCC |
| | | | | | | GGGCTAGCCAACTTGGTTGCCA |
| | | | | | | CATCGGATCCCTCTATATTGCT |
| | | | | | | GTGGTCCTCTTTGCCGATGACA |
| | | | | | | TTCTCCTCTTGTCATCATCTTT |
| | | | | | | CATGGAACTTCAACGTATGATA |
| | | | | | | GACCTTTGCGTGGAGTCAGGTG |
| | | | | | | ACGAAATAGGTTTAAAGTTTAA |
| | | | | | | TGCAGCGAAGTCACACTGCATG |
| | | | | | | ATTATCGGTCCGCACAAGGTCC |
| | | | | | | TGGTTAAGCCCGATATGATGAT |
| | | | | | | GGGGAACTCACCTACCGCCTGG |
| | | | | | | TCGGAGACAATTAAGTATTTAG |
| | | | | | | GGGTGTATATTCAAAGCGATAA |
| | | | | | | GAAGTTCACTGTTGACCTGTCG |
| | | | | | | CTGGTTAGACGGAAATTCTTCG |
| | | | | | | CAAGCGTCAATTGTATACTGCG |
| | | | | | | TAATGCGGCCTTCACCTCAGAC |
| | | | | | | ATTGTGAAGCTGGAATTGGTTG |
| | | | | | | AGAAGCAATGTTTCCCCATTTT |
| | | | | | | ACTGTACGGGTTACAATCTTTC |
| | | | | | | GACTTGAAATCGTCGGTAATCG |
| | | | | | | CAAACGTTAACGCCTGGGTGAA |
| | | | | | | CTCCGTATATCGGAAGATTTTC |
| | | | | | | GGTTACCATAAATGGGAGTCAG |
| | | | | | | TGAAGGAATGCATTTACCTGTT |
| | | | | | | GGGACGTTTGGACGTTTTCCAC |
| | | | | | | ACAATTAAGCTGCGTCGTATTA |
| | | | | | | ATTTCCTTAAGAACATCCAAAA |
| | | | | | | GTGTAACAACGAGGTGGCACGC |
| | | | | | | TCATTATTCAACTACGTCCTGC |
| | | | | | | ACACGCGTGAATTTCAATCTTG |
| | | | | | | CTGCACGTTACCGAACAATAGC |
| | | | | | | GTCATAAATATATCCAAGTCGT |
| | | | | | | GCGACAATGTACGTAAGGCGGT |
| | | | | | | CTTTAACTCCTTCCAATCGAAG |
| | | | | | | GTGGTGGGTCACTACCCATACG |
| | | | | | | ATGTTCCAGATTACGCTCTCGA |
| | | | | | | GCACCACCACCACCACCACTGA |
| MG148 HA- | 59 | MG148-11- | nucleo- | artificial | | ATGGCAAGTCAGACTTTAGATA |
| His tagged | | HA-His | tide | sequence | | CACAAACGGCAAACAATGTGTG |
| genes | | | | | | TAACTCTGTGTCGGTTATGTCT |
| | | | | | | TATAATATGCACGGCTTTAATC |
| | | | | | | AGGGTTATTCATACTTGAACGA |
| | | | | | | TATCTGCATGAAGCAGACGTAC |
| | | | | | | CAAGTGATCCTTATTCAAGAGC |
| | | | | | | ACTGGCTGTACCCAGCTACACT |
| | | | | | | GCATAAGTTAGCCAATATTAGC |
| | | | | | | GAGCACTACTCCTTCTACGGAG |
| | | | | | | CGAGTGCTATGCAAACCGCCTT |
| | | | | | | AGATGCAGGCTTTATTCGCGGA |
| | | | | | | CGTCCCTACGGTGGAACCGGTA |
| | | | | | | TACTGCTTCACAAAGATATTGC |
| | | | | | | TAAATACTGCGTTGAGAGTTGC |
| | | | | | | GCATTCGAGCGAGTCGTTGGGG |
| | | | | | | TACTTTTAGGAGACTTTCTTTT |
| | | | | | | CGTCAATGCATATTTCCCGTGC |
| | | | | | | TGGGACGGGTCCGTTATCAACT |
| | | | | | | TGGACGCCGCAAATGAACTGTT |
| | | | | | | GGCAAACCTCAGTAATGTATTG |
| | | | | | | GATAGCTACCAAGCTAAACACG |
| | | | | | | TCGTATTCGGCGGTGACTTTAA |
| | | | | | | CGCAAACCTTACGAAGAATTCA |
| | | | | | | GTTTACTCTAACATGATTCTCG |
| | | | | | | AGTTCATGTCCGAGCATCAGCT |
| | | | | | | GGATATATGTCGTAAGGTGCTG |
| | | | | | | TTCGGATCTTCCAATGTCATAT |
| | | | | | | TCTCGGACACCTACGTTAACGA |
| | | | | | | GGCACTTAACGCCTCTTCGTGT |
| | | | | | | ATAGACTTTATCTGTATTAGCA |
| | | | | | | GCGGACTGTCAAATAACGTAGC |
| | | | | | | TCAATACGATGTACACGACGTC |
| | | | | | | TTCAACAACCACGCAGACCATC |
| | | | | | | TGCCTGTCAGCCTCAAGCTGTG |
| | | | | | | TTTACCAGTCAGTTCCATCCTG |
| | | | | | | TATAACTGCATAGCAAGTGGTT |
| | | | | | | CTTCAGCTTGCATGTTCAATGA |
| | | | | | | GGCAAAGTCTCGGGATACTCAA |
| | | | | | | TCTTGCGATTCTAAGGGCAATA |
| | | | | | | AGCTGCGCTGGGACAAAGGCAA |
| | | | | | | TACGCAATTATACTCCGACCTT |
| | | | | | | ACCTATGCTCAGTTGTTTCCCA |
| | | | | | | TTTACGAAACCTTACATTCAAT |
| | | | | | | CGATGTAGAAAGCAGCGCTTAC |
| | | | | | | ACCGCTCAGGAACACCGTAGCC |
| | | | | | | TGATAAACGAAACCTACACTCA |
| | | | | | | AGTTGTGAGCGCCCTTCATTAT |
| | | | | | | GCGGCAAATGTTTCCATTCCTG |
| | | | | | | CAATGGCGAGTCACACATTAAA |
| | | | | | | ACACTGGTGGTGTAGCGACCTG |
| | | | | | | AGTGAATTGAAGAAGAAATCGA |
| | | | | | | TGTTATCACACAACGAGTGGAT |
| | | | | | | AAACGCGGGCAAGCCACACGCA |
| | | | | | | GGCCGCATTCACACCGCCAAGC |
| | | | | | | AGCAAAGCAAGTTGCAGTACAA |
| | | | | | | GCGTGCAATCAAGCACGCGAAG |
| | | | | | | GCAAGCGCTGAAAATAGTGTGA |
| | | | | | | GTGACGAGCTGCACCGTAACTT |
| | | | | | | GTGTTCTCGCAACAACGTTAAG |
| | | | | | | TTTTGGAAGACGTGGAAGAACA |
| | | | | | | AAATTAAACGACCGGGTACCGA |
| | | | | | | GAAATTGTACGTGGAAGGTTGT |
| | | | | | | AATACTGACGCTAAGGCCGCTG |
| | | | | | | AGCTGCTCGCCGACTATTTCAA |
| | | | | | | CAAGGCTACGAGCCCGAACAGC |
| | | | | | | AACGAATACAACAACAGCAAGA |
| | | | | | | GAGTTGAGTTCGAGAAGAGTTT |
| | | | | | | CGCCTCTTACGCGATTCAAGCG |
| | | | | | | AACGACATTGACATCTCAGCCG |
| | | | | | | GCCTTGTAGAGTCGGCTGCCTT |
| | | | | | | GAAGATGACTGCCGGGAAGGCT |
| | | | | | | GCTGGGATTGACAATATCTCAA |
| | | | | | | TTGAACATGTACACTACTGCCA |
| | | | | | | CATCGTAATTTACAGCTTATTA |
| | | | | | | GCTAAGCTGTTTAACTTAATGC |
| | | | | | | TTTGTTTCTCTTGCGTGCCGGA |
| | | | | | | TGCCTTCGGTTATGGGGTCACA |
| | | | | | | ACACCGATCCCAAAAGAGGAAT |
| | | | | | | CTCACAAGAAAATACACCCTGT |
| | | | | | | TGAGAACTTCCGTGGTATTACA |
| | | | | | | CTGTCCCCCGTGCTTTCAAAGT |
| | | | | | | TGTTCGAACACTGCTTATTATC |
| | | | | | | TGTGTTTTCGGACTTTCTCCAA |
| | | | | | | ACGAGTGACAACCAATTTGGGT |
| | | | | | | TTAAGCGGAGCACGGGATGTAC |
| | | | | | | CCACGCTATATACGCTTTGCGT |
| | | | | | | AAGGTGACTGAGTTCTTTATAT |
| | | | | | | GCAACGAGAGCACCGTGAACAT |
| | | | | | | GTGCTTCCTTGATATTAGTAAG |
| | | | | | | GGATTCGACAAGGTTAACAACT |
| | | | | | | TCGAGCTTCTTTTGAAACTTAT |
| | | | | | | GAAGCGCAAGGCACCCTCTTGT |
| | | | | | | TTTATAAAGCTCTTACACGACT |
| | | | | | | GGTTCTCCATCTCTTACGGTTC |
| | | | | | | TGTTAAATGGAATACATCGCGA |
| | | | | | | AGTGACTGGTATAAAATAGGTG |
| | | | | | | CAGGAGTACGCCAAGGCGGGGT |
| | | | | | | TTTAAGTCCTATTTTATTCGCC |
| | | | | | | GTATACGTCGATGCGATGCTGG |
| | | | | | | AGAAGGTCAAGACCATGGGATG |
| | | | | | | CCAATACAAAAGTCTGTGCACG |
| | | | | | | GGTGCTTTCATGTACGCTGACG |
| | | | | | | ATCTTGTGTTATTATCTCCAAG |
| | | | | | | TATTTACGAGTTACAACACATG |
| | | | | | | ATTGCCTTATGCCGAAACGAGT |
| | | | | | | TACACAGTTTAGACCTGAAGCT |
| | | | | | | TAATGTTAAGAAGAGCAAGGCG |
| | | | | | | TTGCGGATCGGCAAGCGGTACA |
| | | | | | | AGTGCAAAGCTCTCCCTCTTAT |
| | | | | | | GATAGACGGTCAAGCAGTTCAT |
| | | | | | | TGGTCTAATGAGGCTCGTTACC |
| | | | | | | TGGGTATAGTGATTAGATCTGC |
| | | | | | | ATGTAAGTTCAAGTGTAACTTC |
| | | | | | | GATCCGGCTAAGGTCAAGTTCT |
| | | | | | | ATAAGGCGGCCAATACAATTTT |
| | | | | | | GGCCAAGCTCGGTAATAAATGC |
| | | | | | | AATGTCACCGTCACATTGTATC |
| | | | | | | TGGTCGCGGCTGTCGCCCTTCC |
| | | | | | | GCCCCTTATCTATGGTATAGAG |
| | | | | | | GCATTGACGCTCAACTCAAGCG |
| | | | | | | AGTTGAACTCCCTGAACCACCC |
| | | | | | | GTGGAATAACTGTCTGGGCAAA |
| | | | | | | ATGTTCAACACGTTTGACAAGG |
| | | | | | | AACTGATAAAGAATTGTTGCGA |
| | | | | | | TATCCTGGGTTACGAATCCTGT |
| | | | | | | CAGAACGTGTATGTTAAGAAGG |
| | | | | | | TGGAGAAGTTCTTAAGAAACAT |
| | | | | | | GAAGTTTATTGATAACGCCATA |
| | | | | | | CTCTCTGCGCTTAACACGGAAC |
| | | | | | | AAGTCAACGTCTACCCATACGA |
| | | | | | | TGTTCCAGATTACGCTCTCGAG |
| | | | | | | CACCACCACCACCACCACTGA |
| MG148 HA- | 60 | MG148-12- | nucleo- | artificial | | ATGGCGAACTGTAACGGTACGA |
| His tagged | | HA-His | tide | sequence | | TCATTAGCTATAATATGCATGG |
| genes | | | | | | GTTTAATCAAGGCAGTGAGTTA |
| | | | | | | TTGAAGTCTTACTGTGCGAATA |
| | | | | | | GTTCAGTGGATTTCCTGCTCAT |
| | | | | | | ACAGGAGCACTGGCTTTCTCCT |
| | | | | | | GACGCTTTGCATAAGATCGATG |
| | | | | | | ACATTGCTCAGGACTACTTTTG |
| | | | | | | TTTCTCTGTCTCGAGTATGACG |
| | | | | | | GCCGTTTTAGAGAGCGGACCTT |
| | | | | | | TGCGGGGCAGACCCTTCGGCGG |
| | | | | | | CCTGAGTATCCTTGTTAAGAAC |
| | | | | | | ATGCATCGGCAATTCTGCAGTG |
| | | | | | | TAGTTGCCCTGAACGAGAGATA |
| | | | | | | TATTGCTGTCCAGTACAATGAT |
| | | | | | | ATCTTAATAATTGACGTCTACT |
| | | | | | | TCCCGTGTGTATCAAGTCTCAA |
| | | | | | | CCATAAGGATGAGACAATAGAT |
| | | | | | | CTGTTAGTACAACTGGACACTT |
| | | | | | | TGGTTAATCAATCTGTGGTTAA |
| | | | | | | GGATATCATCATAGGCGGCGAC |
| | | | | | | TTCAACTGTAATCTGGAGATAG |
| | | | | | | AGAGTTGGTCTAGCAAGGTAAT |
| | | | | | | TTGTGACTTCATGGAAGATAAC |
| | | | | | | TCTCTTTCATCATGCAACAAGC |
| | | | | | | TGGCTGTGAATGTGATAGGTGC |
| | | | | | | TGAGTACACGTACAGTAACGAA |
| | | | | | | GTATTGGGTCATTATAGCTACC |
| | | | | | | TTGACTACTTTGTTGTCAGCAA |
| | | | | | | TTCACTTACGAGCTCCGTCATG |
| | | | | | | AGCCTTGAGATTTGTTCAGACG |
| | | | | | | ACTTCAACCTCTCCGACCACTC |
| | | | | | | ACCGGTATGCATCGAAGTGTGC |
| | | | | | | AATATAATCAGCGATTGTGAGA |
| | | | | | | CAACGGTCTTGTGCTCCAACAA |
| | | | | | | GGGCGGCAAGAGTACAAAGATT |
| | | | | | | CCAGACACTTGTTACCAGAACC |
| | | | | | | GTTGGGACCATGCTGACATTGT |
| | | | | | | GGCTTACTATGAACTGACCAGA |
| | | | | | | CTCGGTTTGCAACCGATATTAG |
| | | | | | | CTTACCTTAATGATTGTAGCAA |
| | | | | | | CAGTTTCCCACCTCAGAGTGCA |
| | | | | | | ATCGATTACAAGAGATACATGC |
| | | | | | | GTGCGTGCATAGACTTAGCTTA |
| | | | | | | TAATGACGCGGTGTCCGTTCTG |
| | | | | | | GTAGACGCTGCAAAGAACACAG |
| | | | | | | TTCCGCGCTTTAAGGCCAACGC |
| | | | | | | ATTAAAGCACTGGTGGGACCAG |
| | | | | | | GAGCTCAGTGAATTAAAAGAGA |
| | | | | | | AAGCCTTTGCGTCTCATAAACT |
| | | | | | | CTGGATTGAAGCGGGAAAGCCT |
| | | | | | | CGTAACGGATGTATATTTGACA |
| | | | | | | TCCGGAAGGCAGACAAGTACAA |
| | | | | | | ATACAAGTGTTTAATTAAACGT |
| | | | | | | AAGCAGCTTGAAGTTCGCGACA |
| | | | | | | GTATTACGAATGATCTCCATGA |
| | | | | | | TGCACTCTTACTGAAGGATGCT |
| | | | | | | GACCATTTCTGGAAAGTGTGGA |
| | | | | | | AGTCAAAGTTTCCCGCGAAGCG |
| | | | | | | GCCGAATAAGTACGCGCTGATT |
| | | | | | | GAGGGTAACAGCGATCCCAACA |
| | | | | | | TTATTAGTAACACGTTCTGCAA |
| | | | | | | CTATTTCAGTGAGATTTGTACG |
| | | | | | | GCGCACCCAAGTGTTAATGCCA |
| | | | | | | GTTCGAACAATATCTTCTTGAA |
| | | | | | | TAGATTCGACGGATACATAGGT |
| | | | | | | GACCTCAATCAATCAACCAAGA |
| | | | | | | ACATCATCAGCATCGATTTGAT |
| | | | | | | AGAGGACTTTATACTTTCGATG |
| | | | | | | AAGCGTGGAAAGGCCGCAGGCC |
| | | | | | | TGGATTCCCTTACAATAGAACA |
| | | | | | | CATCCAATTTGCTCACCCAGCA |
| | | | | | | ATAATCTGTATACTGAAGCTGC |
| | | | | | | TGTTTAACTACATGTTAGAGTT |
| | | | | | | CGGAATTGTGCCCGAGGGCTTC |
| | | | | | | CGGAACGGCCTTGTCATTCCCC |
| | | | | | | TTCCAAAAGAGGATTCCATCAA |
| | | | | | | GAAGAACGTGAAGCTGGAGAAC |
| | | | | | | TTCCGCTGTATTACAATTTCTC |
| | | | | | | CCGTTATCTCCAAGATCTTTGA |
| | | | | | | GCACTGTCTGATGCGCCTGTTC |
| | | | | | | GCAAAGTATCTGAACTCCGACG |
| | | | | | | ATGCGCAATTGGGTTTCAAGAA |
| | | | | | | GAAGTGCGGGTGCAGTCACGCG |
| | | | | | | ATATACTGCGTCAAGCAAGTGG |
| | | | | | | TCGATTACTATGTACGCGGTGG |
| | | | | | | AAGTACTGTGAATGTGTGTACA |
| | | | | | | CTTGACATTTCAAAGGCGTTCG |
| | | | | | | ACAAGGTTAACTTATTCGTCCT |
| | | | | | | GCTGTGCAAGCTCATGGATCGT |
| | | | | | | AATATTCCTAACTACGTCATAA |
| | | | | | | ATGTGCTTTACGATTGGTTCAG |
| | | | | | | CAACAATTATATCACCGTTAAA |
| | | | | | | TGGCTTAACATCCTTTCCAGTC |
| | | | | | | GATGTCCAGTAAATTCGGGAGT |
| | | | | | | TCGGCAGGGTGGAGTGCTGAGC |
| | | | | | | CCGGTCCTGTTCGCTATATACG |
| | | | | | | TAGATGACATTCTCGTAAAGCT |
| | | | | | | GCGTAAGAGTCGTCTGGGTTGT |
| | | | | | | ACAATCCAGGGCCTGTCTGTTA |
| | | | | | | ATGCTTACATGTATGCGGATGA |
| | | | | | | TCTGATTATCCTGAGTGGTTCA |
| | | | | | | GTTACTGATTTGCAAAAGCTTA |
| | | | | | | TTACGCTTTCAATTGAAGAGCT |
| | | | | | | GAAGTGTATTCACCTGTCTATA |
| | | | | | | AATCCTAAGAAGTGCTTTTGCA |
| | | | | | | TGCGAGTCGGTAAACGGTTCAA |
| | | | | | | GGTAAACTGTAACAATGTGGTG |
| | | | | | | GTCGACAATTATTCAATCCAAT |
| | | | | | | GGTCTTCTGAAATACGTTACCT |
| | | | | | | GGGAGTTTACTTAACGGCGGGC |
| | | | | | | CACGTTCTGAAGTTCAATTTAG |
| | | | | | | ATTATGGGAAGAAAAGTTCCAT |
| | | | | | | TGCAGCTTTAAATTCGATTATG |
| | | | | | | AGCAAAACGGGCAACAAGGCTA |
| | | | | | | TCGACATAGTTCTTAGTCTTAC |
| | | | | | | ACAATCATACTGTATCCCAATC |
| | | | | | | CTCTTGTACGCAGTCGAAAGCA |
| | | | | | | TGTGCCTGACCACAACAGAGCG |
| | | | | | | CCAGCGTTTAGGGTCTTCATTT |
| | | | | | | AATAAGCTGTATAACAAGTTAT |
| | | | | | | TCTCTACTTTCGATACCCAGAC |
| | | | | | | TATCGCCTACTGCCAATACTAC |
| | | | | | | ACTGGCTATCTTCCTCTTGACT |
| | | | | | | ACGTCATTGATCTGAGATGCTG |
| | | | | | | GAACTTCTTACAAAAGCTCTCT |
| | | | | | | ATATCTAATAATCTCGTACTGA |
| | | | | | | ACAAGCTCTTCCGTTTGAACGG |
| | | | | | | TAACAATACTATCGATTCGTTG |
| | | | | | | TGCTTAAAGTACAACTGCGAAT |
| | | | | | | TCAAGAACTATCCGTCTCTTAA |
| | | | | | | GGTGAGTATGTGGGAGAAGTTC |
| | | | | | | AAGTTCGTTTACTCCCTGTGCC |
| | | | | | | CATACCCATACGATGTTCCAGA |
| | | | | | | TTACGCTCTCGAGCACCACCAC |
| | | | | | | CACCACCACTGA |
| MG148 | 61 | MG148-12 | protein | unknown | uncultivated | MANCNGTIISYNMHGENQGSEL |
| reverse | | reverse | | | organism | LKSYCANSSVDELLIQEHWLSP |
| transcriptase | | transcriptase | | | | DALHKIDDIAQDYFCFSVSSMT |
| proteins | | | | | | AVLESGPLRGRPFGGLSILVKN |
| | | | | | | MHRQFCSVVALNERYIAVQYND |
| | | | | | | ILIIDVYFPCVSSLNHKDETID |
| | | | | | | LLVQLDTLVNQSVVKDIIIGGD |
| | | | | | | ENCNLEIESWSSKVICDFMEDN |
| | | | | | | SLSSCNKLAVNVIGAEYTYSNE |
| | | | | | | VLGHYSYLDYFVVSNSLTSSVM |
| | | | | | | SLEICSDDENLSDHSPVCIEVC |
| | | | | | | NIISDCETTVLCSNKGGKSTKI |
| | | | | | | PDTCYQNRWDHADIVAYYELTR |
| | | | | | | LGLQPILAYLNDCSNSFPPQSA |
| | | | | | | IDYKRYMRACIDLAYNDAVSVL |
| | | | | | | VDAAKNTVPREKANALKHWWDQ |
| | | | | | | ELSELKEKAFASHKLWIEAGKP |
| | | | | | | RNGCIFDIRKADKYKYKCLIKR |
| | | | | | | KQLEVRDSITNDLHDALLLKDA |
| | | | | | | DHFWKVWKSKFPAKRPNKYALI |
| | | | | | | EGNSDPNIISNTFCNYFSEICT |
| | | | | | | AHPSVNASSNNIFLNREDGYIG |
| | | | | | | DLNQSTKNIISIDLIEDFILSM |
| | | | | | | KRGKAAGLDSLTIEHIQFAHPA |
| | | | | | | IICILKLLFNYMLEFGIVPEGF |
| | | | | | | RNGLVIPLPKEDSIKKNVKLEN |
| | | | | | | FRCITISPVISKIFEHCLMRLF |
| | | | | | | AKYLNSDDAQLGFKKKCGCSHA |
| | | | | | | IYCVKQVVDYYVRGGSTVNVCT |
| | | | | | | LDISKAFDKVNLFVLLCKLMDR |
| | | | | | | NIPNYVINVLYDWESNNYITVK |
| | | | | | | WLNILSSRCPVNSGVRQGGVLS |
| | | | | | | PVLFAIYVDDILVKLRKSRLGC |
| | | | | | | TIQGLSVNAYMYADDLIILSGS |
| | | | | | | VTDLQKLITLSIEELKCIHLSI |
| | | | | | | NPKKCFCMRVGKRFKVNCNNVV |
| | | | | | | VDNYSIQWSSEIRYLGVYLTAG |
| | | | | | | HVLKFNLDYGKKSSIAALNSIM |
| | | | | | | SKTGNKAIDIVLSLTQSYCIPI |
| | | | | | | LLYAVESMCLTTTERQRLGSSF |
| | | | | | | NKLYNKLFSTEDTQTIAYCQYY |
| | | | | | | TGYLPLDYVIDLRCWNFLQKLS |
| | | | | | | ISNNLVLNKLERLNGNNTIDSL |
| | | | | | | CLKYNCEFKNYPSLKVSMWEKF |
| | | | | | | KFVYSLCP |
| MG148 | 33 | MG148-38 | protein | unknown | uncultivated | MRGFFQGLVVVNDLISGYQSPD |
| reverse | | reverse | | | organism | VILLQEHWLTPANMNLFDEKIT |
| transcriptase | | transcriptase | | | | THFVVGKSAMSDRVSAGPLVGR |
| proteins | | | | | | PYGGAAILIRNELRADTECVFC |
| | | | | | | SDRFAVVRICNLLIMSVYLPCA |
| | | | | | | ATAARMFIVEDLLQEIWSLRLK |
| | | | | | | YSECSVVIGGDFNADLNKQNDV |
| | | | | | | SNFINSFLTTCLLIRCDSKELS |
| | | | | | | RQQSTYVNESLGQSSTIDFFVC |
| | | | | | | DVVDDIIDYCVIDPDVNESDHL |
| | | | | | | PVAVRCKWSRTDNLKRSKSSNS |
| | | | | | | SKVKHLRWDHGDLLSYYSSTMS |
| | | | | | | RLYPLYEYLIRIEGWLVEPSNA |
| | | | | | | VQRETVIRLVDHVYDQLVVALR |
| | | | | | | ESANSYIPKHAKKFFKFWWNQE |
| | | | | | | LDALKHNAIASSSVWKNAGKPR |
| | | | | | | GGALYLQYKRDKLLYKKRLREE |
| | | | | | | QKAEAACYSNDLHDALLRKSGQ |
| | | | | | | DFWKCWNSKFERRSNKVVQVDG |
| | | | | | | ITDSAVIADKFAEYFESVCRPE |
| | | | | | | NADRNNLIKSKYNELRSTYTGT |
| | | | | | | PIVEEQWENVELLSKLVDSMSK |
| | | | | | | GKAAGLDELSSEHLKYSHPVVV |
| | | | | | | CILSKLENLFVYYSHIPASFGR |
| | | | | | | SFTVPIPKHDGRTHALHVDDER |
| | | | | | | GISISPVISKLFEMAILDRESV |
| | | | | | | FFSTSDHQFGFKKNLSCRHAIY |
| | | | | | | CVRSVIDNFVLHGSTVNVCALD |
| | | | | | | LSKAFDRMNHYALFIKLMERSF |
| | | | | | | PCELLAILETWFYISVSCVKWN |
| | | | | | | DNLSSFFVLTAGVRQGGVLSPY |
| | | | | | | LFAIFIDDLVYKVKSLNVGCYI |
| | | | | | | SLTCAAIFMYADDILLLSPTVD |
| | | | | | | GLQQLLHVCENELEQLDMKLNV |
| | | | | | | NKSVCIRVGPRENADCAELRSR |
| | | | | | | NGAVLKWTDSCRYLGVYFVRGR |
| | | | | | | TLKCSFSNAKSRFFRAFNAVEG |
| | | | | | | KVGRAASEETVIELIRAKCIPI |
| | | | | | | LLYATEACPFFSRDKQSLEFTV |
| | | | | | | TRLFMKIFQTGSPAVVRDCQRS |
| | | | | | | FNFLPIEMQVIIRTSRFLQAFV |
| | | | | | | ATQNSLCSLFQRSASCQLNDIF |
| | | | | | | SKYDRVQTASQLANRIHHTFAM |
| | | | | | | CDSV |
| MG148 | 34 | MG148-39 | protein | unknown | uncultivated | SYHYAIRAVRRNEQNIIRERVA |
| reverse | | reverse | | | organism | DALLRDPSRDFWTEVKKIRNSK |
| transcriptase | | transcriptase | | | | SGRAVIVDGCSDAPSISQLFAS |
| proteins | | | | | | KYRHLYTSVPYQHNDLQSIVSD |
| | | | | | | VESRISEDGDCFIGSQEVMAAL |
| | | | | | | SKLKLHKNDGDLGLASDYFINS |
| | | | | | | DPALSVHIALLFTGIVIHGFVP |
| | | | | | | SNLLSSTIVPIPKKSNVNATDS |
| | | | | | | DNYRGIALSSVLGKIFDNVILV |
| | | | | | | KYSDKLSTCNLQFGFKRNSSTH |
| | | | | | | MCTMVLKETISYYVNNNSSVFC |
| | | | | | | TFLDASKAFDRVHYCKLERLLL |
| | | | | | | GRGLPVCILRVLIQLYVGHSIR |
| | | | | | | VTWAGLVSSCFTALNGVKQGGV |
| | | | | | | LSPVLFCIYVDELLIRLAESGV |
| | | | | | | GCYIGFSFAGALAYADDIVLIA |
| | | | | | | PTPSAMRKLLAICDTYATEYNI |
| | | | | | | LENAQKSNFIAFVPSSRHSLHK |
| | | | | | | AMTNCVFRIGGVQIEHVESYTH |
| | | | | | | LGHIITSRLDDADDILHRRSSY |
| | | | | | | IGQVNNVVCYFDSLSWTVKLGL |
| | | | | | | HKSYCSSIFGCELWALDSVRDI |
| | | | | | | EKFCVAWRKGLRRVLSLPRAAH |
| | | | | | | SHLLPLLSNSLPVYDEICKRSA |
| | | | | | | KFIVSCQCSDNILVRAVVNYAI |
| | | | | | | AARSKSVLGRNVMLLCRREHLS |
| | | | | | | FDDFVSGRLLLSGDIFVSHYLN |
| | | | | | | SLSEAQLQSVCFALELLCLREQ |
| | | | | | | SFKLNNNMRLNADEISDYLAAV |
| | | | | | | LC |
| MG148 | 35 | MG148-40 | protein | unknown | uncultivated | MHGFTPDCMLLSTVVPIQKGKN |
| reverse | | reverse | | | organism | VNVTDSANYRGISLSSIFAKLF |
| transcriptase | | transcriptase | | | | DLLILQRYSDCLCLSDQQFGER |
| proteins | | | | | | AKRSTDMCSMVLKESISYYVNN |
| | | | | | | GSSVYCTFIDATKAFDRVEYCK |
| | | | | | | LFRQLLSRGLPPVIIRIMLNLY |
| | | | | | | VGHVTRVEWNGIRSRNFSVENG |
| | | | | | | VKQGGIVSPILFCIYLDGLLQS |
| | | | | | | LATSGVGCYIGTIFVGAMAYAD |
| | | | | | | DLVLLAPSANAMRLMLRNCDAF |
| | | | | | | ANEYNIRENANKSKCLFCSARR |
| | | | | | | HSRCNIGAQPVFYIGGNPIEIV |
| | | | | | | DHWPHLGHIISSHLDDEQDIIQ |
| | | | | | | RRNAMAGQINNVLNYFVGLDCF |
| | | | | | | VKQKLLTTYCYSLYGSVLWDLR |
| | | | | | | HLCIDSVCTTWRRGLRRVWGLP |
| | | | | | | HNTHSNLLPLLSCSLPVYDELC |
| | | | | | | KRFVLFAQKCLMSDSSLVSSVA |
| | | | | | | TYAFIYGRSDSVFGRNVSKCCL |
| | | | | | | RENVANDDELSLNKKEMENNYC |
| | | | | | | SNLEQDTMSTVNLLLELICVRD |
| | | | | | | GLFIMPMFADAAELIEIIRLLC |
| | | | | | | TV |
| MG148 | 36 | MG148-41 | protein | unknown | uncultivated | MTITNDAAQLVVISYNLHGLNQ |
| reverse | | reverse | | | organism | GLPGIREFMTELKPDVIMVQEH |
| transcriptase | | transcriptase | | | | WLTSDNLNKLSDISDEYFVIGS |
| proteins | | | | | | SAMDARVSAGPLFGRPFGGTAV |
| | | | | | | FIKNKYINVTVNLVTSERYVVI |
| | | | | | | QLCDWLLINVYLPCIGTSNRIL |
| | | | | | | LYSDTLCELQSIIRAHPECNCL |
| | | | | | | VGGDFNTDLNDTRSHTANTVVN |
| | | | | | | GFIAGCNLNLSDHLPIITVCVF |
| | | | | | | DSNCKLNNPKPTSTEDVTHERW |
| | | | | | | DHAPLQDYYEHTRLGLQPILAD |
| | | | | | | LDELIGNKLSYSNVDFLCQVDC |
| | | | | | | IYNRVVSVLQQCSHAYVPKHKK |
| | | | | | | NFYKFWWNQELDELKDKAIASC |
| | | | | | | KMWKDAGKPRHGAIHAKYRQDK |
| | | | | | | LLYKKRIRQERVQETSSFTNEL |
| | | | | | | HDALLNKSGRDFWKKWNSKFEN |
| | | | | | | KSNKILQVGGTSDTATILNNFA |
| | | | | | | KHFEQVCVPFNATRNEELKSRY |
| | | | | | | KEMRLNYSESSVINDTTVEDVQ |
| | | | | | | LVENLLTNMKNGKAGGLDELTS |
| | | | | | | EHLKFCHPVVVIILCKLENLEV |
| | | | | | | INGHIPDSFGVSYTVPIPKSDG |
| | | | | | | RSRSMTVDDERGISISPVISKL |
| | | | | | | FELCVLDRYSDYLQTSDHQFGE |
| | | | | | | KKQLGCRHAIFSVRSVIEQYIS |
| | | | | | | NGSTVNLCALDLSKAFDRMNQY |
| | | | | | | ALFIKLMERRFPVKILTILEQW |
| | | | | | | FSIAETCVRWGSEFSYFFSLLA |
| | | | | | | GVRQGGVLSPVLFAIFIDGIVN |
| | | | | | | RVNATNVGCYNSTVCVSIFLYA |
| | | | | | | DDILLLSPTVTGLQTLLTVCEN |
| | | | | | | ELCELDMRLNVNKSVCMRFGAR |
| | | | | | | FKSHCANLVSVQGGALQWASSC |
| | | | | | | RYLGVYFVSGRVFRCCFHSAKC |
| | | | | | | NFFRAFNSVYSKIGCCASEEVI |
| | | | | | | LSLLKSKCLP |
| MG148 | 37 | MG148-42 | protein | unknown | uncultivated | MRGFFQGLVVVNDLISGYQSPD |
| reverse | | reverse | | | organism | VILLQEHWLTPANMNLFDEKIT |
| transcriptase | | transcriptase | | | | THFVVGKSAMSDRVSAGPLVGR |
| proteins | | | | | | PYGGAAILIRNELRADTECVEC |
| | | | | | | SDRFAVVRICNLLIMSVYLPCA |
| | | | | | | GTADRMFIVEDLLQEIWSLRLK |
| | | | | | | YSECSVVIGGDFNADLNKQNDV |
| | | | | | | SNFINSFLTTCLLIRCDSKFLS |
| | | | | | | RQQSTYVNESLGQSSTIDFFVC |
| | | | | | | DVVDDIIDYCVIDPDVNFSDHL |
| | | | | | | PVAVRCKWSRTDNLKRSKSSNS |
| | | | | | | SKVKHLRWDHGDLLSYYSSTMS |
| | | | | | | RLYPLYEYLIKFEGELVEPTNA |
| | | | | | | VQRETVIRLVDHVYDQLVVALR |
| | | | | | | ESANSYIPKHAKKFFKFWWNQE |
| | | | | | | LDALKHNAIASSSVWKNAGKPR |
| | | | | | | GGALYLQYKRDKLLYKKRLREE |
| | | | | | | QKAEAACYSNDLHDALLRKSGQ |
| | | | | | | DFWKCWNSKFERRSNKVVQVDG |
| | | | | | | ITDSAVIADKFAEYFESVCRPF |
| | | | | | | NADRNNLIKSKYNELRSTYTGT |
| | | | | | | PIVEEQWENVELLSKLVDSMSK |
| | | | | | | GKAAGLDELSSEHLKYSHPVVV |
| | | | | | | CILSKLENLFVYYSHIPASFGR |
| | | | | | | SFTVPIPKHDGRTHALHVDDER |
| | | | | | | GISISPVISKLFEMAILDRESV |
| | | | | | | FFSTSDHQFGFKKNLSCRHAIY |
| | | | | | | CVRSVIDNFVLHGSTVNVCALD |
| | | | | | | LSKAFDRMNHYALFIKLMERSF |
| | | | | | | PCELLAILETWFYISVSCVKWN |
| | | | | | | DNLSSFFVLTAGVRQGGVLSPY |
| | | | | | | LFAIFIDDLVYKVKSLNVGCYI |
| | | | | | | SLTCAAIFMYADDILLLSPTVD |
| | | | | | | GLQQLLHVCENELEQLDMKLNV |
| | | | | | | NKSVCIRVGPRENADCAELRSR |
| | | | | | | NGAVLKWTDSCRYLGVYFVRGR |
| | | | | | | TLKCSFSNAKSRFFRAFNAVEG |
| | | | | | | KVGRAASEETVIELIRAKCIPI |
| | | | | | | LLYATEACPFFSRDKQSLEFTV |
| | | | | | | TRLFMKIFQTGSPAVVRDCQRS |
| | | | | | | FNFLPIEMQVIIRTSRFLQAFV |
| | | | | | | ATQNSLCSLFQRSASCQLNDIF |
| | | | | | | SKYDRVQTASQLANRIHHTFAM |
| | | | | | | CDSV |
| MG148 | 38 | MG148-43 | protein | unknown | uncultivated | MESAQIDVLLLQEHWLSDAQLN |
| reverse | | reverse | | | organism | ALNNVGANYLNFGVSGFDTSAV |
| transcriptase | | transcriptase | | | | LGGRPYGGCAVLWRSDLLLQVQ |
| proteins | | | | | | PLVVSSRRLSAVIFSTDNWSLI |
| | | | | | | LINVYMPYEGDEIKTDEFIDLL |
| | | | | | | SIIEDLVLSNSASHVIVGGDEN |
| | | | | | | VEFNRNRMHTALLNSFCDNTGL |
| | | | | | | SPVIQHSSCNIDYTYNENMSRF |
| | | | | | | NILDHFLLSGTLFDVCVTSAYV |
| | | | | | | VHDIDNTSDHDPIILRLSLDIK |
| | | | | | | YVSVCNRTSSSRVSWVKASDRD |
| | | | | | | IRNYQYNLASNLQHVTIPSVAL |
| | | | | | | LCKDVNCSNFAHRCQLSRYLTD |
| | | | | | | ISDACLAAGEASIPHTCSRHSG |
| | | | | | | KRIPGWSEKVEPLRQRSLFWHS |
| | | | | | | MWVECGRPRSGVVADCMRRARA |
| | | | | | | SYHYAIRAVRRNEQNIIRERVA |
| | | | | | | DALLRDPSRDFWTEVKKIRNSK |
| | | | | | | SGRAVIVDGCSDAPSISQLFAS |
| | | | | | | KYRHLYTSVPYQHNDLQSIVSD |
| | | | | | | VESRISEDGDCFIGSQEVMAAL |
| | | | | | | SKLKLHKNDGDLGLASDYFINS |
| | | | | | | DPALSVHIALLFTGIVIHGFVP |
| | | | | | | SNLLSSTIVPIPKKSNVNATDS |
| | | | | | | DNYRGIALSSVLGKIFDNVILV |
| | | | | | | KYSDKLSTCNLQFGFKRNSSTH |
| | | | | | | MCTMVLKETISYYVNNNSSVFC |
| | | | | | | TFLDASKAFDRVHYCKLERLLL |
| | | | | | | GRGLPVCILRVLIQLYVGHSIR |
| | | | | | | VTWAGLVSSCFTALNGVKQGGV |
| | | | | | | LSPVLFCIYVDELLIRLAESGV |
| | | | | | | GCYIGFSFAGALAYADDIVLIA |
| | | | | | | PTPSAMRKLLAICDTYATEYNI |
| | | | | | | LFNAQKSNFIAFVPSSRHELHK |
| | | | | | | AMTNCVFRIGGVQIEHVESYTH |
| | | | | | | LGHIITSRLDDADDILHRRSSY |
| | | | | | | IGQVNNVVCYFDSLS |
| MG148 | 39 | MG148-44 | protein | unknown | uncultivated | GSARCPSIGQPTSRVEPWRLTR |
| reverse | | reverse | | | organism | EGNPTASNVVDGCTTPDSISQL |
| transcriptase | | transcriptase | | | | FAAKYKELYTSVPYSADDMSSL |
| proteins | | | | | | NSDINALMSSGFCAGCFVNANE |
| | | | | | | VFAAVKELKLHKNDGNTGLTSD |
| | | | | | | HVKCALPDLSVHIALLLSGVLS |
| | | | | | | HGSIPKDLLLSTVIPIPKNKNG |
| | | | | | | NLADSSNYRGIALSSVECKIED |
| | | | | | | KILLARYNDKFVTSELQFGFKA |
| | | | | | | GRSTHMCSMLLKESMLYYKNNN |
| | | | | | | SLVYCAFLDATKAFDRVNYCKL |
| | | | | | | FRLLIKRGLPPPILRALLNFYV |
| | | | | | | GHTVRVSWFGSVSSYFLALNGM |
| | | | | | | KQGGVISPLLFCVYIDDLLCAL |
| | | | | | | EKSGVGCYIGLHETGALAYADD |
| | | | | | | IVLLAPSPTALRRLLSICDVYA |
| | | | | | | AEYCI |
| MG148 | 40 | MG148-45 | protein | unknown | uncultivated | MGFQAQTAGGMNNTAIMTSDQN |
| reverse | | reverse | | | organism | CTSLSALTFNMHGFNQGSVLLK |
| transcriptase | | transcriptase | | | | EVCEKQIYDLIFIQEHWLYPSN |
| proteins | | | | | | ISKLLEISPHYIGHGISAMESA |
| | | | | | | VDAGFIKGRPYGGCAILVNNKY |
| | | | | | | KNMHADVCTFERVVAILLGDVL |
| | | | | | | LINVYCPCENGSTEGFDALNEI |
| | | | | | | LANIANILDTYKAQYVVIGGDF |
| | | | | | | NTDLSKDTTHSVLIKEFLDEYG |
| | | | | | | IKYCKSEMFGSVNIQFERTFAC |
| | | | | | | ETTGAGSCIDFFCVSDNICNNV |
| | | | | | | MRYKVLDMYSNHSDHRPILCQF |
| | | | | | | CVPVQSSLHQYVSSGCVNEVNE |
| | | | | | | RANANVHASKQSAESEQALRWD |
| | | | | | | LGNVQLYNDLTYQYLYPIYDRL |
| | | | | | | MNYDNMHSVNETNINEGKEAAC |
| | | | | | | VFIEQSYNDLTNALHHCAANSI |
| | | | | | | PRVQRNAIKHWWNSELTELKKC |
| | | | | | | SITSHNNWLESGKPRSGAVEAL |
| | | | | | | KQQDKLRYKQAIKKVKNEADAA |
| | | | | | | ISDELHAKLCAKKTTSFWKTWK |
| | | | | | | NKVSNKAKKQISVEGCNTDEMA |
| | | | | | | VCSLAEHERKATVPNCEIENNE |
| | | | | | | KKTEFVHKLSEYSVNSSPTAVT |
| | | | | | | AEIVYISLSKVHKGKAPGIDNI |
| | | | | | | TAEHIGNSHVVIYSLLAKLENM |
| | | | | | | MLEYGHVPVSFTNGIIVPIPKD |
| | | | | | | ENNKNKYPVDNERGITLSPVLS |
| | | | | | | KIFEHCLLNTFGDYLLTCDNQF |
| | | | | | | GFKTKVGCSHAVYAVRKITDYE |
| | | | | | | VSGESTVNLCFLDISKGFDKVN |
| | | | | | | KYELLLKLMNRRIPVCFIELLN |
| | | | | | | YWLSMSISTVKWNGFFSQPYSL |
| | | | | | | EAGVRQGGVLSPLLFAVYVNDM |
| | | | | | | LCKLRKMGCSYKWLHFGAIMYA |
| | | | | | | DDLVLLAPSVYELQCMIRVCQE |
| | | | | | | QLKLLDLRLNFKKSKALRIGKR |
| | | | | | | YKHKAAQLQLNGEKIPWGTEAK |
| | | | | | | YLGMVIQSAARFKCSFDQAKVK |
| | | | | | | FYRGANAILSKLSNKSNVTLTL |
| | | | | | | YLVSTIALPTLTYGIECLSLNK |
| | | | | | | TEIKSLDYPWYKCFCRVFQTED |
| | | | | | | AGIIQLCQAILGYKSIKELYEN |
| | | | | | | RCQMFLEKIKIYGNEMLREASE |
| | | | | | | L |
| MG148 | 41 | MG148-46 | protein | unknown | uncultivated | MNSYSSTTLSAAESSCTTLSIT |
| reverse | | reverse | | | organism | TFNMHGFNQGSTLLIDVCYKET |
| transcriptase | | transcriptase | | | | YDVIFIQEHWLYPHNIRKLLDI |
| proteins | | | | | | SSKYTGYGISAMQSVVDSGEVK |
| | | | | | | GRPFGGCAILIHNKYINLHVDC |
| | | | | | | SVFERVVAVNVGDFLLTNVYFP |
| | | | | | | CENGSAEGEDTLNEILANVSNI |
| | | | | | | LDTYKAPYAIIGGDENTNLTKR |
| | | | | | | SIHANTVNDELNAHNMSYCKAK |
| | | | | | | VFGSVEISFEDTFACDKTGASS |
| | | | | | | CIDFVCVSNCLMHNVSKYEVLD |
| | | | | | | VYNNHSDHKPLFCQLCVPVSST |
| | | | | | | LHQFVASGCRKLMNGNAGDSKC |
| | | | | | | SNDSNCALRWDLGNVQLYTTLT |
| | | | | | | SERLYPIYEKLLNYRYVVDEHN |
| | | | | | | PHESKQLASQRIDKLYNELILA |
| | | | | | | LHECAATCIPQVKRNAIKHWWN |
| | | | | | | SELNELKTCSIKSHKSWLEAHK |
| | | | | | | PKSGALYLAKQQAKLRYKQAIK |
| | | | | | | RTRLAADASISDALHVNLTTSS |
| | | | | | | TEKFWKTWKTKVCKKNKQNMLL |
| | | | | | | EGCSTDDIAVCKLADFFSKTTA |
| | | | | | | PNSDEYNVKKKSEFLEQITDYS |
| | | | | | | GQTNYDTINAELVYNSMSKLKK |
| | | | | | | GKSPGIDNITVEHICNSHVVIF |
| | | | | | | ALLAKLENLMLEVRYVPAIFGN |
| | | | | | | GIIVPIPKEDTGKKVHPVDNER |
| | | | | | | GITLSPVLSKLFEHCLKCIFAE |
| | | | | | | FMSTCDNQFAFKNNMGCSHAIY |
| | | | | | | SVRKVTEYFVMGDSTVNLCELD |
| | | | | | | ISKGFDKVNKYELLLKLIKRNV |
| | | | | | | PVCFIELMHHWFDISKSTVKWN |
| | | | | | | GHFSQSYELKAGVRQGGVLSPL |
| | | | | | | LFAVYVDDMLRKISKMGCTYNW |
| | | | | | | LHLGAIMYADDLVLIASSVYEL |
| | | | | | | QCMLRVCQAELNLIDLKLNVKK |
| | | | | | | SKAIRIGKRSKLKAASLKLSEG |
| | | | | | | HIAWSSEAKYLGIVVQSAARFK |
| | | | | | | CNFDHVKIKFYRGANAILSKLS |
| | | | | | | SKANVTVTLHLISSIALPSLTY |
| | | | | | | GLECLALNKTELKSLHYPWHKC |
| | | | | | | LCRIFQTFDEQIINLCQAILGY |
| | | | | | | KSVKDLYEIRACMFLSRVKKCD |
| | | | | | | NELLRALAA |
| MG153 | 42 | MG153-22 | protein | unknown | uncultivated | MRQKSEQLELALDNRGEAPTSR |
| reverse | | reverse | | | organism | RSGEAPTTAHEAERSGGGHRLM |
| transcriptase | | transcriptase | | | | EQVVARANALAALRRVKQNRGS |
| proteins | | | | | | PGVDGMTVGELPQYLAKHWETV |
| | | | | | | REQLLAGSYQPEPVRRQAIPKP |
| | | | | | | GGGTRVLGIPTVLDRFIQQCLL |
| | | | | | | QVLQPRFDPSESDHSYGFRPGR |
| | | | | | | NAHDAVCAAQQYIQAGRRWVAD |
| | | | | | | LDLEKFFDRVNHDVLMERLARR |
| | | | | | | IADRRVLRLIRRYLVAGILHGG |
| | | | | | | VVVERREGTPQGGPLSPLLASV |
| | | | | | | LLDEVDRELERRGLAFARYADD |
| | | | | | | LNVYCGSRRAAHDAMATLKRLE |
| | | | | | | AALRLKVNESKSTVARVWERKE |
| | | | | | | LGYSFWVAPGRVIRPRIAPAAL |
| | | | | | | AVMKERVRRITRRTGGRSLEAV |
| | | | | | | AQELREYLTGWKAYERLAGKPR |
| | | | | | | VFRDLDEWTRHRLRAVQLKQWK |
| | | | | | | RGRTVCRELLARGVPEREARAA |
| | | | | | | AAHARRWWAMAAHSALQTALPN |
| | | | | | | SHFDQLGVPRLAGR |
| MG153 | 43 | MG153-23 | protein | unknown | uncultivated | MRADEAEAHASAASTGKGGRNL |
| reverse | | reverse | | | organism | PGTAAGAEVRAAAGGRTKPEAL |
| transcriptase | | transcriptase | | | | RLMEAAVERSNMLGAYERVVKN |
| proteins | | | | | | QGAPGVDGLTVTEFKPWLQAHW |
| | | | | | | PKIRQVLLAGEYMPAAVRKVDI |
| | | | | | | PKPQGGVRTLGIPTVLDRLIQQ |
| | | | | | | ALHQVLQPLFEPEFSESSYGER |
| | | | | | | PGRNAHQAVEAARSYVAEGKRW |
| | | | | | | VVDLEKFFDRVNHDVLMARVAR |
| | | | | | | KVKDERVLKLIRRYLEAGLMEG |
| | | | | | | GMTSARTEGTPQGGPLSPLLSN |
| | | | | | | TLLTDLDRELEKRGHRFCRYAD |
| | | | | | | DCNVYVGSRRSGQRVMAKITAF |
| | | | | | | LEQRLKLQVNADKSAVARPWQR |
| | | | | | | KFLGYSVTWHRNPKLKIAPSSR |
| | | | | | | QRLAEKIRQTLRGARGQSLRQV |
| | | | | | | IAQLNPILRGWVAYFRLTEEKG |
| | | | | | | VLEELDGWVRRKLRALLWRHWK |
| | | | | | | RGYPRAQNLMRAGLRPERAWQS |
| | | | | | | ATNGHGPWWNGGSSHMNAACPK |
| | | | | | | SWFDHMQLVSLLATQRRESLVS |
| MG153 | 44 | MG153-24 | protein | unknown | uncultivated | MKGGKQKISQDTCLQESRAEPE |
| reverse | | reverse | | | organism | GYAGGQTFIWMTENNLTNANKP |
| transcriptase | | transcriptase | | | | EYGLLEQILSPTNLNRAYKGVR |
| proteins | | | | | | SNRGSGGIDKMEVESLKDYLVD |
| | | | | | | NKETLIQSILDGKYRPNPVRRV |
| | | | | | | EISKEKGTRKLGIPTVVDRVIQ |
| | | | | | | QAIAQVLSPTYERQFSENSYGE |
| | | | | | | RPGRNAHQALNRCRDYITDGYI |
| | | | | | | YAVDMDLEKFFDTVNQSKLIEV |
| | | | | | | LSRTVKDGRVVSLIHKYLDAGV |
| | | | | | | VIRNKFEETEMGVPQGSPLSPV |
| | | | | | | LSNIMLNELDKELEKRGHPFVR |
| | | | | | | YADDLIIFCKSKRSADRTLANT |
| | | | | | | VPYIENKLFLKVNREKTTTAYV |
| | | | | | | SGIKFLGYSFYVYKGEGRLRVH |
| | | | | | | PKSIAKMKERIRKLTSRSNGWG |
| | | | | | | YARRKEALRQYITGWVNYFKLA |
| | | | | | | DMKKLLVSVDEWYRRRLRLVIW |
| | | | | | | KQWKRVRTRGRYLMKLGIVKHQ |
| | | | | | | AWEFANTRKGYWHTAKSPILNR |
| | | | | | | SVTSNRLRQAGYVFFVDYYRVV |
| | | | | | | NGIN |
| MG160 | 45 | MG160-7 | protein | unknown | uncultivated | MHNFKSRFELSKGKWVYIQIED |
| reverse | | reverse | | | organism | LANHAKDHIRQIRDLWTPPEYF |
| transcriptase | | transcriptase | | | | FHLQKGGHIAALRLHTPNEWYG |
| proteins | | | | | | KVDLSKFENNITRHRVTRSLKR |
| | | | | | | IGYSFRDAEEFAVASTVLVNHA |
| | | | | | | TRRYVLPYGFVQSPLLASISLD |
| | | | | | | KSDLGNCLRRLHEETVSVSVYV |
| | | | | | | DDIVVSADSERDVAEALSNIYL |
| | | | | | | AAINSRFPINEEKSRGPSSTLN |
| | | | | | | AFNIELDMHELEITAKRYEKMC |
| | | | | | | GEVLINGTGQVSDAILNYVQTV |
| | | | | | | NPAQAEQMLRDFPGVFGTLSSS |
| | | | | | | ASRYDAS |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-91. (canceled)

92. An engineered retrotransposase system comprising:
(a) a double-stranded nucleic acid comprising a cargo nucleotide sequence, wherein said cargo nucleotide sequence is configured to interact with a heterologous retrotransposase; and
(b) said heterologous retrotransposase, wherein:
(i) said heterologous retrotransposase is configured to transpose said cargo nucleotide sequence to a target nucleic acid locus; and
(ii) said heterologous retrotransposase comprises a sequence having at least 80% sequence identity to any one of SEQ ID NOs: 1-16, 32-41, 61, or 42-45.

93. The engineered retrotransposase system of claim 92, wherein said heterologous retrotransposase comprises a sequence having at least 80% sequence identity to any one of SEQ ID NOs: 1-16.

94. The engineered retrotransposase system of claim 92, wherein said heterologous retrotransposase comprises a sequence having at least 80% sequence identity to any one of SEQ ID NOs: 32-41 or 61.

95. The engineered retrotransposase system of claim 92, wherein said heterologous retrotransposase comprises a sequence having at least 80% sequence identity to any one of SEQ ID NOs: 42-44.

96. The engineered retrotransposase system of claim 92, wherein said heterologous retrotransposase comprises a sequence having at least 80% sequence identity to SEQ ID NO: 45.

97. The engineered retrotransposase system of claim 92, wherein said cargo nucleotide sequence is flanked by a 3′ untranslated region (UTR) and a 5′ untranslated region (UTR).

98. The engineered retrotransposase system of claim 92, wherein said engineered retrotransposase system is comprised in a cell, wherein said cell further comprises a deoxyribonucleic acid polynucleotide encoding said heterologous retrotransposase.

99. The engineered retrotransposase system of claim 92, wherein said heterologous retrotransposase comprises a nuclear localization sequence (NLS).

100. The engineered retrotransposase system of claim 99, wherein said NLS comprises a sequence having at least 80% sequence identity to a sequence of any one of SEQ ID NOs: 17-32.

101. The engineered retrotransposase system of claim 98, wherein said cell is a eukaryotic cell.

102. A method for synthesizing complementary deoxyribonucleic acid (cDNA), the method comprising:
(a) providing a ribonucleic acid (RNA) template in a cell, wherein said RNA template comprises a cargo nucleotide sequence and said cell comprises a nucleic acid primer to initiate cDNA synthesis from said RNA template; and
(b) synthesizing cDNA initiated by said nucleic acid primer using said RNA template and a heterologous retrotransposase, wherein said heterologous retrotransposase comprises a sequence having at least 80% sequence identity to any one of SEQ ID NOs: 1-16, 32-41, 61, or 42-45.

103. The method of claim 102, wherein said heterologous retrotransposase comprises a sequence having at least 80% sequence identity to any one of SEQ ID NOs: 1-16.

104. The method of claim 102, wherein said heterologous retrotransposase comprises a sequence having at least 80% sequence identity to any one of SEQ ID NOs: 32-41 or 61.

105. The method of claim 102, wherein said heterologous retrotransposase comprises a sequence having at least 80% sequence identity to any one of SEQ ID NOs: 42-44.

106. The method of claim 102, wherein said heterologous retrotransposase comprises a sequence having at least 80% sequence identity to SEQ ID NO: 45.

107. The method of claim 102, wherein said cargo nucleotide sequence is flanked by a 3′ untranslated region (UTR) and a 5′ untranslated region (UTR).

108. The method of claim 102, wherein said RNA template and said heterologous retrotransposase are encoded by a deoxyribonucleic acid polynucleotide in said cell.

109. The method of claim 102, wherein said heterologous retrotransposase comprises a nuclear localization sequence (NLS).

110. The method of claim 109, wherein said NLS comprises a sequence having at least 80% sequence identity to a sequence of any one of SEQ ID NOs: 17-32.

111. The method of claim 102, wherein said cell is a eukaryotic cell.
